(19) United States
(12) Patent Application Publication
(10) Pub. No.: US 2025/0090585 A1
(43) Pub. Date: Mar. 20, 2025

(54) MODIFIED REGULATORY T CELLS AND METHODS OF USING THE SAME

(71) Applicant: Beam Therapeutics Inc., Cambridge, MA (US)

(72) Inventors: Angelica Messana, Cambridge, MA (US); Colby Maldini, Cambridge, MA (US); Jason Michael Gehrke, Cambridge, MA (US)

(73) Assignee: Beam Therapeutics Inc., Cambridge, MA (US)

(21) Appl. No.: 18/965,215

(22) Filed: Dec. 2, 2024

Related U.S. Application Data
(63) Continuation of application No. PCT/US2023/067780, filed on Jun. 1, 2023.
(60) Provisional application No. 63/348,753, filed on Jun. 3, 2022.

(51) Int. Cl. A61K 35/17; A61K 39/00; C12N 5/0783; C12N 9/22; C12N 9/78; C12N 15/11

(52) U.S. Cl. CPC A61K 35/17; A61K 40/11; A61K 40/31; A61K 40/4203; C12N 5/0637; C12N 9/22; C12N 9/78; C12N 15/111; C07K 2319/09; C12N 2310/20; C12N 2510/00; C12Y 305/04004; C12Y 305/04005

(57) ABSTRACT

The disclosure features modified regulatory T (T_{REG}) cells having increased lineage stability, decreased activation of T cells (e.g., conventional T (T_{CONV}) cells), and/or increased resistance to immune rejection, and methods of producing and using such cells, for example, in the treatment of graft versus host disease (GVHD).

### CROSS REFERENCE TO RELATED APPLICATIONS

This application is a continuation under 35 U.S.C. § 111(a) of PCT International Patent Application No. PCT/US2023/067780, filed Jun. 1, 2023, designating the United States and published in English, which claims priority to and the benefit of U.S. Provisional Application No. 63/348,753 filed Jun. 3, 2022, the entire contents of each of which are incorporated by reference herein.

### SEQUENCE LISTING

This application contains a Sequence Listing which has been submitted electronically in XML format and is hereby incorporated by reference in its entirety. The Sequence Listing XML file, created on May 9, 2023, is named 180802-045902_PCT_SL.xml and is 3,769,384 bytes in size.

### BACKGROUND

There is a growing clinical need for an efficacious, suppressive cellular therapy for autoimmune and alloimmune diseases. For example, chronic graft versus host disease (cGVHD) occurs in 40-50% of all allo hematopoietic stem cell transplant (alloHSCT) recipients. However, current globally immunosuppressive regimens are often associated with undesired off-target toxicities and can be antithetical to immune tolerance; calcineurin inhibitors being key examples of this paradox. In contrast, suppressive cell-based therapies, including regulatory T (T_{REG}) cells, promise fewer off-target effects and induce immune tolerance in animal models. However, strategies remain unavailable for reliably sustaining transplanted T_{REG} cells in a subject and autologous cell therapies in general have numerous disadvantages associated with having to usually obtain the starting material from the patient to be treated, including long manufacturing times and the requirement that the patient cells are suitable despite previous therapies or disease state. Allogeneic cell therapies can overcome some challenges associated with autologous cell therapies, but challenges remain, including the limited persistence of allogeneic cell in the patient.

Therefore, there is a need for improved T_{REG} cells for treatment of autoimmune and alloimmune diseases.

### SUMMARY

As described below, the present disclosure features modified regulatory T (T_{REG}) cells having increased lineage stability, decreased activation of T cells (e.g., conventional T (T_{CONV}) cells), and/or increased resistance to immune rejection, and methods of producing and using such cells, for example, in the treatment of graft versus host disease (GVHD). The embodiments disclosed herein can provide superior methods of creating immune tolerance (e.g., to treat GVHD) without the toxicity and global immunosuppression associated with current regimens. Further, the embodiments provided herein can provide allogeneic cell therapies with lower cost and faster implantation than autologous cell therapies.

In one aspect, the disclosure features a method for producing a functionally enhanced and/or lineage stabilized regulatory T (T_{REG}) cell. The method involves contacting a T_{REG} cell with a base editor, or one or more polynucleotides encoding the base editor, where the base editor contains a polynucleotide programmable DNA binding polypeptide (napDNAbp), and a deaminase, and one or more guide RNAs (gRNAs), or one or more polynucleotides encoding the gRNAs, where the one or more gRNAstarget the base editor to effect an alteration in a nucleic acid molecule, thereby producing the functionally enhanced and/or lineage stabilized T_{REG} cell. The nucleic acid molecule encodes a polypeptide and/or contains a regulatory element associated with expression thereof. The polypeptide is selected from one or more of bone morphogenetic protein/retinoic acid-inducible neural-specific protein 1 (BRINP1), C terminus of HSC70-interacting protein (CHIP), Cluster of Differentiation 70 (CD70), c-JUN kinase 1 (JNK1), protein kinase C theta (PRKCQ), ring finger protein 20 (RNF20), and sirtuin 1 (SIRT1).

In another aspect, the disclosure features a method for producing a functionally enhanced and/or lineage stabilized regulatory T (T_{REG}) cell. The method involves contacting a T_{REG} cell with a base editor, or one or more polynucleotides encoding the base editor, where the base editor contains a polynucleotide programmable DNA binding polypeptide (napDNAbp), and a deaminase, and two or more guide RNAs (gRNAs), or one or more polynucleotides encoding the gRNAs. Each gRNA targets the base editor to effect an alteration in a nucleic acid molecule. Each nucleic acid molecule encodes a polypeptide and/or contains a regulatory element associated with expression of the polypeptide. A first polypeptide is selected from one or more of bone morphogenetic protein/retinoic acid-inducible neural-specific protein 1 (BRINP1), C terminus of HSC70-interacting protein (CHIP), c-JUN kinase 1 (JNK1), protein kinase C theta (PRKCQ), ring finger protein 20 (RNF20), and sirtuin 1 (SIRT1). A second polypeptide is selected from one or more of Cluster of Differentiation 58 (CD58), Cluster of Differentiation 70 (CD70), and programmed cell death 1 (PD-1).

In another aspect, the disclosure features a method for producing a functionally enhanced and/or lineage stabilized regulatory T (T_{REG}) cell. The method involves contacting a T_{REG} cell with a polynucleotide programmable DNA binding polypeptide (napDNAbp), or one or more polynucleotides encoding the napDNAbp, and one or more guide RNAs (gRNAs), or one or more polynucleotides encoding the gRNAs, that target the napDNAbp to cleave a target nucleic acid molecule and effect an alteration in the target nucleic acid molecule, thereby producing the functionally enhanced and/or lineage stabilized T_{REG} cell. The target nucleic acid molecule encodes a polypeptide and/or contains a regulatory element associated with expression thereof. The polypeptide is selected from one or more of bone morphogenetic protein/retinoic acid-inducible neural-specific protein 1 (BRINP1), C terminus of HSC70-interacting protein (CHIP), Cluster of Differentiation 70 (CD70), c-JUN kinase 1 (JNK1), protein kinase C theta (PRKCQ), ring finger protein 20 (RNF20), and sirtuin 1 (SIRT1)

In another aspect, the disclosure features a functionally enhanced and/or lineage stabilized regulatory T (T_{REG}) cell produced according to the method of any of the above aspects, or embodiments thereof.

In another aspect, the disclosure features a functionally enhanced and/or lineage stabilized regulatory T (T_{REG}) cell containing a nucleobase alteration that reduces or eliminates expression of a polypeptide selected from one or more of bone morphogenetic protein/retinoic acid-inducible neural-specific protein 1 (BRINP1), C terminus of HSC70-interacting protein (CHIP), Cluster of Differentiation 70 (CD70), c-JUN kinase 1 (JNK1), protein kinase C theta (PRKCQ), ring finger protein 20 (RNF20), and sirtuin 1 (SIRT1).

In another aspect, the disclosure features a functionally enhanced and/or lineage stabilized regulatory T (T_{REG}) cell containing a nucleobase alteration that reduces or eliminates expression of two or more polypeptides. The first polypeptide is selected from one or more of bone morphogenetic protein/retinoic acid-inducible neural-specific protein 1 (BRINP1), C terminus of HSC70-interacting protein (CHIP), c-JUN kinase 1 (JNK1), protein kinase C theta (PRKCQ), ring finger protein 20 (RNF20), and sirtuin 1 (SIRT1). The second polypeptide is selected from one or more of Cluster of Differentiation 58 (CD58), Cluster of Differentiation 70 (CD70), and programmed cell death 1 (PD-1).

In another aspect, the disclosure features a pharmaceutical composition containing a functionally enhanced and/or lineage stabilized regulatory T (T_{REG}) cell. The T_{REG} cell contains a nucleobase alteration that reduces or eliminates expression of a polypeptide selected from one or more of bone morphogenetic protein/retinoic acid-inducible neural-specific protein 1 (BRINP1), C terminus of HSC70-interacting protein (CHIP), Cluster of Differentiation 70 (CD70), c-JUN kinase 1 (JNK1), protein kinase C theta (PRKCQ), ring finger protein 20 (RNF20), and sirtuin 1 (SIRT1).

In another aspect, the disclosure features a pharmaceutical composition containing a functionally enhanced and/or lineage stabilized regulatory T (T_{REG}) cell. The T_{REG} cell contains a nucleobase alteration that reduces or eliminates expression two or more polypeptides. The first polypeptide is selected from one or more of bone morphogenetic protein/retinoic acid-inducible neural-specific protein 1 (BRINP1), C terminus of HSC70-interacting protein (CHIP), c-JUN kinase 1 (JNK1), protein kinase C theta (PRKCQ), ring finger protein 20 (RNF20), and sirtuin 1 (SIRT1). The second polypeptide is selected from one or more of Cluster of Differentiation 58 (CD58), Cluster of Differentiation 70 (CD70), and programmed cell death 1 (PD-1).

In another aspect, the disclosure features a pharmaceutical composition containing a guide RNA (gRNA) and a polynucleotide encoding a base editor containing a polynucleotide programmable DNA binding polypeptide (napDNAbp) domain and a deaminase domain. The gRNA contains a nucleic acid sequence that is complementary to a polynucleotide. The polynucleotide encodes a polypeptide and/or contains a regulatory element associated with expression of the polypeptide. The polypeptide is selected from one or more of bone morphogenetic protein/retinoic acid-inducible neural-specific protein 1 (BRINP1), C terminus of HSC70-interacting protein (CHIP), Cluster of Differentiation 70, c-JUN kinase 1 (JNK1), protein kinase C theta (PRKCQ), ring finger protein 20 (RNF20), and sirtuin 1 (SIRT1).

In another aspect, the disclosure features a pharmaceutical composition containing two or more guide RNAs (gRNA) and a polynucleotide encoding a base editor containing a polynucleotide programmable DNA binding polypeptide (napDNAbp) domain and a deaminase domain. where each gRNA contains a nucleic acid sequence that is complementary to a nucleic acid molecule. Each nucleic acid molecule encodes a polypeptide and/or contains a regulatory element associated with expression of the polypeptide. The first polypeptide is selected from one or more of bone morphogenetic protein/retinoic acid-inducible neural-specific protein 1 (BRINP1), C terminus of HSC70-interacting protein (CHIP), c-JUN kinase 1 (JNK1), protein kinase C theta (PRKCQ), ring finger protein 20 (RNF20), and sirtuin 1 (SIRT1), and where the second polypeptide is selected from one or more of Cluster of Differentiation 58 (CD58), Cluster of Differentiation 70 (CD70), and programmed cell death 1 (PD-1).

In another aspect, the disclosure features a kit containing an the functionally enhanced and/or stabilized T_{REG} cell of any aspect provided herein, or embodiments thereof.

In another aspect, the disclosure features a method of treating an autoimmune or alloimmune disease in a subject, the method involving administering to the subject an effective amount of a functionally enhanced and/or stabilized T_{REG} cell of any aspect provided herein, or embodiments thereof.

In another aspect, the disclosure features a guide RNA (gRNA), or a polynucleotide encoding the guide RNA, where the guide RNA contains a a nucleotide sequence with at least 70% sequence identity to a sequence selected from one or more of those listed in Tables 1A-1C or 2A-2C, or truncations thereof. The gRNA targets a base editor containing a nucleic acid programmable DNA binding protein (napDNAbp) to effect an alteration in a nucleic acid molecule encoding a polypeptide and/or containing a regulatory element associated with expression thereof, thereby producing a functionally enhanced and/or lineage stabilized T_{REG} cell. The polypeptide is selected from one or more of bone morphogenetic protein/retinoic acid-inducible neural-specific protein 1 (BRINP1), C terminus of HSC70-interacting protein (CHIP), Cluster of Differentiation 70 (CD70), c-JUN kinase 1 (JNK1), protein kinase C theta (PRKCQ), ring finger protein 20 (RNF20), and sirtuin 1 (SIRT1).

In another aspect, the disclosure features a method for producing a functionally enhanced and/or lineage stabilized regulatory T (T_{REG}) cell. The method involves contacting a T_{REG} cell with a base editor, or a polynucleotide encoding the base editor. The base editor contains a polynucleotide programmable DNA binding polypeptide (napDNAbp), and an adenosine deaminase. The method also involves contacting the T_{REG} cell with one or more of the following guide RNAs, or one or more polynucleotides encoding the one or more guide RNAs: a) a guide RNA containing a nucleotide sequence selected from one or more of TSBTx2810, TSBTx2813, and TSBTx2815; b) a guide RNA containing a nucleotide sequence selected from one or more of TSBTx2834 and TSBTx845; c) a guide RNA containing the nucleotide sequence TSBTx025; d) a guide RNA containing the nucleotide sequence TSBTx845; and e) a guide RNA containing a nucleotide sequence selected from one or more of TSBTx2817, TSBTx2818, TSBTx2819, TSBTx2820, TSBTx2821, TSBTx2822, TSBTx2823, TSBTx2824, TSBTx2825, TSBTx2826, TSBTx2827, TSBTx2828, TSBTx2830, and TSBTx2831. The method is associated with a reduction or elimination of expression in the T_{REG} of one or more of the following polypeptides: cluster of differentiation 70 (CD70), cluster of differentiation 58 (CD58), programmed cell death 1 (PD-1), beta-2 microglobulin (B2M), and sirtuin 1 (SIRT1).

In another aspect, the disclosure features a method for producing a functionally enhanced and/or lineage stabilized regulatory T (T_{REG}) cell. The method involves contacting a T_{REG} cell with a base editor, or a polynucleotide encoding the base editor. The base editor contains a polynucleotide programmable DNA binding polypeptide (napDNAbp), and a cytidine deaminase. The method also involves contacting the T_{REG} cell with a guide RNA, or a polynucleotide encoding the guide RNA. The guide RNA contains a nucleotide sequence selected from one or more of TSBTx2813, TSBTx2814, and TSBTx2816. The method is associated with a reduction or elimination of expression in the T_{REG} of a cluster of differentiation 70 (CD70) polypeptide.

In another aspect, the disclosure features a method for producing a functionally enhanced and/or lineage stabilized regulatory T (T_{REG}) cell. The method involves contacting a T_{REG} cell with a Cas12b polypeptide, or a polynucleotide encoding the Cas12b polypeptide. The method also involves contacting the T_{REG} cell with a guide RNA containing a nucleotide sequence selected from one or more of: SEQ ID NOs: 788 to 810, 878 to 903, 696-714, 737, 751-778, and 904-931, or a polynucleotide encoding the guide RNA. The method is associated with a reduction or elimination of C terminus of HSC70-interacting protein (CHIP) and/or ring finger protein 20 (RNF20) in the T_{REG} cell.

In another aspect, the disclosure features a cell prepared according to the method of any aspect provided herein, or embodiments thereof.

In another aspect, the disclosure features a pharmaceutical composition comprising the cell of any aspect provided herein, or embodiments thereof.

In any aspect provided herein, or embodiments thereof, the method involves contacting the T_{REG} cell with gRNAs containing each of the following nucleotide sequences: TSBTx2813, TSBTx2817, TSBTx2834, TSBTx025, and TSBTx845 to thereby reduce or eliminate expression of each of CD70, SIRT1, CD58, PD-1, and B2M in the T_{REG} cell.

In any aspect provided herein, or embodiments thereof, the method further involves contacting the T_{REG} cell with a gRNA that targets the base editor to effect an alteration in a nucleic acid molecule. The nucleic acid molecule encodes a polypeptide and/or contains a regulatory element associated with expression thereof. The polypeptide is selected from one or more of beta-2 microglobulin (B2M), Cluster of Differentiation 58, and programmed cell death 1 (PD-1).

In any aspect provided herein, or embodiments thereof, the method further involves overexpressing in the T_{REG} cell an inhibitory receptor selected from one or more of Human Leukocyte Antigen-E (HLA-E), Human Leukocyte Antigen-G (HLA-G), Programmed Death Ligand 1 (PD-L1), and Cluster of Differentiation 47 (CD47).

In any aspect provided herein, or embodiments thereof, the method further involves effecting an alteration in a third nucleic acid molecule, where the nucleic acid molecule encodes a beta-2 microglobulin (B2M) polypeptide and/or contains a regulatory element associated with expression thereof.

In any aspect provided herein, or embodiments thereof, the method involves editing a combination of nucleic acid molecules encoding a combination of two or more polypeptides and/or a regulatory element associated with expression thereof, where the combination of polypeptides is selected from one or more of: SIRT1, PD-1, CD70, and CD58; SIRT1, PD-1, and CD70; SIRT1, PD-1, and CD58; SIRT1, CD70, and CD58; SIRT1 and PD-1; SIRT1 and CD70; SIRT1 and CD58; SIRT1, PD-1, CD70, CD58, and B2M; SIRT1, PD-1, CD70, and B2M; SIRT1, PD-1, CD58 and B2M; SIRT1, CD70, CD58, and B2M; SIRT1, PD-1, and B2M; SIRT1, CD70, and B2M; SIRT1, CD58, and B2M; RNF20, PD-1, CD70, and CD58; RNF20, PD-1, and CD70; RNF20, PD-1, and CD58; RNF20, CD70, and CD58; RNF20 and PD-1; RNF20 and CD70; RNF20 and CD58; RNF20, PD-1, CD70, CD58, and B2M; RNF20, PD-1, CD70, and B2M; RNF20, PD-1, CD58 and B2M; RNF20, CD70, CD58, and B2M; RNF20, PD-1, and B2M; RNF20, CD70, and B2M; RNF20, CD58, and B2M; SIRT1, RNF20, PD-1, CD70, and CD58; SIRT1, RNF20, PD-1, and CD70; SIRT1, RNF20, PD-1, and CD58; SIRT1, RNF20, CD70, and CD58; SIRT1, RNF20, and PD-1; SIRT1, RNF20, and CD70; SIRT1, RNF20, and CD58; SIRT1, RNF20, PD-1, CD70, CD58, and B2M; SIRT1, RNF20, PD-1, CD70, and B2M; SIRT1, RNF20, PD-1, CD58, and B2M; SIRT1, RNF20, CD70, CD58, and B2M; SIRT1, RNF20, PD-1, and B2M; SIRT1, RNF20, CD70, and B2M; and SIRT1, RNF20, CD58, and B2M.

In any aspect provided herein, or embodiments thereof, FoxP3 expression is increased by at least about 10% relative to a reference cell. In any aspect provided herein, or embodiments thereof, FoxP3 expression is increased by at least about 25% relative to a reference cell. In any aspect provided herein, or embodiments thereof, FoxP3 expression is increased by at least about 50% relative to a reference cell. In any aspect provided herein, or embodiments thereof, helios expression is increased by at least about 2-fold relative to a reference cell. In any aspect provided herein, or embodiments thereof, helios expression is increased by about 3-fold relative to a reference cell. In any aspect provided herein, or embodiments thereof, CTLA4 expression is increased by about 2-fold relative to a reference cell. In any aspect provided herein, or embodiments thereof, CTLA4 expression is increased by about 3-fold relative to a reference cell.

In any aspect provided herein, or embodiments thereof, each guide RNA contains a sequence with at least about 85% sequence identity to a sequence selected from those listed in Table 1A-1C or 2A-2C, or truncations thereof. In any aspect provided herein, or embodiments, thereof, each guide RNA contains a sequence selected from one or more of TSBTx2810, TSBTx2813, TSBTx2815, TSBTx2813, TSBTx2814, TSBTx2816, TSBTx2834, TSBTx845, TSBTx025, TSBTx2817, TSBTx2817, TSBTx2818, TSBTx2819, TSBTx2820, TSBTx2821, TSBTx2822, TSBTx2823, TSBTx2824, TSBTx2825, TSBTx2826, TSBTx2827, TSBTx2828, TSBTx2830, or TSBTx2831, TSBTx1680, TSBTx1681, TSBTx1682, TSBTx1683, TSBTx1684, TSBTx1685, TSBTx1686, TSBTx1687, TSBTx1688, TSBTx1689, TSBTx1690, TSBTx1691, TSBTx1692, TSBTx1693, TSBTx1694, TSBTx1695, TSBTx1696, TSBTx1697, TSBTx1698, or TSBTx2853, TSBTx2813, TSBTx2817, TSBTx2834, TSBTx025, and TSBTx845.

In any aspect provided herein, or embodiments thereof, the alteration is associated with a reduction of g of a T_{CONV} cell by the functionally enhanced and/or lineage stabilized T_{REG} cell relative to a reference cell.

In any aspect provided herein, or embodiments thereof, the deaminase is a cytidine deaminase or an adenosine deaminase.

In any aspect provided herein, or embodiments thereof, the base editor contains a complex containing the deaminase and the polynucleotide programmable DNA binding polypeptide (napDNAbp), or the base editor contains a fusion protein containing the polynucleotide programmable DNA binding polypeptide (napDNAbp) fused to the deaminase. In any aspect provided herein, or embodiments thereof, the napDNAbp is Cas9 or Cas12. In any aspect provided herein, or embodiments thereof, the napDNAbp is a Streptococcus pyogenes Cas9 (SpCas9), a Staphylococcus aureus Cas9 (SaCas9), Streptococcus thermophilus 1 Cas9 (St1Cas9), or a variant thereof. In any aspect provided herein, or embodiments thereof, the napDNAbp contains a nuclease dead Cas9 (dCas9) or a Cas9 nickase (nCas9).

In any aspect provided herein, or embodiments thereof, the base editor further contains one or more uracil glycosylase inhibitors (UGIs). In any aspect provided herein, or embodiments thereof, the base editor further contains one or more nuclear localization signals (NLS). In embodiments, the NLS is a bipartite NLS.

In any aspect provided herein, or embodiments thereof, the alteration encodes a missense mutation and/or is associated with reduced expression of the polypeptide.

In any aspect provided herein, or embodiments thereof, the method further involves expressing a chimeric antigen receptor (CAR) in the T_{REG} cell.

In any aspect provided herein, or embodiments thereof, the T_{REG} cell is obtained from a healthy donor. In embodiments, the donor is a human.

In any aspect provided herein, or embodiments thereof, the guide RNAs contains a sequence selected from those listed in Tables 1A-1C and/or Tables 2A-2C, or truncations thereof.

In any aspect provided herein, or embodiments thereof, the functionally enhanced and/or lineage stabilized T_{REG} cell has increased persistence in a host, increased resistance to immune rejection, decreased risk of eliciting a host-versus-graft reaction.

In any aspect provided herein, or embodiments thereof, the napDNAbp further contains one or more nuclear localization signals (NLS).

In any aspect provided herein, or embodiments thereof, the cleavage disrupts a splice acceptor or splice donor site, promoter, intron, exon, enhancer, or an untranslated region (UTR). In any aspect provided herein, or embodiments thereof, the cleavage introduces a missense mutation and/or is associated with reduced expression of the polypeptide.

In any aspect provided herein, or embodiments thereof, the alteration contains an insertion or a deletion.

In any aspect provided herein, or embodiments thereof, the T_{REG} cell overexpresses an inhibitory receptor selected from one or more of Human Leukocyte Antigen-E (HLA-E), Human Leukocyte Antigen-G (HLA-G), Programmed Death Ligand 1 (PD-L1), and Cluster of Differentiation 47 (CD47).

In any aspect provided herein, or embodiments thereof, the T_{REG} cell contains a nucleobase alteration that reduces or eliminates expression of a polypeptide selected from one or more of beta-2 microglobulin (B2M), Cluster of Differentiation 58 (CD58), and programmed cell death 1 (PD-1). In any aspect provided herein, or embodiments thereof, the T_{REG} cell contains an alteration that reduces or eliminates expression of a combination of polypeptides selected from one or more of: SIRT1, PD-1, CD70, and CD58; SIRT1, PD-1, and CD70; SIRT1, PD-1, and CD58; SIRT1, CD70, and CD58; SIRT1 and PD-1; SIRT1 and CD70; SIRT1 and CD58; SIRT1, PD-1, CD70, CD58, and B2M; SIRT1, PD-1, CD70, and B2M; SIRT1, PD-1, CD58 and B2M; SIRT1, CD70, CD58, and B2M; SIRT1, PD-1, and B2M; SIRT1, CD70, and B2M; SIRT1, CD58, and B2M; RNF20, PD-1, CD70, and CD58; RNF20, PD-1, and CD70; RNF20, PD-1, and CD58; RNF20, CD70, and CD58; RNF20 and PD-1; RNF20 and CD70; RNF20 and CD58; RNF20, PD-1, CD70, CD58, and B2M; RNF20, PD-1, CD70, and B2M; RNF20, PD-1, CD58 and B2M; RNF20, CD70, CD58, and B2M; RNF20, PD-1, and B2M; RNF20, CD70, and B2M; RNF20, CD58, and B2M; SIRT1, RNF20, PD-1, CD70, and CD58; SIRT1, RNF20, PD-1, and CD70; SIRT1, RNF20, PD-1, and CD58; SIRT1, RNF20, CD70, and CD58; SIRT1, RNF20, and PD-1; SIRT1, RNF20, and CD70; SIRT1, RNF20, and CD58; SIRT1, RNF20, PD-1, CD70, CD58, and B2M; SIRT1, RNF20, PD-1, CD70, and B2M; SIRT1, RNF20, PD-1, CD58, and B2M; SIRT1, RNF20, CD70, CD58, and B2M; SIRT1, RNF20, PD-1, and B2M; SIRT1, RNF20, CD70, and B2M; and SIRT1, RNF20, CD58, and B2M.

In any aspect provided herein, or embodiments thereof, the T_{REG} cell overexpresses an inhibitory receptor selected from one or more of Human Leukocyte Antigen-E (HLA-E), Human Leukocyte Antigen-G (HLA-G), Programmed Death Ligand 1 (PD-L1), and Cluster of Differentiation 47 (CD47).

In any aspect provided herein, or embodiments thereof, the pharmaceutical composition further contains a gRNA that targets the base editor to effect an alteration in a nucleic acid molecule that encodes a polypeptide and/or contains a regulatory element associated with expression of the polypeptide. The polypeptide is selected from one or more of beta-2 microglobulin (B2M), Cluster of Differentiation 58 (CD58), and programmed cell death 1 (PD-1).

In any aspect provided herein, or embodiments thereof, the alloimmune disease is a graft-versus-host disease. In any aspect provided herein, or embodiments thereof, the nucleic acid programmable DNA binding protein is a Cas protein, Type VI Cas protein, Type V Cas protein, Cas9 protein, Cas 12 protein, or a Cas12b protein.

In any aspect provided herein, or embodiments thereof, the gRNA contains a spacer sequence containing a sequence having at least 75%, 80%, 85%, 90%, 95%, 98%, 99% or greater identity up to 100% identity to a sequence listed in Tables 2A-2C. In any aspect provided herein, or embodiments thereof, the gRNA contains a spacer sequence containing a sequence selected from those listed in Tables 2A-2C. In any aspect provided herein, or embodiments thereof, the guide RNA contains a sequence having at least 75%, 80%, 85%, 90%, 95%, 98%, 99% or greater identity up to 100% identity to a sequence selected from those listed in Tables 1A-1C. In any aspect provided herein, or embodiments thereof, the guide RNA contains any one of those sequences listed in Tables 1A-1C.

In any aspect provided herein, or embodiments thereof, the guide RNA is bound to a base editor and/or a polynucleotide programmable DNA binding polypeptide (napDNAbp).

In any aspect provided herein, or embodiments thereof, the base editor contains an adenosine deaminase. In any aspect provided herein, or embodiments thereof, the base editor contains a cytidine deaminase.

In any aspect provided herein, or embodiments thereof, the guide RNA contains a spacer sequence about 18-23 nucleotides in length. In any aspect provided herein, or embodiments thereof, the guide RNA contains a spacer sequence about 18 nucleotides in length. In any aspect provided herein, or embodiments thereof, the guide RNA contains a spacer sequence about 19 nucleotides in length. In any aspect provided herein, or embodiments thereof, the guide RNA contains a spacer sequence about 22 nucleotides in length. In any aspect provided herein, or embodiments thereof, the guide RNA contains a spacer sequence about 23 nucleotides in length.

In any aspect provided herein, or embodiments thereof, the method is not a process for modifying the germline genetic identity of human beings.

In any aspect provided herein, or embodiments thereof, the base editor contains two nuclear localization signals. In any aspect provided herein, or embodiments thereof, the adenosine deaminase is ABE8.20. In any aspect provided herein, or embodiments thereof, the Cas12 is Cas12b. In any aspect provided herein, or embodiments thereof, the base editor contains the amino acid sequence of SEQ ID NO: 3357.

In any aspect provided herein, or embodiments thereof, the polypeptide is CHIP or RNF20.

In any aspect provided herein, or embodiments thereof, the base editor is ABE8.20.

In any aspect provided herein, or embodiments thereof, the method involves reducing or eliminating expression in the T_{REG} of each of the following polypeptides: cluster of differentiation 70 (CD70), cluster of differentiation 58 (CD58), programmed cell death 1 (PD-1), beta-2 microglobulin (B2M), and sirtuin 1 (SIRT1).

### Definitions

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “adenine” or “9H-Purin-6-amine” is meant a purine nucleobase with the molecular formula C₅H₅N₅, having the structure

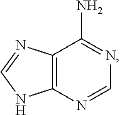

and corresponding to CAS No. 73-24-5.

By “adenosine” or “4-Amino-1-[(2R,3R,4S,5R)-3,4-dihydroxy-5-(hydroxymethyl)oxolan-2-yl]pyrimidin-2(1H)-one” is meant an adenine molecule attached to a ribose sugar via a glycosidic bond, having the structure

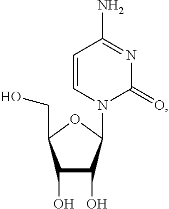

and corresponding to CAS No. 65-46-3. Its molecular formula is C₁₀H₁₃N₅O₄.

By “adenosine deaminase” or “adenine deaminase” is meant a polypeptide or fragment thereof capable of catalyzing the hydrolytic deamination of adenine or adenosine. In some embodiments, the deaminase or deaminase domain is an adenosine deaminase catalyzing the hydrolytic deamination of adenosine to inosine or deoxy adenosine to deoxyinosine. In some embodiments, the adenosine deaminase catalyzes the hydrolytic deamination of adenine or adenosine in deoxyribonucleic acid (DNA). The adenosine deaminases (e.g. engineered adenosine deaminases, evolved adenosine deaminases) provided herein may be from any organism (e.g., eukaryotic, prokaryotic), including but not limited to algae, bacteria, fungi, plants, invertebrates (e.g., insects), and vertebrates (e.g., amphibians, mammals). In some embodiments, the adenosine deaminase is an adenosine deaminase variant with one or more alterations and is capable of deaminating both adenine and cytosine in a target polynucleotide (e.g., DNA, RNA). In some embodiments, the target polynucleotide is single or double stranded. In some embodiments, the adenosine deaminase variant is capable of deaminating both adenine and cytosine in DNA. In embodiments, the adenosine deaminase variant is selected from those described in PCT/US2020/018192, PCT/US2020/049975, PCT/US2017/045381, and PCT/US2020/028568, the full contents of which are each incorporated herein by reference in their entireties for all purposes.

By “adenosine deaminase activity” is meant catalyzing the deamination of adenine or adenosine to guanine in a polynucleotide. In some embodiments, an adenosine deaminase variant as provided herein maintains adenosine deaminase activity (e.g., at least about 30%, 40%, 50%, 60%, 70%, 80%, 90% or more of the activity of a reference adenosine deaminase (e.g., TadA*8.20 or TadA*8.19)).

By “Adenosine Base Editor (ABE)” is meant a base editor comprising an adenosine deaminase.

By “Adenosine Base Editor (ABE) polynucleotide” is meant a polynucleotide encoding an ABE. By “Adenosine Base Editor 8 (ABE8) polypeptide” or “ABE8” is meant a base editor as defined herein comprising an adenosine deaminase or adenosine deaminase variant comprising one or more of the alterations listed in Table 5B, one of the combinations of alterations listed in Table 5B, or an alteration at one or more of the amino acid positions listed in Table 5B, such alterations are relative to the following reference sequence: MSEVEFSHEYWMRHALTLAKRARDEREVPVGAVLVLNNRVIGEGWNRAIGLHDPTAHAEIMA LRQGGLVMQNYRLIDATLYVTFEPCVMCAGAMIHSRIGRVVFGVRNAKTGAAGSLMDVLHYP GMNHRVEITEGILADECAALLCYFFRMPRQVFNAQKKAQSSTD (SEQ ID NO: 1), or a corresponding position in another adenosine deaminase. In embodiments, ABE8 comprises alterations at amino acids 82 and/or 166 of SEQ ID NO: 1 In some embodiments, ABE8 comprises further alterations, as described herein, relative to the reference sequence.

By “Adenosine Base Editor 8 (ABE8) polynucleotide” is meant a polynucleotide encoding an ABE8 polypeptide.

“Administering” is referred to herein as providing one or more compositions described herein to a patient or a subject. By way of example and without limitation, composition administration (e.g., injection) can be performed by intravenous (i.v.) injection, sub-cutaneous (s.c.) injection, intradermal (i.d.) injection, intraperitoneal (i.p.) injection, or intramuscular (i.m.) injection. One or more such routes can be employed. Parenteral administration can be, for example, by bolus injection or by gradual perfusion over time. In some embodiments, parenteral administration includes infusing or injecting intravascularly, intravenously, intramuscularly, intraarterially, intrathecally, intratumorally, intradermally, intraperitoneally, transtracheally, subcutaneously, subcuticularly, intraarticularly, subcapsularly, subarachnoidly and intrasternally. Alternatively, or concurrently, administration can be by the oral route.

By “agent” is meant any small molecule chemical compound, antibody, nucleic acid molecule, or polypeptide, or fragments thereof.

“Allogeneic,” as used herein, refers to cells of the same species that differ genetically and interact antigenically.

By “alteration” is meant a change in the level, structure, or activity of an analyte, gene or polypeptide as detected by standard art known methods such as those described herein. As used herein, an alteration includes a change (e.g., increase or decrease) in expression levels. In embodiments, the increase or decrease in expression levels is by 10%, 25%, 40%, 50%, or greater. In some embodiments, an alteration includes an insertion, deletion, or substitution of a nucleobase or amino acid (by, e.g., genetic engineering).

By “ameliorate” is meant decrease, suppress, attenuate, diminish, arrest, or stabilize the development or progression of a disease.

By “analog” is meant a molecule that is not identical but has analogous functional or structural features. For example, a polypeptide analog retains the biological activity of a corresponding naturally occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

As used herein, the term “antibody” refers to an immunoglobulin molecule that specifically binds to, or is immunologically reactive with, a particular antigen, and includes polyclonal, monoclonal, genetically engineered, and otherwise modified forms of antibodies, including but not limited to chimeric antibodies, humanized antibodies, heteroconjugate antibodies (e.g., bi- tri- and quad-specific antibodies, diabodies, triabodies, and tetrabodies), and antigen binding fragments of antibodies, including, for example, Fab′, F(ab′)2, Fab, Fv, rlgG, and scFv fragments. Unless otherwise indicated, the term “monoclonal antibody” (mAb) is meant to include both intact molecules, as well as antibody fragments (including, for example, Fab and F(ab′)2 fragments) that are capable of specifically binding to a target protein. As used herein, the Fab and F(ab′)2 fragments refer to antibody fragments that lack the Fc fragment of an intact antibody.

Antibodies (immunoglobulins) comprise two heavy chains linked together by disulfide bonds, and two light chains, with each light chain being linked to a respective heavy chain by disulfide bonds in a “Y” shaped configuration. Each heavy chain has at one end a variable domain (VH) followed by a number of constant domains (CH). Each light chain has a variable domain (VL) at one end and a constant domain (CL) at its other end. The variable domain of the light chain (VL) is aligned with the variable domain of the heavy chain (VL), and the light chain constant domain (CL) is aligned with the first constant domain of the heavy chain (CH1). The variable domains of each pair of light and heavy chains form the antigen binding site. The isotype of the heavy chain (gamma, alpha, delta, epsilon or mu) determines the immunoglobulin class (IgG, IgA, IgD, IgE or IgM, respectively). The light chain is either of two isotypes (kappa (κ) or lambda (λ)) found in all antibody classes. The terms “antibody” or “antibodies” include intact antibodies, such as polyclonal antibodies or monoclonal antibodies (mAbs), as well as proteolytic portions or fragments thereof, such as the Fab or F(ab′)2 fragments, that are capable of specifically binding to a target protein. Antibodies may include chimeric antibodies, recombinant and engineered antibodies, and antigen binding fragments thereof. Exemplary functional antibody fragments comprising whole or essentially whole variable regions of both the light and heavy chains are defined as follows: (i) Fv, defined as a genetically engineered fragment consisting of the variable region of the light chain and the variable region of the heavy chain expressed as two chains; (ii) single-chain Fv (“scFv”), a genetically engineered single-chain molecule including the variable region of the light chain and the variable region of the heavy chain, linked by a suitable polypeptide linker; (iii) Fab, a fragment of an antibody molecule containing a monovalent antigen-binding portion of an antibody molecule, obtained by treating an intact antibody with the enzyme papain to yield the intact light chain and the Fd fragment of the heavy chain, which consists of the variable and CH1 domains thereof; (iv) Fab′, a fragment of an antibody molecule containing a monovalent antigen-binding portion of an antibody molecule, obtained by treating an intact antibody with the enzyme pepsin, followed by reduction (two Fab′ fragments are generated per antibody molecule); and (v) F(ab′)2, a fragment of an antibody molecule containing a monovalent antigen-binding portion of an antibody molecule, obtained by treating an intact antibody with the enzyme pepsin (i.e., a dimer of Fab′ fragments held together by two disulfide bonds).

“Autologous,” as used herein, refers to cells from the same subject.

By “beta-2 microglobulin (β2M; B2M) polypeptide” is meant a protein having at least about 85% amino acid sequence identity to UniProt Accession No. P61769, which corresponds to SEQ ID NO: 973 (sp|P61769|B2MG HUMAN Beta-2-microglobulin OS=Homo sapiens OX=9606 GN=B2M PE=1 SV=1), or a fragment thereof having immunomodulatory activity.

By “beta-2-microglobulin (β2M; B2M) polynucleotide” is meant a nucleic acid molecule encoding an β2M polypeptide, as well as the introns, exons, 3′ untranslated regions, 5′ untranslated regions, and regulatory sequences associated with its expression, or fragments thereof. In embodiments, a β2M polynucleotide is the genomic sequence, cDNA, mRNA, or gene associated with and/or required for β2M expression. The beta-2-microglobulin gene encodes a serum protein associated with the major histocompatibility complex. “β2M” is sometimes recited as “B2M” herein. β2M is involved in non-self-recognition by host CD8+ T cells. An exemplary β2M polynucleotide sequence is provided at Genbank Accession No. DQ217933.1, which corresponds to SEQ ID NO: 974 (DQ217933.1 Homo sapiens beta-2-microglobin (β2M) gene, complete cds).

By “base editor (BE),” or “nucleobase editor polypeptide (NBE)” is meant an agent that binds a polynucleotide and has nucleobase modifying activity. In various embodiments, the base editor comprises a nucleobase modifying polypeptide (e.g., a deaminase) and a polynucleotide programmable nucleotide binding domain (e.g., Cas9 or Cpf1) in conjunction with a guide polynucleotide (e.g., guide RNA (gRNA)). Representative nucleic acid and protein sequences of base editors include those sequences having about or at least about 85% sequence identity to any base editor sequence provided in the sequence listing, such as those corresponding to any of SEQ ID NOs: 2-11.

By “base editing activity” is meant acting to chemically alter a base within a polynucleotide. In one embodiment, a first base is converted to a second base. In one embodiment, the base editing activity is cytidine deaminase activity, e.g., converting target C·G to T·A. In another embodiment, the base editing activity is adenosine or adenine deaminase activity, e.g., converting A·T to G·C.

The term “base editor system” refers to an intermolecular complex for editing a nucleobase of a target nucleotide sequence. In various embodiments, the base editor (BE) system comprises (1) a polynucleotide programmable nucleotide binding domain, a deaminase domain (e.g., cytidine deaminase or adenosine deaminase) for deaminating nucleobases in the target nucleotide sequence; and (2) one or more guide polynucleotides (e.g., guide RNA) in conjunction with the polynucleotide programmable nucleotide binding domain. In various embodiments, the base editor (BE) system comprises a nucleobase editor domain selected from an adenosine deaminase or a cytidine deaminase, and a domain having nucleic acid sequence specific binding activity. In some embodiments, the base editor system comprises (1) a base editor (BE) comprising a polynucleotide programmable DNA binding domain and a deaminase domain for deaminating one or more nucleobases in a target nucleotide sequence; and (2) one or more guide RNAs in conjunction with the polynucleotide programmable DNA binding domain. In some embodiments, the polynucleotide programmable nucleotide binding domain is a polynucleotide programmable DNA binding domain. In some embodiments, the base editor is a cytidine base editor (CBE). In some embodiments, the base editor is an adenine or adenosine base editor (ABE). In some embodiments, the base editor is an adenine or adenosine base editor (ABE) or a cytidine or cytosine base editor (CBE). In some embodiments, the base editor system (e.g., a base editor system comprising a cytidine deaminase) comprises a uracil glycosylase inhibitor or other agent or peptide (e.g., a uracil stabilizing protein such as provided in WO2022015969, the disclosure of which is incorporated herein by reference in its entirety for all purposes) that inhibits the inosine base excision repair system. By “BMP/retinoic acid-inducible neural-specific protein 1 (BRINP1) polypeptide” is meant a protein having at least about 85% amino acid sequence identity to UniProt Accession No. 060477, which corresponds to SEQ ID NO: 964 (sp|O60477|BRNP1_HUMAN BMP/retinoic acid-inducible neural-specific protein 1 OS=Homo sapiens OX=9606 GN=BRINP1 PE=1 SV=2), or a fragment thereof having immunomodulatory activity.

By “BMP/retinoic acid-inducible neural-specific protein 1 (BRINP1) polynucleotide” is meant a nucleic acid molecule encoding a BRINP1 polypeptide, as well as the introns, exons, 3′ untranslated regions, 5′ untranslated regions, and regulatory sequences associated with its expression, or fragments thereof. In embodiments, a BRINP1 polynucleotide is the genomic sequence, cDNA, mRNA, or gene associated with and/or required for BRINP1 expression. An exemplary BRINP1 polynucleotide sequence from Homo sapiens is provided at NCBI Reference Sequence Accession No. NM_014618.3 which corresponds to SEQ ID NO: 965 (NM_014618.3: Homo sapiens BMP/retinoic acid inducible neural specific 1 (BRINP1), mRNA), and at NCBI Ref. Seq. Accession No. NC_000019.10, which is provided in the Sequence Listing as SEQ ID NO: 966.

The term “Cas9” or “Cas9 domain” refers to an RNA guided nuclease comprising a Cas9 protein, or a fragment thereof (e.g., a protein comprising an active, inactive, or partially active DNA cleavage domain of Cas9, and/or the gRNA binding domain of Cas9). A Cas9 nuclease is also referred to sometimes as a casn1 nuclease or a CRISPR (clustered regularly interspaced short palindromic repeat) associated nuclease.

By “chimeric antigen receptor” or “CAR” is meant a synthetic or engineered receptor comprising an extracellular antigen binding domain operationally joined to one or more intracellular signaling domains where the CAR confers specificity for an antigen bound by the antigen binding domain onto an immune effector cell. In embodiments, the intracellular signaling domain is a T cell signaling domain. In embodiments, the immune effector cell is a regulatory T (T_{REG}) cell. In embodiments, the CAR is a SUPRA CAR, an anti-tag CAR, a TCR-CAR, or a TCR-like CAR (see, e.g., Guedan, et al. “Engineering and Design of Chimeric Antigen Receptors,” Methods and Clinical Development, 12:145-156 (2019); Poorebrahim, et al., “TCR-like CARs and TCR-CARs targeting neoepitopes: an emerging potential,” Cancer Gene Therapy, 28:581-589 (2021); and Minutolo, et al. “The Emergence of Universal Immune Receptor T Cell Therapy for Cancer,” Front Oncol., 9:176 (2019), the disclosures of which are incorporated herein by reference in their entireties for all purposes).

By “chimeric antigen receptor T cell” or “CAR-T cell” is meant a T cell expressing a CAR that has antigen specificity determined by the antibody-derived targeting domain of the CAR. As used herein, “CAR-T cells” include regulatory T (T_{REG}) cells. As used herein, “CAR-T cells” include cells engineered to express a CAR or a T cell receptor (TCR). Methods of making CARs are publicly available (see, e.g., Park et al., Trends Biotechnol., 29:550-557, 2011; Grupp et al., N Engl J Med., 368:1509-1518, 2013; Han et al., J. Hematol Oncol. 6:47, 2013; Haso et al., (2013) Blood, 121, 1165-1174; Mohseni, et al., (2020) Front. Immunol., 11, art. 1608, doi: 10.3389/fimmu.2020.01608; Eggenhuizen, et al. Int. J. Mol. Sci. (2020), 21:7015, doi: 10.3390/ijms21197015; PCT Pubs. WO2012/079000, WO2013/059593; and U.S. Pub. 2012/0213783, the disclosure of each of which is incorporated herein by reference herein in its entirety for all purposes).

By “class II, major histocompatibility complex, transactivator (CIITA) polypeptide” is meant a protein having at least about 85% amino acid sequence identity to NCBI Ref. Seq. Accession No. NP_001273331.1, or a fragment thereof having immunomodulatory activity.

By “class II, major histocompatibility complex, transactivator (CIITA) polypeptide polynucleotide” is meant a nucleic acid molecule encoding an CIITA polypeptide, as well as the introns, exons, 3′ untranslated regions, 5′ untranslated regions, and regulatory sequences associated with its expression, or fragments thereof. In embodiments, a CIITA polynucleotide is the genomic sequence, cDNA, mRNA, or gene associated with and/or required for CIITA expression. An exemplary CIITA polynucleotide sequence is provided at NCBI Ref. Seq. Accession No. NM_001286402.1. The CIITA gene corresponds to Ensembl: ENSG00000179583.

By “Cluster of Differentiation 47 (CD47) polypeptide” is meant a protein having at least about 85% amino acid sequence identity to NCBI Ref. Seq. Accession No. NP_001768.1, or a fragment thereof having immunomodulatory activity.

By “Cluster of Differentiation 47 (CD47) polynucleotide” is meant a nucleic acid molecule encoding an CD47 polypeptide, as well as the introns, exons, 3′ untranslated regions, 5′ untranslated regions, and regulatory sequences associated with its expression, or fragments thereof. In embodiments, a CD47 polynucleotide is the genomic sequence, cDNA, mRNA, or gene associated with and/or required for CD47 expression. An exemplary CD47 polynucleotide sequence is provided at NCBI Ref. Seq. Accession No. NM_001777.4. The CD47 gene corresponds to Ensembl: ENSG00000196776.

By “Cluster of Differentiation 52 (CD52) polypeptide” is meant a protein having at least about 85% amino acid sequence identity to NCBI Ref. Seq. Accession No. NP_001794.2, or a fragment thereof having immunomodulatory activity.

By “Cluster of Differentiation 52 (CD52) polynucleotide” is meant a nucleic acid molecule encoding a CD52 polypeptide, as well as the introns, exons, 3′ untranslated regions, 5′ untranslated regions, and regulatory sequences associated with its expression, or fragments thereof. In embodiments, a CD52 polynucleotide is the genomic sequence, cDNA, mRNA, or gene associated with and/or required for CD52 expression. An exemplary CD52 polynucleotide sequence is provided at NCBI Ref. Seq. Accession No. NM_001803.3. The CD52 gene corresponds to ENSG00000169442.

By “Cluster of Differentiation 58 (CD58) polypeptide” is meant a protein having at least about 85% amino acid sequence identity to NCBI Reference Sequence Accession No. NP_001770.1, which corresponds to SEQ ID NO: 975, or a fragment thereof that functions in an immune response. CD58 and the immunobiology thereof is described in Zhang, et al. “CD58 Immunobiology at a Glance,” Frontiers in Immunology, vol. 12, article 705260 (2021), the disclosure of which is incorporated herein by reference in its entirety for all purposes.

By “Cluster of Differentiation 58 (CD58) polynucleotide” is meant a nucleic acid molecule encoding an CD58 polypeptide, as well as the introns, exons, 3′ untranslated regions, 5′ untranslated regions, and regulatory sequences associated with its expression, or fragments thereof. In embodiments, a CD58 polynucleotide is the genomic sequence, cDNA, mRNA, or gene associated with and/or required for CD58 expression. An exemplary CD58 polynucleotide is provided at NCBI Accession No. NM_001779.3, which corresponds to SEQ ID NO: 976.

By “CD70 Molecule (CD70) polypeptide” or “Cluster of Differentiation 70 (CD70) polypeptide” is meant a protein having at least about 85% amino acid sequence identity to GenBank Accession No. AAH00725.1, which corresponds to SEQ ID NO: 967 (AAH00725.1 CD70 molecule [Homo sapiens]), or a fragment thereof having immunomodulatory activity.

By “CD70 Molecule (CD70) polynucleotide” or “Cluster of Differentiation 70 (CD70) polynucleotide” is meant a nucleic acid molecule encoding a CD70 polypeptide, as well as the introns, exons, 3′ untranslated regions, 5′ untranslated regions, and regulatory sequences associated with its expression, or fragments thereof. In embodiments, a CD70 polynucleotide is the genomic sequence, cDNA, mRNA, or gene associated with and/or required for CD70 expression. An exemplary CD70 polynucleotide sequence from Homo sapiens is provided at GenBank Accession No. BC000725.2 which corresponds to SEQ ID NO: 968 (BC000725.2:97-678 Homo sapiens CD70 molecule, mRNA (cDNA clone MGC:1597 IMAGE:3506629), complete cds), and at NCBI Ref. Seq. Accession No. NC_000019.10, which is provided in the Sequence Listing as SEQ ID NO: 969.

By “C terminus of HSC70-interacting protein (CHIP) polypeptide” is meant a protein having at least about 85% amino acid sequence identity to GenBank Accession No. AAH63617.1, which corresponds to SEQ ID NO: 970 (AAH63617.1 STIP1 homology and U-box containing protein 1 [Homo sapiens]), or a fragment thereof having ubiquitin ligase activity.

By “C terminus of HSC70-interacting protein (CHIP) polynucleotide” is meant a nucleic acid molecule encoding a CHIP polypeptide, as well as the introns, exons, 3′ untranslated regions, 5′ untranslated regions, and regulatory sequences associated with its expression, or fragments thereof. In embodiments, a CHIP polynucleotide is the genomic sequence, cDNA, mRNA, or gene associated with and/or required for CHIP expression. An exemplary CHIP polynucleotide sequence from Homo sapiens is provided at NCBI Reference Sequence Accession No. NG_034141.1 which corresponds to SEQ ID NO: 971 (NG 034141.1:681-719,1226-1387, 1861-2009,2166-2278, 2349-2449,2529-2589, 2671-2942 Homo sapiens STIP1 homology and U-box containing protein 1 (STUB1), RefSeqGene on chromosome 16), and at NCBI Ref. Seq. Accession No. NG_034141.1, which is provided in the Sequence Listing as SEQ ID NO: 972.

By “Cluster of Differentiation 123 (CD123) polypeptide” is meant a protein having at least about 85% amino acid sequence identity to NCBI Ref. Seq. Accession No. NP_002174.1, or a fragment thereof having immunomodulatory activity.

By “Cluster of Differentiation 123 (CD123) polynucleotide” is meant a nucleic acid molecule encoding a CD123 polypeptide, as well as the introns, exons, 3′ untranslated regions, 5′ untranslated regions, and regulatory sequences associated with its expression, or fragments thereof. In embodiments, a CD123 polynucleotide is the genomic sequence, cDNA, mRNA, or gene associated with and/or required for CD123 expression. An exemplary CD123 polynucleotide sequence is provided at NCBI Ref. Seq. Accession No. NM_002183.4. The CD123 gene corresponds to Ensembl: ENSG00000185291.

By “cluster of differentiation 155 (CD155) polypeptide” is meant a protein having at least about 85% amino acid sequence identity to GenBank Accession No. AAF69803.1, or a fragment thereof having immunomodulatory activity.

By “cluster of differentiation 155 (CD155) polynucleotide” is meant a nucleic acid molecule encoding a CD155 polypeptide, as well as the introns, exons, 3′ untranslated regions, 5′ untranslated regions, and regulatory sequences associated with its expression, or fragments thereof. In embodiments, a CD155 polynucleotide is the genomic sequence, cDNA, mRNA, or gene associated with and/or required for CD155 expression. An exemplary CD155 polynucleotide sequence is provided at GenBank Accession No. AC068948.1. The CD155 gene corresponds to ENSG00000073008.15.

By “Homo sapiens cytotoxic T-lymphocyte associated protein 4 (CTLA4) polypeptide” is meant a protein having at least about 85% amino acid sequence identity to GenBank Accession No. AAL07473.1, or a fragment thereof having immunomodulatory activity.

By “Homo sapiens cytotoxic T-lymphocyte associated protein 4 (CTLA4) polynucleotide” is meant a nucleic acid molecule encoding an CTLA4 polypeptide, as well as the introns, exons, 3′ untranslated regions, 5′ untranslated regions, and regulatory sequences associated with its expression, or fragments thereof. In embodiments, a CTLA4 polynucleotide is the genomic sequence, cDNA, mRNA, or gene associated with and/or required for CTLA4 expression. An exemplary CTLA4 polynucleotide sequence from Homo sapiens is provided at GenBank Accession No. AF414120.1.

The term “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and Schirmer, R. H., Principles of Protein Structure, Springer-Verlag, New York (1979)). According to such analyses, groups of amino acids can be defined where amino acids within a group exchange preferentially with each other, and therefore resemble each other most in their impact on the overall protein structure (Schulz, G. E. and Schirmer, R. H., supra). Non-limiting examples of conservative mutations include amino acid substitutions of amino acids, for example, lysine for arginine and vice versa such that a positive charge can be maintained; glutamic acid for aspartic acid and vice versa such that a negative charge can be maintained; serine for threonine such that a free —OH can be maintained; and glutamine for asparagine such that a free —NH₂ can be maintained.

The term “coding sequence” or “protein coding sequence” as used interchangeably herein refers to a segment of a polynucleotide that codes for a protein. Coding sequences can also be referred to as open reading frames. The region or sequence is bounded nearer the 5′ end by a start codon and nearer the 3′ end with a stop codon. Stop codons useful with the base editors described herein include the following: TAG, TAA, and TGA.

By “complex” is meant a combination of two or more molecules whose interaction relies on inter-molecular forces. Non-limiting examples of inter-molecular forces include covalent and non-covalent interactions. Non-limiting examples of non-covalent interactions include hydrogen bonding, ionic bonding, halogen bonding, hydrophobic bonding, van der Waals interactions (e.g., dipole-dipole interactions, dipole-induced dipole interactions, and London dispersion forces), and π-effects. In an embodiment, a complex comprises polypeptides, polynucleotides, or a combination of one or more polypeptides and one or more polynucleotides. In one embodiment, a complex comprises one or more polypeptides that associate to form a base editor (e.g., base editor comprising a nucleic acid programmable DNA binding protein, such as Cas9, and a deaminase) and a polynucleotide (e.g., a guide RNA). In an embodiment, the complex is held together by hydrogen bonds. It should be appreciated that one or more components of a base editor (e.g., a deaminase, or a nucleic acid programmable DNA binding protein) may associate covalently or non-covalently. As one example, a base editor may include a deaminase covalently linked to a nucleic acid programmable DNA binding protein (e.g., by a peptide bond). Alternatively, a base editor may include a deaminase and a nucleic acid programmable DNA binding protein that associate noncovalently (e.g., where one or more components of the base editor are supplied in trans and associate directly or via another molecule such as a protein or nucleic acid). In an embodiment, one or more components of the complex are held together by hydrogen bonds.

By “cytosine” or “4-Aminopyrimidin-2(1H)-one” is meant a purine nucleobase with the molecular formula C₄H₅N₃O, having the structure

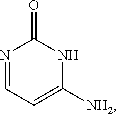

and corresponding to CAS No. 71-30-7.

By “cytidine” is meant a cytosine molecule attached to a ribose sugar via a glycosidic NH₂ bond, having the structure

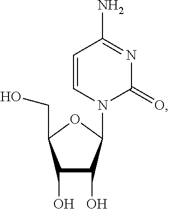

and corresponding to CAS No. 65-46-3. Its molecular formula is C₉H₁₃N₃O₅.

By “Cytidine Base Editor (CBE)” is meant a base editor comprising a cytidine deaminase.

By “Cytidine Base Editor (CBE) polynucleotide” is meant a polynucleotide encoding a CBE.

By “cytidine deaminase” or “cytosine deaminase” is meant a polypeptide or fragment thereof capable of deaminating cytidine or cytosine. In embodiments, the cytidine or cytosine is present in a polynucleotide. In one embodiment, the cytidine deaminase converts cytosine to uracil or 5-methylcytosine to thymine. The terms “cytidine deaminase” and “cytosine deaminase” are used interchangeably throughout the application. Petromyzon marinus cytosine deaminase 1 (PmCDA1) (SEQ ID NO: 13-14), Activation-induced cytidine deaminase (AICDA) (SEQ ID NOs: 15-21), and APOBEC (SEQ ID NOs: 12-61) are exemplary cytidine deaminases. Further exemplary cytidine deaminase (CDA) sequences are provided in the Sequence Listing as SEQ ID NOs: 62-66 and SEQ ID NOs: 67-189. Non-limiting examples of cytidine deaminases include those described in PCT/US20/16288, PCT/US2018/021878, 180802-021804/PCT, PCT/US2018/048969, and PCT/US2016/058344.

By “cytosine deaminase activity” is meant catalyzing the deamination of cytosine or cytidine. In one embodiment, a polypeptide having cytosine deaminase activity converts an amino group to a carbonyl group. In an embodiment, a cytosine deaminase converts cytosine to uracil (i.e., C to U) or 5-methylcytosine to thymine (i.e., 5mC to T). In some embodiments, a cytosine deaminase as provided herein has increased cytosine deaminase activity (e.g., at least 10-fold, 20-fold, 30-fold, 40-fold, 50-fold, 60-fold, 70-fold, 80-fold, 90-fold, 100-fold or more) relative to a reference cytosine deaminase.

The term “deaminase” or “deaminase domain,” as used herein, refers to a protein or fragment thereof that catalyzes a deamination reaction.

“Detect” refers to identifying the presence, absence, or amount of the analyte to be detected. In one embodiment, a sequence alteration in a polynucleotide or polypeptide is detected. In another embodiment, the presence of indels is detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an enzyme linked immunosorbent assay (ELISA)), biotin, digoxigenin, or haptens.

By “disease” is meant any condition or disorder that damages or interferes with the normal function of a cell, tissue, or organ. Exemplary diseases include diseases amenable to treatment with modified T_{REG} cells of the present disclosure (e.g., CAR T_{REG} cells). Non-limiting examples of diseases include autoimmune diseases and alloimmune diseases, such as graft versus host disease (GVHD), acute GVHD, or chronic GVHD. In embodiments, the disease is any disease or disorder associated with an undesired immune activity or response. Further non-limiting examples of diseases include type I diabetes, multiple sclerosis, rheumatoid arthritis, amyotrophic lateral sclerosis (ALS), hemophilia, autoantibody-mediated autoimmune disease, asthma, systemic lupus erythematosus (SLE), Chrohn's disease, cutaneious lupus, and pemphigus.

By “effective amount” is meant the amount of an agent or active compound, e.g., a base editor as described herein, that is required to ameliorate the symptoms of a disease relative to an untreated patient or an individual without disease, i.e., a healthy individual, or is the amount of the agent or active compound sufficient to elicit a desired biological response. The effective amount of active compound(s) used to practice embodiments of the disclosure for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In one embodiment, an effective amount is the amount of a base editor of the disclosure sufficient to introduce an alteration in a gene of interest in a cell (e.g., a cell in vitro or in vivo). In one embodiment, an effective amount is the amount of a base editor required to achieve a therapeutic effect. Such therapeutic effect need not be sufficient to alter a pathogenic gene in all cells of a subject, tissue, or organ, but only to alter the pathogenic gene in about 1%, 5%, 10%, 25%, 50%, 75% or more of the cells present in a subject, tissue, or organ. In one embodiment, an effective amount is the amount of a modified immune cell (e.g., a modified T_{REG} cell or CAR T_{REG} cell as provided herein) required to achieve a therapeutic effect (e.g., reduce or abate graft versus host disease, or suppress an undesirable immune response). In one embodiment, an effective amount is sufficient to ameliorate one or more symptoms of a disease.

The term “exonuclease” refers to a protein or polypeptide capable of digesting a nucleic acid (e.g., RNA or DNA) from free ends. The term “endonuclease” refers to a protein or polypeptide capable of catalyzing (e.g., cleaving) internal regions in a nucleic acid (e.g., DNA or RNA).

By “Forkhead box P3 (FOXP3) polypeptide” is meant a protein having at least about 85% amino acid sequence identity to GenBank Accession No. ABQ15210.1, or a fragment thereof having DNA binding and/or transcriptional regulatory activity. In embodiments, FoxP3 supports the development and/or function of a T_{REG} cell.

By “Forkhead box P3 (FOXP3) polynucleotide” is meant a nucleic acid molecule encoding a FOXP3 polypeptide, as well as the introns, exons, 3′ untranslated regions, 5′ untranslated regions, and regulatory sequences associated with its expression, or fragments thereof. In embodiments, a FOXP3 polynucleotide is the genomic sequence, cDNA, mRNA, or gene associated with and/or required for FOXP3 expression. An exemplary FOXP3 polynucleotide sequence from Homo sapiens is provided at Genbank Accession Number: EF534714.1.

By “fragment” is meant a portion of a polypeptide or nucleic acid molecule. This portion contains, at least about 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids. In some embodiments, the fragment is a functional fragment.

In general, a “gene” is a polynucleotide that is capable of being transcribed to an RNA that either has a regulatory function, a catalytic function, and/or encodes a protein. In embodiments, the polynucleotide is in the genome of a cell. A eukaryotic gene typically has introns and exons, which may organize to produce different RNA splice variants that encode alternative versions of a mature protein. The skilled artisan will appreciate that the present disclosure encompasses all transcripts encoding a polypeptide of interest, including splice variants, allelic variants and transcripts that occur because of alternative promoter sites or alternative poly-adenylation sites. A “full-length” gene or RNA therefore encompasses any naturally occurring splice variants, allelic variants, other alternative transcripts, splice variants generated by recombinant technologies which bear the same function as the naturally occurring variants, and the resulting RNA molecules.

By “IKAROS family zinc finger 2 (IKZF2; helios) polypeptide” is meant a protein having at least about 85% amino acid sequence identity to GenBank Accession No. AAH28936.1, or a fragment thereof functioning as a transcription factor.

By “IKAROS family zinc finger 2 (IKZF2; helios) polynucleotide” is meant a nucleic acid molecule encoding a helios polypeptide, as well as the introns, exons, 3′ untranslated regions, 5′ untranslated regions, and regulatory sequences associated with its expression, or fragments thereof. In embodiments, a helios polynucleotide is the genomic sequence, cDNA, mRNA, or gene associated with and/or required for helios expression. An exemplary helios polynucleotide sequence from Homo sapiens is provided at Genbank Accession No. BC028936.1.

“Host versus graft disease” (HVGD) refers to a pathological condition where the immune system of a host generates an immune response against transplanted cells of a donor. By “guide polynucleotide” is meant a polynucleotide or polynucleotide complex which is specific for a target sequence and can form a complex with a polynucleotide programmable nucleotide binding domain protein (e.g., Cas9 or Cpf1). In an embodiment, the guide polynucleotide is a guide RNA (gRNA). gRNAs can exist as a complex of two or more RNAs, or as a single RNA molecule.

By “heterologous,” or “exogenous” is meant a polynucleotide or polypeptide that 1) has been experimentally incorporated to a polynucleotide or polypeptide sequence to which the polynucleotide or polypeptide is not normally found in nature; or 2) has been experimentally placed into a cell that does not normally comprise the polynucleotide or polypeptide. In some embodiments, “heterologous” means that a polynucleotide or polypeptide has been experimentally placed into a non-native context. In some embodiments, a heterologous polynucleotide or polypeptide is derived from a first species or host organism and is incorporated into a polynucleotide or polypeptide derived from a second species or host organism. In some embodiments, the first species or host organism is different from the second species or host organism. In some embodiments the heterologous polynucleotide is DNA. In some embodiments the heterologous polynucleotide is RNA.

By “Human Leukocyte Antigen-E (HLA-E) polypeptide” is meant a protein having at least about 85% amino acid sequence identity to NCBI Ref. Seq. Accession No. NP_005507.3, or a fragment thereof having immunomodulatory activity.

By “Human Leukocyte Antigen-E (HLA-E) polynucleotide” is meant a nucleic acid molecule encoding an HLA-E polypeptide, as well as the introns, exons, 3′ untranslated regions, 5′ untranslated regions, and regulatory sequences associated with its expression, or fragments thereof. In embodiments, an HLA-E polynucleotide is the genomic sequence, cDNA, mRNA, or gene associated with and/or required for HLA-E expression. An exemplary HLA-E polynucleotide sequence is provided at NCBI Ref. Seq. Accession No. NM_005516.6. The HLA-E gene corresponds to Ensembl: ENSG00000116815.

By “Human Leukocyte Antigen-G (HLA-G) polypeptide” is meant a protein having at least about 85% amino acid sequence identity to NCBI Ref. Seq. Accession No. NP_001350496.1, or a fragment thereof having immunomodulatory activity.

By “Human Leukocyte Antigen-G (HLA-G) polynucleotide” is meant a nucleic acid molecule encoding an HLA-G polypeptide, as well as the introns, exons, 3′ untranslated regions, 5′ untranslated regions, and regulatory sequences associated with its expression, or fragments thereof. In embodiments, an HLA-G polynucleotide is the genomic sequence, cDNA, mRNA, or gene associated with and/or required for HLA-G expression. An exemplary HLA-G polynucleotide sequence is provided at NCBI Ref. Seq. Accession No. NM_001363567.2. The HLA-G gene corresponds to Ensemble: ENSG00000230413, ENSG00000233095, ENSG00000237216, ENSG00000276051 and ENSG00000204632.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “immune cell” is meant a cell of the immune system capable of generating or participating in an immune response. Exemplary immune cells include, but are not limited to, T cells, such as regulatory T (T_{REG}) cells or conventional T (T_{CONV}) cells. By “immune effector cell” is meant a lymphocyte, once activated, capable of modulating or effecting an immune response. In some embodiments, immune effector cells are T cells, such as regulatory T (T_{REG}) cells or conventional T (T_{CONV}) cells. In some embodiments, the immune effector cell has been genetically modified. In some embodiments, the effector T cells are thymocytes, immature T lymphocytes, mature T lymphocytes, resting T lymphocytes, or activated T lymphocytes. In embodiments, the effector cells are viable CD4+, CD25+, and CD127− cells (T_{REG}).

By “immune response regulation polypeptide” is meant a protein that modulates an immune response. An immune response regulation polypeptide may directly or indirectly modulate an immune response. For example, an immune response regulation polypeptide may increase or decrease the activation of an immune cell, e.g., a regulatory T (T_{REG}) cell or a conventional T (T_{CONV}) cell. An immune response regulation polypeptide may increase or decrease the activation threshold of an immune cell. In some embodiments, the immune response regulation polypeptide modulates a signal.

By “immune response regulation polynucleotide” or “immune response regulator polynucleotide” or “immune response regulation gene” is meant a nucleic acid molecule that encodes a polypeptide that modulates an immune response.

By “immunogen” is meant a polypeptide or fragment thereof capable of inducing an immune response. Exemplary immunogens include CD2, CD3e, CD3 delta, CD3 gamma, TRAC, TRBC1, TRBC2, CD4, CD5, CD7, CD8, CD19, CD23, CD27, CD28, CD30, CD33, CD52, CD58, CD70, CD127, CD122, CD130, CD132, CD38, CD69, CD11a, CD58, CD99, CD103, CCR4, CCR5, CCR6, CCR9, CCR10, CXCR3, CXCR4, CLA, CD161, PD-1, β2M, and CIITA polypeptides and antigenic fragments thereof.

By “immunogen encoding polynucleotide” is meant a nucleic acid molecule that encodes an immunogen.

By “increases” is meant a positive alteration of at least 10%, 25%, 50%, 75%, or 100%, or about 1.5-fold, about 2-fold, about 3-fold, about 4-fold, about 5-fold, about 6-fold, about 7-fold, about 8-fold, about 9-fold, about 10-fold, about 15-fold, about 20-fold, about 25-fold, about 30-fold, about 35-fold, about 40-fold, about 45-fold, about 50-fold, or about 100-fold.

The terms “inhibitor of base repair”, “base repair inhibitor”, “IBR” or their grammatical equivalents refer to a protein that is capable in inhibiting the activity of a nucleic acid repair enzyme, for example a base excision repair enzyme.

An “intein” is a fragment of a protein that is able to excise itself and join the remaining fragments (the exteins) with a peptide bond in a process known as protein splicing.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of the disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally occurring genome of the organism from which the nucleic acid molecule of the disclosure is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the disclosure that has been separated from components that naturally accompany it. Typically, the polypeptide is isolated when it is at least 60%, by weight, free from the proteins and naturally occurring organic molecules with which it is naturally associated. In embodiments, the preparation is at least 75%, at least 90%, or at least 99%, by weight, a polypeptide of the disclosure. An isolated polypeptide of the disclosure may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “c-JUN kinase 1 (JNK1) polypeptide” is meant a protein having at least about 85% amino acid sequence identity to Uniprot Accession No. P45983, which corresponds to SEQ ID NO: 977 (sp|P45983|MK08_HUMAN Mitogen-activated protein kinase 8 OS=Homo sapiens OX=9606 GN=MAPK8 PE=1 SV=2), or a fragment thereof and having kinase activity.

By “c-JUN kinase 1 (JNK1) polynucleotide” is meant a nucleic acid molecule encoding an JNK1 polypeptide, as well as the introns, exons, 3′ untranslated regions, 5′ untranslated regions, and regulatory sequences associated with its expression, or fragments thereof. In embodiments, a JNK1 polynucleotide is the genomic sequence, cDNA, mRNA, or gene associated with and/or required for JNK1 expression. An exemplary JNK1 polynucleotide sequence from Homo sapiens is provided at GenBank Accession No. BC144063.1, which corresponds to SEQ ID NO: 980 (BC144063.1:63-1217 Homo sapiens mitogen-activated protein kinase 8, mRNA (cDNA clone MGC:177600 IMAGE:9052583), complete cds) and at NCBI Ref. Seq. Accession No. NC_000010.11:48306673-48439360, which is provided in the Sequence Listing as SEQ ID NO: 979.

By “lineage stability” is meant the tendency of an immune cell to avoid differentiating into an alternative cell type. For example, a lineage stabilized regulatory T (T_{REG}) cell has a reduced tendency relative to a reference cell of differentiating into a Th1, Th2, or Th17 cell. In embodiments, the reference cell is an unmodified T_{REG} cell under substantially the same culture or in vivo conditions. In some instances, lineage stability of a T_{REG} cell can be measured using Foxp3 as a marker. For example, in some cases a lineage stabilized T_{REG} cell has higher levels of FoxP3 than a reference cell (e.g., an unmodified T_{REG} cell). In some cases, the FoxP3 expression is increased by at least 10%, 25%, 50%, 75%, or 100%, or about 1.5-fold, about 2-fold, about 3-fold, about 4-fold, about 5-fold, about 6-fold, about 7-fold, about 8-fold, about 9-fold, about 10-fold, about 15-fold, about 20-fold, about 25-fold, about 30-fold, about 35-fold, about 40-fold, about 45-fold, about 50-fold, or about 100-fold. In various embodiments, a lineage stabilized T_{REG} cell has been modified to reduce expression of SIRT1 or RNF20 and increase levels of FoxP3 relative to a reference cell. In some cases, the SIRT1 and/or RNF20 expression is reduced by 10%, 25%, 50%, 75%, or 100%.

The term “linker”, as used herein, refers to a molecule that links two moieties. In one embodiment, the term “linker” refers to a covalent linker (e.g., covalent bond) or a non-covalent linker. The linker can be a peptide or a non-peptide linker. For example, the linker can be an XTEN, (GGGS)ₙ (SEQ ID NO: 246), (GGGGS)ₙ (SEQ ID NO: 247), (G)ₙ, (EAAAK)n (SEQ ID NO: 248), (GGS)ₙ, SGSETPGTSESATPES (SEQ ID NO: 249).

By “major histocompatibility complex, class I, A (HLA-A) polypeptide” is meant a protein having at least about 85% amino acid sequence identity to GenBank. Accession No. BAA07530.1, or a fragment thereof having immunomodulatory activity.

By “major histocompatibility complex, class I, A (HLA-A) polynucleotide” is meant a nucleic acid molecule encoding an HLA-A polypeptide, as well as the introns, exons, 3′ untranslated regions, 5′ untranslated regions, and regulatory sequences associated with its expression, or fragments thereof. In embodiments, an HLA-A polynucleotide is the genomic sequence, cDNA, mRNA, or gene associated with and/or required for HLA-A expression. An exemplary HLA-A polynucleotide sequence is provided at GenBank Accession No. D38525.1. The HLA-A gene corresponds to Ensemble ENSG00000206503.

By “major histocompatibility complex, class I, B (HLA-B) polypeptide” is meant a protein having at least about 85% amino acid sequence identity to GenBank Accession No. CAD30340.1, or a fragment thereof having immunomodulatory activity.

By “major histocompatibility complex, class I, B (HLA-B) polynucleotide” is meant a nucleic acid molecule encoding an HLA-B polypeptide, as well as the introns, exons, 3′ untranslated regions, 5′ untranslated regions, and regulatory sequences associated with its expression, or fragments thereof. In embodiments, an HLA-B polynucleotide is the genomic sequence, cDNA, mRNA, or gene associated with and/or required for HLA-B expression. An exemplary HLA-B polynucleotide sequence is provided at GenBank Accession No. AJ458992.1. The HLA-B gene corresponds to Ensembl: ENSG00000234745.

By “major histocompatibility complex, class I, C (HLA-C) polypeptide” is meant a protein having at least about 85% amino acid sequence identity to GenBank. Accession No. BB094058.1, or a fragment thereof having immunomodulatory activity.

By “major histocompatibility complex, class I, C (HLA-C) polynucleotide” is meant a nucleic acid molecule encoding an HLA-C polypeptide, as well as the introns, exons, 3′ untranslated regions, 5′ untranslated regions, and regulatory sequences associated with its expression, or fragments thereof. In embodiments, an HLA-C polynucleotide is the genomic sequence, cDNA, mRNA, or gene associated with and/or required for HLA-C expression. An exemplary HLA-C polynucleotide sequence is provided at GenBank Accession No. LC508210.1. The HLA-C gene corresponds to Ensembl: ENSG00000204525.

By “MHC class I polypeptide-related sequence A (MICA) polypeptide” is meant a protein having at least about 85% amino acid sequence identity to GenBank Accession No. AAA21718.1, or a fragment thereof having immunomodulatory activity.

By “MHC class I polypeptide-related sequence A (MICA) polynucleotide” is meant a nucleic acid molecule encoding a MICA polypeptide, as well as the introns, exons, 3′ untranslated regions, 5′ untranslated regions, and regulatory sequences associated with its expression, or fragments thereof. In embodiments, a MICA polynucleotide is the genomic sequence, cDNA, mRNA, or gene associated with and/or required for MICA expression. An exemplary MICA polynucleotide sequence is provided at GenBank Accession No. L14848.1. The MICA gene corresponds to Ensembl: ENSG00000204520.

By “MHC class I polypeptide-related sequence B (MICB) polypeptide” is meant a protein having at least about 85% amino acid sequence identity to NCBI Ref. Seq. Accession No. NP_005922.2, or a fragment thereof having immunomodulatory activity.

By “MHC class I polypeptide-related sequence B (MICB) polynucleotide” is meant a nucleic acid molecule encoding an MICB polypeptide, as well as the introns, exons, 3′ untranslated regions, 5′ untranslated regions, and regulatory sequences associated with its expression, or fragments thereof. In embodiments, a MICB polynucleotide is the genomic sequence, cDNA, mRNA, or gene associated with and/or required for MICB expression. An exemplary MICB polynucleotide sequence is provided at NCBI Ref. Seq. Accession No. NM_005931.5. The MICB gene corresponds to Ensembl: ENSG00000204516.

By “marker” is meant any protein or polynucleotide having an alteration in expression, level, structure, or activity that is associated with a disease or disorder. In embodiments, the disease is graft versus host disease (GVHD).

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

By “nectin cell adhesion molecule 2 (Nectin-2) polypeptide” is meant a protein having at least about 85% amino acid sequence identity to NCBI Ref. Seq. Accession No. NP_001036189.1, or a fragment thereof having immunomodulatory activity.

By “nectin cell adhesion molecule 2 (Nectin-2) polynucleotide” is meant a nucleic acid molecule encoding a Nectin-2 polypeptide, as well as the introns, exons, 3′ untranslated regions, 5′ untranslated regions, and regulatory sequences associated with its expression, or fragments thereof. In embodiments, a Nectin-2 polynucleotide is the genomic sequence, cDNA, mRNA, or gene associated with and/or required for Nectin-2 expression. An exemplary Nectin-2 polynucleotide sequence is provided at NCBI Ref. Seq. Accession No. NP_001036189.1. The Nectin-2 gene corresponds to Ensembl: ENSG00000130202.10.

By “NLR family CARD domain containing 5 (class-I transcriptional activator) (NLRC5 (CITA)) polypeptide” is meant a protein having at least about 85% amino acid sequence identity to NCBI Ref. Seq. Accession No. NP_115582.4, or a fragment thereof having immunomodulatory activity.

By “NLR family CARD domain containing 5 (class-I transcriptional activator) (NLRC5 (CITA)) polynucleotide” is meant a nucleic acid molecule encoding an NLRC5 polypeptide, as well as the introns, exons, 3′ untranslated regions, 5′ untranslated regions, and regulatory sequences associated with its expression, or fragments thereof. In embodiments, a NLRC5 polynucleotide is the genomic sequence, cDNA, mRNA, or gene associated with and/or required for NLRC5 expression. An exemplary NLRC5 polynucleotide sequence is provided at NCBI Ref. Seq. Accession No. NM_032206.5. The NLRC5 gene corresponds to Ensembl: ENST00000539144.5.

By “Nerve growth factor receptor (NGFR) polypeptide” is meant a protein having at least about 85% amino acid sequence identity to GenBank Accession No. AAB59544.1 or a fragment thereof that is capable of binding a neutrophin.

By “Nerve growth factor receptor (NGFR) polynucleotide” is meant a nucleic acid molecule encoding an NGFR polypeptide, as well as the introns, exons, 3′ untranslated regions, 5′ untranslated regions, and regulatory sequences associated with its expression, or fragments thereof. In embodiments, a NGFR polynucleotide is the genomic sequence, cDNA, mRNA, or gene associated with and/or required for NGFR expression. An exemplary NGFR polynucleotide sequence from Homo sapiens is provided at GenBank Accession No. M14764.1

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g., nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some instances, a DNA polynucleotide is contained within the genome of a cell and may, therefore, be referred to as “genomic DNA.” In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (2′—e.g., fluororibose, ribose, 2′-deoxyribose, arabinose, and hexose); and/or modified phosphate groups (e.g., phosphorothioates and 5′-N-phosphoramidite linkages).

The term “nuclear localization sequence,” “nuclear localization signal,” or “NLS” refers to an amino acid sequence that promotes import of a protein into the cell nucleus. Nuclear localization sequences are known in the art and described, for example, in Plank et al., International PCT application, PCT/EP2000/011690, filed Nov. 23, 2000, published as WO/2001/038547 on May 31, 2001, the contents of which are incorporated herein by reference for their disclosure of exemplary nuclear localization sequences. In other embodiments, the NLS is an optimized NLS described, for example, by Koblan et al., Nature Biotech. 2018 doi:10.1038/nbt.4172. In some embodiments, an NLS comprises the amino acid sequence KRTADGSEFESPKKKRKV (SEQ ID NO: 190), KRPAATKKAGQAKKKK (SEQ ID NO: 191), KKTELQTTNAENKTKKL (SEQ ID NO: 192), KRGINDRNFWRGENGRKTR (SEQ ID NO: 193), RKSGKIAAIVVKRPRK (SEQ ID NO: 194), PKKKRKV (SEQ ID NO: 195), MDSLLMNRRKFLYQFKNVRWAKGRRETYLC (SEQ ID NO: 196), PKKKRKVEGADKRTADGSE FES PKKKRKV (SEQ ID NO: 328), RKSGKIAAIVVKRPRKPKKKRKV (SEQ ID NO: 329), or MDSLLMNRRKFLYQFKNVRWAKGRRETYLC (SEQ ID NO: 196).

The term “nucleobase,” “nitrogenous base,” or “base,” used interchangeably herein, refers to a nitrogen-containing biological compound that forms a nucleoside, which in turn is a component of a nucleotide. The ability of nucleobases to form base pairs and to stack one upon another leads directly to long-chain helical structures such as ribonucleic acid (RNA) and deoxyribonucleic acid (DNA). Five nucleobases—adenine (A), cytosine (C), guanine (G), thymine (T), and uracil (U)—are called primary or canonical. Adenine and guanine are derived from purine, and cytosine, uracil, and thymine are derived from pyrimidine. DNA and RNA can also contain other (non-primary) bases that are modified. Non-limiting exemplary modified nucleobases can include hypoxanthine, xanthine, 7-methylguanine, 5,6-dihydrouracil, 5-methylcytosine (m5C), and 5-hydromethylcytosine. Hypoxanthine and xanthine can be created through mutagen presence, both of them through deamination (replacement of the amine group with a carbonyl group). Hypoxanthine can be modified from adenine. Xanthine can be modified from guanine. Uracil can result from deamination of cytosine. A “nucleoside” consists of a nucleobase and a five-carbon sugar (either ribose or deoxyribose). Examples of a nucleoside include adenosine, guanosine, uridine, cytidine, 5-methyluridine (m5U), deoxyadenosine, deoxyguanosine, thymidine, deoxyuridine, and deoxycytidine. Examples of a nucleoside with a modified nucleobase includes inosine (I), xanthosine (X), 7-methylguanosine (m7G), dihydrouridine (D), 5-methylcytidine (m5C), and pseudouridine (Ψ). A “nucleotide” consists of a nucleobase, a five-carbon sugar (either ribose or deoxyribose), and at least one phosphate group. Non-limiting examples of modified nucleobases and/or chemical modifications that a modified nucleobase may include are the following: pseudo-uridine, 5-Methyl-cytosine, 2′-O-methyl-3′-phosphonoacetate, 2′-O-methyl thioPACE (MSP), 2′-O-methyl-PACE (MP), 2′-fluoro RNA (2′-F-RNA), constrained ethyl (S-cEt), 2′-O-methyl (‘M’), 2′-O-methyl-3′-phosphorothioate (‘MS’), 2′-O-methyl-3′-thiophosphonoacetate (‘MSP’), 5-methoxyuridine, phosphorothioate, and N1-Methylpseudouridine.

The term “nucleic acid programmable DNA binding protein” or “napDNAbp” may be used interchangeably with “polynucleotide programmable nucleotide binding domain” to refer to a protein that associates with a nucleic acid (e.g., DNA or RNA), such as a guide nucleic acid or guide polynucleotide (e.g., gRNA), that guides the napDNAbp to a specific nucleic acid sequence. In some embodiments, the polynucleotide programmable nucleotide binding domain is a polynucleotide programmable DNA binding domain. In some embodiments, the polynucleotide programmable nucleotide binding domain is a polynucleotide programmable RNA binding domain. In some embodiments, the polynucleotide programmable nucleotide binding domain is a Cas9 protein. A Cas9 protein can associate with a guide RNA that guides the Cas9 protein to a specific DNA sequence that is complementary to the guide RNA. In some embodiments, the napDNAbp is a Cas9 domain, for example a nuclease active Cas9, a Cas9 nickase (nCas9), or a nuclease inactive Cas9 (dCas9). Non-limiting examples of nucleic acid programmable DNA binding proteins include, Cas9 (e.g., dCas9 and nCas9), Cas12a/Cpf1, Cas12b/C2c1, Cas12c/C2c3, Cas12d/CasY, Cas12e/CasX, Cas12g, Cas12h, Cas12i, and Cas12j/CasΦ (Cas12j/Casphi). Non-limiting examples of Cas enzymes include Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (also known as Csn1 or Csx12), Cas10, Cas10d, Cas12a/Cpf1, Cas12b/C2c1, Cas12c/C2c3, Cas12d/CasY, Cas12e/CasX, Cas12g, Cas12h, Cas12i, Cas12j/CasD, Cpf1, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, Type II Cas effector proteins, Type V Cas effector proteins, Type VI Cas effector proteins, CARF, DinG, homologues thereof, or modified or engineered versions thereof. Other nucleic acid programmable DNA binding proteins are also within the scope of this disclosure, although they may not be specifically listed in this disclosure. See, e.g., Makarova et al. “Classification and Nomenclature of CRISPR-Cas Systems: Where from Here?” CRISPR J. 2018 October; 1:325-336. doi: 10.1089/crispr.2018.0033; Yan et al., “Functionally diverse type V CRISPR-Cas systems” Science. 2019 Jan. 4; 363(6422):88-91. doi: 10.1126/science.aav7271, the entire contents of each are hereby incorporated by reference. Exemplary nucleic acid programmable DNA binding proteins and nucleic acid sequences encoding nucleic acid programmable DNA binding proteins are provided in the Sequence Listing as SEQ ID NOs: 197-230, and 378.

The terms “nucleobase editing domain” or “nucleobase editing protein,” as used herein, refers to a protein or enzyme that can catalyze a nucleobase modification in RNA or DNA, such as cytosine (or cytidine) to uracil (or uridine) or thymine (or thymidine), and adenine (or adenosine) to hypoxanthine (or inosine) deaminations, as well as non-templated nucleotide additions and insertions. In some embodiments, the nucleobase editing domain is a deaminase domain (e.g., an adenine deaminase or an adenosine deaminase; or a cytidine deaminase or a cytosine deaminase).

As used herein, “obtaining” as in “obtaining an agent” includes synthesizing, purchasing, or otherwise acquiring the agent.

By “subject” or “patient” is meant a mammal, including, but not limited to, a human or non-human mammal. In embodiments, the mammal is a bovine, equine, canine, ovine, rabbit, rodent, nonhuman primate, or feline. In an embodiment, “patient” refers to a mammalian subject with a higher-than-average likelihood of developing a disease or a disorder. Exemplary patients can be humans, non-human primates, cats, dogs, pigs, cattle, cats, horses, camels, llamas, goats, sheep, rodents (e.g., mice, rabbits, rats, or guinea pigs) and other mammalians that can benefit from the therapies disclosed herein. Exemplary human patients can be male and/or female. “Patient in need thereof” or “subject in need thereof” is referred to herein as a patient diagnosed with, at risk or having, predetermined to have, or suspected of having a disease or disorder.

The terms “pathogenic mutation”, “pathogenic variant”, “disease causing mutation”, “disease causing variant”, “deleterious mutation”, or “predisposing mutation” refers to a genetic alteration or mutation that is associated with a disease or disorder or that increases an individual's susceptibility or predisposition to a certain disease or disorder. In some embodiments, the pathogenic mutation comprises at least one wild-type amino acid substituted by at least one pathogenic amino acid in a protein encoded by a gene. In some embodiments, the pathogenic mutation is in a terminating region (e.g., stop codon). In some embodiments, the pathogenic mutation is in a non-coding region (e.g., intron, promoter, etc.).

By “Programmed cell death 1 (PDCD1; PD-1, PD1) polypeptide” is meant a protein having at least about 85% amino acid sequence identity to GenBank Accession No. AA063583.1, which corresponds to SEQ ID NO. 982 (AA063583.1 programmed cell death 1 [Homo sapiens]), or a fragment thereof having immunomodulatory activity.

By “Programmed cell death 1 (PDCD1; PD-1) polynucleotide” is meant a nucleic acid molecule encoding an PDCD1 polypeptide, as well as the introns, exons, 3′ untranslated regions, 5′ untranslated regions, and regulatory sequences associated with its expression, or fragments thereof. In embodiments, a PDCD1 polynucleotide is the genomic sequence, cDNA, mRNA, or gene associated with and/or required for PDCD1 expression. An exemplary PDCD1 polynucleotide sequence from Homo sapiens is provided at GenBank Accession No. AY238517.1, which corresponds to SEQ ID NO: 983 (AY238517.1 Homo sapiens programmed cell death 1 (PDCD1) mRNA, complete cds), and at NCBI Ref. Seq. NC_000002.12:c241858908-241849881, which is provided in the Sequence Listing as SEQ ID NO: 981.

By “Programmed Cell Death-Ligand 1 (PD-L1)” is meant a protein having at least about 85% amino acid sequence identity to NCBI Accession No. NP_054862.1, or a fragment thereof capable of modulating an immune response.

By “Programmed Cell Death-Ligand 1 (PD-L1)” is meant a nucleic acid molecule encoding an PD-L1 polypeptide, as well as the introns, exons, 3′ untranslated regions, 5′ untranslated regions, and regulatory sequences associated with its expression, or fragments thereof. An exemplary PD-L1 polynucleotide is provided at NCBI Accession No. NM_014143.4. The PD-L1 gene corresponds to Ensembl: ENSG00000120217. By “Protein kinase C theta (PRKCQ) polypeptide” is meant a protein or fragment thereof having at least about 85% amino acid sequence identity to GenBank Accession No. AAI13360.1, which corresponds to SEQ ID NO: 984 (AAI13360.1 Protein kinase C, theta [Homo sapiens]), having kinase activity.

By “Protein kinase C theta (PRKCQ) polynucleotide” is meant a nucleic acid molecule encoding an PRKCQ polypeptide, as well as the introns, exons, 3′ untranslated regions, 5′ untranslated regions, and regulatory sequences associated with its expression, or fragments thereof. In embodiments, a PRKCQ polynucleotide is the genomic sequence, cDNA, mRNA, or gene associated with and/or required for PRKCQ expression. An exemplary PRKCQ polynucleotide sequence from Homo sapiens is provided at GenBank Accession No. BC113359.1, which corresponds to SEQ ID NO: 986 (BC113359.1:20-2140 Homo sapiens protein kinase C, theta, mRNA (cDNA clone MGC:141919 IMAGE:8322411), complete cds), and at NCBI Ref. Seq. NC_000010.11:c6580646-6393038, which is provided in the Sequence Listing as SEQ ID NO: 985.

The terms “protein”, “peptide”, “polypeptide”, and their grammatical equivalents are used interchangeably herein, and refer to a polymer of amino acid residues linked together by peptide (amide) bonds. A protein, peptide, or polypeptide can be naturally occurring, recombinant, or synthetic, or any combination thereof.

The term “fusion protein” as used herein refers to a hybrid polypeptide which comprises protein domains from at least two different proteins.

By “rBE4 polypeptide” is meant a polypeptide sharing at least 85% amino acid sequence identity to SEQ ID NO. 987 and having cytidine base editor activity.

By “rBE4 polynucleotide” is meant a polynucleotide encoding a rBE4 polypeptide.

The term “recombinant” as used herein in the context of proteins or nucleic acids refers to proteins or nucleic acids that do not occur in nature but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

By “reduces” is meant a negative alteration of at least 10%, 25%, 50%, 75%, or 100%. In embodiments, a reduction in is measured as a percent of expression or activity relative to a reference cell (e.g., an unmodified immune cell under substantially the same conditions). In embodiments, the reduced expression or activity is about, or less than about 0.001%, 0.01%, 0.1%, 1%, 2%, 3%, 4%, 5%, 10%, 15%, 20%, 25%, 30%, 35%, 40%, 45%, 50%, 55%, 60%, 65%, 70%, 75%, 80%, 85%, 90%, or 95% of that relative to that in a reference cell. In embodiments, the reduced expression or activity is greater than about 0.001%, 0.01%, 0.1%, 1%, 2%, 3%, 4%, 5%, 10%, 15%, 20%, 25%, 30%, 35%, 40%, 45%, 50%, 55%, 60%, 65%, 70%, 75%, 80%, 85%, 90%, or 95% of that relative to that in a reference cell.

By “reference” is meant a standard or control condition. In one embodiment, the reference is a wild-type or healthy cell (e.g., immune cell, such as a T_{REG} cell). In one embodiment, the reference is an unedited cell (e.g., an immune cell, such as a T_{REG} cell). In embodiments, the reference cell is an unmodified T_{REG} cell under substantially the same culture or in vivo conditions. In other embodiments and without limitation, a reference is an untreated cell that is not subjected to a test condition, or is subjected to placebo or normal saline, medium, buffer, and/or a control vector that does not harbor a polynucleotide of interest.

A “reference sequence” is a defined sequence used as a basis for sequence comparison. A reference sequence may be a subset of or the entirety of a specified sequence, for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, about 35 amino acids, about 50 amino acids, or about 100 amino acids. For nucleic acids, the length of the reference nucleic acid sequence will generally be at least about 50 nucleotides, at least about 60 nucleotides, at least about 75 nucleotides, about 100 nucleotides or about 300 nucleotides or any integer thereabout or therebetween. In some embodiments, a reference sequence is a wild-type sequence of a protein of interest. In other embodiments, a reference sequence is a polynucleotide sequence encoding a wild-type protein.

The term “RNA-programmable nuclease,” and “RNA-guided nuclease” refer to a nuclease that forms a complex with (e.g., binds or associates with) one or more RNA(s) that is not a target for cleavage. In some embodiments, an RNA-programmable nuclease, when in a complex with an RNA, may be referred to as a nuclease: RNA complex. Typically, the bound RNA(s) is referred to as a guide RNA (gRNA). In some embodiments, the RNA-programmable nuclease is the (CRISPR-associated system) Cas9 endonuclease, for example, Cas9 (Csn1) from Streptococcus pyogenes (e.g., SEQ ID NO: 197), Cas9 from Neisseria meningitidis (NmeCas9; SEQ ID NO: 208), Nme2Cas9 (SEQ ID NO: 209), Streptococcus constellatus (ScoCas9) or derivatives thereof (e.g., a sequence with at least about 85% sequence identity to a Cas9, such as Nme2Cas9 or spCas9).

By “Ring finger protein 20 (RNF20) polypeptide” is meant a protein or fragment thereof having at least about 85% amino acid sequence identity to GenBank Accession No. AA152310.1, which corresponds to SEQ ID NO: 988 (AA152310.1 Ring finger protein 20 [Homo sapiens]), and having ubiquitin ligase activity. RNF20 is a modulator of FoxP3 expression acting as a negative regulator of FoxP3. RNF20 is an E3 ubiquitin ligase. By “Ring finger protein 20 (RNF20) polynucleotide” is meant a nucleic acid molecule encoding an RNF20 polypeptide, as well as the introns, exons, 3′ untranslated regions, 5′ untranslated regions, and regulatory sequences associated with its expression, or fragments thereof. In embodiments, a RNF20 polynucleotide is the genomic sequence, cDNA, mRNA, or gene associated with and/or required for RNF20 expression. An exemplary RNF20 polynucleotide sequence from Homo sapiens is provided at GenBank Accession No. BC152309.1, which corresponds to SEQ ID NO: 990 (BC152309.1:86-3013 Homo sapiens ring finger protein 20, mRNA (cDNA clone MGC:133065 IMAGE:40009034), complete cds), and at NCBI Ref. Seq. NG_047002.1, which is provided in the Sequence Listing as SEQ ID NO: 989.

As used herein, the term “scFv” refers to a single chain Fv antibody in which the variable domains of the heavy chain and the light chain from an antibody have been joined to form one chain. scFv fragments contain a single polypeptide chain that includes the variable region of an antibody light chain (VL) (e.g., CDR-L1, CDR-L2, and/or CDR-L3) and the variable region of an antibody heavy chain (VH) (e.g., CDR-H1, CDR-H2, and/or CDR-H3) separated by a linker. The linker that joins the VL and VH regions of a scFv fragment can be a peptide linker composed of proteinogenic amino acids. Alternative linkers can be used to so as to increase the resistance of the scFv fragment to proteolytic degradation (for example, linkers containing D-amino acids), in order to enhance the solubility of the scFv fragment (for example, hydrophilic linkers such as polyethylene glycol-containing linkers or polypeptides containing repeating glycine and serine residues), to improve the biophysical stability of the molecule (for example, a linker containing cysteine residues that form intramolecular or intermolecular disulfide bonds), or to attenuate the immunogenicity of the scFv fragment (for example, linkers containing glycosylation sites). It will also be understood by one of ordinary skill in the art that the variable regions of the scFv molecules described herein can be modified such that they vary in amino acid sequence from the antibody molecule from which they were derived. For example, nucleotide or amino acid substitutions leading to conservative substitutions or changes at amino acid residues can be made (e.g., in CDR and/or framework residues) so as to preserve or enhance the ability of the scFv to bind to the antigen recognized by the corresponding antibody.

The term “single nucleotide polymorphism (SNP)” is a variation in a single nucleotide that occurs at a specific position in the genome, where each variation is present to some appreciable degree within a population (e.g., >1%). SNPs can fall within coding regions of genes, non-coding regions of genes, or in the intergenic regions (regions between genes). In some embodiments, SNPs within a coding sequence do not necessarily change the amino acid sequence of the protein that is produced, due to degeneracy of the genetic code. SNPs in the coding region are of two types: synonymous and nonsynonymous SNPs. Synonymous SNPs do not affect the protein sequence, while nonsynonymous SNPs change the amino acid sequence of protein. The nonsynonymous SNPs are of two types: missense and nonsense. SNPs that are not in protein-coding regions can still affect gene splicing, transcription factor binding, messenger RNA degradation, or the sequence of noncoding RNA. Gene expression affected by this type of SNP is referred to as an eSNP (expression SNP) and can be upstream or downstream from the gene. A single nucleotide variant (SNV) is a variation in a single nucleotide without any limitations of frequency and can arise in somatic cells. A somatic single nucleotide variation can also be called a single-nucleotide alteration.

By “specifically binds” is meant a nucleic acid molecule, polypeptide, polypeptide/polynucleotide complex, compound, or molecule that recognizes and binds a polypeptide and/or nucleic acid molecule of the disclosure, but which does not substantially recognize and bind other molecules in a sample, for example, a biological sample.

By “Sirtuin 1 (SIRT1) polypeptide” is meant a protein having at least about 85% amino acid sequence identity to GenBank Accession No. AAH12499.1, which corresponds to SEQ ID NO: 991 (AAH12499.1 SIRT1 protein [Homo sapiens]), or a fragment thereof having nicotinamide adenine dinucleotide-dependent histone deacetylase activity. SIRT1 targets histone and non-histone proteins and can function as a transcription factor.

By “Sirtuin 1 (SIRT1) polynucleotide” is meant a nucleic acid molecule encoding an SIRT1 polypeptide, as well as the introns, exons, 3′ untranslated regions, 5′ untranslated regions, and regulatory sequences associated with its expression, or fragments thereof. In embodiments, a SIRT1 polynucleotide is the genomic sequence, cDNA, mRNA, or gene associated with and/or required for SIRT1 expression. An exemplary SIRT1 polynucleotide sequence from Homo sapiens is provided at GenBank Accession No. BC012499.1, which corresponds to SEQ ID NO: 993 (BC012499.1:210-1877 Homo sapiens sirtuin (silent mating type information regulation 2 homolog) 1 (S. cerevisiae), mRNA (cDNA clone MGC:21066 IMAGE:4518906), complete cds), and at NCBI Ref. Seq. NC_000010.11:67884656-67918390, which is provided in the Sequence Listing as SEQ ID NO: 992.

By “substantially identical” is meant a polypeptide or nucleic acid molecule exhibiting at least 50% identity to a reference amino acid sequence. In one embodiment, a reference sequence is a wild-type amino acid or nucleic acid sequence. In another embodiment, a reference sequence is any one of the amino acid or nucleic acid sequences described herein. In one embodiment, such a sequence is at least about 60%, 80%, 85%, 90%, 95%, 96%, 97%, 98%, 99%, 99.9%, or even 99.99%, identical at the amino acid level or nucleic acid level to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

Nucleic acid molecules useful in the methods of the disclosure include any nucleic acid molecule that encodes a polypeptide of the disclosure or a functional fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the disclosure include any nucleic acid molecule that encodes a polypeptide of the disclosure or a functional fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

By “split” is meant divided into two or more fragments.

A “split polypeptide” or “split protein” refers to a protein that is provided as an N-terminal fragment and a C-terminal fragment translated as two separate polypeptides from a nucleotide sequence(s). The polypeptides corresponding to the N-terminal portion and the C-terminal portion of the split protein may be spliced in some embodiments to form a “reconstituted” protein. In embodiments, the split polypeptide is a nucleic acid programmable DNA binding protein (e.g. a Cas9) or a base editor.

By “TAP-associated glycoprotein (Tapasin; TAPBP) polypeptide” is meant a protein having at least about 85% amino acid sequence identity to NCBI Ref. Seq. Accession No. NP_003181.3, or a fragment thereof having immunomodulatory activity.

By “TAP-associated glycoprotein (Tapasin; TAPBP) polynucleotide” is meant a nucleic acid molecule encoding an Tapasin polypeptide, as well as the introns, exons, 3′ untranslated regions, 5′ untranslated regions, and regulatory sequences associated with its expression, or fragments thereof. In embodiments, a Tapasin polynucleotide is the genomic sequence, cDNA, mRNA, or gene associated with and/or required for Tapasin expression. An exemplary Tapasin polynucleotide sequence is provided at NCBI Ref. Seq. Accession No. NM_003190.5. The tapasin gene corresponds to Ensembl: ENSG00000231925, ENSG00000236490, ENSG00000206281, ENSG00000206208, and ENSG00000112493.

By “T Cell Receptor Alpha Constant (TRAC) polypeptide” is meant a protein having at least about 85% amino acid sequence identity to UniProtKB/Swiss-Prot Accession No. P01848.2, or a fragment thereof having immunomodulatory activity.

By “T Cell Receptor Alpha Constant (TRAC) polynucleotide” is meant a nucleic acid molecule encoding an TRAC polypeptide, as well as the introns, exons, 3′ untranslated regions, 5′ untranslated regions, and regulatory sequences associated with its expression, or fragments thereof. In embodiments, a TRAC polynucleotide is the genomic sequence, cDNA, mRNA, or gene associated with and/or required for TRAC expression. An exemplary TRAC polynucleotide is provided at Ensembl: ENSG00000277734.8.

By “Transporter associated with antigen processing I (TAP1) polypeptide” is meant a protein having at least about 85% amino acid sequence identity to NCBI Ref. Seq. Accession No. NP_000584.3, or a fragment thereof having immunomodulatory activity.

By “Transporter associated with antigen processing I (TAP1) polynucleotide” is meant a nucleic acid molecule encoding an TAP1 polypeptide, as well as the introns, exons, 3′ untranslated regions, 5′ untranslated regions, and regulatory sequences associated with its expression, or fragments thereof. In embodiments, a TAP1 polynucleotide is the genomic sequence, cDNA, mRNA, or gene associated with and/or required for TAP1 expression. An exemplary TAP1 polynucleotide sequence is provided at NCBI Ref. Seq. Accession No. NM_000593.6. The TAP1 gene corresponds to Ensembl: ENSG00000168394.

By “Transporter associated with antigen processing II (TAP2) polypeptide” is meant a protein having at least about 85% amino acid sequence identity to NCBI Ref. Seq. Accession No. NP_000535.3, or a fragment thereof having immunomodulatory activity.

By “Transporter associated with antigen processing II (TAP2) polynucleotide” is meant a nucleic acid molecule encoding an TAP2 polypeptide, as well as the introns, exons, 3′ untranslated regions, 5′ untranslated regions, and regulatory sequences associated with its expression, or fragments thereof. In embodiments, a TAP2 polynucleotide is the genomic sequence, cDNA, mRNA, or gene associated with and/or required for TAP2 expression. An exemplary TAP2 polynucleotide sequence is provided at NCBI Ref. Seq. Accession No. NM_000544.3. The TAP2 gene corresponds to Ensembl: ENSG00000204267.

The term “target site” refers to a nucleotide sequence or nucleobase of interest within a nucleic acid molecule that is modified. In embodiments, the modification is deamination of a base. The deaminase can be a cytidine or an adenine deaminase. The fusion protein or base editing complex comprising a deaminase may comprise a dCas9-adenosine deaminase fusion protein, a Cas12b-adenosine deaminase fusion, or a base editor disclosed herein.

By “T cell receptor beta constant 1 polypeptide (TRBC1) polypeptide” is meant a protein having at least about 85% amino acid sequence identity to UniProtKB/Swiss-Prot. Accession No. P01850, or a fragment thereof having immunomodulatory activity.

By “T cell receptor beta constant 1 polypeptide (TRBC1) polynucleotide” is meant a nucleic acid molecule encoding a TRBC1 polypeptide, as well as the introns, exons, 3′ untranslated regions, 5′ untranslated regions, and regulatory sequences associated with its expression, or fragments thereof. In embodiments, a TRBC1 polynucleotide is the genomic sequence, cDNA, mRNA, or gene associated with and/or required for TRBC1 expression. An exemplary TRBC1 polynucleotide sequence is provided at GenBank Accession No. X00437.1. The TRBC1 gene corresponds to Ensembl: ENSG00000211751.

By “T cell receptor beta constant 2 (TRBC2) polypeptide” is meant a protein having at least about 85% amino acid sequence identity to UniProtKB/Swiss-Prot Accession No. A0A5B9, or a fragment thereof having immunomodulatory activity.

By “T cell receptor beta constant 2 (TRBC2) polynucleotide” is meant a nucleic acid molecule encoding a TRBC2 polypeptide, as well as the introns, exons, 3′ untranslated regions, 5′ untranslated regions, and regulatory sequences associated with its expression, or fragments thereof. In embodiments, a TRBC2 polynucleotide is the genomic sequence, cDNA, mRNA, or gene associated with and/or required for TRBC2 expression. An exemplary TRBC2 polynucleotide sequence is provided at NCBI Ref. Seq. Accession No. NG_001333.2:655095-656583. The TRBC2 gene corresponds to Ensembl: ENSG00000211772.

As used herein, the terms “treat,” “treating,” “treatment,” and the like refer to reducing or ameliorating a disorder and/or symptoms associated therewith or obtaining a desired pharmacologic and/or physiologic effect. It will be appreciated that, although not precluded, treating a disorder or condition does not require that the disorder, condition, or symptoms associated therewith be completely eliminated. In some embodiments, the effect is therapeutic, i.e., without limitation, the effect partially or completely reduces, diminishes, abrogates, abates, alleviates, decreases the intensity of, or cures a disease and/or adverse symptom attributable to the disease. In some embodiments, the effect is preventative, i.e., the effect protects or prevents an occurrence or reoccurrence of a disease or condition. To this end, the presently disclosed methods comprise administering a therapeutically effective amount of a composition as described herein.

By “regulatory T (T_{REG}) cell” is meant an immune cell that acts to suppress an immune response. In embodiments, T_{REG} cells inhibit T cell proliferation and/or cytokine production. In embodiments, T_{REG} cells have the immunophenotype CD4⁺, CD25⁺, and CD127⁻. In embodiments, the T_{REG} cells have one or more of the following immunophenotypes: CD3⁺, CD4⁺, CD5⁺, CD14⁻, CD19⁻, CD25/IL-2 R alpha⁺, CD39/ENTPD1⁺, 5′ Nucleotidase/CD73⁺, CD103/Integrin alpha E⁺, CD127⁺, CTLA-4⁺, Folate Receptor 4⁺, FoxP3⁺, LRRC32/GARP⁺, GITR⁺, IL-7 R alpha/CD127^{low}, Helios^{+/-}, LAG-3/CD223⁺, LAP⁺, Neuropilin-1/BDCA-4⁺, OX40/CD134⁺, L-Selectin/CD26L⁺, and STAT5+. In some instances, a T_{REG} cell secretes one or more cytokines selected from Galectin-1, TGF-beta, IL-10, and IL-35. In some cases, the T_{REG} cells have an immunophenotype detected as the presence or absence of one or more of the following markers: CD8, CD28, CD45RA, CD45RO, Qa-1, HLA-E, or any polypeptide targeted by and/or edited using the base editors and methods provided herein.

By “UL16 binding protein 1-6 (ULBP) polypeptide” is meant a protein having at least about 85% amino acid sequence identity to GenBank Accession No. AAK13081.1 (ULBP1), AAK13082.1 (ULBP2), AAK13083.1 (ULBP3), or AVP72463.1 (ULBP4), or NCBI Ref. Seq. No. NP_001001788.2 (ULBP5) or NP_570970.2 (ULBP6), or a fragment thereof having immunomodulatory activity.

By “UL16 binding protein 1-6 (ULBP) polynucleotide” is meant a nucleic acid molecule encoding an ULBP polypeptide, as well as the introns, exons, 3′ untranslated regions, 5′ untranslated regions, and regulatory sequences associated with its expression, or fragments thereof. In embodiments, a ULBP polynucleotide is the genomic sequence, cDNA, mRNA, or gene associated with and/or required for ULBP expression. An exemplary ULBP polynucleotide sequence is provided at GenBank Accession Nos. AF304379.1 (ULBP3), AF304378.1 (ULBP2), AF304377.1 (ULBP1), MH020173.1 (ULBP4), and NCBI Ref. Seq. Nos. NM_001001788.4 (ULBP5) and NM_130900.3 (ULBP6). ULBP1 gene corresponds to Ensembl: ENSG00000111981; ULBP2 gene corresponds to Ensembl: ENSG00000131015; ULBP3 gene corresponds to Ensembl: ENSG00000131019; ULBP4 gene corresponds to Ensembl: ENSG00000164520; ULBP5 gene corresponds to Ensembl: ENSG00000203722; and ULBP6 corresponds to Ensembl: ENSG00000155918.

By “uracil glycosylase inhibitor” or “UGI” is meant an agent that inhibits the uracil-excision repair system. Base editors comprising a cytidine deaminase convert cytosine to uracil, which is then converted to thymine through DNA replication or repair. In various embodiments, a uracil DNA glycosylase (UGI) prevent base excision repair which changes the U back to a C. In some instances, contacting a cell and/or polynucleotide with a UGI and a base editor prevents base excision repair which changes the U back to a C. An exemplary UGI comprises an amino acid sequence as follows:

>sp|P14739|UNGI_BPPB2 Uracil-DNA glycosylase
inhibitor
(SEQ ID NO: 231)
MTNLSDIIEKETGKQLVIQESILMLPEEVEEVIGNKPESDILVHTAYDE
STDENVMLLTSDAPEYKPWALVIQDSNGENKIKML.

In some embodiments, the agent inhibiting the uracil-excision repair system is a uracil stabilizing protein (USP). See, e.g., WO 2022015969 A1, incorporated herein by reference.

As used herein, the term “vector” refers to a means of introducing a nucleic acid sequence into a cell, resulting in a transformed cell. Vectors include plasmids, transposons, phages, viruses, liposomes, lipid nanoparticles, and episomes.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All terms are intended to be understood as they would be understood by a person skilled in the art. Unless defined otherwise, all technical and scientific terms used herein have the same meaning as commonly understood by one of ordinary skill in the art to which the disclosure pertains

In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended. This wording indicates that specified elements, features, components, and/or method steps are present, but does not exclude the presence of other elements, features, components, and/or method steps. Any embodiments specified as “comprising” a particular component(s) or element(s) are also contemplated as “consisting of” or “consisting essentially of” the particular component(s) or element(s) in some embodiments. It is contemplated that any embodiment discussed in this specification can be implemented with respect to any method or composition of the present disclosure, and vice versa. Furthermore, compositions of the present disclosure can be used to achieve methods of the present disclosure.

The term “about” or “approximately” means within an acceptable error range for the particular value as determined by one of ordinary skill in the art, which will depend in part on how the value is measured or determined, i.e., the limitations of the measurement system.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

### BRIEF DESCRIPTION OF THE DRAWINGS

FIG. 1 provides a schematic diagram providing an overview of the preparation of peripheral blood mononuclear cells (PBMCs) and regulatory T cells (T_{REG}'S) for expansion and/or editing. The preparation involved seven steps: 1) Receive fresh leukopak (HemaCare); 2) Process leukopak by ficoll density gradient centrifugation to isolate PBMCs; 3) Cryopreserve PBMCs for convenient use; 4) Thaw cryopreserved PBMCs; 5) Isolate CD4+ T cells by magnetic selection (i.e., CD4 Microbeads, Miltenyi); 6) Stain CD4+ T cells with fluorochrome conjugated antibodies and FACS sort; and 7) Sort T_{REG} cells: Viable/CD4⁺/CD25⁺/CD127⁻. T_{REG} cells were sorted out based upon high CD4 expression, high CD25 expression, and low CD127 expression. The gating strategy used to select for viable T_{REG} cells is presented in the lower portion of FIG. 1 as a series of representative flow cytometry scatter plots.

FIG. 2 provides a schematic diagram showing an overview of a multiplex base editing strategy to knock-out surface receptors implicated in T_{REG} function. The HLA specific CAR of the modified Treg cells binds to donor Tconv cells, reducing or preventing GVHD. FIG. 2 shows how disruption of surface receptors can be used to improve chimeric antigen receptor (CAR) T_{REG} function. PD-1 is an inhibitory receptor that dampens T_{REG} activation. CD70 is a costimulatory ligand for CD27, which is widely expressed on conventional T cells (T_{CONV}). CD58 is a costimulatory ligand for CD2, which is widely expressed on (T_{CONV}). Knockout of CD70 and CD58 can reduce potential activation of T_{CONV} by the edited T_{REG}.

FIGS. 3A and 3B provide schematic diagrams showing an overview of how T_{REG} cell function can be improved by editing polynucleotides encoding T_{CONV} costimulatory ligands to reduce the expression thereof in the T_{REG} cells. FIG. 3A provides a schematic diagram showing how the CD27/CD70 signaling axis promotes T_{CONV} activation and proliferation. FIG. 3B provides a schematic diagram showing how CD70 expression by T_{REG} can counter suppressive activity of the T_{REG} by binding a CD27 costimulatory receptor on T_{CONV}. In embodiments, the T_{REG} cells are chimeric antigen receptor (CAR) T_{REG} cells and surface-express a CAR that targets an HLA.

FIGS. 4A-4D provide flow cytometry scatter plots showing that CD70+ T_{REG} cells exhibited a unique phenotype as compared to CD70− counterparts. FIG. 4A provides a flow cytometry scatter plot showing D14 of expansion for a population containing edited (CD70−) and unedited (CD70+) cells, which are the same edited and unedited cells that were evaluated in FIGS. 4B-4D. FIG. 4B provides flow cytometry scatter plots showing that CD70− cells co-expressed FoxP3 and helios, which are both markers of T_{REG} function. CD70− cells had higher expression levels of the transcription factors FoxP3 and helios, which was consistent with the knockout of CD70 improving T_{REG} cell function. FIG. 4C provides flow cytometry scatter plots showing that CD70− cells expressed higher levels of CTLA4 than CD70+ cells.

FIG. 4D provides flow cytometry scatter plots showing that CD70− cells (left of plot) expressed higher levels of GARP and FoxP3 than CD70+ cells (right of plot) when stimulated with T Cell TransAct™, which is a reagent used to activate and expand human T cells via CD3 and CD28.

FIGS. 5A and 5B provide bar graphs showing maximum editing of CD70 using the indicated guides (see Tables 1A-2C). FIG. 5A is a bar graph showing total on-target editing A4G and C4T editing using the indicated guides. FIG. 5B shows total bystander A4G and C4T editing using the indicated guides.

FIG. 6 provides flow cytometry scatter plots showing successful CD70 knock-out in T_{REG} cells using base editing. A large reduction in CD70 expression was observed in base-edited cells.

FIGS. 7A and 7B provide flow cytometry scatter plots and FIG. 7C provides a bar graph showing that CD70 knockout (KO) T_{REG} cells were phenotypically indistinguishable from unedited control cells. The edited cells showed little-to-no reduction in FoxP3. Limited evidence of CD7-CD70 reverse signaling was observed. In FIG. 7C, “Untx” represents “untransduced cells.”

FIGS. 8A and 8B provide a schematic diagram and a collection of histograms showing that disrupting CD70 in T_{REG} cells inhibited T_{CONV} costimulation. FIG. 8A provides a schematic diagram showing how CD70 of T_{REG} cells binds to CD27 in T_{CONV} cells leading to the activation of the T_{CONV} cells by inducing conventional T-cell costimulation. Knockout of CD70 in T_{REG} cells prevents activation of T_{CONV} cells by CD70 and leads to a reduction in T_{CONV} proliferation in the presence of the T_{REG} cells, as shown. FIG. 8B provides histograms showing dilution (i.e., lowering of fluorescence intensity as indicated on the x-axis) of CellTrace™ Violet dye signal peak (i.e., cell proliferation) in T_{CONV} cultured in the presence of unedited and CD70 knockout (KO) T_{REG}. Each cell division led to a reduction by about half of the CellTrace™ Violet dye signal. Lower levels of T_{CONV} proliferation were observed in the presence of edited T_{REG} cells than in the presence of unedited T_{REG} cells.

FIG. 9 provides a bar graph showing max on-target A4G base editing of CD58 using the indicated guide RNA (see Tables 1A-2C).

FIGS. 10A-10D provide flow cytometry scatter plots and a schematic diagram showing that inhibitory receptor (i.e., PD-1) editing enhanced T_{REG} cell function. PD-1 attenuates T_{CONV} activation by dephosphorylating kinases in the T cell receptor (TCR) signal cascade. The PD-1/PD-L1 axis restrains T_{REG} activation. FIG. 10A provides a flow cytometry scatter plot confirming low surface expression of PD-1 in edited T_{REG} cells. FIG. 10B provides a schematic diagram showing that knockout of PD-1 in the T_{REG} cells prevents inhibition of the T_{REG} cells by PD-L1. FIG. 10C provides a flow cytometry scatter plot showing PD-1 expression in unedited T_{REG} and T_{CONV} cells. FIG. 10D provides a flow cytometry scatter plot showing PD-L1 expression in T cells from spleen and bone marrow samples.

FIG. 11 provides a bar graph showing max on-target A4G editing of PD-1 in T_{REG} cells using the indicated guide RNA (see Tables 1A-2C).

FIG. 12 provides a bar graph showing max on-target A4G editing of SIRT1 in T_{REG} cells using the indicated guide RNA (see Tables 1A-2C). Knockout of SIRT1 expression promotes expression of FoxP3, an important transcription factor in T_{REG} cells. Histones are involved in silencing FoxP3 expression, so, in some embodiments, knockout of SIRT1 can reduce or eliminate such silencing.

FIG. 13 provides a bar graph showing maximum A→G and C→T editing of SIRT1 in T_{CONV} cells using the indicated guide RNAs (see Tables 1A-2C).

FIG. 14 provides a bar graph showing maximum on-target A→G and C→T editing of B2M in T_{REG} cells using the indicated guide RNAs (see Tables 1A-2C).

FIG. 15 provides a bar graph showing maximum A→G and C→T editing of RNF20 in T cells using the indicated guide RNAs (see Tables 1A-2C).

FIG. 16 provides a bar graph showing maximum on-target A→G editing of RNF20 in T_{REG} using the indicated guide RNAs (see Tables 1A-2C).

FIGS. 17A and 17B provide flow cytometry scatter plots and a bar graph showing that RNF20 edited T_{REG} cells had increased expression of FoxP3. FIGS. 17A and 17B provide scatter plots and a bar graph showing that RNF20 edited T_{REG} cells showed a shift in FoxP3 mean fluorescence intensity after editing using guide RNAs (see Tables 1A-2C). In FIG. 17A, the x-axis indicates level of FoxP3, and the y-axis indicates the level of Helios. In FIG. 17B, the x-axis identifies the guide and editor, and the y-axis indicates the geometric mean MFI.

FIG. 18 provides a schematic diagram showing how chimeric antigen receptor (CAR) T regulatory cell (T_{REG} or T_{REG} cell) lineage stability and suppressive function can be improved through multiplex base editing to knock-out surface receptors implicated in T_{REG} function. In embodiments, multiplex editing involves editing of one or more of SIRT1, RNF20, PD-1, CD70, CD58, and B2M. The editing results in the reduction or elimination of expression and/or activity of the target gene.

FIG. 19 provides a bar graph showing 5-plex multiplex editing of T_{REG} cells using the indicated guide RNAs (see Tables 1A-2C) to knockout SIRT1, PD-1, CD70, CD58, and B2M.

FIG. 20 provides a collection of flow cytometry scatter plots showing successful multiplex base-editing of T_{REG} cells to reduce and/or eliminate surface-expression of CD58, CD70, PD-1, and B2M. The left panel of FIG. 20 illustrates a gating strategy used to sort for T_{REG} cells surface-expression anti-NGFR chimeric antigen receptors (i.e., NGFR+). The right panel of FIG. 20 shows measured surface-expression of the indicated target genes (i.e., CD58, CD70, PD-1, and B2M) in edited and unedited anti-NGFR-CAR T_{REG} cells.

### DETAILED DESCRIPTION

The disclosure features modified regulatory T (T_{REG}) cells (e.g., chimeric antigen receptor (CAR) T_{REG} cells) having increased lineage stability, decreased activation of T cells (e.g., conventional T (T_{CONV}) cells), and/or increased resistance to immune rejection (i.e., functionally enhanced and/or lineage stabilized regulatory T (T_{REG}) cells), and methods of producing and using such cells, for example, in the treatment of graft versus host disease (GVHD).

The embodiments of the disclosure are based, at least in part, on the discovery that the functionality and lineage stability of CAR T_{REG} cells can be increased by modifying one or more genes encoding BRINP1, CD58, CD70, CHIP, JNK1, PRKCQ, PD-1, RNF20, and/or SIRT1 to reduce activity and/or expression of the encoded polypeptides in the cells. PD-1 is an inhibitory receptor that dampens T cell activation, so reducing expression and/or activity of PD-1 in a T_{REG} cell can improve function of the cell by eliminating such dampening. CD70 and CD58 are costimulatory ligands for CD27 and CD2, respectively, that are expressed on the surface of conventional T (T_{CONV}) cells. Therefore, eliminating activity and/or expression of CD70 and/or CD58 in the T_{REG} cells prevents stimulation/activation of T_{CONV} cells (e.g., proliferation). In some embodiments, reducing expression and/or activity of BRINP1, CHIP, JNK1, PRKCQ, RNF20, and/or SIRT1 is associated with an increase in T_{REG} lineage stability and an increase in expression of FoxP3.

Accordingly, the disclosure provides modified CAR T_{REG} cells comprising one or more gene modifications that result in improved lineage stability and/or suppressive functionality. The disclosure also provides methods for treatment of alloimmune (e.g., graft versus host disease) or autoimmune diseases using the T_{REG} cells. The T_{REG} cells are suitable in embodiments for the treatment of any disease associated with an undesired immune activity or response (e.g., to increase organ transplant tolerance). In various instances, the methods of the disclosure stabilize or increase FoxP3 expression in a T_{REG} cell and reduce pathogenic T_{REG}-to-Th17 transition in response to stimulation.

In some embodiments, the CAR-T_{REG} cells have increased lineage stability and/or enhanced functionality (e.g., less activation of T_{CONV} cells and/or lower allorecognition) as compared to a similar CAR-T_{REG} cell without further modifications to one or more genes as described herein. In some embodiments, the CAR-T_{REG} cells have reduced immunogenicity as compared to a similar CAR-T_{REG} cell without further modifications to one more genes as described herein. In some embodiments, the CAR-T_{REG} cells have increased T_{CONV} cell inhibition activity as compared to a similar CAR-T_{REG} cell without further modifications to one or more genes as described herein.

The one or more genes may be edited by base editing or through use of a nuclease (e.g., a Cas12b). In some embodiments the one or more genes, or one or more regulatory elements thereof, or combinations thereof, may be selected from a group consisting of: BRINP1, JNK1, PRKCQ, CHIP, CD70, CD58, PD-1, SIRT1, and RNF20. In some embodiments, the one or more genes, or regulatory elements thereof, comprise a combination of targets including one or more of BRINP1, CHIP, JNK1, PRKCQ, SIRT1, and RNF20, and one or more of PD-1, CD70, and CD58. In embodiments, the combination of targets further includes β2M (B2M). In some embodiments, the one or more genes comprise a combination of targets selected from the following: SIRT1, PD-1, CD70, and CD58; SIRT1, PD-1, and CD70; SIRT1, PD-1, and CD58; SIRT1, CD70, and CD58; SIRT1 and PD-1; SIRT1 and CD70; SIRT1 and CD58; SIRT1, PD-1, CD70, CD58, and B2M; SIRT1, PD-1, CD70, and B2M; SIRT1, PD-1, CD58 and B2M; SIRT1, CD70, CD58, and B2M; SIRT1, PD-1, and B2M; SIRT1, CD70, and B2M; SIRT1, CD58, and B2M; RNF20, PD-1, CD70, and CD58; RNF20, PD-1, and CD70; RNF20, PD-1, and CD58; RNF20, CD70, and CD58; RNF20 and PD-1; RNF20 and CD70; RNF20 and CD58; RNF20, PD-1, CD70, CD58, and B2M; RNF20, PD-1, CD70, and B2M; RNF20, PD-1, CD58 and B2M; RNF20, CD70, CD58, and B2M; RNF20, PD-1, and B2M; RNF20, CD70, and B2M; RNF20, CD58, and B2M; SIRT1, RNF20, PD-1, CD70, and CD58; SIRT1, RNF20, PD-1, and CD70; SIRT1, RNF20, PD-1, and CD58; SIRT1, RNF20, CD70, and CD58; SIRT1, RNF20, and PD-1; SIRT1, RNF20, and CD70; SIRT1, RNF20, and CD58; SIRT1, RNF20, PD-1, CD70, CD58, and B2M; SIRT1, RNF20, PD-1, CD70, and B2M; SIRT1, RNF20, PD-1, CD58, and B2M; SIRT1, RNF20, CD70, CD58, and B2M; SIRT1, RNF20, PD-1, and B2M; SIRT1, RNF20, CD70, and B2M; and SIRT1, RNF20, CD58, and B2M. In some instances, the combination of targets includes one or more of TAP1, TAP2, Tapasin, NLRC5, CD155, HLA-A, HLA-B, HLA-C, MICA, MICB, Nectin-2, TRAC, ULBP, CIITA, TRBC1, TRBC2, and CD52.

In various instances, the methods of the present disclosure involve overexpressing in the cell an inhibitory receptor, or fragment thereof, selected from one or more of Human Leukocyte Antigen-E (HLA-E), Human Leukocyte Antigen-G (HLA-G), Programmed Death Ligand 1 (PD-L1), and Cluster of Differentiation 47 (CD47). In some instances, the methods of the present disclosure involve modifying an immune cell (e.g., a T_{REG} cell) to increase or cause expression of one or more polypeptides selected from HLA-E, HLA-G, PD-L1, and/or CD47 (e.g., by editing a promoter or regulatory sequence thereof, or by introducing into the cell a polynucleotide encoding the polypeptide(s)). Expression of one or more of these polypeptides can increase the persistence of a modified immune cell in a subject.

### Editing of Target Genes

To produce the gene edits described herein, T_{REG} cells are collected from a subject and contacted with one, two, or more guide RNAs and a nucleobase editor polypeptide comprising a nucleic acid programmable DNA binding protein (napDNAbp) and a cytidine deaminase or adenosine deaminase, or comprising one or more deaminases with cytidine deaminase and/or adenosine deaminase activity. In some embodiments, cells to be edited are contacted with at least one nucleic acid, wherein the at least one nucleic acid encodes two or more guide RNAs and a nucleobase editor polypeptide comprising a nucleic acid programmable DNA binding protein (napDNAbp) and a cytidine deaminase. In some embodiments, the guide RNA comprises nucleotide analogs. In various instances, the gRNA is added directly to a cell. These nucleotide analogs can inhibit degradation of the gRNA from cellular processes. Tables 2A-2C provide target sequences to be used for gRNAs. Exemplary guide RNAs are provided in Tables 1A-1C. Further non-limiting examples of target sequences, spacer, sequences, and guide RNAs suitable for use in the compositions and methods of the present disclosure include those described in PCT/US20/13964, PCT/US20/52822, PCT/US20/18178, and/or PCT/US21/52035.

In embodiments, the guide RNAs comprise a scaffold sequence. Non-limiting examples of scaffold sequences include the following:

GUUUUAGAGCUAGAAAUAGCAAGUUAAAAUAAGGCUAGUCCGUUAUCAA
CUUGAAAAAGUGGCACCGAGUCGGUGCUUUU (Base Editor
(BE) Scaffold Sequence; SEQ ID NO: 994);
and
GUUCUGUCUUUUGGUCAGGACAACCGUCUAGCUAUAAGUGCUGCAGGGU
GUGAGAAACUCCUAUUGCUGGACGAUGUCUCUUACGAGGCAUUAGCAC
(Cas12b Scaffold Sequence; SEQ ID NO: 995).

In various instances, it is advantageous for a spacer sequence to include a 5′ and/or a 3′ “G” nucleotide. In some cases, for example, any spacer sequence or guide polynucleotide provided herein comprises or further comprises a 5′ “G”, where, in some embodiments, the 5′ “G” is or is not complementary to a target sequence. In some embodiments, the 5′ “G” is added to a spacer sequence that does not already contain a 5′ “G.” For example, it can be advantageous for a guide RNA to include a 5′ terminal “G” when the guide RNA is expressed under the control of a U6 promoter or the like because the U6 promoter prefers a “G” at the transcription start site (see Cong, L. et al. “Multiplex genome engineering using CRISPR/Cas systems. Science 339:819-823 (2013) doi: 10.1126/science.1231143). In some cases, a 5′ terminal “G” is added to a guide polynucleotide that is to be expressed under the control of a promoter but is optionally not added to the guide polynucleotide if or when the guide polynucleotide is not expressed under the control of a promoter.

TABLE 1A
Exemplary guide RNAs

| | SEQ ID NOs | |
|---|---|---|
| | (respectively corresponding | Editor |
| gRNA Names | to the listed guides) | Options |
| TSBTx2811; TSBTx2812; TSBTx2829; TSBTx1062; TSBTx2813; | 429; 430; 447; 581; 431; | ABE/ |
| TSBTx2818; TSBTx3635; TSBTx1089; TSBTx1090; TSBTx1092; | 436; 494; 545; 546; 548; | CBE |
| TSBTx1103; TSBTx1108; Guide 415; Guide 419; Guide 420; | 559; 518; 645; 649; 650; | |
| Guide 422; Guide 423; Guide 424; Guide 425; Guide 426; Guide | 652; 653; 654; 655; 656; | |
| 428; Guide 429; Guide 430; Guide 431; Guide 432; Guide 433; | 658; 659; 660; 661; 662; | |
| Guide 434; Guide 435; Guide 436; Guide 437; Guide 438; Guide | 663; 664; 665; 666; 667; | |
| 439; Guide 440; Guide 441; Guide 442; TSBTx1660; TSBTx1661; | 668; 669; 670; 671; 672; | |
| TSBTx1665; TSBTx1669; TSBTx1670; TSBTx1673; TSBTx1681; | 389; 390; 394; 398; 399; | |
| TSBTx1685; TSBTx1689; TSBTx1697; TSBTx025; TSBTx845; | 402; 410; 414; 418; 426; | |
| TSBTx1118; TSBTx1124; TSBTx1130; TSBTx1152; TSBTx1146; | 499; 500; 508; 511; 513; | |
| TSBTx1142; TSBTx1097; TSBTx1074; TSBTx1069; TSBTx1063; | 525; 531; 534; 553; 569; | |
| TSBTx1057; TSBTx1054; TSBTx1053 | 574; 580; 586; 589; 590 | |
| CD58.1; CD58.2 (TSBTx2834); CD58.3 | 673; 674; 675 | ABE/ |
| | | CBE/ |
| | | Cas9 |
| TSBTx1666; TSBTx1667; TSBTx1686; TSBTx1687; TSBTx1690; | 395; 396; 415; 416; 419; | ABE |
| TSBTx1694; TSBTx2810; TSBTx2815; TSBTx2817; TSBTx2821; | 423; 428; 433; 435; 439; | |
| TSBTx2823; TSBTx3633; TSBTx3634; TSBTx1111; TSBTx1112; | 441; 492; 493; 519; 520; | |
| TSBTx1120; TSBTx1131; TSBTx1151; TSBTx1140; TSBTx1088; | 521; 522; 526; 536; 544; | |
| TSBTx1091; TSBTx1093; TSBTx1096; TSBTx1101; TSBTx1104; | 547; 549; 551; 557; 560; | |
| TSBTx1073; TSBTx1072; TSBTx1132; Guide 421 | 570; 571; 598; 651 | |
| TSBTx3325; TSBTx3326; TSBTx3327; TSBTx3328; TSBTx3329; | 451; 452; 453; 454; 455; | Cas12b |
| TSBTx3330; TSBTx3331; TSBTx3332; TSBTx3333; TSBTx3334; | 456; 457; 458; 459; 460; | |
| TSBTx3335; TSBTx3336; TSBTx3337; TSBTx3353; TSBTx3354; | 461; 462; 463; 464; 465; | |
| TSBTx3355; TSBTx3356; TSBTx3357; TSBTx3358; TSBTx3359; | 466; 467; 468; 469; 470; | |
| TSBTx3360; TSBTx3361; TSBTx3362; TSBTx3363; TSBTx3364; | 471; 472; 473; 474; 475; | |
| TSBTx3365; TSBTx3366; TSBTx3367; TSBTx3368; TSBTx3369; | 476; 477; 478; 479; 480; | |
| TSBTx3370; TSBTx3371; TSBTx3372; TSBTx3373; TSBTx3374; | 481; 482; 483; 484; 485; | |
| TSBTx3375; TSBTx3376; TSBTx3377; TSBTx3378; TSBTx3379; | 486; 487; 488; 489; 490; | |
| TSBTx3380; TSBTx3338; TSBTx3339; TSBTx3340; TSBTx3341; | 491; 602; 603; 604; 605; | |
| TSBTx3342; TSBTx3343; TSBTx3344; TSBTx3345; TSBTx3346; | 606; 607; 608; 609; 610; | |
| TSBTx3347; TSBTx3348; TSBTx3349; TSBTx3350; TSBTx3351; | 611; 612; 613; 614; 615; | |
| TSBTx3352; TSBTx3353; TSBTx3354; TSBTx3355; TSBTx3356; | 616; 617; 618; 619; 620; | |
| TSBTx3357; TSBTx3358; TSBTx3359; TSBTx3360; TSBTx3361; | 621; 622; 623; 624; 625; | |
| TSBTx3362; TSBTx3363; TSBTx3364; TSBTx3365; TSBTx3366; | 626; 627; 628; 629; 630; | |
| TSBTx3367; TSBTx3368; TSBTx3369; TSBTx3370; TSBTx3371; | 631; 632; 633; 634; 635; | |
| TSBTx3372; TSBTx3373; TSBTx3374; TSBTx3375; TSBTx3376; | 636; 637; 638; 639; 640; | |
| TSBTx3377; TSBTx3378; TSBTx3379; TSBTx3380 | 641; 642; 643; 644 | |
| TSBTx1662; TSBTx1663; TSBTx1664; TSBTx1668; TSBTx1671; | 391; 392; 393; 397; 400; | CBE |
| TSBTx1672; TSBTx1674; TSBTx1675; TSBTx1676; TSBTx1677; | 401; 403; 404; 405; 406; | |
| TSBTx1678; TSBTx1679; TSBTx1680; TSBTx1682; TSBTx1683; | 407; 408; 409; 411; 412; | |
| TSBTx1684; TSBTx1688; TSBTx1691; TSBTx1692; TSBTx1693; | 413; 417; 420; 421; 422; | |
| TSBTx1695; TSBTx1696; TSBTx1698; TSBTx2814; TSBTx2816; | 424; 425; 427; 432; 434; | |
| TSBTx2819; TSBTx2820; TSBTx2822; TSBTx2824; TSBTx2825; | 437; 438; 440; 442; 443; | |
| TSBTx2826; TSBTx2827; TSBTx2828; TSBTx2830; TSBTx2831; | 444; 445; 446; 448; 449; | |
| TSBTx2853; TSBTx3636; TSBTx3637; TSBTx3638; TSBTx3639; | 450; 495; 496; 497; 498; | |
| TSBTx1105; TSBTx1109; TSBTx1110; TSBTx1113; TSBTx1114; | 501; 502; 503; 504; 505; | |
| TSBTx1116; TSBTx1117; TSBTx1122; TSBTx1121; TSBTx1129; | 506; 507; 509; 510; 512; | |
| TSBTx1133; TSBTx1136; TSBTx1137; TSBTx1107; TSBTx1106; | 514; 515; 516; 517; 523; | |
| TSBTx1153; TSBTx1149; TSBTx1150; TSBTx1148; TSBTx1147; | 524; 527; 528; 529; 530; | |
| TSBTx1145; TSBTx1144; TSBTx1143; TSBTx1139; TSBTx1083; | 532; 533; 535; 538; 539; | |
| TSBTx1084; TSBTx1086; TSBTx1085; TSBTx1087; TSBTx1094; | 540; 541; 542; 543; 550; | |
| TSBTx1095; TSBTx1098; TSBTx1100; TSBTx1099; TSBTx1102; | 552; 554; 555; 556; 558; | |
| TSBTx1082; TSBTx1081; TSBTx1079; TSBTx1080; TSBTx1078; | 561; 562; 563; 564; 565; | |
| TSBTx1075; TSBTx1076; TSBTx1077; TSBTx1071; TSBTx1070; | 566; 567; 568; 572; 573; | |
| TSBTx1068; TSBTx1067; TSBTx1066; TSBTx1065; TSBTx1064; | 575; 576; 577; 578; 579; | |
| TSBTx1060; TSBTx1059; TSBTx1058; TSBTx1056; TSBTx1055; | 583; 584; 585; 587; 588; | |
| TSBTx1115; TSBTx1119; TSBTx1123; TSBTx1126; TSBTx1125; | 591; 592; 593; 594; 595; | |
| TSBTx1127; TSBTx1128; TSBTx1134; TSBTx1135; TSBTx1138; | 596; 597; 599; 600; 601; | |
| Guide 416; Guide 417; Guide 418; Guide 427; TSBTx1141; | 646; 647; 648; 657; 537; | |
| TSBTx1061 | 582 | |

TABLE 1B
Exemplary guide RNAs

| Target | Exemplary guide | |
|---|---|---|
| Gene | RNA SEQ ID NOs | Editor |
| TRAC | 996-999 | BhCas12b nuclease |
| TRAC | 1000-1017 | BhCas12b |
| TRAC | 1018-1028 | BvCas12B |
| TRAC | 1029-1034 | CBE, spCas9 |

TABLE 1C
Exemplary guide RNAs suitable for use with one or more
of the following editors: CBE, ABE, Cas9, and Cas12b

| | | | Guide with Guide |
|---|---|---|---|
| | | Spacer SEQ ID NOS | Scaffold SEQ ID NOS |
| | | (respectively | (respectively |
| | | corresponding to | corresponding to |
| Guides No. | Gene | the listed guides) | the listed guides) |
| b2m_825 (TSBTx845); b2m_630; b2m_227; | B2M | 1324; 1330; 1335, | 1631; 1637; 1642; 1644; |
| b2m_596; Guide 416; Guide 417; Guide 418; | | 1337; 1103-1108; | 1410-1415; 1418-1436; |
| Guide 419; Guide 420; Guide 421; Guide | | 1111-1129; 1110; | 1417; 1416; 1409 |
| 424; Guide 425; Guide 426; Guide 427; | | 1109; 1102 | |
| Guide 428; Guide 429; Guide 430; Guide | | | |
| 431; Guide 432; Guide 433; Guide 434; | | | |
| Guide 435; Guide 436; Guide 437; Guide | | | |
| 438; Guide 439; Guide 440; Guide 441; | | | |
| Guide 442; Guide 423; Guide 422; Guide | | | |
| 415 | | | |
| TSBTx3679; TSBTx3680; TSBTx3680; | CD155 | 1183-1196 | 1490-1503 |
| TSBTx3681; TSBTx3682; TSBTx3682; | | | |
| TSBTx3683; TSBTx3684; TSBTx3684; | | | |
| TSBTx3685; TSBTx3686; TSBTx3687; | | | |
| TSBTx3688; TSBTx3689 | | | |
| TSBTx3633; TSBTx3634; TSBTx3635; | CD48 | 1130-1137 | 1437-1444 |
| TSBTx3635; TSBTx3636; TSBTx3637; | | | |
| TSBTx3638; TSBTx3639 | | | |
| CD58.1; CD58.2 (TSBTx2834); CD58.3 | CD58 | 1312-1314 | 1619-1621 |
| TSBTx4186 (846); TSBTx4187 (847); | HLA-A | 1265-1270; 1274-1278; | 1572-1577; 1581-1585; |
| TSBTx4188 (848); TSBTx4189 (849); | | 1296 | 1603 |
| TSBTx4190 (850); TSBTx4191 (851); | | | |
| TSBTx4195 (855); TSBTx4196 (856); | | | |
| TSBTx4197 (857); TSBTx4198 (858); | | | |
| TSBTx4199 (859); TSBTx4168 (877) | | | |
| TSBTx4192 (852); TSBTx4193 (853); | HLA-A, | 1271-1273; 1293 | 1578-1480; 1600 |
| TSBTx4194 (854); TSBTx4168 (874) | HLA-B, | | |
| | HLA-C | | |
| TSBTx4201 (861); TSBTx4202 (862); | HLA-B | 1280-1282; 1295 | 1587-1589; 1602 |
| TSBTx4203 (863); TSBTx4170 (876) | | | |
| TSBTx4200 (860); TSBTx4204 (864); | HLA-B, | 1279; 1283-1285 | 1586; 1590-1592 |
| TSBTx4205 (865); TSBTx4206 (866) | HLA-C | | |
| TSBTx4207 (867); TSBTx4208 (868); | HLA-C | 1286-1292; 1294 | 1593-1599; 1601 |
| TSBTx4209 (869); TSBTx4210 (870); | | | |
| TSBTx4211 (871); TSBTx4212 (872); | | | |
| TSBTx4167 (873); TSBTx4169 (875) | | | |
| TSBTx3690; TSBTx3691; TSBTx3691; | MICA | 1197-1202; 1204-1217 | 1504-1509; 1511-1524 |
| TSBTx3692; TSBTx3692; TSBTx3693; | | | |
| TSBTx3694; TSBTx3694; TSBTx3695; | | | |
| TSBTx3696; TSBTx3696; TSBTx3697; | | | |
| TSBTx3698; TSBTx3699; TSBTx3700; | | | |
| TSBTx3701; TSBTx3702; TSBTx3703; | | | |
| TSBTx3704; TSBTx3705 | | | |
| TSBTx3693; TSBTx3706; TSBTx3707; | MICB | 1203; 1218-1229 | 1510; 1525-1536 |
| TSBTx3707; TSBTx3708; TSBTx3708; | | | |
| TSBTx3709; TSBTx3709; TSBTx3710; | | | |
| TSBTx3711; TSBTx3711; TSBTx3712; | | | |
| TSBTx3712 | | | |
| TSBTx4213; TSBTx4214; TSBTx4215; | Nectin-2 | 1297-1311 | 1604-1618 |
| TSBTx4216; TSBTx4217; TSBTx4218; | | | |
| TSBTx4219; TSBTx4220; TSBTx4221; | | | |
| TSBTx4222; TSBTx4223; TSBTx4224; | | | |
| TSBTx4225; TSBTx4226; TSBTx4227 | | | |
| TSBTx3640; TSBTx3641; TSBTx3642; | NLRC5 | 1138-1164 | 1445-1471 |
| TSBTx3643; TSBTx3644; TSBTx3645; | (CITA) | | |
| TSBTx3645; TSBTx3646; TSBTx3647; | | | |
| TSBTx3648; TSBTx3649; TSBTx3650; | | | |
| TSBTx3651; TSBTx3652; TSBTx3653; | | | |
| TSBTx3654; TSBTx3654; TSBTx3655; | | | |
| TSBTx3656; TSBTx3657; TSBTx3658; | | | |
| TSBTx3659; TSBTx3660; TSBTx3661; | | | |
| TSBTx3661; TSBTx3662; TSBTx3662 | | | |
| TSBTx3724; TSBTx3724; TSBTx3725; | PDIA3 | 1230-1244 | 1537-1551 |
| TSBTx3725; TSBTx3726; TSBTx3726; | (ERp57) | | |
| TSBTx3727; TSBTx3728; TSBTx3729; | | | |
| TSBTx3730; TSBTx3731; TSBTx3732; | | | |
| TSBTx3732; TSBTx3733; TSBTx3733 | | | |
| Guide 443; Guide 444; Guide 445; Guide | TAP1 | 1035-1057; 1245-1254; | 1342-1364; 1552-1561; |
| 446; Guide 447; Guide 448; Guide 449; | | 1315; 1318; 1322; | 1622; 1625; 1629; 1632; |
| Guide 450; Guide 451; Guide 452; Guide | | 1325; 1328; 1329; | 1635; 1636; 1639; 1640; |
| 453; Guide 454; Guide 455; Guide 456; | | 1332; 1333; 1338-1340 | 1645-1647; |
| Guide 457; Guide 458; Guide 459; Guide | | | |
| 460; Guide 461; Guide 462; Guide 463; | | | |
| Guide 464; Guide 465; TSBTx3923; | | | |
| TSBTx3924; TSBTx3925; TSBTx3926; | | | |
| TSBTx3927; TSBTx3928; TSBTx3929; | | | |
| TSBTx3930; TSBTx3931; TSBTx3932; | | | |
| tap1_93; tap1_210; tap1_498; tap1_140; | | | |
| tap1_485; tap1_142; tap1_139; tap1_38; | | | |
| tap1_444; tap1_161; tap1_454 | | | |
| Guide 518 | TAP1.1 | 1082 | 1389 |
| Guide 527 | TAP1.10 | 1091 | 1398 |
| Guide 528 | TAP1.11 | 1092 | 1399 |
| Guide 529 | TAP1.12 | 1093 | 1400 |
| Guide 519 | TAP1.2 | 1083 | 1390 |
| | AND | | |
| | TAP2 | | |
| Guide 520 | TAP1.3 | 1084 | 1391 |
| | AND | | |
| | TAP 2 | | |
| Guide 521 | TAP1.4 | 1085 | 1392 |
| Guide 522 | TAP1.5 | 1086 | 1393 |
| Guide 523 | TAP1.6 | 1087 | 1394 |
| Guide 524 | TAP1.7 | 1088 | 1395 |
| Guide 525 | TAP1.8 | 1089 | 1396 |
| Guide 526 | TAP1.9 | 1090 | 1397 |
| Guide 466; Guide 467; Guide 468; Guide | TAP2 | 1058-1081; 1255-1264; | 1365-1388; 1562-1571; |
| 469; Guide 470; Guide 471; Guide 472; | | 1317; 1320; 1323; | 1624; 1627; 1630; 1633; |
| Guide 473; Guide 474; Guide 475; Guide | | 1326; 1331; 141 | 1638; 1648 |
| 476; Guide 477; Guide 478; Guide 479; | | | |
| Guide 480; Guide 481; Guide 482; Guide | | | |
| 483; Guide 484; Guide 485; Guide 486; | | | |
| Guide 487; Guide 488; Guide 489; | | | |
| TSBTx3933; TSBTx3934; TSBTx3935; | | | |
| TSBTx3936; TSBTx3937; TSBTx3938; | | | |
| TSBTx3939; TSBTx3940; TSBTx3941; | | | |
| TSBTx3942; tap2_5; tap2_4; tap2_181; | | | |
| tap1_202; tap2_325; tap2_137 | | | |
| Guide 530 | TAP2.1 | 1094 | 1401 |
| Guide 537 | TAP2.10 | 1101 | 1408 |
| Guide 531 | TAP2.4 | 1095 | 1402 |
| Guide 532 | TAP2.5 | 1096 | 1403 |
| Guide 533 | TAP2.6 | 1097 | 1404 |
| Guide 534 | TAP2.7 | 1098 | 1405 |
| Guide 535 | TAP2.8 | 1099 | 1406 |
| Guide 536 | TAP2.9 | 1100 | 1407 |
| tapbp_53; tapbp_18; tapbp_5; tapbp_64; | TAPBP | 1316; 1319; 1321; | 1623; 1626; 1628; 1634; |
| tapbp_79; tapbp_80; TSBTx3663; | (Tapasin) | 1327; 1334; 1336; | 1641; 1643; 1472-1489 |
| TSBTx3663; TSBTx3664; TSBTx3664; | | 1165-1182 | |
| TSBTx3665; TSBTx3666; TSBTx3667; | | | |
| TSBTx3668; TSBTx3669; TSBTx3670; | | | |
| TSBTx3671; TSBTx3672; TSBTx3673; | | | |
| TSBTx3674; TSBTx3675; TSBTx3676; | | | |
| TSBTx3677; TSBTx3678 | | | |

TABLE 2A
Exemplary Spacer Sequences. Boldfaced nucleotides represent
nucleotides corresponding to target nucleotides in the target DNA
sequence corresponding to spacer sequences.

| | | | SEQ | |
|---|---|---|---|---|
| gRNA Name | Gene | Spacer Sequence | ID NO | PAM |
| TSBTx1660 | PIM1 | GCUCUCACCGGGCGCCAGCU | 676 | TGG |
| TSBTx1661 | PIM1 | CUCACCGGGCGCCAGCUUGG | 677 | TGG |
| TSBTx1662 | PIM1 | CCCUUUCCUAGGCAAGGAGA | 678 | AGG |
| TSBTx1663 | PIM1 | GCCUAGGAAAGGGGGAAGCA | 679 | CGG |
| TSBTx1664 | PIM1 | CCUGGAGUCGCAGUACCAGG | 680 | TGG |
| TSBTx1665 | PIM1 | GUACCAGGUGGGCCCGCUAC | 681 | TGG |
| TSBTx1666 | PIM1 | CCCACUCACCGGCAAGUUGU | 682 | CGG |
| TSBTx1667 | PIM1 | CCAGGUGGCCAUCAAACACG | 683 | TGG |
| TSBTx1668 | PIM1 | UGAUGGCCACCUGGAAGCCC | 684 | AGG |
| TSBTx1669 | PIM1 | AUGGCCACCUGGAAGCCCAG | 685 | GGG |
| TSBTx1670 | PIM1 | ACUCACCAGCUCUCCCCAGU | 686 | CGG |
| TSBTx1671 | PIM1 | CAUUAGGCUGCAGGGGCGAG | 687 | GGG |
| TSBTx1672 | PIM1 | AUUAGGCUGCAGGGGCGAGG | 688 | GGG |
| TSBTx1673 | PIM1 | GGCAGGUGCUGGAGGCCGUG | 689 | CGG |
| TSBTx1674 | PIM1 | ACCAUCGAAGUCCGUGUAGA | 690 | CGG |
| TSBTx1675 | PIM1 | CUCGGGUCCCUGUGAGCCAA | 691 | GGG |
| TSBTx1676 | PIM1 | GCCAGGUUUUCUUCAGGCAG | 692 | AGG |
| TSBTx1677 | PIM1 | GCUGACAUUCUGCAGAAAGG | 693 | AGG |
| TSBTx1678 | PIM1 | AAUGUCAGCAUCUCAUUAGA | 694 | TGG |
| TSBTx1679 | PIM1 | GCAAGAUGUUCUCCUGCCCC | 695 | AGG |
| TSBTx1680 | RNF20 | GCAAACCAAAAAUCGCAAGC | 696 | TGG |
| TSBTx1681 | RNF20 | UAGCUACCUGACUCCAGUAU | 697 | CGG |
| TSBTx1682 | RNF20 | UCAUCAAACUGGAGAAGGGU | 698 | AGG |
| TSBTx1683 | RNF20 | GUCUCAGCUGCAGGAACGUG | 699 | TGG |
| TSBTx1684 | RNF20 | GAUAAAUUGCAAGAAAAAG | 700 | TGG |
| TSBTx1685 | RNF20 | CACCUCCACUGUUUAGCUUC | 701 | CGG |
| TSBTx1686 | RNF20 | UUCCCCAGAUAAUCUGAUAG | 702 | TGG |
| TSBTx1687 | RNF20 | CCCAGAUAAUCUGAUAGUGG | 703 | AGG |
| TSBTx1688 | RNF20 | UCCUCGCACAGGAGAAUAUG | 704 | AGG |
| TSBTx1689 | RNF20 | GUUAAGUACCUCCUGAGACA | 705 | TGG |
| TSBTx1690 | RNF20 | UCUCUGUAGGUGAAUUCCAA | 706 | AGG |
| TSBTx1691 | RNF20 | UCAGAACCGUCUCUGUGAGC | 707 | TGG |
| TSBTx1692 | RNF20 | ACUUCGGCAAGACUUUGAGG | 708 | AGG |
| TSBTx1693 | RNF20 | UACACAAAAUGAAAAGCUGA | 709 | AGG |
| TSBTx1694 | RNF20 | GCCCUCCUAAGGUGGAAUUG | 710 | CGG |
| TSBTx1695 | RNF20 | CCUACAGUUGAAAGCACACU | 711 | TGG |
| TSBTx1696 | RNF20 | GCACCAGGUUGAGCUUAUUG | 712 | AGG |
| TSBTx1697 | RNF20 | UACCUCAAUAAGCUCAACC | 713 | TGG |
| TSBTx1698 | RNF20 | AUCUCGCUACACACAGAUAA | 714 | AGG |
| TSBTx2810 | CD70 | GGCGAUGCCGGAGGAGGGUU | 715 | CGG |
| TSBTx2811 | CD70 | CGGUGCGGCGCAGGCCCUAU | 716 | GGG |
| TSBTx2812 | CD70 | UCGGUGCGGCGCAGGCCCUA | 717 | TGG |
| TSBTx2813 | CD70 | CUCACCCCAAGUGACUCGAG | 718 | CGG |
| TSBTx2814 | CD70 | UCCUGGGGGCACAGGGUUAG | 719 | AGG |
| TSBTx2815 | CD70 | CAGGACCUCAGCAGGACCCC | 720 | AGG |
| TSBTx2816 | CD70 | GGUACACAUCCAGGUGACGC | 721 | TGG |
| TSBTx2817 | SIRT1 | UUUGCAGAUAACCUUCUGUU | 722 | CGG |
| TSBTx2818 | SIRT1 | GCCAUACCUAUCCGUGGCCU | 723 | TGG |
| TSBTx2819 | SIRT1 | GACCUACAAUAAGGGGGAAA | 724 | AGG |
| TSBTx2820 | SIRT1 | ACAGACACCUAAAAUGUGCA | 725 | TGG |
| TSBTx2821 | SIRT1 | UUUUAGGUGUCUGUUUCAUG | 726 | TGG |
| TSBTx2822 | SIRT1 | UAUUUCCUGUAUCAAGCAAA | 727 | TGG |
| TSBTx2823 | SIRT1 | UUGAUACAGGAAAUAUAUCC | 728 | TGG |
| TSBTx2824 | SIRT1 | AACAGGUUGCGGGAAUCCAA | 729 | AGG |
| TSBTx2825 | SIRT1 | CAAAGGAUAAUUCAGUGUCA | 730 | TGG |
| TSBTx2826 | SIRT1 | ACGAGGAGAUAUUUUUAAUC | 731 | AGG |
| TSBTx2827 | SIRT1 | ACCUACAUAUUUUCAGAUUU | 732 | TGG |
| TSBTx2828 | SIRT1 | UUACUUGGAAUUAGUGCUAC | 733 | TGG |
| TSBTx2829 | SIRT1 | AAUUCCAAGUAAGUUGGUGA | 734 | TGG |
| TSBTx2830 | SIRT1 | CACAGGAAGUACAAACUUCU | 735 | AGG |
| TSBTx2831 | SIRT1 | UGCUGAACAGAUGGAAAAUC | 736 | CGG |
| TSBTx2853 | RNF20 | AAUUCAGCUAGAAGAUACAU | 737 | TGG |
| TSBTx3325 | FOXP3 | UCUACGCAGCCUGCCCUUGGAC | 738 | ATTG |
| TSBTx3326 | FOXP3 | GCUGGGACAUGUCCCGAGGGGC | 739 | ATTG |
| TSBTx3327 | FOXP3 | UCUUGGCCCUGCAACAUCUGCA | 740 | ATTT |
| TSBTx3328 | FOXP3 | CCCUCAUAGAGGACACAUCCAC | 741 | ATTG |
| TSBTx3329 | FOXP3 | UCUCAUUGAUACCUCUCACCUC | 742 | ATTA |
| TSBTx3330 | FOXP3 | AUACCUCUCACCUCUGUGGUGA | 743 | ATTG |
| TSBTx3331 | FOXP3 | CUUCCCCUCACCACAGAGGUGA | 744 | ATTT |
| TSBTx3332 | FOXP3 | UCAGAUGACUCGUAAAGGGCAA | 745 | ATTT |
| TSBTx3333 | FOXP3 | UGGGUUUUUUUCUUUGCCCUUU | 746 | ATTT |
| TSBTx3334 | FOXP3 | UGAAAUUUUGGGUUUUUUUCUU | 747 | ATTT |
| TSBTx3335 | FOXP3 | UGAGACUUAAACGGAAAUUUUG | 748 | ATTA |
| TSBTx3336 | FOXP3 | CAAAAUUUCCGUUUAAGUCUCA | 749 | ATTT |
| TSBTx3337 | FOXP3 | CCGUUUAAGUCUCAUAAUCAAG | 750 | ATTT |
| TSBTx3353 | RNF20 | CCAAUUCCUGACAUUUUGGCAG | 751 | ATTT |
| TSBTx3354 | RNF20 | GAAAUAAAAGAGCAGCUGGAGA | 752 | ATTG |
| TSBTx3355 | RNF20 | AGGGACCACAGUGGAAACAAUU | 753 | ATTC |
| TSBTx3356 | RNF20 | UUUCCACUGUGGUCCCUGAAUC | 754 | ATTG |
| TSBTx3357 | RNF20 | AGCUAGGAGGUGUCUCUUCAAC | 755 | ATTA |
| TSBTx3358 | RNF20 | GAACACUGCAAACCAAAAAUCG | 756 | ATTA |
| TSBTx3359 | RNF20 | UUGGUUUGCAGUGUUCUAAUGU | 757 | ATTT |
| TSBTx3360 | RNF20 | CUGCCAGCUUGCGAUUUUUGGU | 758 | ATTT |
| TSBTx3361 | RNF20 | AAGAUGAACUUCGUGAGCACAU | 759 | ATTG |
| TSBTx3362 | RNF20 | AAAAACUGGAACGACGACAGGC | 760 | ATTG |
| TSBTx3363 | RNF20 | AUUGUCAACCGAUACUGGAGUC | 761 | ATTG |
| TSBTx3364 | RNF20 | UCAACCGAUACUGGAGUCAGGU | 762 | ATTG |
| TSBTx3365 | RNF20 | CUAUCAGAGUCUGGUUCUGGUU | 763 | ATTG |
| TSBTx3366 | RNF20 | AUCAUAAACAGUCACAAUCUGG | 764 | ATTT |
| TSBTx3367 | RNF20 | UGACUGUUUAUGAUAAAUUGCA | 765 | ATTG |
| TSBTx3368 | RNF20 | CAAGAAAAAGUGGAGCUCUUAU | 766 | ATTG |
| TSBTx3369 | RNF20 | UCCUGUGCGAGGAAAGAGUUCA | 767 | ATTC |
| TSBTx3370 | RNF20 | CUGUAGCCUCAUAUUCUCCUGU | 768 | ATTC |
| TSBTx3371 | RNF20 | ACAGAUCUUCUUCAGGAAAAGC | 769 | ATTG |
| TSBTx3372 | RNF20 | GGCUGUCUCCACUUUACUCUGC | 770 | ATTC |
| TSBTx3373 | RNF20 | AUGACCUGCAGUGGGAUAUUGA | 771 | ATTG |
| TSBTx3374 | RNF20 | UGUCAAUAUCCCACUGCAGGUC | 772 | ATTT |
| TSBTx3375 | RNF20 | ACAAAAUUCGAAAGAGGGAACA | 773 | ATTG |
| TSBTx3376 | RNF20 | GAAAGAGGGAACAGCGACUCAA | 774 | ATTC |
| TSBTx3377 | RNF20 | UUGGUUUGCAGUGUUCGAAUGU | 775 | ATTT |
| TSBTx3378 | RNF20 | AUCAUAGACAGUCACAAUCUGG | 776 | ATTT |
| TSBTx3379 | RNF20 | UGACUGUCUAUGAUAAAUUGCA | 777 | ATTG |
| TSBTx3380 | RNF20 | CAAGAAAAAGUGGAACUCUUAU | 778 | ATTG |
| TSBTx3633 | CD48 | AAGCAUGUGCUCCAGAGGUU | 779 | GGG |
| TSBTx3634 | CD48 | GAAGCAUGUGCUCCAGAGGU | 780 | TGG |
| TSBTx3635 | CD48 | UUCUUACCCAGGGUACAGGG | 781 | TGG |
| TSBTx3636 | CD48 | CGACCAGAAGAUUGUAGAAU | 782 | GGG |
| TSBTx3637 | CD48 | UCGACCAGAAGAUUGUAGAA | 783 | TGG |
| TSBTx3638 | CD48 | AAUCCCAUUCUACAAUCUUC | 784 | TGG |
| TSBTx3639 | CD48 | UGCAAUCCAUUCUACUCCAA | 785 | AGG |
| TSBTx025 | PD-1 | CACCUACCUAAGAACCAUCCUGG | 786 | TGG |
| TSBTx845 | B2M | ACUCACGCUGGAUAGCCUCC | 787 | AGG |
| TSBTx1105 | CHIP | CGCGCAGGAGCUCAAGGAGC | 788 | |
| TSBTx1109 | CHIP | CGGGUCUAGGGACCAAGGCC | 789 | |
| TSBTx1110 | CHIP | CCGGGUCUAGGGACCAAGGC | 790 | |
| TSBTx1113 | CHIP | GAAGAUGCAGCAGCACGAGC | 791 | |
| TSBTx1114 | CHIP | GCAGCAGCACGAGCAGGCCC | 792 | |
| TSBTx1116 | CHIP | GGGGCAGUGCCAGCUGGAGA | 793 | |
| TSBTx1117 | CHIP | GGCAGUGCCAGCUGGAGA | 794 | |
| TSBTx1118 | CHIP | CAGCCAACCUCGCUGCAGAU | 795 | |
| TSBTx1122 | CHIP | AGGAGCAGCGGCUGAACUUC | 796 | |
| TSBTx1121 | CHIP | AAGGAGCAGCGGCUGAACUU | 797 | |
| TSBTx1124 | CHIP | GUCCCACCUCUCACGCUCCG | 798 | |
| TSBTx1129 | CHIP | GGCCCAGCAGGCCUGCAUUG | 799 | |
| TSBTx1130 | CHIP | CACGUGCUUGGCCUCAAUGC | 800 | |
| TSBTx1133 | CHIP | UUUCUCAGGUGGAUGAGAAG | 801 | |
| TSBTx1136 | CHIP | ACACGCUGCAGUGACAAGAA | 802 | |
| TSBTx1137 | CHIP | CACACGCUGCAGUGACAAGA | 803 | |
| TSBTx1107 | CHIP | GGGCCGAAAGUACCCGGAGG | 804 | |
| TSBTx1108 | CHIP | CACGAUCGCGCGGCCGUAGC | 805 | |
| TSBTx1111 | CHIP | UCCCUAGACCCGGAACCCGC | 806 | |
| TSBTx1112 | CHIP | CUAGACCCGGAACCCGCUGG | 807 | |
| TSBTx1120 | CHIP | CCCAGCUUACAGCCUGGCCA | 808 | |
| TSBTx1131 | CHIP | CUCUUCACAGGACAAGUACA | 809 | |
| TSBTx1106 | CHIP | GCGCAGGAGCUCAAGGAGC | 810 | |
| TSBTx1153 | BRINP1 | GACAGACCAACAUGUCUCCA | 811 | |
| TSBTx1152 | BRINP1 | UUACCUGUAUAUUUUAUAUC | 812 | |
| TSBTx1151 | BRINP1 | UGCCCAGGGAGUUUGCCCGU | 813 | |
| TSBTx1149 | BRINP1 | CUUCCAACGGGCAAACUCCC | 814 | |
| TSBTx1150 | BRINP1 | UUCCAACGGGCAAACUCCCU | 815 | |
| TSBTx1148 | BRINP1 | CAUGAGAUCCAGAUAUCAAC | 816 | |
| TSBTx1147 | BRINP1 | CCUUCAAGGUAGGAAGCCAG | 817 | |
| TSBTx1146 | BRINP1 | ACUCUCUACCUGAAUUCUCC | 818 | |
| TSBTx1145 | BRINP1 | ACUCAUCUAGUGAGAGACAA | 819 | |
| TSBTx1144 | BRINP1 | GCUCCUGCAGAGUGCCACGG | 820 | |
| TSBTx1142 | BRINP1 | CUUACCUCUCUCUAGGCAGC | 821 | |
| TSBTx1143 | BRINP1 | ACCACCAGCUGCCUAGAGAG | 822 | |
| TSBTx1140 | BRINP1 | CUCCCAGGACAAUUCAGCAG | 823 | |
| TSBTx1141 | BRINP1 | UGAAUUGUCCUGGGAGAUAA | 824 | |
| TSBTx1139 | BRINP1 | AAGCCACUGCUGAAUUGUCC | 825 | |
| TSBTx1083 | JNK1 | UAUCAGAAUUUAAAACCUAU | 826 | |
| TSBTx1084 | JNK1 | CAGGAGCUCAAGGAAUAGUA | 827 | |
| TSBTx1086 | JNK1 | GAAUCAGACUCAUGCCAAGC | 828 | |
| TSBTx1085 | JNK1 | AGAAUCAGACUCAUGCCAAG | 829 | |
| TSBTx1087 | JNK1 | CUUCUCUAUCAGAUGCUGUG | 830 | |
| TSBTx1088 | JNK1 | UAUAGUGGAUUUAUGGUCUG | 831 | |
| TSBTx1089 | JNK1 | UACAGUCCCUUCCUGGAAAG | 832 | |
| TSBTx1090 | JNK1 | ACAAGGAUACAGUCCCUUCC | 833 | |
| TSBTx1091 | JNK1 | UCAGUUGACAUUUGGUCAGU | 834 | |
| TSBTx1092 | JNK1 | AUAAGGAUACGAUCUGUACC | 835 | |
| TSBTx1093 | JNK1 | UGAUAUUAGAUAUUGAUCAG | 836 | |
| TSBTx1094 | JNK1 | AGAAACUGCAACCAACAGUA | 837 | |
| TSBTx1096 | JNK1 | UGUUUGCAGCCAGUCAGGCA | 838 | |
| TSBTx1095 | JNK1 | CUGACUGGCUGCAAACAUAA | 839 | |
| TSBTx1097 | JNK1 | CUUACAGCUUCUGCUUCAGA | 840 | |
| TSBTx1098 | JNK1 | GGUGGUGGCUGAAAAACACA | 841 | |
| TSBTx1100 | JNK1 | UGACAAGCAGUUAGAUGAAA | 842 | |
| TSBTx1099 | JNK1 | CUGACAAGCAGUUAGAUGAA | 843 | |
| TSBTx1101 | JNK1 | UUUUACAGAAUUGAUAUAUA | 844 | |
| TSBTx1102 | JNK1 | CGGGGGCAGCCCUCUCCUUU | 845 | |
| TSBTx1103 | JNK1 | UGUAACCAACCUAAAGGAGA | 846 | |
| TSBTx1104 | JNK1 | ACAGGUGCAGCAGUGAUCAA | 847 | |
| TSBTx1082 | PRKCQ | GGGUCCUGCCAGUCUUGUCA | 848 | |
| TSBTx1081 | PRKCQ | CUGCCAGUCUUGUCAGGGCG | 849 | |
| TSBTx1079 | PRKCQ | GCUGUCCCAGGGUGGGUACA | 850 | |
| TSBTx1080 | PRKCQ | UCCCAGGGUGGGUACAUGGU | 851 | |
| TSBTx1078 | PRKCQ | GUCAUGCAGAUCAUUGUGAA | 852 | |
| TSBTx1075 | PRKCQ | UUGUGUCUACAGGAAGAGAG | 853 | |
| TSBTx1076 | PRKCQ | UGUGUCUACAGGAAGAGAGA | 854 | |
| TSBTx1077 | PRKCQ | GUGUCUACAGGAAGAGAGAG | 855 | |
| TSBTx1074 | PRKCQ | UACUGUACCAGACAAACUCG | 856 | |
| TSBTx1073 | PRKCQ | UUACAGGGGCCUGAACAAAC | 857 | |
| TSBTx1072 | PRKCQ | UACAGGGGCCUGAACAAACA | 858 | |
| TSBTx1071 | PRKCQ | CCAGUGCCGACGUAAGUAAG | 859 | |
| TSBTx1070 | PRKCQ | CAGUGCCGACGUAAGUAAGA | 860 | |
| TSBTx1069 | PRKCQ | AAAUACUCACCAUGGUUUCU | 861 | |
| TSBTx1068 | PRKCQ | GGAACUGAAAGAAAGGCAGA | 862 | |
| TSBTx1067 | PRKCQ | CGGCAAGGACUCAAGUGUGA | 863 | |
| TSBTx1066 | PRKCQ | UCAUAGAUGCCAGACAAAGG | 864 | |
| TSBTx1065 | PRKCQ | ACAGGUAGGAGCAUGUGCCG | 865 | |
| TSBTx1064 | PRKCQ | ACUGAACAGAUCUUCAGAGA | 866 | |
| TSBTx1063 | PRKCQ | CUACCUCUUUUUCCCGGUGU | 867 | |
| TSBTx1062 | PRKCQ | CAGAGCCUCAGGGCAUUUCC | 868 | |
| TSBTx1061 | PRKCQ | AGAGCCUCAGGGCAUUUCCU | 869 | |
| TSBTx1060 | PRKCQ | GCAGAUUAAACUAAAAAUUG | 870 | |
| TSBTx1059 | PRKCQ | CAAUCAAUUUUUCGCAAUAA | 871 | |
| TSBTx1058 | PRKCQ | GUACAUUCCAGACCAAGGUA | 872 | |
| TSBTx1057 | PRKCQ | CCUCUUACGUCGCUCUGGAA | 873 | |
| TSBTx1056 | PRKCQ | ACCUGGGGGAAGGAGAACCA | 874 | |
| TSBTx1055 | PRKCQ | CUGCAGUUCCUUCAUUCCAA | 875 | |
| TSBTx1054 | PRKCQ | UUACCUGUAGACUAUUCCUU | 876 | |
| TSBTx1053 | PRKCQ | ACCUCUGGGGCGAUGUAGUC | 877 | |
| TSBTx1115 | CHIP1 | AGCAGGCCCUGGCCGACUGC | 878 | |
| TSBTx1119 | CHIP1 | GGCUGUAAGCUGGGGAACGG | 879 | |
| TSBTx1123 | CHIP1 | GGAGCAGCGGCUGAACUUCG | 880 | |
| TSBTx1126 | CHIP1 | CAGCUCCCUGGGGGUUGACC | 881 | |
| TSBTx1125 | CHIP1 | CCUGGGGGUUGACCAGGAGC | 882 | |
| TSBTx1127 | CHIP1 | AGAGUGCCAGCGAAACCACG | 883 | |
| TSBTx1128 | CHIP1 | GAGUGCCAGCGAAACCACGA | 884 | |
| TSBTx1132 | CHIP1 | UUCACAGGACAAGUACAUGG | 885 | |
| TSBTx1134 | CHIP1 | GUCUCGCUUCUGUGGCACAG | 886 | |
| TSBTx1135 | CHIP1 | GCACCUGCAGGUGAGGCCUG | 887 | |
| TSBTx1138 | CHIP1 | GGAACAGCUCAUCCCCAACU | 888 | |
| TSBTx3338 | STUB1 (CHIP) | CCCUGCUCCUUGAGCUCCUGCG | 889 | ATTG |
| TSBTx3339 | STUB1 (CHIP) | CACCAACCGGGCCUUGUGCUAC | 890 | ATTA |
| TSBTx3340 | STUB1 (CHIP) | GCGAUGGCCUCAUCAUAGCUCU | 891 | ATTG |
| TSBTx3341 | STUB1 (CHIP) | GAAGAGCGCUGGGGAUGUCGUC | 892 | ATTC |
| TSBTx3342 | STUB1 (CHIP) | AGGAGCGGCGCAUCCACCAGGA | 893 | ATTG |
| TSBTx3343 | STUB1 (CHIP) | CCGCGGAGCGUGAGAGGUGGGA | 894 | ATTG |
| TSBTx3344 | STUB1 (CHIP) | AGGCCAAGCACGUGAGGGUGCC | 895 | ATTG |
| TSBTx3345 | STUB1 (CHIP) | UGACCCCGUGACCCGGAGCCCC | 896 | ATTT |
| TSBTx3346 | STUB1 (CHIP) | ACGCAUUCAUCUCUGAGAAUGG | 897 | ATTG |
| TSBTx3347 | STUB1 (CHIP) | AUCUCUGAGAAUGGCUGGGUGG | 898 | ATTC |
| TSBTx3348 | STUB1 (CHIP) | UCAGAGAUGAAUGCGUCAAUAA | 899 | ATTC |
| TSBTx3349 | STUB1 (CHIP) | CACCAACCGUGCCCUGUGCUAC | 900 | ATTA |
| TSBTx3350 | STUB1 (CHIP) | CAGCGCUUCUUCUUCGCGAUUC | 901 | ATTC |
| TSBTx3351 | STUB1 (CHIP) | AGGCUAAGCAUGUGAGGGUGCC | 902 | ATTG |
| TSBTx3352 | STUB1 (CHIP) | UCAGAGAUGAAUGCGUCAAUGA | 903 | ATTC |
| TSBTx3353 | RNF20 | CCAAUUCCUGACAUUUUGGCAG | 904 | ATTT |
| TSBTx3354 | RNF20 | GAAAUAAAAGAGCAGCUGGAGA | 905 | ATTG |
| TSBTx3355 | RNF20 | AGGGACCACAGUGGAAACAAUU | 906 | ATTC |
| TSBTx3356 | RNF20 | UUUCCACUGUGGUCCCUGAAUC | 907 | ATTG |
| TSBTx3357 | RNF20 | AGCUAGGAGGUGUCUCUUCAAC | 908 | ATTA |
| TSBTx3358 | RNF20 | GAACACUGCAAACCAAAAAUCG | 909 | ATTA |
| TSBTx3359 | RNF20 | UUGGUUUGCAGUGUUCUAAUGU | 910 | ATTT |
| TSBTx3360 | RNF20 | CUGCCAGCUUGCGAUUUUUGGU | 911 | ATTT |
| TSBTx3361 | RNF20 | AAGAUGAACUUCGUGAGCACAU | 912 | ATTG |
| TSBTx3362 | RNF20 | AAAAACUGGAACGACGACAGGC | 913 | ATTG |
| TSBTx3363 | RNF20 | AUUGUCAACCGAUACUGGAGUC | 914 | ATTG |
| TSBTx3364 | RNF20 | UCAACCGAUACUGGAGUCAGGU | 915 | ATTG |
| TSBTx3365 | RNF20 | CUAUCAGAGUCUGGUUCUGGUU | 916 | ATTG |
| TSBTx3366 | RNF20 | AUCAUAAACAGUCACAAUCUGG | 917 | ATTT |
| TSBTx3367 | RNF20 | UGACUGUUUAUGAUAAAUUGCA | 918 | ATTG |
| TSBTx3368 | RNF20 | CAAGAAAAAGUGGAGCUCUUAU | 919 | ATTG |
| TSBTx3369 | RNF20 | UCCUGUGCGAGGAAAGAGUUCA | 920 | ATTC |
| TSBTx3370 | RNF20 | CUGUAGCCUCAUAUUCUCCUGU | 921 | ATTC |
| TSBTx3371 | RNF20 | ACAGAUCUUCUUCAGGAAAAGC | 922 | ATTG |
| TSBTx3372 | RNF20 | GGCUGUCUCCACUUUACUCUGC | 923 | ATTC |
| TSBTx3373 | RNF20 | AUGACCUGCAGUGGGAUAUUGA | 924 | ATTG |
| TSBTx3374 | RNF20 | UGUCAAUAUCCCACUGCAGGUC | 925 | ATTT |
| TSBTx3375 | RNF20 | ACAAAAUUCGAAAGAGGGAACA | 926 | ATTG |
| TSBTx3376 | RNF20 | GAAAGAGGGAACAGCGACUCAA | 927 | ATTC |
| TSBTx3377 | RNF20 | UUGGUUUGCAGUGUUCGAAUGU | 928 | ATTT |
| TSBTx3378 | RNF20 | AUCAUAGACAGUCACAAUCUGG | 929 | ATTT |
| TSBTx3379 | RNF20 | UGACUGUCUAUGAUAAAUUGCA | 930 | ATTG |
| TSBTx3380 | RNF20 | CAAGAAAAAGUGGAACUCUUAU | 931 | ATTG |
| Guide 415 | B2M | ACUCACGCUGGAUAGCCUCC | 932 | |
| Guide 416 | B2M | CACAGCCCAAGAUAGUUAAG | 933 | |
| Guide 417 | B2M | ACAGCCCAAGAUAGUUAAGU | 934 | |
| Guide 418 | B2M | CAGCCCAAGAUAGUUAAGUG | 935 | |
| Guide 419 | B2M | UUACCCCACUUAACUAUCUU | 936 | |
| Guide 420 | B2M | CUUACCCCACUUAACUAUCU | 937 | |
| Guide 421 | B2M | UCGAUCUAUGAAAAAGACAG | 938 | |
| Guide 422 | B2M | CUCACGCUGGAUAGCCUCC | 939 | |
| Guide 423 | B2M | UCACGCUGGAUAGCCUCC | 940 | |
| Guide 424 | B2M | CGCGAGCACAGCUAAGGCCA | 941 | |
| Guide 425 | B2M | GAGUAGCGCGAGCACAGCUA | 942 | |
| Guide 426 | B2M | GCUACUCUCUCUUUCUGGCC | 943 | |
| Guide 427 | B2M | CGUGAGUAAACCUGAAUCUU | 944 | |
| Guide 428 | B2M | UUUGACUUUCCAUUCUCUGC | 945 | |
| Guide 429 | B2M | ACCCAGACACAUAGCAAUUC | 946 | |
| Guide 430 | B2M | AAGUCAACUUCAAUGUCGGA | 947 | |
| Guide 431 | B2M | CAGUAAGUCAACUUCAAUGU | 948 | |
| Guide 432 | B2M | GAAGUUGACUUACUGAAGAA | 949 | |
| Guide 433 | B2M | UGGAGAGAGAAUUGAAAAAG | 950 | |
| Guide 434 | B2M | UUCAGACUUGUCUUUCAGCA | 951 | |
| Guide 435 | B2M | ACUUGUCUUUCAGCAAGGAC | 952 | |
| Guide 436 | B2M | AUACUCAUCUUUUUCAGUGG | 953 | |
| Guide 437 | B2M | CAUACUCAUCUUUUUCAGUG | 954 | |
| Guide 438 | B2M | GCAUACUCAUCUUUUUCAGU | 955 | |
| Guide 439 | B2M | GGCAUACUCAUCUUUUUCAG | 956 | |
| Guide 440 | B2M | AGUCACAUGGUUCACACGGC | 957 | |
| Guide 441 | B2M | ACAAAGUCACAUGGUUCACA | 958 | |
| Guide 442 | B2M | UGGGCUGUGACAAAGUCACA | 959 | |
| CD58.1 | CD58 | GCAACCAUGGCUCGUCGGGC | 960 | CGG |
| CD58.2 | CD58 | ACUCACCAAAGCAGUGCAGC | 961 | AGG |
| (TSBTx2834) | | | | |
| CD58.3 | CD58 | CUCACCGCUGCUUGGGAUAC | 962 | AGG |

TABLE 2B
Exemplary Spacer Sequences. In the following table, the right portion
of the table is a continuation of the bottom of the left portion.

| Target | SEQ ID NOs for Spacers | Target | SEQ ID NOs for Spacers |
|---|---|---|---|
| ACAT1 | 3251-3261 | LDHA | 2335-2337 |
| ACLY | 1778-1799 | LDLR | 2888 |
| ADORA2A | 1800-1811 | LIF | 2338-2344 |
| AKT1 | 1674-1677 | LYN | 2345-2356 |
| AKT2 | 1678; 1679 | MAP4K4 | 2357-2388 |
| AXL | 1812-1839 | MAPK14 | 2389-2405 |
| B2M | 2883; 2885; 2889; 3057-3060 | MCJ | 3309 |
| BATF | 1841-1847 | MEF2D | 2406-2424 |
| BCL2L11 | 1848-1853 | MGAT5 | 2425-2439 |
| BLIMP1 | 1680-1684 | NFATC1 | 1649-1661 |
| BTLA | 3052-3056 | NFATC2 | 1662-1672 |
| CAMK2D | 1854-1865 | NFATC4 | 1673 |
| cAMP | 1866-1870 | NR4A1 | 3271-3289 |
| CASP8 | 1871-1876 | NR4A2 | 3290-3300 |
| CBLB | 2887; 3031-3051 | NR4A3 | 3301-3308 |
| CBL-B | 1688-1692 | NT5E (CD73) | 2440-2449 |
| CCR5 | 1877-1880 | ODC1 | 2450-2456 |
| CD123 | 3199-3224 | OTULINL (FAM105A) | 2457-2469 |
| CD160 | 1973-1978 | PAG1 | 2470-2478 |
| CD2 | 1760-1767; 1881-1883 | PD-1 (PDCD1) | 1697; 1698; 1768-1773; |
| | | | 2981-2994; 1323; 1324 |
| CD244 | 1979-1986 | PDIA3 | 2479-2486 |
| CD276 | 1987-1998 | PHD1 (EGLN2) | 2487-2502 |
| CD33 | 3169-3193 | PHD2 (EGLN1) | 2503-2507 |
| CD38 | 1942-1951 | PHD3 (EGLN3) | 2508-2517 |
| CD3D | 1884-1891 | PIK3CD | 2518-2581 |
| CD3E | 1892-1906 | PIKFYVE | 2582-2597 |
| CD3G | 1907-1918 | PPARa | 2598-2607 |
| CD4 | 1919-1929 | PPARd | 2608-2626 |
| CD5 | 1718-1759; 1930-1935 | PRDMI1 | 2627-2637 |
| CD52 | 3194-3198; 2882 | PRKACA | 2638-2661 |
| CD7 | 2881; 2886; 3160-3168 | PTEN | 2662-2666 |
| CD70 | 1952-1958 | PTPN11 | 2698-2700 |
| CD82 | 1959-1966 | PTPN2 | 2667-2669 |
| CD86 | 1967-1972 | PTPN6 | 2670-2697 |
| CD8A | 1936-1941 | PVRIG (CD112R) | 2953-2980 |
| CD96 | 3015-3030 | RASA2 | 2701-2710 |
| CDK4 | 1712 | RFXANK | 2933-2952 |
| CDK8 | 1999-2005 | SELPG/PSGL1 | 3310-3318 |
| CDKN1B | 2006-2014 | SHP1 | 1693 |
| Chi3l1 | 2996-3014 | SIGLEC15 | 2711-2725 |
| CIITA | 3104-3159; 2884 | SLA | 2726-2732 |
| CISH | 3237-3250 | SLAMF7 | 2733-2740 |
| CSF2 | 2015-2020 | SMARCA4 | 1702-1711 |
| CSK | 2021-2035 | SOCS1 | 2741; 2742 |
| CTLA-4 | 2036-2045; 2995 | Spry1 | 3091-3103 |
| CUL3 | 2046-2053 | STK4 | 2743-2748 |
| Cyp11a1 | 2054-2065 | SUV39H1 | 2749-2755 |
| DCK | 2066-2077 | TET2 | 1699-1701; 3061-3090 |
| DGKA | 2078-2098 | TGFbRII | 2925-2932; 3319-3322 |
| DGKZ | 2099-2148 | TIGIT | 2919-2924 |
| DHX37 | 2149-2194 | Tim-3 | 2910-2918 |
| ELOB (TCEB2) | 2195-2199 | TMEM222 | 2756-2769 |
| ENTPD1 (CD39) | 2200-2216 | TNFAIP3 | 2770-2775 |
| FADD | 2217-2223 | TNFRSF10B | 2806-2812 |
| GATA3 | 3262-3270 | TNFRSF8 (CD30) | 2776-2805 |
| GCN2 kinase (IDO pathway) | 1685-1687 | TOX | 2813-2832 |
| IL10 | 2248-2252 | TOX2 | 2833-2859 |
| IL10RA | 2253-2271 | TRAC | 1774-1777; 2907-2909 |
| IL6 | 2224-2236 | TRBC1/2 | 3225-3236 |
| IL6R | 2237-2247 | UBASH3A | 2860-2872 |
| IRF4 | 1694-1696; 2272-2311 | VHL | 2873-2879 |
| JUNB | 2312-2322 | VISTA | 2890-2906 |
| LAIR-1 (CD305) | 2323-2334 | XBP1 | 2880 |
| | | ZAP70 | 1713-1717 + C54AA1:D65 |

TABLE 2C
Exemplary Target Sequences.

| Guide Name | PAM | gRNA Target + PAM (bold) | SEQ ID NO |
|---|---|---|---|
| TSBTx4186 (846) | TGG | TGACGGCCATCCTCGGCGTCTGG | 3325 |
| TSBTx4187 (847) | GGG | GACGGCCATCCTCGGCGTCTGGG | 3326 |
| TSBTx4188 (848) | GGG | ACGGCCATCCTCGGCGTCTGGGG | 3327 |
| TSBTx4189 (849) | TGG | CGCACTCACCCGCCCAGGTCTGG | 3328 |
| TSBTx4190 (850) | GGG | GCACTCACCCGCCCAGGTCTGGG | 3329 |
| TSBTx4191 (851) | AGG | CCCAGGCTCGCACTCCATGAGG | 3330 |
| TSBTx4192 (852) | TGG | GTCACTCACCGGCCTCGCTCTGG | 3331 |
| TSBTx4193 (853) | AGG | CCTTACCCCATCTCAGGGTGAGG | 3332 |
| TSBTx4194 (854) | GGG | CTTACCCCATCTCAGGGTGAGGG | 3333 |
| TSBTx4195 (855) | AGG | GGCTCTGGGAAAAGAGGGGAAGG | 3334 |
| TSBTx4196 (856) | AGG | CTTCCCACAGATAGAAAAGGAGG | 3335 |
| TSBTx4197 (857) | GGG | TTCCCACAGATAGAAAAGGAGGG | 3336 |
| TSBTx4198 (858) | AGG | CCCAGGCAGTGACAGTGCCCAGG | 3337 |
| TSBTx4199 (859) | GGG | CCAGGCAGTGACAGTGCCCAGGG | 3338 |
| TSBTx4200 (860) | AGG | GACCCGCATCTCGGCGTCTGAGG | 3339 |
| TSBTx4201 (861) | CGG | GCACTCACCGGCCCAGGTCTCGG | 3340 |
| TSBTx4202 (862) | GGG | TGGGAGCCTGGGGGCGAGGAGGG | 3341 |
| TSBTx4203 (863) | GGG | GGGAGCCTGGGGGCGAGGAGGGG | 3342 |
| TSBTx4204 (864) | AGG | CCCCAGGCTCCCACTCCATGAGG | 3343 |
| TSBTx4205 (865) | GGG | TTACCCCATCTCAGGGTGAGGGG | 3344 |
| TSBTx4206 (866) | GGG | TTCCCACAGGTGGAAAAGGAGGG | 3345 |
| TSBTx4207 (867) | GGG | TGACCCGCATCTCGGCGTCTGGG | 3346 |
| TSBTx4208 (868) | CGG | GCACTCACAGGCCCAGGTCTCGG | 3347 |
| TSBTx4209 (869) | AGG | CACAGGCCCAGGTCTCGGTCAGG | 3348 |
| TSBTx4210 (870) | TGG | AGACCCTGGCCCCGCCCCCGTGG | 3349 |
| TSBTx4211 (871) | AGG | GGCTCTGGGAAAGGAGGGGAAGG | 3350 |
| TSBTx4212 (872) | AGG | CCCAGCCAGCAACAGTGCCCAGG | 3351 |
| TSBTx4167 (873) | ATTG | ATTGGGACCGGGAGACACAGAAGTAC | 3352 |
| TSBTx4168 (874) | ATTA | ATTACATCGCCCTGAACGAGGACCTG | 3353 |
| TSBTx4169 (875) | ATTA | ATTACATCGCCCTGAACGAGGATCTG | 3354 |
| TSBTx4170 (876) | ATTG | ATTGTTGCTGGCCTGGCTGTCCTAGC | 3355 |
| TSBTx4168 (877) | ATTA | ATTACATCGCCCTGAACGAGGACCTG | 3356 |

### Nucleobase Editors

Useful in the methods and compositions described herein are nucleobase editors that edit, modify or alter a target nucleotide sequence of a polynucleotide. Nucleobase editors described herein typically include a polynucleotide programmable nucleotide binding domain and a nucleobase editing domain (e.g., adenosine deaminase, cytidine deaminase). A polynucleotide programmable nucleotide binding domain, when in conjunction with a bound guide polynucleotide (e.g., gRNA), can specifically bind to a target polynucleotide sequence and thereby localize the base editor to the target nucleic acid sequence desired to be edited.

### Polynucleotide Programmable Nucleotide Binding Domain

Polynucleotide programmable nucleotide binding domains bind polynucleotides (e.g., RNA, DNA). A polynucleotide programmable nucleotide binding domain of a base editor can itself comprise one or more domains (e.g., one or more nuclease domains). In some embodiments, the nuclease domain of a polynucleotide programmable nucleotide binding domain comprises an endonuclease or an exonuclease.

Disclosed herein are base editors comprising a polynucleotide programmable nucleotide binding domain comprising all or a portion (e.g., a functional portion) of a CRISPR protein (i.e., a base editor comprising as a domain all or a portion (e.g., a functional portion) of a CRISPR protein (e.g., a Cas protein), also referred to as a “CRISPR protein-derived domain” of the base editor). A CRISPR protein-derived domain incorporated into a base editor can be modified compared to a wild-type or natural version of the CRISPR protein. A CRISPR protein-derived domain can comprise one or more mutations, insertions, deletions, rearrangements and/or recombinations relative to a wild-type or natural version of the CRISPR protein.

Cas proteins that can be used herein include class 1 and class 2. Non-limiting examples of Cas proteins include Cas1, Cas1B, Cas2, Cas3, Cas4, Cas5, Cas5d, Cas5t, Cas5h, Cas5a, Cas6, Cas7, Cas8, Cas9 (also known as Csn1 or Csx12), Cas10, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, Cas12a/Cpf1, Cas12b/C2c1 (e.g., SEQ ID NO: 232), Cas12c/C2c3, Cas12d/CasY, Cas12e/CasX, Cas12g, Cas12h, Cas12i, and Cas12j/CasΦ, CARF, DinG, homologues thereof, or modified versions thereof. A CRISPR enzyme can direct cleavage of one or both strands at a target sequence, such as within a target sequence and/or within a complement of a target sequence. For example, a CRISPR enzyme can direct cleavage of one or both strands within about 1, 2, 3, 4, 5, 6, 7, 8, 9, 10, 15, 20, 25, 50, 100, 200, 500, or more base pairs from the first or last nucleotide of a target sequence.

A vector that encodes a CRISPR enzyme that is mutated to with respect to a corresponding wild-type enzyme such that the mutated CRISPR enzyme lacks the ability to cleave one or both strands of a target polynucleotide containing a target sequence can be used. A Cas protein (e.g., Cas9, Cas12) or a Cas domain (e.g., Cas9, Cas12) can refer to a polypeptide or domain with at least or at least about 50%, 60%, 70%, 80%, 90%, 91%, 92%, 93%, 94%, 95%, 96%, 97%, 98%, 99%, or 100% sequence identity and/or sequence homology to a wild-type exemplary Cas polypeptide or Cas domain. Cas (e.g., Cas9, Cas12) can refer to the wild-type or a modified form of the Cas protein that can comprise an amino acid change such as a deletion, insertion, substitution, variant, mutation, fusion, chimera, or any combination thereof.

In some embodiments, a CRISPR protein-derived domain of a base editor can include all or a portion (e.g., a functional portion) of Cas9 from Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquis (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); Neisseria meningitidis (NCBI Ref: YP_002342100.1), Streptococcus pyogenes, or Staphylococcus aureus.

Some aspects of the disclosure provide high fidelity Cas9 domains. High fidelity Cas9 domains are known in the art and described, for example, in Kleinstiver, B. P., et al. “High-fidelity CRISPR-Cas9 nucleases with no detectable genome-wide off-target effects.” Nature 529, 490-495 (2016); and Slaymaker, I. M., et al. “Rationally engineered Cas9 nucleases with improved specificity.” Science 351, 84-88 (2015); the entire contents of each of which are incorporated herein by reference. An Exemplary high fidelity Cas9 domain is provided in the Sequence Listing as SEQ ID NO: 233.

In some embodiments, any of the Cas9 fusion proteins or complexes provided herein comprise one or more of a D10A, N497X, a R661X, a Q695X, and/or a Q926X mutation, or a corresponding mutation in any of the amino acid sequences provided herein, wherein X is any amino acid.

Typically, Cas9 proteins, such as Cas9 from S. pyogenes (spCas9), require a “protospacer adjacent motif (PAM)” or PAM-like motif, which is a 2-6 base pair DNA sequence immediately following the DNA sequence targeted by the Cas9 nuclease in the CRISPR bacterial adaptive immune system. The presence of an NGG PAM sequence is required to bind a particular nucleic acid region, where the “N” in “NGG” is adenosine (A), thymidine (T), or cytosine (C), and the G is guanosine. In some embodiments, any of the fusion proteins or complexes provided herein may contain a Cas9 domain that is capable of binding a nucleotide sequence that does not contain a canonical (e.g., NGG) PAM sequence. Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); the entire contents of each are hereby incorporated by reference.

In some embodiments, the napDNAbp is a circular permutant (e.g., SEQ ID NO: 238).

In some embodiments, the polynucleotide programmable nucleotide binding domain comprises a nickase domain. Herein the term “nickase” refers to a polynucleotide programmable nucleotide binding domain comprising a nuclease domain that is capable of cleaving only one strand of the two strands in a duplexed nucleic acid molecule (e.g., DNA). For example, where a polynucleotide programmable nucleotide binding domain comprises a nickase domain derived from Cas9, the Cas9-derived nickase domain can include a D10A mutation and a histidine at position 840. In another example, a Cas9-derived nickase domain comprises an H840A mutation, while the amino acid residue at position 10 remains a D.

In some embodiments, a Cas9 nuclease has an inactive (e.g., an inactivated) DNA cleavage domain, that is, the Cas9 is a nickase, referred to as an “nCas9” protein (for “nickase” Cas9; SEQ ID NO: 201). The Cas9 nickase may be a Cas9 protein that is capable of cleaving only one strand of a duplexed nucleic acid molecule (e.g., a duplexed DNA molecule). In some embodiments the Cas9 nickase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the Cas9 nickases provided herein. Additional suitable Cas9 nickases will be apparent to those of skill in the art based on this disclosure and knowledge in the field and are within the scope of this disclosure.

Also provided herein are base editors comprising a polynucleotide programmable nucleotide binding domain which is catalytically dead (i.e., incapable of cleaving a target polynucleotide sequence). For example, in the case of a base editor comprising a Cas9 domain, the Cas9 can comprise both a D10A mutation and an H840A mutation. In further embodiments, a catalytically dead polynucleotide programmable nucleotide binding domain comprises a point mutation (e.g., D10A or H840A) as well as a deletion of all or a portion (e.g., a functional portion) of a nuclease domain. dCas9 domains are known in the art and described, for example, in Qi et al., “Repurposing CRISPR as an RNA-guided platform for sequence-specific control of gene expression.” Cell. 2013; 152(5):1173-83, the entire contents of which are incorporated herein by reference.

The term “protospacer adjacent motif (PAM)” or PAM-like motif refers to a 2-6 base pair DNA sequence immediately following the DNA sequence targeted by a nucleic acid programmable DNA binding protein. In some embodiments, the PAM can be a 5′ PAM (i.e., located upstream of the 5′ end of the protospacer). In other embodiments, the PAM can be a 3′ PAM (i.e., located downstream of the 5′ end of the protospacer). The PAM sequence can be any PAM sequence known in the art. Suitable PAM sequences include, but are not limited to, NGG, NGA, NGC, NGN, NGT, NGTT, NGCG, NGAG, NGAN, NGNG, NGCN, NGCG, NGTN, NNGRRT, NNNRRT, NNGRR(N), TTTV, TYCV, TYCV, TATV, NNNNGATT, NNAGAAW, or NAAAAC. Y is a pyrimidine; N is any nucleotide base; W is A or T.

A base editor provided herein can comprise a CRISPR protein-derived domain that is capable of binding a nucleotide sequence that contains a canonical or non-canonical protospacer adjacent motif (PAM) sequence.

In some embodiments, the PAM is an “NRN” PAM where the “N” in “NRN” is adenine (A), thymine (T), guanine (G), or cytosine (C), and the R is adenine (A) or guanine (G); or the PAM is an “NYN” PAM, wherein the “N” in NYN is adenine (A), thymine (T), guanine (G), or cytosine (C), and the Y is cytidine (C) or thymine (T), for example, as described in R. T. Walton et al., 2020, Science, 10.1126/science.aba8853 (2020), the entire contents of which are incorporated herein by reference.

Several PAM variants are described in Table 3 below.

TABLE 3
Cas9 proteins and corresponding PAM sequences.
N is A, C, T, or G; and V is A, C, or G.

| Variant | PAM |
|---|---|
| spCas9 | NGG |
| spCas9-VRQR | NGA |
| spCas9-VRER | NGCG |
| xCas9 (sp) | NGN |
| saCas9 | NNGRRT |
| saCas9-KKH | NNNRRT |
| spCas9-MQKSER | NGCG |
| spCas9-MQKSER | NGCN |
| spCas9-LRKIQK | NGTN |
| spCas9-LRVSQK | NGTN |
| spCas9-LRVSQL | NGTN |
| spCas9-MQKFRAER | NGC |
| Cpf1 | 5′ (TTTV) |
| SpyMac | 5′-NAA-3′ |

In some embodiments, a CRISPR protein-derived domain of a base editor comprises all or a portion (e.g., a functional portion) of a Cas9 protein with a canonical PAM sequence (NGG). In other embodiments, a Cas9-derived domain of a base editor can employ a non-canonical PAM sequence. Such sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); R. T. Walton et al. “Unconstrained genome targeting with near-PAMless engineered CRISPR-Cas9 variants” Science 10.1126/science.aba8853 (2020); Hu et al. “Evolved Cas9 variants with broad PAM compatibility and high DNA specificity,” Nature, 2018 Apr. 5, 556(7699), 57-63; Miller et al., “Continuous evolution of SpCas9 variants compatible with non-G PAMs” Nat. Biotechnol., 2020 April; 38(4):471-481; the entire contents of each are hereby incorporated by reference.

Fusion Proteins or Complexes Comprising a NapDNAbp and a Cytidine Deaminase and/or Adenosine Deaminase

Some aspects of the disclosure provide fusion proteins or complexes comprising a Cas9 domain or other nucleic acid programmable DNA binding protein (e.g., Cas12) and one or more cytidine deaminase, adenosine deaminase, or cytidine adenosine deaminase domains. It should be appreciated that the Cas9 domain may be any of the Cas9 domains or Cas9 proteins (e.g., dCas9 or nCas9) provided herein. In some embodiments, any of the Cas9 domains or Cas9 proteins (e.g., dCas9 or nCas9) provided herein may be fused with any of the cytidine deaminases and/or adenosine deaminases provided herein. The domains of the base editors disclosed herein can be arranged in any order.

In some embodiments, the fusion proteins or complexes comprising a cytidine deaminase or adenosine deaminase and a napDNAbp (e.g., Cas9 or Cas12 domain) do not include a linker sequence. In some embodiments, a linker is present between the cytidine or adenosine deaminase and the napDNAbp. In some embodiments, cytidine or adenosine deaminase and the napDNAbp are fused via any of the linkers provided herein. For example, in some embodiments the cytidine or adenosine deaminase and the napDNAbp are fused via any of the linkers provided herein.

It should be appreciated that the fusion proteins or complexes of the present disclosure may comprise one or more additional features. For example, in some embodiments, the fusion protein or complex may comprise inhibitors, cytoplasmic localization sequences, export sequences, such as nuclear export sequences, or other localization sequences, as well as sequence tags that are useful for solubilization, purification, or detection of the fusion proteins or complexes. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein or complex comprises one or more His tags.

Exemplary, yet nonlimiting, fusion proteins are described in International PCT Application Nos. PCT/US2017/045381, PCT/US2019/044935, and PCT/US2020/016288, each of which is incorporated herein by reference for its entirety.

Fusion Proteins or Complexes with Internal Insertions

Provided herein are fusion proteins or complexes comprising a heterologous polypeptide fused to a nucleic acid programmable nucleic acid binding protein, for example, a napDNAbp. The heterologous polypeptide can be fused to the napDNAbp at a C-terminal end of the napDNAbp, an N-terminal end of the napDNAbp, or inserted at an internal location of the napDNAbp. In some embodiments, the heterologous polypeptide is a deaminase (e.g., cytidine or adenosine deaminase) or a functional fragment thereof. For example, a fusion protein can comprise a deaminase flanked by an N-terminal fragment and a C-terminal fragment of a Cas9 or Cas12 (e.g., Cas12b/C2c1), polypeptide.

The deaminase can be a circular permutant deaminase. In some embodiments, the deaminase is a circular permutant TadA, circularly permutated at amino acid residue 116, 136, or 65 as numbered in a TadA reference sequence.

The fusion protein or complexes can comprise more than one deaminase. The fusion protein or complex can comprise, for example, 1, 2, 3, 4, 5 or more deaminases. The deaminases in a fusion protein or complex can be adenosine deaminases, cytidine deaminases, or a combination thereof.

In some embodiments, the napDNAbp in the fusion protein or complex contains a Cas9 polypeptide or a fragment thereof. The Cas9 polypeptide can be a variant Cas9 polypeptide. The Cas9 polypeptide can be a circularly permuted Cas9 protein.

The heterologous polypeptide (e.g., deaminase) can be inserted in the napDNAbp (e.g., Cas9 or Cas12 (e.g., Cas12b/C2c1)) at a suitable location, for example, such that the napDNAbp retains its ability to bind the target polynucleotide and a guide nucleic acid. A deaminase (e.g., adenosine deaminase, cytidine deaminase) can be inserted into a napDNAbp without compromising function of the deaminase (e.g., base editing activity) or the napDNAbp (e.g., ability to bind to target nucleic acid and guide nucleic acid).

In some embodiments, the deaminase (e.g., adenosine deaminase, cytidine deaminase, or adenosine deaminase and cytidine deaminase) is inserted in regions of the Cas9 polypeptide comprising higher than average B-factors (e.g., higher B factors compared to the total protein or the protein domain comprising the disordered region). Cas9 polypeptide positions comprising a higher than average B-factor can include, for example, residues 768, 792, 1052, 1015, 1022, 1026, 1029, 1067, 1040, 1054, 1068, 1246, 1247, and 1248 as numbered in the above Cas9 reference sequence. Cas9 polypeptide regions comprising a higher than average B-factor can include, for example, residues 792-872, 792-906, and 2-791 as numbered in the above Cas9 reference sequence.

In some embodiments, a heterologous polypeptide (e.g., deaminase) is inserted in a flexible loop of a Cas9 polypeptide. The flexible loop portions can be selected from the group consisting of 530-537, 569-570, 686-691, 943-947, 1002-1025, 1052-1077, 1232-1247, or 1298-1300 as numbered in the above Cas9 reference sequence, or a corresponding amino acid residue in another Cas9 polypeptide. The flexible loop portions can be selected from the group consisting of: 1-529, 538-568, 580-685, 692-942, 948-1001, 1026-1051, 1078-1231, or 1248-1297 as numbered in the above Cas9 reference sequence, or a corresponding amino acid residue in another Cas9 polypeptide.

A heterologous polypeptide (e.g., adenine deaminase) can be inserted into a Cas9 polypeptide region corresponding to amino acid residues: 1017-1069, 1242-1247, 1052-1056, 1060-1077, 1002-1003, 943-947, 530-537, 568-579, 686-691, 1242-1247, 1298-1300, 1066-1077, 1052-1056, or 1060-1077 as numbered in the above Cas9 reference sequence, or a corresponding amino acid residue in another Cas9 polypeptide.

A heterologous polypeptide (e.g., adenine deaminase) can be inserted in place of a deleted region of a Cas9 polypeptide. The deleted region can correspond to an N-terminal or C-terminal portion of the Cas9 polypeptide. Exemplary internal fusions base editors are provided in Table 4A below:

TABLE 4
Insertion loci in Cas9 proteins

| BE ID | Modification | Other ID |
|---|---|---|
| IBE001 | Cas9 TadA ins 1015 | ISLAY01 |
| IBE002 | Cas9 TadA ins 1022 | ISLAY02 |
| IBE003 | Cas9 TadA ins 1029 | ISLAY03 |
| IBE004 | Cas9 TadA ins 1040 | ISLAY04 |
| IBE005 | Cas9 TadA ins 1068 | ISLAY05 |
| IBE006 | Cas9 TadA ins 1247 | ISLAY06 |
| IBE007 | Cas9 TadA ins 1054 | ISLAY07 |
| IBE008 | Cas9 TadA ins 1026 | ISLAY08 |
| IBE009 | Cas9 TadA ins 768 | ISLAY09 |
| IBE020 | delta HNH TadA 792 | ISLAY20 |
| IBE021 | N-term fusion single TadA helix truncated 165-end | ISLAY21 |
| IBE029 | TadA-Circular Permutant116 ins1067 | ISLAY29 |
| IBE031 | TadA- Circular Permutant 136 ins1248 | ISLAY31 |
| IBE032 | TadA- Circular Permutant 136ins 1052 | ISLAY32 |
| IBE035 | delta 792-872 TadA ins | ISLAY35 |
| IBE036 | delta 792-906 TadA ins | ISLAY36 |
| IBE043 | TadA-Circular Permutant 65 ins1246 | ISLAY43 |
| IBE044 | TadA ins C-term truncate2 791 | ISLAY44 |

A heterologous polypeptide (e.g., deaminase) can be inserted within a structural or functional domain of a Cas9 polypeptide. A heterologous polypeptide (e.g., deaminase) can be inserted between two structural or functional domains of a Cas9 polypeptide. A heterologous polypeptide (e.g., deaminase) can be inserted in place of a structural or functional domain of a Cas9 polypeptide, for example, after deleting the domain from the Cas9 polypeptide. The structural or functional domains of a Cas9 polypeptide can include, for example, RuvC I, RuvC II, RuvC III, Rec1, Rec2, PI, or HNH.

A fusion protein can comprise a linker between the deaminase and the napDNAbp polypeptide. The linker can be a peptide or a non-peptide linker. For example, the linker can be an XTEN, (GGGS)n (SEQ ID NO: 246), (GGGGS)n (SEQ ID NO: 247), (G)n, (EAAAK)n (SEQ ID NO: 248), (GGS)n, SGSETPGTSESATPES (SEQ ID NO: 249). In other embodiments, the amino acid sequence of the linker is GGSGGS (SEQ ID NO: 250) or GSSGSETPGTSESATPESSG (SEQ ID NO: 251). In other embodiments, the linker is a rigid linker. In other embodiments of the above aspects, the linker is encoded by GGAGGCTCTGGAGGAAGC (SEQ ID NO: 252) or GGCTCTTCTGGATCTGAAACACCTGGCACAAGCGAGAGCGCCACCCCTGAGAGCTCTGGC (SEQ ID NO: 253). In some embodiments, the fusion protein comprises a linker between the N-terminal Cas9 fragment and the deaminase. In some embodiments, the fusion protein comprises a linker between the C-terminal Cas9 fragment and the deaminase. In some embodiments, the N-terminal and C-terminal fragments of napDNAbp are connected to the deaminase with a linker. In some embodiments, the N-terminal and C-terminal fragments are joined to the deaminase domain without a linker. In some embodiments, the fusion protein comprises a linker between the N-terminal Cas9 fragment and the deaminase but does not comprise a linker between the C-terminal Cas9 fragment and the deaminase. In some embodiments, the fusion protein comprises a linker between the C-terminal Cas9 fragment and the deaminase but does not comprise a linker between the N-terminal Cas9 fragment and the deaminase.

In other embodiments, the fusion protein or complex contains a nuclear localization signal (e.g., a bipartite nuclear localization signal). In other embodiments, the amino acid sequence of the nuclear localization signal is MAPKKKRKVGIHGVPAA (SEQ ID NO: 261). In other embodiments of the above aspects, the nuclear localization signal is encoded by the following sequence: ATGCCCCAAAGAAGAAGCGGAAGGTCGGTATCCACGGAGTCCCAGCAGCC (SEQ ID NO: 262). In some embodiments, the base editing system described herein is an ABE with TadA inserted into a Cas9. Polypeptide sequences of relevant ABEs with TadA inserted into a Cas9 are provided in the attached Sequence Listing as SEQ ID NOs: 263-308.

Exemplary, yet nonlimiting, fusion proteins are described in International PCT Application Nos. PCT/US2020/016285 and U.S. Provisional Application Nos. 62/852,228 and 62/852,224, the contents of which are incorporated by reference herein in their entireties.

### A to G Editing

In some embodiments, a base editor described herein comprises an adenosine deaminase domain. Such an adenosine deaminase domain of a base editor can facilitate the editing of an adenine (A) nucleobase to a guanine (G) nucleobase by deaminating the A to form inosine (I), which exhibits base pairing properties of G. In some embodiments, an A-to-G base editor further comprises an inhibitor of inosine base excision repair, for example, a uracil glycosylase inhibitor (UGI) domain or a catalytically inactive inosine specific nuclease. Without wishing to be bound by any particular theory, the UGI domain or catalytically inactive inosine specific nuclease can inhibit or prevent base excision repair of a deaminated adenosine residue (e.g., inosine), which can improve the activity or efficiency of the base editor.

A base editor comprising an adenosine deaminase can act on any polynucleotide, including DNA, RNA and DNA-RNA hybrids. In an embodiment an adenosine deaminase domain of a base editor comprises all or a portion (e.g., a functional portion) of an ADAT comprising one or more mutations which permit the ADAT to deaminate a target A in DNA. For example, the base editor can comprise all or a portion (e.g., a functional portion) of an ADAT from Escherichia coli (EcTadA) comprising one or more of the following mutations: D108N, A106V, D147Y, E155V, L84F, H123Y, I156F, or a corresponding mutation in another adenosine deaminase. Exemplary ADAT homolog polypeptide sequences are provided in the Sequence Listing as SEQ ID NOs: 1 and 309-315.

The adenosine deaminase can be derived from any suitable organism (e.g., E. coli). In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenine deaminase is a naturally-occurring adenosine deaminase that includes one or more mutations corresponding to any of the mutations provided herein (e.g., mutations in ecTadA). The corresponding residue in any homologous protein can be identified by e.g., sequence alignment and determination of homologous residues. The mutations in any naturally-occurring adenosine deaminase (e.g., having homology to ecTadA) that correspond to any of the mutations described herein (e.g., any of the mutations identified in ecTadA) can be generated accordingly.

In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any of the adenosine deaminases provided herein. It should be appreciated that adenosine deaminases provided herein may include one or more mutations (e.g., any of the mutations provided herein). The disclosure provides any deaminase domains with a certain percent identify plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to a reference sequence, or any of the adenosine deaminases provided herein.

It should be appreciated that any of the mutations provided herein (e.g., based on a TadA reference sequence, such as TadA*7.10 (SEQ ID NO: 1)) can be introduced into other adenosine deaminases, such as E. coli TadA (ecTadA), S. aureus TadA (saTadA), or other adenosine deaminases (e.g., bacterial adenosine deaminases). In some embodiments, the TadA reference sequence is TadA*7.10 (SEQ ID NO: 1). It would be apparent to the skilled artisan that additional deaminases may similarly be aligned to identify homologous amino acid residues that can be mutated as provided herein. Thus, any of the mutations identified in a TadA reference sequence can be made in other adenosine deaminases (e.g., ecTada) that have homologous amino acid residues. It should also be appreciated that any of the mutations provided herein can be made individually or in any combination in a TadA reference sequence or another adenosine deaminase.

In some embodiments, the adenosine deaminase comprises an alteration or set of alterations selected from those listed in Tables 5A-5E below:

TABLE 5A
Adenosine Deaminase Variants. Residue positions in the E. coli TadA variant (TadA*) are indicated.

| | 23 | 26 | 36 | 37 | 48 | 49 | 51 | 72 | 84 | 87 | 106 | 108 | 123 | 125 | 142 | 146 | 147 | 152 | 155 | 156 | 157 | 161 |
|---|---|---|---|---|---|---|---|---|---|---|---|---|---|---|---|---|---|---|---|---|---|---|
| TadA*0.1 | W | R | H | N | P | | R | N | L | S | A | D | H | G | A | S | D | R | E | I | K | K |
| TadA*0.2 | W | R | H | N | P | | R | N | L | S | A | D | H | G | A | S | D | R | E | I | K | K |
| TadA*1.1 | W | R | H | N | P | | R | N | L | S | A | N | H | G | A | S | D | R | E | I | K | K |
| TadA*1.2 | W | R | H | N | P | | R | N | L | S | V | N | H | G | A | S | D | R | E | I | K | K |
| TadA*2.1 | W | R | H | N | P | | R | N | L | S | V | N | H | G | A | S | Y | R | V | I | K | K |
| TadA*2.2 | W | R | H | N | P | | R | N | L | S | V | N | H | G | A | S | Y | R | V | I | K | K |
| TadA*2.3 | W | R | H | N | P | | R | N | L | S | V | N | H | G | A | S | Y | R | V | I | K | K |
| TadA*2.4 | W | R | H | N | P | | R | N | L | S | V | N | H | G | A | S | Y | R | V | I | K | K |
| TadA*2.5 | W | R | H | N | P | | R | N | L | S | V | N | H | G | A | S | Y | R | V | I | K | K |
| TadA*2.6 | W | R | H | N | P | | R | N | L | S | V | N | H | G | A | S | Y | R | V | I | K | K |
| TadA*2.7 | W | R | H | N | P | | R | N | L | S | V | N | H | G | A | S | Y | R | V | I | K | K |
| TadA*2.8 | W | R | H | N | P | | R | N | L | S | V | N | H | G | A | S | Y | R | V | I | K | K |
| TadA*2.9 | W | R | H | N | P | | R | N | L | S | V | N | H | G | A | S | Y | R | V | I | K | K |
| TadA*2.10 | W | R | H | N | P | | R | N | L | S | V | N | H | G | A | S | Y | R | V | I | K | K |
| TadA*2.11 | W | R | H | N | P | | R | N | L | S | V | N | H | G | A | S | Y | R | V | I | K | K |
| TadA*2.12 | W | R | H | N | P | | R | N | L | S | V | N | H | G | A | S | Y | R | V | I | K | K |
| TadA*3.1 | W | R | H | N | P | | R | N | F | S | V | N | Y | G | A | S | Y | R | V | F | K | K |
| TadA*3.2 | W | R | H | N | P | | R | N | F | S | V | N | Y | G | A | S | Y | R | V | F | K | K |
| TadA*3.3 | W | R | H | N | P | | R | N | F | S | V | N | Y | G | A | S | Y | R | V | F | K | K |
| TadA*3.4 | W | R | H | N | P | | R | N | F | S | V | N | Y | G | A | S | Y | R | V | F | K | K |
| TadA*3.5 | W | R | \|H | N | P | | R | N | F | S | V | N | Y | G | A | S | Y | R | V | F | K | K |
| TadA*3.6 | W | R | H | N | P | | R | N | F | S | V | N | Y | G | A | S | Y | R | V | F | K | K |
| TadA*3.7 | W | R | H | N | P | | R | N | F | S | V | N | Y | G | A | S | Y | R | V | F | K | K |
| TadA*3.8 | W | R | H | N | P | | R | N | F | S | V | N | Y | G | A | S | Y | R | V | F | K | K |
| TadA*4.1 | W | R | H | N | P | | R | N | L | S | V | N | H | G | N | S | Y | R | V | I | K | K |
| TadA*4.2 | W | G | H | N | P | | R | N | L | S | V | N | H | G | N | S | Y | R | V | I | K | K |
| TadA*4.3 | W | R | H | N | P | | R | N | F | S | V | N | Y | G | N | S | Y | R | V | F | K | K |
| TadA*5.1 | W | R | L | N | P | | L | N | F | S | V | N | Y | G | A | C | Y | R | V | F | N | K |
| TadA*5.2 | W | R | H | S | P | | R | N | F | S | V | N | Y | G | A | S | Y | R | V | F | K | T |
| TadA*5.3 | W | R | L | N | P | | L | N | I | S | V | N | Y | G | A | C | Y | R | V | F | N | K |
| TadA*5.4 | W | R | H | S | P | | R | N | F | S | V | N | Y | G | A | S | Y | R | V | F | K | T |
| TadA*5.5 | W | R | L | N | P | | L | N | F | S | V | N | Y | G | A | C | Y | R | V | F | N | K |
| TadA*5.6 | W | R | L | N | P | | L | N | F | S | V | N | Y | G | A | C | Y | R | V | F | N | K |
| TadA*5.7 | W | R | L | N | P | | L | N | F | S | V | N | Y | G | A | C | Y | R | V | F | N | K |
| TadA*5.8 | W | R | L | N | P | | L | N | F | S | V | N | Y | G | A | C | Y | R | V | F | N | K |
| TadA*5.9 | W | R | L | N | P | | L | N | F | S | V | N | Y | G | A | C | Y | R | V | F | N | K |
| TadA*5.10 | W | R | L | N | P | | L | N | F | S | V | N | Y | G | A | C | Y | R | V | F | N | K |
| TadA*5.11 | W | R | L | N | P | | L | N | F | S | V | N | Y | G | A | C | Y | R | V | F | N | K |
| TadA*5.12 | W | R | L | N | P | | L | N | F | S | V | N | Y | G | A | C | Y | R | V | F | N | K |
| TadA*5.13 | W | R | H | N | P | | L | D | F | S | V | N | Y | A | A | S | Y | R | V | F | K | K |
| TadA*5.14 | W | R | H | N | S | | L | N | F | C | V | N | Y | G | A | S | Y | R | V | F | K | K |
| TadA*6.1 | W | R | H | N | S | | L | N | F | S | V | N | Y | G | N | S | Y | R | V | F | K | K |
| TadA*6.2 | W | R | H | N | T | V | L | N | F | S | V | N | Y | G | N | S | Y | R | V | F | N | K |
| TadA*6.3 | W | R | L | N | S | | L | N | F | S | V | N | Y | G | A | C | Y | R | V | F | N | K |
| TadA*6.4 | W | R | L | N | S | | L | N | F | S | V | N | Y | G | N | C | Y | R | V | F | N | K |
| TadA*6.5 | W | R | L | N | T | V | L | N | F | S | V | N | Y | G | A | C | Y | R | V | F | N | K |
| TadA*6.6 | W | R | L | N | T | V | L | N | F | S | V | N | Y | G | N | C | Y | R | V | F | N | K |
| TadA*7.1 | W | R | L | N | A | | L | N | F | S | V | N | Y | G | A | C | Y | R | V | F | N | K |
| TadA*7.2 | W | R | L | N | A | | L | N | F | S | V | N | Y | G | N | C | Y | R | V | F | N | K |
| TadA*7.3 | L | R | L | N | A | | L | N | F | S | V | N | Y | G | A | C | Y | R | V | F | N | K |
| TadA*7.4 | R | R | L | N | A | | L | N | F | S | V | N | Y | G | A | C | Y | R | V | F | N | K |
| TadA*7.5 | W | R | L | N | A | | L | N | F | S | V | N | Y | G | A | C | Y | H | V | F | N | K |
| TadA*7.6 | W | R | L | N | A | | L | N | I | S | V | N | Y | G | A | C | Y | P | V | F | N | K |
| TadA*7.7 | L | R | L | N | A | | L | N | F | S | V | N | Y | G | A | C | Y | P | V | F | N | K |
| TadA*7.8 | L | R | L | N | A | | L | N | F | S | V | N | Y | G | N | C | Y | R | V | F | N | K |
| TadA*7.9 | L | R | L | N | A | | L | N | F | S | V | N | Y | G | N | C | Y | P | V | F | N | K |
| TadA*7.10 | R | R | L | N | A | | L | N | F | S | V | N | Y | G | A | C | Y | P | V | F | N | K |

TABLE 5B
Adenosine Deaminase Variants. Residue positions in the E. coli TadA variant
(TadA*) are indicated. Alterations are referenced to TadA*7.10 (first row).

| | 23 | 36 | 48 | 51 | 76 | 82 | 84 | 106 | 108 | 123 | 146 | 147 | 152 | 154 | 155 | 156 | 157 | 166 |
|---|---|---|---|---|---|---|---|---|---|---|---|---|---|---|---|---|---|---|
| TadA*7.10 | R | L | A | L | I | V | F | V | N | Y | C | Y | P | Q | V | F | N | T |
| TadA*8.1 | | | | | | | | | | | | T | | | | | | |
| TadA*8.2 | | | | | | | | | | | | R | | | | | | |
| TadA*8.3 | | | | | | | | | | | | | | S | | | | |
| TadA*8.4 | | | | | | | | | | H | | | | | | | | |
| TadA*8.5 | | | | | | S | | | | | | | | | | | | |
| TadA*8.6 | | | | | | | | | | | | | | | | | | R |
| TadA*8.7 | | | | | | | | | | | | | | R | | | | |
| TadA*8.8 | | | | | | | | | | H | | R | | R | | | | |
| TadA*8.9 | | | | | Y | | | | | | | R | | R | | | | |
| TadA*8.10 | | | | | | | | | | | | R | | R | | | | R |
| TadA*8.11 | | | | | | | | | | | | T | | R | | | | |
| TadA*8.12 | | | | | | | | | | | | T | | S | | | | |
| TadA*8.13 | | | | | Y | | | | | H | | R | | R | | | | |
| TadA*8.14 | | | | | Y | S | | | | | | | | | | | | |
| TadA*8.15 | | | | | | S | | | | | | R | | | | | | |
| TadA*8.16 | | | | | | S | | | | H | | R | | | | | | |
| TadA*8.17 | | | | | | S | | | | | | | | R | | | | |
| TadA*8.18 | | | | | | S | | | | H | | | | R | | | | |
| TadA*8.19 | | | | | | S | | | | H | | R | | R | | | | |
| TadA*8.20 | | | | | Y | S | | | | H | | R | | R | | | | |
| TadA*8.21 | | | | | | | | | | | | R | | S | | | | |
| TadA*8.22 | | | | | | S | | | | | | | | S | | | | |
| TadA*8.23 | | | | | | S | | | | H | | | | | | | | |
| TadA*8.24 | | | | | | S | | | | H | | T | | | | | | |

TABLE 5C
Adenosine Deaminase Variants. Alterations are referenced
to TadA*7.10. Additional details of TadA*9 adenosine
deaminases are described in International PCT Application
No. PCT/US2020/049975, which is incorporated herein
by reference in its entirety for all purposes.

| TadA*9 | |
|---|---|
| Description | Alterations |
| TadA*9.1 | E25F, V82S, Y123H, T133K, Y147R, Q154R |
| TadA*9.2 | E25F, V82S, Y123H, Y147R, Q154R |
| TadA*9.3 | V82S, Y123H, P124W, Y147R, Q154R |
| TadA*9.4 | L51W, V82S, Y123H, C146R, Y147R, Q154R |
| TadA*9.5 | P54C, V82S, Y123H, Y147R, Q154R |
| TadA*9.6 | Y73S, V82S, Y123H, Y147R, Q154R |
| TadA*9.7 | N38G, V82T, Y123H, Y147R, Q154R |
| TadA*9.8 | R23H, V82S, Y123H, Y147R, Q154R |
| TadA*9.9 | R21N, V82S, Y123H, Y147R, Q154R |
| TadA*9.10 | V82S, Y123H, Y147R, Q154R, A158K |
| TadA*9.11 | N72K, V82S, Y123H, D139L, Y147R, Q154R, |
| TadA*9.12 | E25F, V82S, Y123H, D139M, Y147R, Q154R |
| TadA*9.13 | M70V, V82S, M94V, Y123H, Y147R, Q154R |
| TadA*9.14 | Q71M, V82S, Y123H, Y147R, Q154R |
| TadA*9.15 | E25F, V82S, Y123H, T133K, Y147R, Q154R |
| TadA*9.16 | E25F, V82S, Y123H, Y147R, Q154R |
| TadA*9.17 | V82S, Y123H, P124W, Y147R, Q154R |
| TadA*9.18 | L51W, V82S, Y123H, C146R, Y147R, Q154R |
| TadA*9.19 | P54C, V82S, Y123H, Y147R, Q154R |
| TadA*9.2 | Y73S, V82S, Y123H, Y147R, Q154R |
| TadA*9.21 | N38G, V82T, Y123H, Y147R, Q154R |
| TadA*9.22 | R23H, V82S, Y123H, Y147R, Q154R |
| TadA*9.23 | R21N, V82S, Y123H, Y147R, Q154R |
| TadA*9.24 | V82S, Y123H, Y147R, Q154R, A158K |
| TadA*9.25 | N72K, V82S, Y123H, D139L, Y147R, Q154R, |
| TadA*9.26 | E25F, V82S, Y123H, D139M, Y147R, Q154R |
| TadA*9.27 | M70V, V82S, M94V, Y123H, Y147R, Q154R |
| TadA*9.28 | Q71M, V82S, Y123H, Y147R, Q154R |
| TadA*9.29 | E25F_I76Y_V82S_Y123H_Y147R_Q154R |
| TadA*9.30 | I76Y_V82T_Y123H_Y147R_Q154R |
| TadA*9.31 | N38G_I76Y_V82S_Y123H_Y147R_Q154R |
| TadA*9.32 | N38G_I76Y_V82T_Y123H_Y147R_Q154R |
| TadA*9.33 | R23H_I76Y_V82S_Y123H_Y147R_Q154R |
| TadA*9.34 | P54C_I76Y_V82S_Y123H_Y147R_Q154R |
| TadA*9.35 | R21N_I76Y_V82S_Y123H_Y147R_Q154R |
| TadA*9.36 | I76Y_V82S_Y123H_D138M_Y147R_Q154R |
| TadA*9.37 | Y72S_I76Y_V82S_Y123H_Y147R_Q154R |
| TadA*9.38 | E25F_I76Y_V82S_Y123H_Y147R_Q154R |
| TadA*9.39 | I76Y_V82T_Y123H_Y147R_Q154R |
| TadA*9.40 | N38G_I76Y_V82S_Y123H_Y147R_Q154R |
| TadA*9.41 | N38G_I76Y_V82T_Y123H_Y147R_Q154R |
| TadA*9.42 | R23H_I76Y_V82S_Y123H_Y147R_Q154R |
| TadA*9.43 | P54C_I76Y_V82S_Y123H_Y147R_Q154R |
| TadA*9.44 | R21N_I76Y_V82S_Y123H_Y147R_Q154R |
| TadA*9.45 | I76Y_V82S_Y123H_D138M_Y147R_Q154R |
| TadA*9.46 | Y72S_I76Y_V82S_Y123H_Y147R_Q154R |
| TadA*9.47 | N72K V82S, Y123H, Y147R, Q154R |
| TadA*9.48 | Q71M_V82S, Y123H, Y147R, Q154R |
| TadA*9.49 | M70V, V82S, M94V, Y123H, Y147R, Q154R |
| TadA*9.50 | V82S, Y123H, T133K, Y147R, Q154R |
| TadA*9.51 | V82S, Y123H, T133K, Y147R, Q154R, A158K |
| TadA*9.52 | M70V, Q71M, N72K, V82S, Y123H, Y147R, Q154R |
| TadA*9.53 | N72K_V82S, Y123H, Y147R, Q154R |
| TadA*9.54 | Q71M_V82S, Y123H, Y147R, Q154R |
| TadA*9.55 | M70V, V82S, M94V, Y123H, Y147R, Q154R |
| TadA*9.56 | V82S, Y123H, T133K, Y147R, Q154R |
| TadA*9.57 | V82S, Y123H, T133K, Y147R, Q154R, A158K |
| TadA*9.58 | M70V, Q71M, N72K, V82S, Y123H, Y147R, Q154R |

In some embodiments, the adenosine deaminase comprises one or more of M1, S2A, S2E, W4D, W4E, V4M, F76S, H8E, H8Y, E9Y, M12S, R13H, R131, R13Y, T17L, T17S, L18A, L18E, A19N, R21N, K20K, K20R, R21A, G22P, W23D), R23H, W23G, W23Q, W23L, W23R, D24E, D24G, E25F7, E25M, E25D), E25A, E25G, E25R, E25V, E25S, E25Y, R26D), R26E, R26G, R26N, R26Q, R26C, R26L, R26K, R26W, E27V, E27D), P29V, V30G, L34S, L34V, L36H, H36L, H36N, N37N, N37T, N37S, N38G, N38R, W45A, W45L, W45N, N46N, R46W, R46F7, R46Q, R46M, R47A, R47Q, R47F7, R47K, R47P, R47W, R47M, P48T, P48L, P48A, P48I, P48S, I49G, I49H, I49V, I49F, I49H, G50L, R51H, R51L, R51N, L51W, R51Y, H52D, H52Y, D53P, P54C, P54T, A55H, T55A, A56E, A56S, E59A, E59G, E591, E59Q, E59W, M61A, M61I, M61L, M61V, L63S, L63V, Q65V, G66C, G67D), G67L, G67V, L68Q, M70H, M70Q, L84F7, M70V, M70L, E70A, M70V, Q71M, Q71N, Q71L, Q71R, N72A, N72K, N72S, N72D), N72Y, Y73G, Y73I, Y73K, Y73R, Y73S, R74A, R74Q, R74G, R74K, R74L, R74N, I761D, I76F, 1761, 176N, I76T, I76Y, D77G, A78I, T79M, L80M, L80Y, V82A, V82S, V82G, V82T, L84E, L84F, L84Y, E85K, E85G, E85P, E85S, S87C, S87L, S87V, V88A, V88M, C90S, A91A, A91G, A91S, A91V, A91T, G92T, A93I, M94A, M94V, M94L, M94I, M94H, I95S, I95G, I95L, I95H, I95V, H96A, H96L, H96R, H96S, S97C, S97G, S97I, S97M, S97R, S97S, R98K, R98I, R98N, R98Q, G100R, G100V, R101V, R101R, V102A, V102F, V102I, V102V, D103A, F104G, D104N, F104V, F104I, F104L, A106T, V106Q, V106F, V106W, V106M, A106A, A106Q, A106F, A106G, A106W, A106M, A106V, A106R, R107C, R107G, R107P, R107K, R107A, R107N, R107W, R107H, R107S, D108N, D108F, D108G, D108V, D108A, D108Y, D108H, D108I, D108K, D108L, D108M, D108Q, N108Q, N108F, N108W, N108M, N108K, D108K, D108F, D108M, D108Q, D108R, D108W, D108S, A109H, A109K, A109R, A109S, A109T, A109V, K110G, K110H, K110I, K110R, K110T, T111A, T111G, T111H, T111R, G112A, A114G, A114H, A114V, G115S, L117M, L117N, L117V, M118D, M118G, M118K, M118N, M118V, D119L, D119N, D119S, D119V, V120H, V120L, H122H, H122N, H122P, H122R, H122S, H122Y, H123C, H123G, H123P, H123V, H123Y, Y123H, P124G, P124I, P124L, P124W, G125H, G125I, G125A, G125M, G125K, M126D, M126H, M126K, M126I, M126N, M1260, M126S, M126Y, N127H, N127S, N127D, N127K, N127R, H128R, R129H, R129Q, R129V, R129I, R129E, R129V, I1321, I132F, T133V, T133E, T133G, T133K, E134A, E134E, E134G, E134I, G135G, G135V, I136G, I136L, I136T, I137A, I137D, I137E, L137M, I1375, A138D, A138E, A138G, S138A, A138N, A138S, A138T, A138V, A138Y, D139E, D139I, D139C, D139L, D139M, E140A, E140C, E140L, E140R, A142N, A142D, A142G, A142A, A142L, A142S, A142T, A142N, A142S, A142V, A143D, A143E, A143G, A143D, A143G, A143E, A143L, A143W, A143M, A143S, A143Q, A143R, C146R, S146A, S146C, S146D, S146F, S146R, S146T, D147D, D147L, D147F, D147G, D147Y, Y147T, Y147R, Y147D, D147R, F148L, F148F, F148R, F148Y, F149C, F149M, F149R, F149Y, M151F, M151P, M151R, M151V, R152C, R152F, R152H, R152P, R152R, R153C, R153Q, R153R, R153V, Q154E, Q154H, Q154M, Q154R, Q154L, Q154S, Q154V, E155F, E155G, E155I, E155K, E155P, E155V, E155D, I156A, I156F, I156D, I156K, I156N, I156R, I156Y, E157A, E157F, E157I, E157P, E157T, E157V, N157K, K157N, K157R, A158Q, A158K, A158V, Q159F, Q159K, Q159L, Q159N, K160A, K160S, K160E, K160K, K160N, K161I, K161A, K161N, K161Q, K161S, K161T, A162D, A162Q, R162H, R162P, A162S, Q163G, Q163H, Q163N, Q163R, S164I, S164R, S164Y, S165A, S165D, S165I, S165T, S165Y, T166D, T166K, T166I, T166N, T166P, T166R, D167S and/or D167N mutation in a TadA reference sequence (e.g., TadA*7.10, ecTadA, or TadA8e), and any alternative mutation at the corresponding position, or any substitution from R26, W23, E27, H36, R47, P48, R51, H52, R74, 176, V82, V88, M94, 195, H96, A106, D108, A109, K110, T111, A114, D119, H122, H123, M126, N127, A142, S146, D147, F149, R152, Q154, E155, 1156, E157, K161, T166, and/or D167, with respect to a TadA reference sequence, or a substitution of 2-50 amino acids in a TadA reference sequence, which may be selected from W23R, E27D, H36L, R47K, P48A, R51H, R51L, I76F, I76Y, V82S, A106V, D108G, A109S, K110R, T111H, A114V, D119N, H122R, H122N, H123Y, M126I, N127K, S146C, D147R, R152P, Q154R, E155V, 1156F, K157N, K161N, T166I, and D167N, or one or more corresponding mutations in another adenosine deaminase. Additional mutations are described in U.S. Patent Application Publication No. 2022/0307003 A1 and International Patent Application Publications No. WO 2023/288304 A2 and WO 2023/034959 A2, the disclosures of which are incorporated herein by reference in their entirety for all purposes.

In embodiments, a variant of TadA*7.10 comprises one or more alterations selected from any of those alterations provided herein.

In particular embodiments, an adenosine deaminase heterodimer comprises a TadA*8 domain and an adenosine deaminase domain selected from Staphylococcus aureus (S. aureus) TadA, Bacillus subtilis (B. subtilis) TadA, Salmonella typhimurium (S. typhimurium) TadA, Shewanella putrefaciens (S. putrefaciens) TadA, Haemophilus influenzae F3031 (H influenzae) TadA, Caulobacter crescentus (C. crescentus) TadA, Geobacter sulfurreducens (G. sulfurreducens) TadA, or TadA*7.10.

In some embodiments, the TadA*8 is a variant as shown in Table 5D. Table 5D shows certain amino acid position numbers in the TadA amino acid sequence and the amino acids present in those positions in the TadA-7.10 adenosine deaminase. Table 5D also shows amino acid changes in TadA variants relative to TadA-7.10 following phage-assisted non-continuous evolution (PANCE) and phage-assisted continuous evolution (PACE), as described in M. Richter et al., 2020, Nature Biotechnology, doi.org/10.1038/s41587-020-0453-z, the entire contents of which are incorporated by reference herein. In some embodiments, the TadA*8 is TadA*8a, TadA*8b, TadA*8c, TadA*8d, or TadA*8e. In some embodiments, the TadA*8 is TadA*8e. In one embodiment, an adenosine deaminase is a TadA*8 that comprises or consists essentially of SEQ ID NO: 316 or a fragment thereof having adenosine deaminase activity.

TABLE 5D
Select TadA*8 Variants

| | | TadA amino acid number | | | | | | | | | |
|---|---|---|---|---|---|---|---|---|---|---|---|
| | TadA | 26 | 88 | 109 | 111 | 119 | 122 | 147 | 149 | 166 | 167 |
| | TadA-7.10 | R | V | A | T | D | H | Y | F | T | D |
| PANCE 1 | | | | | R | | | | | | |
| PANCE 2 | | | | S/T | R | | | | | | |
| PACE | TadA-8a | C | | S | R | N | N | D | Y | I | N |
| | TadA-8b | | A | S | R | N | N | | Y | I | N |
| | TadA-8c | C | | S | R | N | N | | Y | I | N |
| | TadA-8d | | A | | R | N | | | Y | | |
| | TadA-8e | | | S | R | N | N | D | Y | I | N |

In some embodiments, the TadA variant is a variant as shown in Table 5E. Table 5E shows certain amino acid position numbers in the TadA amino acid sequence and the amino acids present in those positions in the TadA*7.10 adenosine deaminase. In some embodiments, the TadA variant is MSP605, MSP680, MSP823, MSP824, MSP825, MSP827, MSP828, or MSP829. In some embodiments, the TadA variant is MSP828. In some embodiments, the TadA variant is MSP829.

TABLE 5E
TadA Variants

| | TadA Amino Acid Number | | | | | | | |
|---|---|---|---|---|---|---|---|---|
| Variant | 36 | 76 | 82 | 147 | 149 | 154 | 157 | 167 |
| TadA-7.10 | L | I | V | Y | F | Q | N | D |
| MSP605 | | | G | T | | S | | |
| MSP680 | | Y | G | T | | S | | |
| MSP823 | H | | G | T | | S | K | |
| MSP824 | | | G | D | Y | S | | N |
| MSP825 | H | | G | D | Y | S | K | N |
| MSP827 | H | Y | G | T | | S | K | |
| MSP828 | | Y | G | D | Y | S | | N |
| MSP829 | H | Y | G | D | Y | S | K | N |

In particular embodiments, the fusion proteins or complexes comprise a single (e.g., provided as a monomer) TadA* (e.g., TadA*8 or TadA*9). Throughout the present disclosure, an adenosine deaminase base editor that comprises a single TadA* domain is indicates using the terminology ABEm or ABE #m, where “#” is an identifying number (e.g., ABE8.20m), where “m” indicates “monomer.” In some embodiments, the TadA* is linked to a Cas9 nickase. In some embodiments, the fusion proteins or complexes of the disclosure comprise as a heterodimer of a wild-type TadA (TadA(wt)) linked to a TadA*. Throughout the present disclosure, an adenosine deaminase base editor that comprises a single TadA* domain and a TadA(wt) domain is indicates using the terminology ABEd or ABE #d, where “#” is an identifying number (e.g., ABE8.20d), where “d” indicates “dimer.” In other embodiments, the fusion proteins or complexes of the disclosure comprise as a heterodimer of a TadA*7.10 linked to a TadA*. In some embodiments, the base editor is ABE8 comprising a TadA* variant monomer. In some embodiments, the base editor is ABE comprising a heterodimer of a TadA* and a TadA(wt). In some embodiments, the base editor is ABE comprising a heterodimer of a TadA* and TadA*7.10. In some embodiments, the base editor is ABE comprising a heterodimer of a TadA*. In some embodiments, the TadA* is selected from Tables 5A-5E.

In some embodiments, the adenosine deaminase is expressed as a monomer. In other embodiments, the adenosine deaminase is expressed as a heterodimer. In some embodiments, the deaminase or other polypeptide sequence lacks a methionine, for example when included as a component of a fusion protein. This can alter the numbering of positions. However, the skilled person will understand that such corresponding mutations refer to the same mutation.

Any of the mutations provided herein and any additional mutations (e.g., based on the ecTadA amino acid sequence) can be introduced into any other adenosine deaminases. Any of the mutations provided herein can be made individually or in any combination in a TadA reference sequence or another adenosine deaminase (e.g., ecTadA).

Details of A to G nucleobase editing proteins are described in International PCT Application No. PCT/US2017/045381 (WO2018/027078) and Gaudelli, N. M., et al., “Programmable base editing of A·T to G·C in genomic DNA without DNA cleavage” Nature, 551, 464-471 (2017), the entire contents of which are hereby incorporated by reference.

### C to T Editing

In some embodiments, a base editor disclosed herein comprises a fusion protein or complex comprising cytidine deaminase capable of deaminating a target cytidine (C) base of a polynucleotide to produce uridine (U), which has the base pairing properties of thymine. In some embodiments, for example where the polynucleotide is double-stranded (e.g., DNA), the uridine base can then be substituted with a thymidine base (e.g., by cellular repair machinery) to give rise to a C:G to a T:A transition. In other embodiments, deamination of a C to U in a nucleic acid by a base editor cannot be accompanied by substitution of the U to a T.

The deamination of a target C in a polynucleotide to give rise to a U is a non-limiting example of a type of base editing that can be executed by a base editor described herein. In another example, a base editor comprising a cytidine deaminase domain can mediate conversion of a cytosine (C) base to a guanine (G) base. For example, a U of a polynucleotide produced by deamination of a cytidine by a cytidine deaminase domain of a base editor can be excised from the polynucleotide by a base excision repair mechanism (e.g., by a uracil DNA glycosylase (UDG) domain), producing an abasic site. The nucleobase opposite the abasic site can then be substituted (e.g., by base repair machinery) with another base, such as a C, by for example a translesion polymerase. Although it is typical for a nucleobase opposite an abasic site to be replaced with a C, other substitutions (e.g., A, G or T) can also occur.

Accordingly, in some embodiments a base editor described herein comprises a deamination domain (e.g., cytidine deaminase domain) capable of deaminating a target C to a U in a polynucleotide. Further, as described below, the base editor can comprise additional domains which facilitate conversion of the U resulting from deamination to, in some embodiments, a T or a G. For example, a base editor comprising a cytidine deaminase domain can further comprise a uracil glycosylase inhibitor (UGI) domain to mediate substitution of a U by a T, completing a C-to-T base editing event. In another example, the base editor can comprise a uracil stabilizing protein as described herein. In another example, a base editor can incorporate a translesion polymerase to improve the efficiency of C-to-G base editing, since a translesion polymerase can facilitate incorporation of a C opposite an abasic site (i.e., resulting in incorporation of a G at the abasic site, completing the C-to-G base editing event).

A base editor comprising a cytidine deaminase as a domain can deaminate a target C in any polynucleotide, including DNA, RNA and DNA-RNA hybrids.

In some embodiments, a cytidine deaminase of a base editor comprises all or a portion (e.g., a functional portion) of an apolipoprotein B mRNA editing complex (APOBEC) family deaminase. APOBEC is a family of evolutionarily conserved cytidine deaminases. Members of this family are C-to-U editing enzymes. The N-terminal domain of APOBEC like proteins is the catalytic domain, while the C-terminal domain is a pseudocatalytic domain. More specifically, the catalytic domain is a zinc dependent cytidine deaminase domain and is important for cytidine deamination. APOBEC family members include APOBEC1, APOBEC2, APOBEC3A, APOBEC3B, APOBEC3C, APOBEC3D (“APOBEC3E” now refers to this), APOBEC3F, APOBEC3G, APOBEC3H, APOBEC4, and Activation-induced (cytidine) deaminase.

Other exemplary deaminases that can be fused to Cas9 according to aspects of this disclosure are provided below. In embodiments, the deaminases are activation-induced deaminases (AID). It should be understood that, in some embodiments, the active domain of the respective sequence can be used, e.g., the domain without a localizing signal (nuclear localization sequence, without nuclear export signal, cytoplasmic localizing signal).

Some aspects of the present disclosure are based on the recognition that modulating the deaminase domain catalytic activity of any of the fusion proteins or complexes described herein, for example by making point mutations in the deaminase domain, affect the processivity of the fusion proteins (e.g., base editors) or complexes. For example, mutations that reduce, but do not eliminate, the catalytic activity of a deaminase domain within a base editing fusion protein or complexes can make it less likely that the deaminase domain will catalyze the deamination of a residue adjacent to a target residue, thereby narrowing the deamination window. The ability to narrow the deamination window can prevent unwanted deamination of residues adjacent to specific target residues, which can decrease or prevent off-target effects.

In some embodiments, an APOBEC deaminase incorporated into a base editor can comprise one or more mutations selected from the group consisting of H121R, H122R, R126A, R126E, R118A, W90A, W90Y, and R132E of rAPOBEC1; D316R, D317R, R320A, R320E, R313A, W285A, W285Y, and R326E of hAPOBEC3G; and any alternative mutation at the corresponding position, or one or more corresponding mutations in another APOBEC deaminase.

A number of modified cytidine deaminases are commercially available, including, but not limited to, SaBE3, SaKKH-BE3, VQR-BE3, EQR-BE3, VRER-BE3, YE1-BE3, EE-BE3, YE2-BE3, and YEE-BE3, which are available from Addgene (plasmids 85169, 85170, 85171, 85172, 85173, 85174, 85175, 85176, 85177). In some embodiments, a deaminase incorporated into a base editor comprises all or a portion (e.g., a functional portion) of an APOBEC1 deaminase.

In some embodiments, the fusion proteins or complexes of the disclosure comprise one or more cytidine deaminase domains. In some embodiments, the cytidine deaminases provided herein are capable of deaminating cytosine or 5-methylcytosine to uracil or thymine. In some embodiments, the cytidine deaminases provided herein are capable of deaminating cytosine in DNA. The cytidine deaminase may be derived from any suitable organism. In some embodiments, the cytidine deaminase is a naturally-occurring cytidine deaminase that includes one or more mutations corresponding to any of the mutations provided herein. One of skill in the art will be able to identify the corresponding residue in any homologous protein, e.g., by sequence alignment and determination of homologous residues. Accordingly, one of skill in the art would be able to generate mutations in any naturally-occurring cytidine deaminase that corresponds to any of the mutations described herein. In some embodiments, the cytidine deaminase is from a prokaryote. In some embodiments, the cytidine deaminase is from a bacterium. In some embodiments, the cytidine deaminase is from a mammal (e.g., human).

In some embodiments, the cytidine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the cytidine deaminase amino acid sequences set forth herein. It should be appreciated that cytidine deaminases provided herein may include one or more mutations (e.g., any of the mutations provided herein). Some embodiments provide a polynucleotide molecule encoding the cytidine deaminase nucleobase editor polypeptide of any previous aspect or as delineated herein. In some embodiments, the polynucleotide is codon optimized.

In embodiments, a fusion protein of the disclosure comprises two or more nucleic acid editing domains.

Details of C to T nucleobase editing proteins are described in International PCT Application No. PCT/US2016/058344 (WO2017/070632) and Komor, A. C., et al., “Programmable editing of a target base in genomic DNA without double-stranded DNA cleavage” Nature 533, 420-424 (2016), the entire contents of which are hereby incorporated by reference.

### Guide Polynucleotides

A polynucleotide programmable nucleotide binding domain, when in conjunction with a bound guide polynucleotide (e.g., gRNA), can specifically bind to a target polynucleotide sequence (i.e., via complementary base pairing between bases of the bound guide nucleic acid and bases of the target polynucleotide sequence) and thereby localize the base editor to the target nucleic acid sequence desired to be edited. In some embodiments, the target polynucleotide sequence comprises single-stranded DNA or double-stranded DNA. In some embodiments, the target polynucleotide sequence comprises RNA. In some embodiments, the target polynucleotide sequence comprises a DNA-RNA hybrid.

In an embodiment, a guide polynucleotide described herein can be RNA or DNA. In one embodiment, the guide polynucleotide is a gRNA.

In some embodiments, the guide polynucleotide is at least one single guide RNA (“sgRNA” or “gRNA”). In some embodiments, a guide polynucleotide comprises two or more individual polynucleotides, which can interact with one another via for example complementary base pairing (e.g., a dual guide polynucleotide, dual gRNA). For example, a guide polynucleotide can comprise a CRISPR RNA (crRNA) and a trans-activating CRISPR RNA (tracrRNA) or can comprise one or more trans-activating CRISPR RNA (tracrRNA).

A guide polynucleotide may include natural or non-natural (or unnatural) nucleotides (e.g., peptide nucleic acid or nucleotide analogs). In some cases, the targeting region of a guide nucleic acid sequence (e.g., a spacer) can be at least 15, 16, 17, 18, 19, 20, 21, 22, 23, 24, 25, 26, 27, 28, 29, or 30 nucleotides in length.

In some embodiments, the methods described herein can utilize an engineered Cas protein. A guide RNA (gRNA) is a short synthetic RNA composed of a scaffold sequence necessary for Cas-binding and a user-defined ˜20 nucleotide spacer that defines the genomic target to be modified. Exemplary gRNA scaffold sequences are provided in the sequence listing as SEQ ID NOs: 317-327 and 3394. Thus, a skilled artisan can change the genomic target of the Cas protein specificity is partially determined by how specific the gRNA targeting sequence is for the genomic target compared to the rest of the genome. In embodiments, the spacer is about 10, 11, 12, 13, 14, 15, 16, 17, 18, 19, 20, 22, 23, 24, 25, or more nucleotides in length. The spacer of a gRNA can be or can be about 19, 20, or 21 nucleotides in length. A gRNA or a guide polynucleotide can target any exon or intron of a gene target. In some embodiments, a composition comprises multiple gRNAs that all target the same exon or multiple gRNAs that target different exons. An exon and/or an intron of a gene can be targeted. A gRNA or a guide polynucleotide can target a nucleic acid sequence of about 20 nucleotides or less than about 20 nucleotides (e.g., at least about 5, 10, 15, 16, 17, 18, 19, 20, 21, 22, 23, 24, 25, 30 nucleotides), or anywhere between about 1-100 nucleotides (e.g., 5, 10, 15, 16, 17, 18, 19, 20, 21, 22, 23, 24, 25, 30, 40, 50, 60, 70, 80, 90, 100). A target nucleic acid sequence can be or can be about 20 bases immediately 5′ of the first nucleotide of the PAM. A gRNA can target a nucleic acid sequence. A target nucleic acid can be at least or at least about 1-10, 1-20, 1-30, 1-40, 1-50, 1-60, 1-70, 1-80, 1-90, or 1-100 nucleotides.

The guide polynucleotides can comprise standard ribonucleotides, modified ribonucleotides (e.g., pseudouridine), ribonucleotide isomers, and/or ribonucleotide analogs.

In some embodiments, a base editor system may comprise multiple guide polynucleotides, e.g., gRNAs. For example, the gRNAs may target to one or more target loci (e.g., at least 1 gRNA, at least 2 gRNA, at least 5 gRNA, at least 10 gRNA, at least 20 gRNA, at least 30 g RNA, at least 50 gRNA) comprised in a base editor system. The multiple gRNA sequences can be tandemly arranged and may be separated by a direct repeat.

### Modified Polynucleotides

To enhance expression, stability, and/or genomic/base editing efficiency, and/or reduce possible toxicity, the base editor-coding sequence (e.g., mRNA) and/or the guide polynucleotide (e.g., gRNA) can be modified to include one or more modified nucleotides and/or chemical modifications, e.g. using pseudo-uridine, 5-Methyl-cytosine, 2′-O-methyl-3′-phosphonoacetate, 2′-O-methyl thioPACE (MSP), 2′-O-methyl-PACE (MP), 2′-fluoro RNA (2′-F-RNA), =constrained ethyl (S-cEt), 2′-O-methyl (‘M’), 2′-O-methyl-3′-phosphorothioate (‘MS’), 2′-O-methyl-3′-thiophosphonoacetate (‘MSP’), 5-methoxyuridine, phosphorothioate, and N1-Methylpseudouridine. Chemically protected gRNAs can enhance stability and editing efficiency in vivo and ex vivo. Methods for using chemically modified mRNAs and guide RNAs are known in the art and described, for example, by Jiang et al., Chemical modifications of adenine base editor mRNA and guide RNA expand its application scope. Nat Commun 11, 1979 (2020). doi.org/10.1038/s41467-020-15892-8, Callum et al., N1-Methylpseudouridine substitution enhances the performance of synthetic mRNA switches in cells, Nucleic Acids Research, Volume 48, Issue 6, 6 Apr. 2020, Page e35, and Andries et al., Journal of Controlled Release, Volume 217, 10 Nov. 2015, Pages 337-344, each of which is incorporated herein by reference in its entirety.

In some embodiments, the guide polynucleotide comprises one or more modified nucleotides at the 5′ end and/or the 3′ end of the guide. In some embodiments, the guide polynucleotide comprises two, three, four or more modified nucleosides at the 5′ end and/or the 3′ end of the guide. In some embodiments, the guide polynucleotide comprises two, three, four or more modified nucleosides at the 5′ end and/or the 3′ end of the guide.

In some embodiments, the guide comprises at least about 50%-75% modified nucleotides. In some embodiments, the guide comprises at least about 85% or more modified nucleotides. In some embodiments, at least about 1-5 nucleotides at the 5′ end of the gRNA are modified and at least about 1-5 nucleotides at the 3′ end of the gRNA are modified. In some embodiments, at least about 3-5 contiguous nucleotides at each of the 5′ and 3′ termini of the gRNA are modified. In some embodiments, at least about 20% of the nucleotides present in a direct repeat or anti-direct repeat are modified. In some embodiments, at least about 50% of the nucleotides present in a direct repeat or anti-direct repeat are modified. In some embodiments, at least about 50-75% of the nucleotides present in a direct repeat or anti-direct repeat are modified. In some embodiments, at least about 100 of the nucleotides present in a direct repeat or anti-direct repeat are modified. In some embodiments, at least about 20% or more of the nucleotides present in a hairpin present in the gRNA scaffold are modified. In some embodiments, at least about 50% or more of the nucleotides present in a hairpin present in the gRNA scaffold are modified. In some embodiments, the guide comprises a variable length spacer. In some embodiments, the guide comprises a 20-40 nucleotide spacer. In some embodiments, the guide comprises a spacer comprising at least about 20-25 nucleotides or at least about 30-35 nucleotides. In some embodiments, the spacer comprises modified nucleotides. In some embodiments, the guide comprises two or more of the following:
at least about 1-5 nucleotides at the 5′ end of the gRNA are modified and at least about 1-5 nucleotides at the 3′ end of the gRNA are modified;
at least about 20% of the nucleotides present in a direct repeat or anti-direct repeat are modified;
at least about 50-75% of the nucleotides present in a direct repeat or anti-direct repeat are modified;
at least about 20% or more of the nucleotides present in a hairpin present in the gRNA scaffold are modified;
a variable length spacer; and
a spacer comprising modified nucleotides.

In embodiments, the gRNA contains numerous modified nucleotides and/or chemical modifications (“heavy mods”). Such heavy mods can increase base editing ˜2 fold in vivo or in vitro. In embodiments, the gRNA comprises 2′-O-methyl or phosphorothioate modifications. In an embodiment, the gRNA comprises 2′-O-methyl and phosphorothioate modifications. In an embodiment, the modifications increase base editing by at least about 2 fold.

A guide polynucleotide can comprise one or more modifications to provide a nucleic acid with a new or enhanced feature. A guide polynucleotide can comprise a nucleic acid affinity tag. A guide polynucleotide can comprise synthetic nucleotide, synthetic nucleotide analog, nucleotide derivatives, and/or modified nucleotides.

A gRNA or a guide polynucleotide can also be modified by 5′ adenylate, 5′ guanosine-triphosphate cap, 5′ N7-Methylguanosine-triphosphate cap, 5′ triphosphate cap, 3′ phosphate, 3′ thiophosphate, 5′ phosphate, 5′ thiophosphate, Cis-Syn thymidine dimer, trimers, C12 spacer, C3 spacer, C6 spacer, dSpacer, PC spacer, rSpacer, Spacer 18, Spacer 9, 3′-3′ modifications, 2′-O-methyl thioPACE (MSP), 2′-O-methyl-PACE (MP), and constrained ethyl (S-cEt), 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′ DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′-deoxyribonucleoside analog purine, 2′-deoxyribonucleoside analog pyrimidine, ribonucleoside analog, 2′-O-methyl ribonucleoside analog, sugar modified analogs, wobble/universal bases, fluorescent dye label, 2′-fluoro RNA, 2′-O-methyl RNA, methylphosphonate, phosphodiester DNA, phosphodiester RNA, phosphothioate DNA, phosphorothioate RNA, UNA, pseudouridine-5′-triphosphate, 5′-methylcytidine-5′-triphosphate, or any combination thereof.

In some cases, a phosphorothioate enhanced RNA gRNA can inhibit RNase A, RNase T1, calf serum nucleases, or any combinations thereof. These properties can allow the use of PS-RNA gRNAs to be used in applications where exposure to nucleases is of high probability in vivo or in vitro. For example, phosphorothioate (PS) bonds can be introduced between the last 3-5 nucleotides at the 5′- or 3′-end of a gRNA which can inhibit exonuclease degradation. In some cases, phosphorothioate bonds can be added throughout an entire gRNA to reduce attack by endonucleases.

### Fusion Proteins or Complexes Comprising a Nuclear Localization Sequence (NLS)

In some embodiments, the fusion proteins or complexes provided herein further comprise one or more (e.g., 2, 3, 4, 5) nuclear targeting sequences, for example a nuclear localization sequence (NLS). In one embodiment, a bipartite NLS is used. In some embodiments, a NLS comprises an amino acid sequence that facilitates the importation of a protein, that comprises an NLS, into the cell nucleus (e.g., by nuclear transport). In some embodiments, the NLS is fused to the N-terminus or the C-terminus of the fusion protein. In some embodiments, the NLS is fused to the C-terminus or N-terminus of an nCas9 domain or a dCas9 domain. In some embodiments, the NLS is fused to the N-terminus or C-terminus of the Cas12 domain. In some embodiments, the NLS is fused to the N-terminus or C-terminus of the cytidine or adenosine deaminase. In some embodiments, the NLS is fused to the fusion protein via one or more linkers. In some embodiments, the NLS is fused to the fusion protein without a linker. In some embodiments, the NLS comprises an amino acid sequence of any one of the NLS sequences provided or referenced herein. Additional nuclear localization sequences are known in the art and would be apparent to the skilled artisan. For example, NLS sequences are described in Plank et al., PCT/EP2000/011690, the contents of which are incorporated herein by reference for their disclosure of exemplary nuclear localization sequences.

In some embodiments, the NLS is present in a linker or the NLS is flanked by linkers, for example described herein. A bipartite NLS comprises two basic amino acid clusters, which are separated by a relatively short spacer sequence (hence bipartite—2 parts, while monopartite NLSs are not). The NLS of nucleoplasmin, KR [PAATKKAGQA]KKKK (SEQ ID NO: 191), is the prototype of the ubiquitous bipartite signal: two clusters of basic amino acids, separated by a spacer of about 10 amino acids. The sequence of an exemplary bipartite NLS follows:

(SEQ ID NO: 328)
PKKKRKVEGADKRTADGSEFESPKKKRKV

### Additional Domains

A base editor described herein can include any domain which helps to facilitate the nucleobase editing, modification or altering of a nucleobase of a polynucleotide. In some embodiments, a base editor comprises a polynucleotide programmable nucleotide binding domain (e.g., Cas9), a nucleobase editing domain (e.g., deaminase domain), and one or more additional domains. In some embodiments, the additional domain can facilitate enzymatic or catalytic functions of the base editor, binding functions of the base editor, or be inhibitors of cellular machinery (e.g., enzymes) that could interfere with the desired base editing result. In some embodiments, a base editor comprises a nuclease, a nickase, a recombinase, a deaminase, a methyltransferase, a methylase, an acetylase, an acetyltransferase, a transcriptional activator, or a transcriptional repressor domain.

In some embodiments, a base editor comprises an uracil glycosylase inhibitor (UGI) domain. In some embodiments, cellular DNA repair response to the presence of U: G heteroduplex DNA can be responsible for a decrease in nucleobase editing efficiency in cells. In such embodiments, uracil DNA glycosylase (UDG) can catalyze removal of U from DNA in cells, which can initiate base excision repair (BER), mostly resulting in reversion of the U:G pair to a C:G pair. In such embodiments, BER can be inhibited in base editors comprising one or more domains that bind the single strand, block the edited base, inhibit UGI, inhibit BER, protect the edited base, and/or promote repairing of the non-edited strand. Thus, this disclosure contemplates a base editor fusion protein or complex comprising a UGI domain and/or a uracil stabilizing protein (USP) domain.

### Base Editor System

Provided herein are systems, compositions, and methods for editing a nucleobase using a base editor system. In some embodiments, the base editor system comprises (1) a base editor (BE) comprising a polynucleotide programmable nucleotide binding domain and a nucleobase editing domain (e.g., a deaminase domain) for editing the nucleobase; and (2) a guide polynucleotide (e.g., guide RNA) in conjunction with the polynucleotide programmable nucleotide binding domain. In some embodiments, the base editor system is a cytidine base editor (CBE) or an adenosine base editor (ABE). In some embodiments, the polynucleotide programmable nucleotide binding domain is a polynucleotide programmable DNA or RNA binding domain. In some embodiments, the nucleobase editing domain is a deaminase domain. In some embodiments, a deaminase domain can be a cytidine deaminase or an cytosine deaminase. In some embodiments, a deaminase domain can be an adenine deaminase or an adenosine deaminase. In some embodiments, the adenosine base editor can deaminate adenine in DNA. In some embodiments, the base editor is capable of deaminating a cytidine in DNA.

Use of the base editor system provided herein comprises the steps of: (a) contacting a target nucleotide sequence of a polynucleotide (e.g., double- or single stranded DNA or RNA) of a subject with a base editor system comprising a nucleobase editor (e.g., an adenosine base editor or a cytidine base editor) and a guide polynucleotide (e.g., gRNA), wherein the target nucleotide sequence comprises a targeted nucleobase pair; (b) inducing strand separation of said target region; (c) converting a first nucleobase of said target nucleobase pair in a single strand of the target region to a second nucleobase; and (d) cutting no more than one strand of said target region, where a third nucleobase complementary to the first nucleobase base is replaced by a fourth nucleobase complementary to the second nucleobase. It should be appreciated that in some embodiments, step (b) is omitted. In some embodiments, said targeted nucleobase pair is a plurality of nucleobase pairs in one or more genes. In some embodiments, the base editor system provided herein is capable of multiplex editing of a plurality of nucleobase pairs in one or more genes. In some embodiments, the plurality of nucleobase pairs is located in the same gene. In some embodiments, the plurality of nucleobase pairs is located in one or more genes, wherein at least one gene is located in a different locus.

The components of a base editor system (e.g., a deaminase domain, a guide RNA, and/or a polynucleotide programmable nucleotide binding domain) may be associated with each other covalently or non-covalently. For example, in some embodiments, the deaminase domain can be targeted to a target nucleotide sequence by a polynucleotide programmable nucleotide binding domain, optionally where the polynucleotide programmable nucleotide binding domain is complexed with a polynucleotide (e.g., a guide RNA). In some embodiments, a polynucleotide programmable nucleotide binding domain can be fused or linked to a deaminase domain. In some embodiments, a polynucleotide programmable nucleotide binding domain can target a deaminase domain to a target nucleotide sequence by non-covalently interacting with or associating with the deaminase domain. For example, in some embodiments, the nucleobase editing component (e.g., the deaminase component) comprises an additional heterologous portion or domain that is capable of interacting with, associating with, or capable of forming a complex with a corresponding heterologous portion, antigen, or domain that is part of a polynucleotide programmable nucleotide binding domain and/or a guide polynucleotide (e.g., a guide RNA) complexed therewith. In some embodiments, the polynucleotide programmable nucleotide binding domain, and/or a guide polynucleotide (e.g., a guide RNA) complexed therewith, comprises an additional heterologous portion or domain that is capable of interacting with, associating with, or capable of forming a complex with a corresponding heterologous portion, antigen, or domain that is part of a nucleobase editing domain (e.g., the deaminase component). In some embodiments, the additional heterologous portion may be capable of binding to, interacting with, associating with, or forming a complex with a polypeptide. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion is capable of binding to a polynucleotide linker. An additional heterologous portion may be a protein domain. In some embodiments, an additional heterologous portion comprises a polypeptide, such as a 22 amino acid RNA-binding domain of the lambda bacteriophage antiterminator protein N (N22p), a 2G12 IgG homodimer domain, an ABI, an antibody (e.g. an antibody that binds a component of the base editor system or a heterologous portion thereof) or fragment thereof (e.g. heavy chain domain 2 (CH2) of IgM (MHD2) or IgE (EHD2), an immunoglobulin Fc region, a heavy chain domain 3 (CH3) of IgG or IgA, a heavy chain domain 4 (CH4) of IgM or IgE, an Fab, an Fab2, miniantibodies, and/or ZIP antibodies), a barnase-barstar dimer domain, a Bcl-xL domain, a Calcineurin A (CAN) domain, a Cardiac phospholamban transmembrane pentamer domain, a collagen domain, a Com RNA binding protein domain (e.g. SfMu Com coat protein domain, and SfMu Com binding protein domain), a Cyclophilin-Fas fusion protein (CyP-Fas) domain, a Fab domain, an Fc domain, a fibritin foldon domain, an FK506 binding protein (FKBP) domain, an FKBP binding domain (FRB) domain of mTOR, a foldon domain, a fragment X domain, a GAI domain, a GID1 domain, a Glycophorin A transmembrane domain, a GyrB domain, a Halo tag, an HIV Gp41 trimerisation domain, an HPV45 oncoprotein E7 C-terminal dimer domain, a hydrophobic polypeptide, a K Homology (KH) domain, a Ku protein domain (e.g., a Ku heterodimer), a leucine zipper, a LOV domain, a mitochondrial antiviral-signaling protein CARD filament domain, an MS2 coat protein domain (MCP), a non-natural RNA aptamer ligand that binds a corresponding RNA motif/aptamer, a parathyroid hormone dimerization domain, a PP7 coat protein (PCP) domain, a PSD95-Dlgl-zo-1 (PDZ) domain, a PYL domain, a SNAP tag, a SpyCatcher moiety, a SpyTag moiety, a streptavidin domain, a streptavidin-binding protein domain, a streptavidin binding protein (SBP) domain, a telomerase Sm7 protein domain (e.g. Sm7 homoheptamer or a monomeric Sm-like protein), and/or fragments thereof. In embodiments, an additional heterologous portion comprises a polynucleotide (e.g., an RNA motif), such as an MS2 phage operator stem-loop (e.g., an MS2, an MS2 C-5 mutant, or an MS2 F-5 mutant), a non-natural RNA motif, a PP7 operator stem-loop, an SfMu phate Com stem-loop, a steril alpha motif, a telomerase Ku binding motif, a telomerase Sm7 binding motif, and/or fragments thereof. Non-limiting examples of additional heterologous portions include polypeptides with at least about 85% sequence identity to any one or more of SEQ ID NOs: 380, 382, 384, 386-388, or fragments thereof. Non-limiting examples of additional heterologous portions include polynucleotides with at least about 85% sequence identity to any one or more of SEQ ID NOs: 379, 381, 383, 385, or fragments thereof.

In some instances, components of the base editing system are associated with one another through the interaction of leucine zipper domains (e.g., SEQ ID NOs: 387 and 388). In some cases, components of the base editing system are associated with one another through polypeptide domains (e.g., FokI domains) that associate to form protein complexes containing about, at least about, or no more than about 1, 2 (i.e., dimerize), 3, 4, 5, 6, 7, 8, 9, polypeptide domain units, optionally the polypeptide domains may include alterations that reduce or eliminate an activity thereof.

In some instances, components of the base editing system are associated with one another through the interaction of multimeric antibodies or fragments thereof (e.g., IgG, IgD, IgA, IgM, IgE, a heavy chain domain 2 (CH2) of IgM (MHD2) or IgE (EHD2), an immunoglobulin Fc region, a heavy chain domain 3 (CH3) of IgG or IgA, a heavy chain domain 4 (CH4) of IgM or IgE, an Fab, and an Fab2). In some instances, the antibodies are dimeric, trimeric, or tetrameric. In embodiments, the dimeric antibodies bind a polypeptide or polynucleotide component of the base editing system.

In some cases, components of the base editing system are associated with one another through the interaction of a polynucleotide-binding protein domain(s) with a polynucleotide(s). In some instances, components of the base editing system are associated with one another through the interaction of one or more polynucleotide-binding protein domains with polynucleotides that are self-complementary and/or complementary to one another so that complementary binding of the polynucleotides to one another brings into association their respective bound polynucleotide-binding protein domain(s).

In some instances, components of the base editing system are associated with one another through the interaction of a polypeptide domain(s) with a small molecule(s) (e.g., chemical inducers of dimerization (CIDs), also known as “dimerizers”). Non-limiting examples of CIDs include those disclosed in Amara, et al., “A versatile synthetic dimerizer for the regulation of protein-protein interactions,” PNAS, 94:10618-10623 (1997); and Voβ, et al. “Chemically induced dimerization: reversible and spatiotemporal control of protein function in cells,” Current Opinion in Chemical Biology, 28:194-201 (2015), the disclosures of each of which are incorporated herein by reference in their entireties for all purposes. In some embodiments, the base editor inhibits base excision repair (BER) of the edited strand. In some embodiments, the base editor protects or binds the non-edited strand. In some embodiments, the base editor comprises UGI activity or USP activity. In some embodiments, the base editor comprises a catalytically inactive inosine-specific nuclease.

The base editors of the present disclosure can comprise any domain, feature or amino acid sequence which facilitates the editing of a target polynucleotide sequence. For example, in some embodiments, the base editor comprises a nuclear localization sequence (NLS). In some embodiments, an NLS of the base editor is localized between a deaminase domain and a polynucleotide programmable nucleotide binding domain. In some embodiments, an NLS of the base editor is localized C-terminal to a polynucleotide programmable nucleotide binding domain.

Protein domains included in the fusion protein can be a heterologous functional domain. Non-limiting examples of protein domains which can be included in the fusion protein include a deaminase domain (e.g., cytidine deaminase and/or adenosine deaminase), a uracil glycosylase inhibitor (UGI) domain, epitope tags, and reporter gene sequences. In some embodiments, the adenosine base editor (ABE) can deaminate adenine in DNA. In some embodiments, ABE is generated by replacing APOBEC1 component of BE3 with natural or engineered E. coli TadA, human ADAR2, mouse ADA, or human ADAT2. In some embodiments, ABE comprises an evolved TadA variant. In some embodiments, the base editor is ABE8.1, which comprises or consists essentially of the following sequence or a fragment thereof having adenosine deaminase activity: SEQ ID NO: 331. Other ABE8 sequences are provided in the attached sequence listing (SEQ ID NOs: 332-354).

In some embodiments, the base editor includes an adenosine deaminase variant comprising an amino acid sequence, which contains alterations relative to an ABE 7*10 reference sequence, as described herein. The term “monomer” as used in Table 7 refers to a monomeric form of TadA*7.10 comprising the alterations described. The term “heterodimer” as used in Table 7 refers to the specified wild-type E. co/i TadA adenosine deaminase fused to a TadA*7.10 comprising the alterations as described.

TABLE 7
Adenosine Deaminase Base Editor Variants

| | Adenosine | |
|---|---|---|
| ABE | Deaminase | Adenosine Deaminase Description |
| ABE-605m | MSP605 | monomer_TadA*7.10 + V82G + Y147T + Q154S |
| ABE-680m | MSP680 | monomer_TadA*7.10 + I76Y + V82G + Y147T + Q154S |
| ABE-823m | MSP823 | monomer_TadA*7.10 + L36H + V82G + Y147T + Q154S + |
| | | N157K |
| ABE-824m | MSP824 | monomer_TadA*7.10 + V82G + Y147D + F149Y + Q154S + |
| | | D167N |
| ABE-825m | MSP825 | monomer_TadA*7.10 + L36H + V82G+ Y147D + F149Y + |
| | | Q154S + N157K + D167N |
| ABE-827m | MSP827 | monomer_TadA*7.10 + L36H + I76Y + V82G + Y147T + |
| | | Q154S + N157K |
| ABE-828m | MSP828 | monomer_TadA*7.10 + I76Y + V82G + Y147D + F149Y + |
| | | Q154S + D167N |
| ABE-829m | MSP829 | monomer_TadA*7.10 + L36H + I76Y + V82G + Y147D + |
| | | F149Y+ Q154S + N157K + D167N |
| ABE-605d | MSP605 | heterodimer_(WT) + (TadA*7.10 + V82G + Y147T + Q154S) |
| ABE-680d | MSP680 | heterodimer_(WT) + (TadA*7.10 + I76Y + V82G + Y147T + |
| | | Q154S) |
| ABE-823d | MSP823 | heterodimer_(WT) + (TadA*7.10 + L36H + V82G + Y147T + |
| | | Q154S + N157K) |
| ABE-824d | MSP824 | heterodimer_(WT) + (TadA*7.10 + V82G + Y147D + F149Y + |
| | | Q154S + D167N) |
| ABE-825d | MSP825 | heterodimer_(WT) + (TadA*7.10 + L36H + V82G + Y147D + |
| | | F149Y + Q154S + N157K + D167N) |
| ABE-827d | MSP827 | heterodimer_(WT) + (TadA*7.10 + L36H + I76Y + V82G + |
| | | Y147T + Q154S + N157K) |
| ABE-828d | MSP828 | heterodimer_(WT) + (TadA*7.10 + I76Y + V82G + Y147D + |
| | | F149Y + Q154S + D167N) |
| ABE-829d | MSP829 | heterodimer_(WT) + (TadA*7.10 + L36H + I76Y + V82G + |
| | | Y147D + F149Y + Q154S + N157K + D167N) |

In some embodiments, the base editor comprises a domain comprising all or a portion (e.g., a functional portion) of a uracil glycosylase inhibitor (UGI) or a uracil stabilizing protein (USP) domain.

### Linkers

In certain embodiments, linkers may be used to link any of the peptides or peptide domains of the disclosure. The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.).

In some embodiments, any of the fusion proteins provided herein, comprise a cytidine or adenosine deaminase and a Cas9 domain that are fused to each other via a linker. Various linker lengths and flexibilities between the cytidine or adenosine deaminase and the Cas9 domain can be employed (e.g., ranging from very flexible linkers of the form (GGGS) n (SEQ ID NO: 246), (GGGGS)n (SEQ ID NO: 247), and (G)n to more rigid linkers of the form (EAAAK)n (SEQ ID NO: 248), (SGGS)n (SEQ ID NO: 355), SGSETPGTSESATPES (SEQ ID NO: 249) (see, e.g., Guilinger J P, et al. Fusion of catalytically inactive Cas9 to FokI nuclease improves the specificity of genome modification. Nat. Biotechnol. 2014; 32(6): 577-82; the entire contents are incorporated herein by reference) and (XP)n) in order to achieve the optimal length for activity for the cytidine or adenosine deaminase nucleobase editor. In some embodiments, n is 1, 2, 3, 4, 5, 6, 7, 8, 9, 10, 11, 12, 13, 14, or 15. In some embodiments, the linker comprises a (GGS)n motif, wherein n is 1, 3, or 7. In some embodiments, cytidine deaminase or adenosine deaminase and the Cas9 domain of any of the fusion proteins provided herein are fused via a linker comprising the amino acid sequence SGSETPGTSESATPES (SEQ ID NO: 249), which can also be referred to as the XTEN linker.

In some embodiments, the domains of the base editor are fused via a linker that comprises the amino acid sequence of:

(SEQ ID NO: 356)
SGGSSGSETPGTSESATPESSGGS,
(SEQ ID NO: 357)
SGGSSGGSSGSETPGTSESATPESSGGSSGGS,
or
(SEQ ID NO: 358)
GGSGGSPGSPAGSPTSTEEGTSESATPESGPGTSTEPSEGSAPGSPAGS
PTSTEEGTSTEPSEGSAPGTSTEPSEGSAPGTSESATPESGPGSEPATS
GGSGGS.

In some embodiments, domains of the base editor are fused via a linker comprising the amino acid sequence SGSETPGTSESATPES (SEQ ID NO: 249), which may also be referred to as the XTEN linker. In some embodiments, a linker comprises the amino acid sequence SGGS (SEQ ID NO: 355). In some embodiments, the linker is 24 amino acids in length. In some embodiments, the linker comprises the amino acid sequence SGGSSGGSSGSETPGTSESATPES (SEQ ID NO: 359). In some embodiments, the linker is amino acids in length. In some embodiments, the linker comprises the amino acid sequence: SGGSSGGSSGSETPGTSESATPESSGGSSGGSSGGSSGGS (SEQ ID NO: 360). In some embodiments, the linker is 64 amino acids in length. In some embodiments, the linker comprises the amino acid sequence: SGGSSGGSSGSETPGTSESATPESSGGSSGGSSGGSSGGSSGSETPGTSESATPESSGGSSG GS (SEQ ID NO: 361). In some embodiments, the linker is 92 amino acids in length. In some embodiments, the linker comprises the amino acid sequence:

(SEQ ID NO: 362)
PGSPAGSPTSTEEGTSESATPESGPGTSTEPSEGSAPGSPAGSPTSTEE
GTSTEPSEGSAPGTSTEPSEGSAPGTSESATPESGPGSEPATS.

In some embodiments, a linker comprises a plurality of proline residues and is 5-21, 5-14, 5-9, 5-7 amino acids in length, e.g., PAPAP (SEQ ID NO: 363), PAPAPA (SEQ ID NO: 364), PAPAPAP (SEQ ID NO: 365), PAPAPAPA (SEQ ID NO: 366), P(AP)4 (SEQ ID NO: 367), P(AP)7 (SEQ ID NO: 368), P(AP)10 (SEQ ID NO: 369) (see, e.g., Tan J, Zhang F, Karcher D, Bock R. Engineering of high-precision base editors for site-specific single nucleotide replacement. Nat Commun. 2019 Jan. 25; 10(1):439; the entire contents are incorporated herein by reference). Such proline-rich linkers are also termed “rigid” linkers.

Nucleic Acid Programmable DNA Binding Proteins with Guide RNAs

Provided herein are compositions and methods for base editing in cells. Further provided herein are compositions comprising a guide polynucleotide sequence, e.g., a guide RNA sequence, or a combination of 2, 3, 4, 5, 6, 7, 8, 9, 10, 11, 12, 13, 14, 15, 16, 17, 18, 19, 20, or more guide RNAs as provided herein. In some embodiments, a composition for base editing as provided herein further comprises a polynucleotide that encodes a base editor, e.g., a C-base editor or an A-base editor. For example, a composition for base editing may comprise a mRNA sequence encoding a BE, a BE4, an ABE, and a combination of one or more guide RNAs as provided. A composition for base editing may comprise a base editor polypeptide and a combination of one or more of any guide RNAs provided herein. Such a composition may be used to effect base editing in a cell through different delivery approaches, for example, electroporation, nucleofection, viral transduction or transfection. In some embodiments, the composition for base editing comprises an mRNA sequence that encodes a base editor and a combination of one or more guide RNA sequences provided herein for electroporation.

Some aspects of this disclosure provide systems comprising any of the fusion proteins or complexes provided herein, and a guide RNA bound to a nucleic acid programmable DNA binding protein (napDNAbp) domain (e.g., a Cas9 (e.g., a dCas9, a nuclease active Cas9, or a Cas9 nickase) or Cas12) of the fusion protein or complex. These complexes are also termed ribonucleoproteins (RNPs). In some embodiments, the guide nucleic acid (e.g., guide RNA) is from 15-100 nucleotides long and comprises a sequence of at least 10 contiguous nucleotides that is complementary to a target sequence. In some embodiments, the target sequence is a DNA sequence. In some embodiments, the target sequence is an RNA sequence. In some embodiments, the target sequence is a sequence in the genome of a bacteria, yeast, fungi, insect, plant, or animal. In some embodiments, the target sequence is a sequence in the genome of a human. In some embodiments, the 3′ end of the target sequence is immediately adjacent to a canonical PAM sequence (NGG). In some embodiments, the 3′ end of the target sequence is immediately adjacent to a non-canonical PAM sequence (e.g., a sequence listed in Table 3 or 5′-NAA-3′). In some embodiments, the guide nucleic acid (e.g., guide RNA) is complementary to a sequence in a gene of interest (e.g., a gene associated with a disease or disorder).

Some aspects of this disclosure provide methods of using the fusion proteins, or complexes provided herein. For example, some aspects of this disclosure provide methods comprising contacting a DNA molecule with any of the fusion proteins or complexes provided herein, and with at least one guide RNA, wherein the guide RNA is about 15-100 nucleotides long and comprises a sequence of at least 10 contiguous nucleotides that is complementary to a target sequence.

The domains of the base editor disclosed herein can be arranged in any order.

A defined target region can be a deamination window. A deamination window can be the defined region in which a base editor acts upon and deaminates a target nucleotide. In some embodiments, the deamination window is within a 2, 3, 4, 5, 6, 7, 8, 9, or 10 base regions. In some embodiments, the deamination window is 5, 6, 7, 8, 9, 10, 11, 12, 13, 14, 15, 16, 17, 18, 19, 20, 21, 22, 23, 24, or 25 bases upstream of the PAM.

The base editors of the present disclosure can comprise any domain, feature or amino acid sequence which facilitates the editing of a target polynucleotide sequence.

### Methods of Using Fusion Proteins or Complexes Comprising a Cytidine or Adenosine Deaminase and a Cas9 Domain

Some aspects of this disclosure provide methods of using the fusion proteins, or complexes provided herein. For example, some aspects of this disclosure provide methods comprising contacting a DNA molecule with any of the fusion proteins or complexes provided herein, and with at least one guide RNA described herein.

In some embodiments, a fusion protein or complex of the disclosure is used for editing a target gene of interest. In particular, a cytidine deaminase or adenosine deaminase nucleobase editor described herein is capable of making multiple mutations within a target sequence. These mutations may affect the function of the target. For example, when a cytidine deaminase or adenosine deaminase nucleobase editor is used to target a regulatory region the function of the regulatory region is altered and the expression of the downstream protein is reduced or eliminated.

### Multiplex Editing

In some embodiments, the base editor system provided herein is capable of multiplex editing of a plurality of nucleobase pairs in one or more genes or polynucleotide sequences. In some embodiments, the plurality of nucleobase pairs is located in the same gene or in one or more genes, wherein at least one gene is located in a different locus. In some embodiments, the multiplex editing comprises one or more guide polynucleotides. In some embodiments, the multiplex editing comprises one or more base editor systems. In some embodiments, the multiplex editing comprises one or more base editor systems with a single guide polynucleotide or a plurality of guide polynucleotides. In some embodiments, the multiplex editing comprises one or more guide polynucleotides with a single base editor system. It should be appreciated that the characteristics of the multiplex editing using any of the base editors as described herein can be applied to any combination of methods using any base editor provided herein. It should also be appreciated that the multiplex editing using any of the base editors as described herein can comprise a sequential editing of a plurality of nucleobase pairs.

In some embodiments, the base editor system capable of multiplex editing of a plurality of nucleobase pairs in one or more genes comprises one of ABE7, ABE8, and/or ABE9 base editors.

### Expression of Fusion Proteins or Complexes in a Host Cell

Polypeptides of the present disclosure may be expressed in virtually any host cell of interest, including mammalian cells (e.g., human cells). In some embodiments, the host cell is an immune cell (e.g., a T cell, such as a regulatory T (T_{REG}) cell). In some embodiments, the host cell is an allogeneic immune cell (e.g., a T_{REG} cell). In some embodiments, the host cell is a CAR T_{REG} cell.

For example, a DNA encoding a polypeptide of the present disclosure can be cloned by designing suitable primers for the upstream and downstream of CDS based on the cDNA sequence. The cloned DNA may be directly, or after digestion with a restriction enzyme when desired, or after addition of a suitable linker and/or a nuclear localization signal, ligated with a DNA encoding one or more additional components of a base editing system. The base editing system is translated in a host cell to form a complex.

A DNA encoding a protein domain described herein can be obtained by chemically synthesizing the DNA, or by connecting synthesized partly overlapping oligoDNA short chains by utilizing the PCR method and the Gibson Assembly method to construct a DNA encoding the full length thereof. The advantage of constructing a full-length DNA by chemical synthesis or a combination of PCR method or Gibson Assembly method is that the codon to be used can be designed in CDS full-length according to the host into which the DNA is introduced. In the expression of a heterologous DNA, the protein expression level is expected to increase by converting the DNA sequence thereof to a codon highly frequently used in the host organism. As the data of codon use frequency in host to be used, for example, the genetic code use frequency database (kazusa.or.jp/codon/index.html) disclosed in the home page of Kazusa DNA Research Institute can be used, or documents showing the codon use frequency in each host may be referred to. By reference to the obtained data and the DNA sequence to be introduced, codons showing low use frequency in the host from among those used for the DNA sequence may be converted to a codon coding the same amino acid and showing high use frequency.

An expression vector containing a DNA encoding a nucleic acid sequence-recognizing module and/or a nucleic acid base converting enzyme can be produced, for example, by linking the DNA to the downstream of a promoter in a suitable expression vector.

As the expression vector, animal cell expression plasmids (e.g., pA1-11, pXT1, pRc/CMV, pRc/RSV, pcDNAI/Neo); and animal virus vectors such as retrovirus, vaccinia virus, adenovirus and the like, and the like are used.

Regarding the promoter to be used, any promoter appropriate for a host to be used for gene expression can be used. In a conventional method using double-stranded breaks, since the survival rate of the host cell sometimes decreases markedly due to the toxicity, it is desirable to increase the number of cells by the start of the induction by using an inductive promoter. However, since sufficient cell proliferation can also be afforded by expressing the nucleic acid-modifying enzyme complex of the present disclosure, a constitutive promoter can be used without limitation.

For example, when the host is an animal cell, an SRα promoter, SV40 promoter, LTR promoter, cytomegalovirus (CMV) promoter, Rous sarcoma virus (RSV) promoter, Moloney mouse leukemia virus (MoMuLV), LTR, herpes simplex virus thymidine kinase (HSV-TK) promoter, and the like can be used. Of these, CMV promoter, SR.alpha. promoter and the like may be used.

Expression vectors for use in embodiments of the present disclosure, besides those mentioned above, can comprise an enhancer, a splicing signal, a terminator, a polyA addition signal, a selection marker such as drug resistance gene, an auxotrophic complementary gene and the like, a replication origin, and the like can be used.

An RNA encoding a protein domain described herein can be prepared by, for example, in vitro transcription of a nucleic acid sequence encoding any of the polypeptides disclosed herein. A polypeptide of the present disclosure can be intracellularly expressed by introducing into the cell an expression vector comprising a nucleic acid sequence encoding the polypeptide.

Animal cells contemplated in the present disclosure include, but are not limited to, cell lines such as monkey COS-7 cells, monkey Vero cells, Chinese hamster ovary (CHO) cells, dhfr gene-deficient CHO cells, mouse L cells, mouse AtT-20 cells, mouse myeloma cells, rat GH3 cells, human FL cells and the like, pluripotent stem cells such as iPS cells, ES cells derived humans and other mammals, and primary cultured cells prepared from various tissues. Furthermore, zebrafish embryo, Xenopus oocyte, and the like can also be used.

All the above-mentioned host cells may be haploid (monoploid), or polyploid (e.g., diploid, triploid, tetraploid, etc.). Using conventional methods, mutations, in principle, introduced into only one homologous chromosome produce a heterogenous cell. Therefore, the desired phenotype is not expressed unless the mutation is dominant. For recessive mutations, acquiring a homozygous cell can be inconvenient due to labor and time requirements. In contrast, according to the present disclosure, since a mutation can be introduced into any allele on the homologous chromosome in the genome, the desired phenotype can be expressed in a single generation even in the case of recessive mutation, thereby solving the problem associated with conventional mutagenesis methods.

An expression vector can be introduced by a known method (e.g., the lysozyme method, the competent method, the PEG method, the CaCl₂) coprecipitation method, electroporation, microinjection, particle gun method, lipofection, Agrobacterium-mediated delivery, etc.) according to the kind of the host.

A vector can be introduced into an animal cell according to the methods described in, for example, Cell Engineering additional volume 8, New Cell Engineering Experiment Protocol, 263-267 (1995) (published by Shujunsha), and Virology, 52, 456 (1973).

As a medium for culturing an animal cell, for example, minimum essential medium (MEM) containing about 5 to about 20% of fetal bovine serum [Science, 122, 501 (1952)], Dulbecco's modified Eagle medium (DMEM) [Virology, 8, 396 (1959)], RPMI 1640 medium [The Journal of the American Medical Association, 199, 519 (1967)], 199 medium [Proceeding of the Society for the Biological Medicine, 73, 1 (1950)] and the like are used. The pH of the medium may be from about 6 to about 8. The culture is performed at generally about 30° C. to about 40° C. Where necessary, aeration and stirring may be performed.

When a higher eukaryotic cell, such as animal cell, is used as a host cell, a DNA encoding a base editing system of the present disclosure is introduced into a host cell under the regulation of an inducible promoter (e.g., metallothionein promoter (induced by heavy metal ion), heat shock protein promoter (induced by heat shock), Tet-ON/Tet-OFF system promoter (induced by addition or removal of tetracycline or a derivative thereof), steroid-responsive promoter (induced by steroid hormone or a derivative thereof) etc.), the induction substance is added to the medium (or removed from the medium) at an appropriate stage to induce expression of the nucleic acid-modifying enzyme complex, culture is performed for a given period to carry out a base editing and, introduction of a mutation into a target gene, transient expression of the base editing system can be realized.

Alternatively, the above-mentioned inductive promoter can also be utilized as a vector removal mechanism when higher eukaryotic cells, such as animal cells, and the like are used as a host cell. That is, a vector is mounted with a replication origin that functions in a host cell, and a nucleic acid encoding a protein necessary for replication (e.g., SV40 on and large T antigen, oriP and EBNA-1 etc. for animal cells), of the expression of the nucleic acid encoding the protein is regulated by the above-mentioned inducible promoter. As a result, while the vector is autonomously replicable in the presence of an induction substance, when the induction substance is removed, autonomous replication is not available, and the vector naturally falls off along with cell division (autonomous replication is not possible by the addition of tetracycline and doxycycline in Tet-OFF system vector).

### Chimeric Antigen Receptors and Car-T Cells

The disclosure provides immune cells modified using nucleobase editors described herein that express chimeric antigen receptors (CARs). Modification of immune cells to express a chimeric antigen receptor can enhance an immune cell's immunoreactive activity, wherein the chimeric antigen receptor has an affinity for an epitope on an antigen, wherein the antigen is associated with an altered fitness of an organism. For example, the chimeric antigen receptor can have an affinity for an epitope on a protein expressed in a target cell associated with graft versus host disease (GVHD). Because the CAR-T_{REG} cells can inhibit or prevent activation and/or proliferation of T_{CONV} near the CAR-T_{REG} cells when the CAR-T_{REG} cell are activated (e.g., through binding of the CAR to an antigen). In embodiments, the antigen is a human leukocyte antigen, such as HLA-A*02.

In embodiments, the CAR binds an antigen associated with a population of autoreactive T cells. In various instances, the antigen targeted by the CAR is associated with one or more diseases. Non-limiting examples of diseases include autoimmune diseases and alloimmune diseases, such as graft versus host disease (GVHD), acute GVHD, or chronic GVHD. In embodiments, the disease is any disease or disorder associated with an undesired immune activity or response. Further non-limiting examples of diseases include type I diabetes, multiple sclerosis, rheumatoid arthritis, amyotrophic lateral sclerosis (ALS), hemophilia, autoantibody-mediated autoimmune disease, asthma, systemic lupus erythematosus (SLE), Chrohn's disease, cutaneious lupus, and pemphigus.

Some embodiments comprise autologous immune cell immunotherapy, wherein immune cells are obtained from a subject having a disease or altered fitness characterized by an autoimmune or alloimmune response, such as graft versus host disease (GVHD). The obtained immune cells are genetically modified to express a chimeric antigen receptor and are effectively redirected against specific antigens. Thus, in some embodiments, immune cells are obtained from a subject in need of CAR-T_{REG} immunotherapy. In some embodiments, these autologous immune cells are cultured and modified shortly after they are obtained from the subject. In other embodiments, the autologous cells are obtained and then stored for future use. In allogeneic immune cell immunotherapy, immune cells can be obtained from a donor other than the subject who will be receiving treatment. In some embodiments, immune cells are obtained from a healthy subject or donor and are genetically modified to express a chimeric antigen receptor and are effectively redirected against specific antigens. The immune cells, after modification to express a chimeric antigen receptor, are administered to a subject for treating an autoimmune or alloimmune disorder (e.g., graft versus host disease). In some embodiments, immune cells to be modified to express a chimeric antigen receptor can be obtained from pre-existing stock cultures of immune cells.

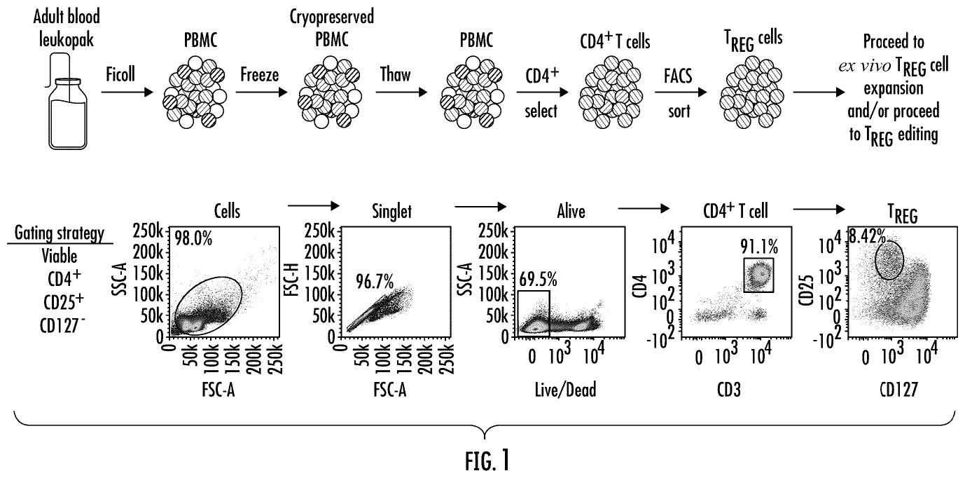

Cells (e.g., immune cells, immune effector cells, pluripotent stem cells, etc.) can be isolated or purified from a sample collected from a subject or a donor using standard techniques known in the art (see, e.g., FIG. 1). In embodiments, the cell is a precursor to a T_{REG} cell (e.g., an induced pluripotent stem cell (iPSC) or an embryonic stem cell (ESC)) and/or is capable of differentiating into a T_{REG} cell. In some embodiments, the T_{REG} cell (or a T_{REG} cell equivalent) is obtained from an induced pluripotent stem cell. In some embodiments, the T_{REG} cell (or a T_{REG} cell equivalent) is obtained from a T_{REG} precursor cell (e.g., an induced pluripotent stem cell (iPSC) or an embryonic stem cell (ESC)). For example, immune effector cells can be isolated or purified from a whole blood sample by lysing red blood cells and removing peripheral mononuclear blood cells by centrifugation. The immune effector cells can be further isolated or purified using a selective purification method that isolates the immune effector cells based on cell-specific markers such as CD25, CD3, CD4, CD8, CD25, CD28, CD45RA, CD45RO, or CD127. In one embodiment, CD4⁺ is used as a marker to select T_{REG} cells. In one embodiment, CD25⁺ is used as a marker to select T_{REG} cells. In one embodiment, CD127⁻ is used as a marker to select T_{REG} cells. In one embodiment, CD4⁺, CD8⁺⁺, and CD127⁻ are used as a marker to select regulatory T_{REG} cells. In embodiments, T_{REG} cells and/or subpopulations thereof are selected using one or more of the following markers: CD3⁺, CD4⁺, CD5⁺, CD14⁻, CD19⁻, CD25/IL-2 R alpha⁺, CD39/ENTPD1⁺, 5′ Nucleotidase/CD73⁺, CD103/Integrin alpha E⁺, CD127⁺, CTLA-4⁺, Folate Receptor 4⁺, FoxP3⁺, LRRC32/GARP⁺, GITR⁺, IL-7 R alpha/CD127^{low}, Helios^{+/-}, LAG-3/CD223⁺, LAP⁺, Neuropilin-1/BDCA-4⁺, OX40/CD134⁺, L-Selectin/CD26L⁺, and STAT5Y. In some instances, a T_{REG} cell is selected using one or more markers selected from Galectin-1, TGF-beta, IL-10, and IL-35. In some cases, the T_{REG} cells are selected using one or more of the following markers: CD8, CD28, CD45RA, CD45RO, Qa-1, HLA-E, or any polypeptide targeted by and/or edited using the base editors and methods provided herein.

A technique for isolating or purifying immune effector cells is flow cytometry (see, e.g., FIG. 1). In fluorescence activated cell sorting a fluorescently labelled antibody with affinity for an immune effector cell marker is used to label immune effector cells in a sample. A gating strategy appropriate for the cells expressing the marker is used to segregate the cells. For example, T lymphocytes can be separated from other cells in a sample by using, for example, a fluorescently labeled antibody specific for an immune effector cell marker (e.g., CD4⁺CD8⁺⁺, and CD127⁺) and corresponding gating strategy. In one embodiment, a CD4 gating strategy is employed. In one embodiment, a CD8 gating strategy is employed. In one embodiment, a CD127 gating strategy is employed. In another embodiment, a CD4, CD8, and CD127 gating strategy is employed. In some embodiments, a gating strategy for other markers specific to an immune effector cell is employed instead of, or in combination with, the CD4, CD8, and/or CD127 gating strategy.

The immune effector cells contemplated in the disclosure are effector T cells. In some embodiments, the effector T cell is a naïve CD8+ T cell, a cytotoxic T cell, a natural killer T (NKT) cell, a natural killer (NK) cell, or a regulatory T (Treg) cell. In some embodiments, the effector T cells are thymocytes, immature T lymphocytes, mature T lymphocytes, resting T lymphocytes, or activated T lymphocytes. In some embodiments the immune effector cell is a CD4⁺ CD8+, and CD127⁻ T_{REG} cell. In some embodiments, the immune effector cell is any other subset of T cells. The modified immune effector cell may express, in addition to the chimeric antigen receptor, an exogenous cytokine, a different chimeric receptor, or any other agent that would enhance immune effector cell signaling or function. For example, co-expression of the chimeric antigen receptor and a cytokine may enhance the CAR-T_{REG} cell's ability to inhibit activity of an immune cell (e.g., a T_{CONV} cell).

Chimeric antigen receptors as contemplated in the present disclosure comprise an extracellular binding domain, a transmembrane domain, and an intracellular domain. Binding of an antigen to the extracellular binding domain can activate the CAR-T_{REG} cell and generate an effector response, which includes inhibition of T cell (e.g., T_{CONV} cells) proliferation, cytokine production, and other processes associated with T cell activation. In some embodiments of the present disclosure, the chimeric antigen receptor further comprises a linker. In some embodiments, the linker is a (GGGGS)ₙ linker (SEQ ID NO: 247). In some embodiments, a CAR of the present disclosure includes a leader peptide sequence (e.g., N-terminal to the antigen binding domain). An exemplary leader peptide amino acid sequence is:

(SEQ ID NO: 963)
METDTLLLWVLLLWVPGSTG.

In various embodiments, the CAR-T specifically targets a human leukocyte antigen (e.g., HLA-A*02).

Provided herein are also nucleic acids that encode the chimeric antigen receptors described herein. In some embodiments, the nucleic acid is isolated or purified. Delivery of the nucleic acids ex vivo can be accomplished using methods known in the art. For example, immune cells obtained from a subject may be transformed with a nucleic acid vector encoding the chimeric antigen receptor. The vector may then be used to transform recipient immune cells so that these cells will then express the chimeric antigen receptor. Efficient means of transforming immune cells include transfection and transduction. Such methods are well known in the art. For example, applicable methods for delivery the nucleic acid molecule encoding the chimeric antigen receptor (and the nucleic acid(s) encoding the base editor) can be found in International Application No. PCT/US2009/040040 and U.S. Pat. Nos. 8,450,112; 9,132,153; and 9,669,058, each of which is incorporated herein in its entirety. Additionally, those methods and vectors described herein for delivering the nucleic acid encoding the base editor are applicable to delivering the nucleic acid encoding the chimeric antigen receptor.

Some aspects of the present disclosure provide for immune cells comprising a chimeric antigen and an altered endogenous gene that enhances immune cell function, increases lineage stability, increases resistance to immunosuppression or inhibition, reduces activation and/or proliferation of T_{CONV} cells or a combination thereof. In some embodiments, the altered endogenous gene may be created by base editing. In some embodiments, the base editing may reduce or attenuate the gene expression. In some embodiments, the base editing may reduce or attenuate the gene activation. In some embodiments, the base editing may reduce or attenuate the functionality of the gene product. In some other embodiments, the base editing may activate or enhance the gene expression. In some embodiments, the base editing may increase the functionality of the gene product. In some embodiments, the altered endogenous gene may be modified or edited in an exon, an intron, an exon-intron injunction, or a regulatory element thereof. The modification may be edit to a single nucleobase in a gene or a regulatory element thereof. The modification may be in an exon, more than one exon, an intron, or more than one intron, or a combination thereof. The modification may be in an open reading frame of a gene. The modification may be in an untranslated region of the gene, for example, a 3′-UTR or a 5′-UTR. In some embodiments, the modification is in a regulatory element of an endogenous gene. In some embodiments, the modification is in a promoter, an enhancer, an operator, a silencer, an insulator, a terminator, a transcription initiation sequence, a translation initiation sequence (e.g., a Kozak sequence), or any combination thereof.

In some embodiments, the immune cell may comprise a chimeric antigen receptor (CAR) and one or more edited genes, one or more regulatory elements thereof, or combinations thereof, wherein expression of the edited gene is either knocked out or knocked down. In some embodiments, the CAR-T_{REG} cells have increased lineage stability and/or enhanced functionality (e.g., less activation of T_{CONV} cells and/or lower allorecognition) as compared to a similar reference CAR-T_{REG} cell not having the one or more edited genes as described herein. In some embodiments, the CAR-T_{REG} cells have reduced immunogenicity as compared to a similar reference CAR-T_{REG} cell not having the one or more edited genes as described herein. In some embodiments, the CAR-T_{REG} cells have increased T_{CONV} cell inhibition activity as compared to a similar reference CAR-T_{REG} cell not having the one or more edited genes as described herein.

The one or more genes may be edited by base editing or through use of a nuclease (e.g., a Cas12b). In some embodiments, at least one or more genes or regulatory elements thereof are modified in an immune cell with the base editing compositions and methods provided herein.

In some embodiments the one or more genes, or one or more regulatory elements thereof, or combinations thereof, may be selected from a group consisting of: BRINP1, JNK1, PRKCQ, CHIP, CD70, CD58, PD-1, SIRT1, and RNF20. In some embodiments, the one or more genes, or regulatory elements thereof, comprise a combination of targets including one or more of SIRT1 and RNF20, and one or more of PD-1, CD70, and CD58. In embodiments, the combination of targets further includes β2M (β2M). In some embodiments, the one or more genes comprise a combination of targets selected from the following: SIRT1, PD-1, CD70, and CD58; SIRT1, PD-1, and CD70; SIRT1, PD-1, and CD58; SIRT1, CD70, and CD58; SIRT1 and PD-1; SIRT1 and CD70; SIRT1 and CD58; SIRT1, PD-1, CD70, CD58, and B2M; SIRT1, PD-1, CD70, and B2M; SIRT1, PD-1, CD58 and B2M; SIRT1, CD70, CD58, and B2M; SIRT1, PD-1, and B2M; SIRT1, CD70, and B2M; SIRT1, CD58, and B2M; RNF20, PD-1, CD70, and CD58; RNF20, PD-1, and CD70; RNF20, PD-1, and CD58; RNF20, CD70, and CD58; RNF20 and PD-1; RNF20 and CD70; RNF20 and CD58; RNF20, PD-1, CD70, CD58, and B2M; RNF20, PD-1, CD70, and B2M; III RNF20, PD-1, CD58 and B2M; RNF20, CD70, CD58, and B2M; RNF20, PD-1, and B2M; RNF20, CD70, and B2M; RNF20, CD58, and B2M; SIRT1, RNF20, PD-1, CD70, and CD58; SIRT1, RNF20, PD-1, and CD70; SIRT1, RNF20, PD-1, and CD58; SIRT1, RNF20, CD70, and CD58; SIRT1, RNF20, and PD-1; SIRT1, RNF20, and CD70; SIRT1, RNF20, and CD58; SIRT1, RNF20, PD-1, CD70, CD58, and B2M; SIRT1, RNF20, PD-1, CD70, and B2M; SIRT1, RNF20, PD-1, CD58, and B2M; SIRT1, RNF20, CD70, CD58, and B2M; SIRT1, RNF20, PD-1, and B2M; SIRT1, RNF20, CD70, and B2M; and SIRT1, RNF20, CD58, and B2M.

In some embodiments, the at least one or more genes or regulatory elements thereof include one or more genes, or one or more regulatory elements thereof, or combinations thereof including those described in PCT/US20/13964, PCT/US20/52822, PCT/US20/18178, and/or PCT/US21/52035.

In some embodiments, an immune cell comprises a chimeric antigen receptor and one or more edited genes, a regulatory element thereof, or combinations thereof. An edited gene may be an immune response regulation gene (e.g., coding for an immune response regulation peptide), an immunogenic gene, a checkpoint inhibitor gene, a gene involved in immune responses, a gene affecting lineage stabilization (e.g., SIRT1 and/or RNF20), a cell surface marker e.g., a T_{REG} cell surface protein (e.g., PD-1, CD70, or CD58), or any combination thereof. In some embodiments, an immune cell comprises a chimeric antigen receptor and an edited gene that is associated with activated T cell proliferation, alpha-beta T cell activation, gamma-delta T cell activation, positive regulation of T cell proliferation, negative regulation of T-helper cell proliferation or differentiation, or their regulatory elements thereof, or combinations thereof.

In some embodiments, provided herein is an immune cell with an edited B2M gene, such that the immune cell does not express an endogenous functional Beta-2-microglobulin.

In some embodiments, provided herein is a CAR-T_{REG} cell with an edited B2Mgene, such that the CAR-T_{REG} cell exhibits reduced or negligible expression or no expression of endogenous Beta-2-microglobulin. In embodiments, the reduced expression is about, or less than about 0.001%, 0.01%, 0.1%, 1%, 2%, 3%, 4%, 5%, 10%, 15%, 20%, 25%, 30%, 35%, 40%, 45%, 50%, 55%, 60%, 65%, 70%, 75%, 80%, 85%, 90%, or 95% of that relative to that in a reference cell. In embodiments, the reduced expression greater than about 0.001%, 0.01%, 0.1%, 1%, 2%, 3%, 4%, 5%, 10%, 15%, 20%, 25%, 30%, 35%, 40%, 45%, 50%, 55%, 60%, 65%, 70%, 75%, 80%, 85%, 90%, or 95% of that relative to that in a reference cell.

In some embodiments, provided herein is an immune cell with an edited BRINP1 gene, such that the immune cell does not express an endogenous functional BRINP1. In some embodiments, provided herein is a CAR-T_{REG} cell with an edited BRINP1 gene, such that the CAR-T_{REG} cell exhibits reduced or negligible expression or no expression of endogenous BRINP1. In embodiments, the reduced expression is about, or less than about 0.001%, 0.01%, 0.1%, 1%, 2%, 3%, 4%, 5%, 10%, 15%, 20%, 25%, 30%, 35%, 40%, 45%, 50%, 55%, 60%, 65%, 70%, 75%, 80%, 85%, 90%, or 95% of that relative to that in a reference cell. In embodiments, the reduced expression greater than about 0.001%, 0.01%, 0.1%, 1%, 2%, 3%, 4%, 5%, 10%, 15%, 20%, 25%, 30%, 35%, 40%, 45%, 50%, 55%, 60%, 65%, 70%, 75%, 80%, 85%, 90%, or 95% of that relative to that in a reference cell.

In some embodiments, provided herein is an immune cell with an edited JNK1 gene, such that the immune cell does not express an endogenous functional JNK1. In some embodiments, provided herein is a CAR-T_{REG} cell with an edited JNK1 gene, such that the CAR-T_{REG} cell exhibits reduced or negligible expression or no expression of endogenous JNK1. In embodiments, the reduced expression is about, or less than about 0.001%, 0.01%, 0.1%, 1%, 2%, 3%, 4%, 5%, 10, 1%, 20%, 25%, 30%, 35%, 40%, 45%, 50%, 55%, 60%, 65%, 70%, 75%, 80%, 85%, 90%, or 95% of that relative to that in a reference cell. In embodiments, the reduced expression greater than about 0.001%, 0.01%, 0.1%, 1%, 2%, 3%, 4%, 5%, 10%, 15%, 20%, 25%, 30%, 35%, 40%, 45%, 50%, 55%, 60%, 65%, 70%, 75%, 80%, 85%, 90%, or 95% of that relative to that in a reference cell.

In some embodiments, provided herein is an immune cell with an edited PRKCQ gene, such that the immune cell does not express an endogenous functional PRKCQ. In some embodiments, provided herein is a CAR-T_{REG} cell with an edited PRKCQ gene, such that the CAR-T_{REG} cell exhibits reduced or negligible expression or no expression of endogenous PRKCQ. In embodiments, the reduced expression is about, or less than about 0.001%, 0.01%, 0.1%, 1%, 2%, 3%, 4% 5%, 10, 15%, 20%, 25%, 30%, 35%, 40%, 45%, 50%, 55%, 60%, 65%, 70%, 75%, 80%, 85%, 90%, or 95% of that relative to that in a reference cell. In embodiments, the reduced expression greater than about 0.001%, 0.01%, 0.1%, 1%, 2%, 3%, 4%, 5%, 10%, 15%, 20%, 25%, 30%, 35%, 40%, 45%, 50%, 55%, 60%, 65%, 70%, 75%, 80%, 85%, 90%, or 95% of that relative to that in a reference cell.

In some embodiments, provided herein is an immune cell with an edited CHIP gene, such that the immune cell does not express an endogenous functional CHIP. In some embodiments, provided herein is a CAR-T_{REG} cell with an edited CHIP gene, such that the CAR-T_{REG} cell exhibits reduced or negligible expression or no expression of endogenous CHIP. In embodiments, the reduced expression is about, or less than about 0.001%, 0.01%, 0.1%, 1%, 2%, 3%, 4%, 5%, 1%, 15%, 20%, 25%, 30%, 35%, 40%, 45%, 50%, 55%, 60%, 65%, 70%, 75%, 80%, 85%, 90%, or 95% of that relative to that in a reference cell. In embodiments, the reduced expression greater than about 0.001%, 0.01%, 0.1%, 1%, 2%, 3%, 4%, 5%, 10%, 15%, 20%, 25%, 30%, 35%, 40%, 45%, 50%, 55%, 60%, 65%, 70%, 75%, 80%, 85%, 90%, or 95% of that relative to that in a reference cell.

In some embodiments, provided herein is an immune cell with an edited CD58 gene, such that the immune cell does not express an endogenous functional CD58. In some embodiments, provided herein is a CAR-T_{REG} cell with an edited CD58 gene, such that the CAR-T_{REG} cell exhibits reduced or negligible expression or no expression of endogenous CD58. In embodiments, the reduced expression is about, or less than about 0.001%, 0.01%, 0.1%, 1%, 2%, 3%, 4%, 5%, 10%, 15%, 20%, 25%, 30%, 35%, 40%, 45%, 50%, 55%, 60%, 65%, 70%, 75%, 80%, 85%, 90%, or 95% of that relative to that in a reference cell. In embodiments, the reduced expression greater than about 0.001%, 0.01%, 0.1%, 1%, 2%, 3%, 4%, 5%, 10%, 15%, 20%, 25%, 30%, 35%, 40%, 45%, 50%, 55%, 60%, 65%, 70%, 75%, 80%, 85%, 90%, or 95% of that relative to that in a reference cell.

In some embodiments, provided herein is an immune cell with an edited PD-1 gene, such that the immune cell does not express an endogenous functional PD-1. In some embodiments, provided herein is a CAR-T_{REG} cell with an edited PD-1 gene, such that the CAR-T_{REG} cell exhibits reduced or negligible expression or no expression of endogenous PD-1. In embodiments, the reduced expression is about, or less than about 0.001%, 0.01%, 0.1%, 1%, 2%, 3%, 4%, 5%, 10%, 15%, 20%, 25%, 30%, 35%, 40%, 45%, 50%, 55%, 60%, 65%, 70%, 75%, 80%, 85%, 90%, or 95% of that relative to that in a reference cell. In embodiments, the reduced expression greater than about 0.001%, 0.01%, 0.1%, 1%, 2%, 3%, 4%, 5%, 10%, 15%, 20%, 25%, 30%, 35%, 40%, 45%, 50%, 55%, 60%, 65%, 70%, 75%, 80%, 85%, 90%, or 95% of that relative to that in a reference cell.

In some embodiments, provided herein is an immune cell with an edited SIRT1 gene, such that the immune cell does not express an endogenous functional SIRT1. In some embodiments, provided herein is a CAR-T_{REG} cell with an edited SIRT1 gene, such that the CAR-T_{REG} cell exhibits reduced or negligible expression or no expression of endogenous SIRT1. In embodiments, the reduced expression is about, or less than about 0.001%, 0.01%, 0.1%, 1%, 2%, 3%, 4%, 5%, 10%, 15%, 20%, 25%, 30%, 35%, 40%, 45%, 50%, 55%, 60%, 65%, 70%, 75%, 80%, 85%, 90%, or 95% of that relative to that in a reference cell. In embodiments, the reduced expression greater than about 0.001%, 0.01%, 0.1%, 1%, 2%, 3%, 4%, 5%, 10%, 15%, 20%, 25%, 30%, 35%, 40%, 45%, 50%, 55%, 60%, 65%, 70%, 75%, 80%, 85%, 90%, or 95% of that relative to that in a reference cell.

In some embodiments, provided herein is an immune cell with an edited RNF20 gene, such that the immune cell does not express an endogenous functional RNF20. In some embodiments, provided herein is a CAR-T_{REG} cell with an edited RNF20 gene, such that the CAR-T_{REG} cell exhibits reduced or negligible expression or no expression of endogenous RNF20. In embodiments, the reduced expression is about, or less than about 0.001%, 0.01%, 0.1%, 1%, 2%, 3%, 4%, 5%, 10, 1%, 20%, 25%, 30%, 35%, 40%, 45%, 50%, 55%, 60%, 65%, 70%, 75%, 80%, 85%, 90%, or 95% of that relative to that in a reference cell. In embodiments, the reduced expression greater than about 0.001%, 0.01%, 0.1%, 1%, 2%, 3%, 4%, 5%, 10%, 15%, 20%, 25%, 30%, 35%, 40%, 45%, 50%, 55%, 60%, 65%, 70%, 75%, 80%, 85%, 90%, or 95% of that relative to that in a reference cell.

In some embodiments, each edited gene may comprise a single base edit, an insertion, and/or a deletion. In some embodiments, each edited gene may comprise multiple base edits at different regions of the gene. In some embodiments, a single modification event (such as electroporation), may introduce one or more gene edits. In some embodiments at least one, two, three, four, five, six, seven, eight, nine, ten, eleven, twelve, thirteen, fourteen, fifteen, sixteen, seventeen, eighteen, nineteen, twenty or more edits may be introduced in one or more genes simultaneously. In some embodiments, an immune cell, including but not limited to any immune cell comprising an edited gene selected from any of the aforementioned gene edits, can be edited to generate mutations in other genes that enhance the CAR-T_{REG}'s function and/or lineage stability.

### Extracellular Binding Domain

The chimeric antigen receptors of the disclosure include an extracellular binding domain. The extracellular binding domain of a chimeric antigen receptor contemplated herein comprises an amino acid sequence of an antibody, or an antigen binding fragment thereof, that has an affinity for a specific antigen. In some embodiments, the antigen is HLA-A*02.

In some embodiments the chimeric antigen receptor comprises an amino acid sequence of an antibody. In some embodiments, the chimeric antigen receptor comprises the amino acid sequence of an antigen binding fragment of an antibody. The antibody (or fragment thereof) portion of the extracellular binding domain recognizes and binds to an epitope of an antigen. In some embodiments, the antibody fragment portion of a chimeric antigen receptor is a single chain variable fragment (scFv). An scFv comprises the light and variable fragments of a monoclonal antibody. In other embodiments, the antibody fragment portion of a chimeric antigen receptor is a multichain variable fragment, which comprises more than one extracellular binding domains and therefore bind to more than one antigen simultaneously. In a multiple chain variable fragment embodiment, a hinge region may separate the different variable fragments, providing necessary spatial arrangement and flexibility.

In other embodiments, the antibody portion of a chimeric antigen receptor comprises at least one heavy chain and at least one light chain. In some embodiments, the antibody portion of a chimeric antigen receptor comprises two heavy chains, joined by disulfide bridges and two light chains, wherein the light chains are each joined to one of the heavy chains by disulfide bridges. In some embodiments, the light chain comprises a constant region and a variable region. Complementarity determining regions residing in the variable region of an antibody are responsible for the antibody's affinity for a particular antigen. Thus, antibodies that recognize different antigens comprise different complementarity determining regions. Complementarity determining regions reside in the variable domains of the extracellular binding domain, and variable domains (i.e., the variable heavy and variable light) can be linked with a linker or, in some embodiments, with disulfide bridges. In some embodiments, the variable heavy chain and variable light chain are linked by a (GGGGS)ₙ linker (SEQ ID NO: 247), wherein the n is an integer from 1 to 10.

In some embodiments, the antigen recognized and bound by the extracellular domain is a protein or peptide, a nucleic acid, a lipid, or a polysaccharide. Antigens can be heterologous, such as those expressed in a pathogenic bacteria or virus. Antigens can also be synthetic; for example, some individuals have extreme allergies to synthetic latex and exposure to this antigen can result in an extreme immune reaction. In some embodiments, the antigen is autologous, and is expressed on a diseased or otherwise altered cell.

For example, in some embodiments, the antigen (e.g., HLA-A*02) is expressed in an autologous or allogenic cell.

Antibody-antigen interactions are noncovalent interactions resulting from hydrogen bonding, electrostatic or hydrophobic interactions, or from van der Waals forces. The affinity of extracellular binding domain of the chimeric antigen receptor for an antigen can be calculated with the following formula:

K_{A}=[Antibody−Antigen]/[Antibody][Antigen], wherein
[Ab]=molar concentration of unoccupied binding sites on the antibody;
[Ag]=molar concentration of unoccupied binding sites on the antigen; and
[Ab-Ag]=molar concentration of the antibody-antigen complex.

The antibody-antigen interaction can also be characterized based on the dissociation of the antigen from the antibody. The dissociation constant (K_{D}) is the ratio of the association rate to the dissociation rate and is inversely proportional to the affinity constant. Thus, K_{D}=1/K_{A}. Those skilled in the art will be familiar with these concepts and will know that traditional methods, such as ELISA assays, can be used to calculate these constants.

### Transmembrane Domain

The chimeric antigen receptors of the disclosure include a transmembrane domain. The transmembrane domain of the chimeric antigen receptors described herein spans the CAR-T_{REG} cell's lipid bilayer cellular membrane and separates the extracellular binding domain and the intracellular signaling domain. In some embodiments, this domain is derived from other receptors having a transmembrane domain, while in other embodiments, this domain is synthetic. In some embodiments, the transmembrane domain may be derived from a non-human transmembrane domain and, in some embodiments, humanized. By “humanized” is meant having the sequence of the nucleic acid encoding the transmembrane domain optimized such that it is more reliably or efficiently expressed in a human subject. In some embodiments, the transmembrane domain is derived from another transmembrane protein expressed in a human immune effector cell. Examples of such proteins include, but are not limited to, subunits of the T cell receptor (TCR) complex, PD-1, or any of the Cluster of Differentiation proteins, or other proteins, that are expressed in the immune effector cell and that have a transmembrane domain. In some embodiments, the transmembrane domain will be synthetic, and such sequences will comprise many hydrophobic residues.

Transmembrane domains for use in the disclosed CARs can include at least the transmembrane region(s) of) the alpha, beta, or zeta chain of the T-cell receptor, CD28, CD3 epsilon, CD45, CD4, CD5, CD8, CD9, CD16, CD22, CD33, CD37, CD64, CD80, CD86, CD134, CD137, CD154. In some embodiments, the transmembrane domain is derived from CD4, CD8a, CD28, and CD3ζ.

The chimeric antigen receptor is designed, in some embodiments, to comprise a spacer between the transmembrane domain and the extracellular domain, the intracellular domain, or both. Such spacers can be 1, 2, 3, 4, 5, 6, 7, 8, 9, 10, 11, 12, 13, 14, 15, 16, 17, 18, 19, or 20 amino acids in length. In some embodiments, the spacer can be 20, 30, 40, 50, 60, 70, 80, 90, or 100 amino acids in length. In still other embodiments the spacer can be between 100 and 500 amino acids in length. The spacer can be any polypeptide that links one domain to another and are used to position such linked domains to enhance or optimize chimeric antigen receptor function.

### Intracellular Signaling Domain

The chimeric antigen receptors of the disclosure include an intracellular signaling domain. The intracellular signaling domain is the intracellular portion of a protein expressed in a T_{REG} cell that transduces a T_{REG} cell effector function signal (e.g., an activation signal) and directs the T_{REG} cell to perform a specialized function. T_{REG} cell activation can be induced by a number of factors, including binding of cognate antigen to the T cell receptor on the surface of T cells and binding of cognate ligand to costimulatory molecules on the surface of the T_{REG} cell. A T_{REG} cell co-stimulatory molecule is a cognate binding partner on a T_{REG} cell that specifically binds with a co-stimulatory ligand, thereby mediating a co-stimulatory response by the T_{REG} cell, such as, but not limited to, proliferation. Co-stimulatory molecules include but are not limited to an MHC class I molecule. Activation of a T_{REG} cell leads to immune response, such as T_{REG} cell proliferation and differentiation (see, e.g., Smith-Garvin et al., Annu. Rev. Immunol., 27:591-619, 2009). Exemplary T_{REG} cell signaling domains are known in the art. Non-limiting examples include the CD3ζ, CD8, CD28, CD27, CD154, GITR (TNFRSF18), CD134 (OX40), and CD137 (4-1BB) signaling domains.

The intracellular signaling domain of the chimeric antigen receptor contemplated herein comprises a primary signaling domain. In some embodiments, the chimeric antigen receptor comprises the primary signaling domain and a secondary, or co-stimulatory, signaling domain.

In some embodiments, the primary signaling domain comprises one or more immunoreceptor tyrosine-based activation motifs, or ITAMs. In some embodiments, the primary signaling domain comprises more than one ITAM. ITAMs incorporated into the chimeric antigen receptor may be derived from ITAMs from other cellular receptors. In some embodiments, the primary signaling domain comprising an ITAM may be derived from subunits of the TCR complex, such as CD3γ, CD3ε, CD3ζ, or CD3δ. In some embodiments, the primary signaling domain comprising an ITAM may be derived from FcRγ, FcRβ, CD5, CD22, CD79a, CD79b, or CD66d.

In some embodiments, the primary signaling domain is selected from the group consisting of CD8, CD28, CD134 (OX40), CD137 (4-1BB), and CD3ζ.

In some embodiments, the secondary, or co-stimulatory, signaling domain is derived from CD2, CD4, CDS, CD8α, CD28, CD83, CD134, CD137 (4-1BB), ICOS, or CD154, or a combination thereof.

### Editing of Target Genes in Immune Cells

In some embodiments, provided herein is an immune cell with at least one modification in an endogenous gene or regulatory elements thereof. In one embodiment, the modification enhances the persistence of a T_{REG}, enhances the function of a T_{REG}, and/or enhances the lineage stability of a T_{REG}. In some embodiments, the immune cell may comprise a further modification in at least one, two, three, four, five, six, seven, eight, nine, ten, eleven, twelve, thirteen, fourteen, fifteen, sixteen, seventeen, eighteen, nineteen, twenty or more endogenous genes or regulatory elements thereof. In some embodiments, the at least one modification is a single nucleobase modification. In some embodiments, the at least one modification is by base editing. In some instances, the at least one modification is an insertion or deletion carried out using a nuclease (e.g., Cas12b). The base editing may be positioned at any suitable position of the gene, or in a regulatory element of the gene. Thus, it may be appreciated that a single base editing at a start codon, for example, can completely abolish the expression of the gene. In some embodiments, the base editing may be performed at a site within an exon. In some embodiments, the base editing may be performed at a site on more than one exon. In some embodiments, the base editing may be performed at any exon of the multiple exons in a gene. In some embodiments, base editing may introduce a premature STOP codon into an exon, resulting in either lack of a translated product or in a truncated that may be misfolded and thereby eliminated by degradation, or may produce an unstable mRNA that is readily degraded. In some embodiments, the immune cell is a T_{REG} cell. In some embodiments, the immune cell is a CAR-T_{REG} cell.

In some embodiments, an edited gene may be an immune response regulation gene (e.g., coding for an immune response regulation peptide), may be an immune response regulation gene, an immunogenic gene, a checkpoint inhibitor gene, a gene involved in immune responses, a gene affecting lineage stabilization (e.g., SIRT1 and/or RNF20), a cell surface marker e.g., a T_{REG} cell surface protein (e.g., PD-1, CD70, or CD58), or any combination thereof. In some embodiments, the edited gene is associated with activated T cell proliferation, alpha-beta T cell activation, gamma-delta T cell activation, positive regulation of T cell proliferation, negative regulation of T-helper cell proliferation or differentiation, or their regulatory elements thereof, or combinations thereof.

In some embodiments the edited gene is selected from a group consisting of: BRINP1, JNK1, PRKCQ, CHIP, CD70, CD58, PD-1, SIRT1, and RNF20. In some embodiments, a combination of genes is edited that comprises one or more of SIRT1 and RNF20, one or more of PD-1, CD70, and CD58, and/or β2M (B2M). In some embodiments, one or more of the following combinations of genes is edited: SIRT1, PD-1, CD70, and CD58; SIRT1, PD-1, and CD70; SIRT1, PD-1, and CD58; SIRT1, CD70, and CD58; SIRT1 and PD-1; SIRT1 and CD70; SIRT1 and CD58; SIRT1, PD-1, CD70, CD58, and B2M; SIRT1, PD-1, CD70, and B2M; SIRT1, PD-1, CD58 and B2M; SIRT1, CD70, CD58, and B2M; SIRT1, PD-1, and B2M; SIRT1, CD70, and B2M; SIRT1, CD58, and B2M; RNF20, PD-1, CD70, and CD58; RNF20, PD-1, and CD70; RNF20, PD-1, and CD58; RNF20, CD70, and CD58; RNF20 and PD-1; RNF20 and CD70; RNF20 and CD58; RNF20, PD-1, CD70, CD58, and B2M; RNF20, PD-1, CD70, and B2M; RNF20, PD-1, CD58 and B2M; RNF20, CD70, CD58, and B2M; RNF20, PD-1, and B2M; RNF20, CD70, and B2M; RNF20, CD58, and B2M; SIRT1, RNF20, PD-1, CD70, and CD58; SIRT1, RNF20, PD-1, and CD70; SIRT1, RNF20, PD-1, and CD58; SIRT1, RNF20, CD70, and CD58; SIRT1, RNF20, and PD-1; SIRT1, RNF20, and CD70; SIRT1, RNF20, and CD58; SIRT1, RNF20, PD-1, CD70, CD58, and B2M; SIRT1, RNF20, PD-1, CD70, and B2M; SIRT1, RNF20, PD-1, CD58, and B2M; SIRT1, RNF20, CD70, CD58, and B2M; SIRT1, RNF20, PD-1, and B2M; SIRT1, RNF20, CD70, and B2M; and SIRT1, RNF20, CD58, and B2M.

In some embodiments, the editing of the endogenous gene reduces expression of the gene. In some embodiments, the editing of the endogenous gene reduces expression of the gene by at least 50% as compared to a control cell without the modification. In some embodiments, the editing of the endogenous gene reduces expression of the gene by at least 60% as compared to a control cell without the modification. In some embodiments, the editing of the endogenous gene reduces expression of the gene by at least 70% as compared to a control cell without the modification. In some embodiments, the editing of the endogenous gene reduces expression of the gene by at least 80% as compared to a control cell without the modification. In some embodiments, the editing of the endogenous gene reduces expression of the gene by at least 90% as compared to a control cell without the modification. In some embodiments, the editing of the endogenous gene reduces expression of the gene by at least 100% as compared to a control cell without the modification. In some embodiments, the editing of the endogenous gene eliminates gene expression.

In some embodiments, base editing may be performed on an intron. For example, base editing may be performed on an intron. In some embodiments, the base editing may be performed at a site within an intron. In some embodiments, the base editing may be performed at a site one or more introns. In some embodiments, the base editing may be performed at any exon of the multiple introns in a gene. In some embodiments, one or more base editing may be performed on an exon, an intron or any combination of exons and introns.

In some embodiments, the modification or base edit may be within a promoter site. In some embodiments, the base edit may be introduced within an alternative promoter site. In some embodiments, the base edit may be in a 5′ regulatory element, such as an enhancer. In some embodiment, base editing may be introduced to disrupt the binding site of a nucleic acid binding protein. Exemplary nucleic acid binding proteins may be a polymerase, nuclease, gyrase, topoisomerase, methylase or methyl transferase, transcription factors, enhancer, PABP, zinc finger proteins, among many others.

In some embodiments, base editing may be used for splice disruption to silence target protein expression. In some embodiments, base editing may generate a splice acceptor-splice donor (SA-SD) site. Targeted base editing generating a SA-SD, or at a SA-SD site can result in reduced expression of a gene. In some embodiments, base editors (e.g., ABE, CBE) are used to target dinucleotide motifs that constitute splice acceptor and splice donor sites, which are the first and last two nucleotides of each intron. In some embodiments, splice disruption is achieved with an adenosine base editor (ABE). In some embodiments, splice disruption is achieved with a cytidine base editor (CBE). In some embodiments, base editors (e.g., ABE, CBE) are used to edit exons by creating STOP codons.

In some embodiments, provided herein is an immune cell with at least one modification in one or more endogenous genes. In some embodiments, the immune cell may have at least one modification in one, two, three, four, five, six, seven, eight, nine, ten, eleven, twelve, thirteen, fourteen, fifteen, sixteen, seventeen, eighteen, nineteen, twenty or more endogenous genes. In some embodiments, the modification generates a premature stop codon in the endogenous genes. In some embodiments, the STOP codon silences target protein expression. In some embodiments, the modification is a single base modification. In some embodiments, the modification is generated by base editing. The premature stop codon may be generated in an exon, an intron, or an untranslated region. In some embodiments, base editing may be used to introduce more than one STOP codon, in one or more alternative reading frames. In some embodiments, the stop codon is generated by an adenosine base editor (ABE). In some embodiments, the stop codon is generated by a cytidine base editor (CBE). In some embodiments, the CBE generates any one of the following edits (shown in underlined font) to generate a STOP codon: CAG→TAG; CAA→TAA; CGA→TGA; TGG→TGA: TGG→TAG: or TGG→TAA.

In some embodiments, modification/base edits may be introduced at a 3′-UTR, for example, in a poly adenylation (poly-A) site. In some embodiments, base editing may be performed on a 5′-UTR region.

### Delivery Systems

### Nucleic Acid-Based Delivery of Base Editor Systems

Nucleic acid molecules encoding a base editor system according to the present disclosure can be administered to subjects or delivered into cells in vitro or in vivo by art-known methods or as described herein. For example, a base editor system comprising a deaminase (e.g., cytidine or adenine deaminase) can be delivered by vectors (e.g., viral or non-viral vectors), or by naked DNA, DNA complexes, lipid nanoparticles, or a combination of the aforementioned compositions. A base editor system may be delivered to a cell using any methods available in the art including, but not limited to, physical methods (e.g., electroporation, particle gun, calcium phosphate transfection), viral methods, non-viral methods (e.g., liposomes, cationic methods, lipid nanoparticles, polymeric nanoparticles), or biological non-viral methods (e.g., attenuated bacterial, engineered bacteriophages, mammalian virus-like particles, biological liposomes, erythrocyte ghosts, exosomes).

Nanoparticles, which can be organic or inorganic, are useful for delivering a base editor system or component thereof. Nanoparticles are well known in the art and any suitable nanoparticle can be used to deliver a base editor system or component thereof, or a nucleic acid molecule encoding such components. In one example, organic (e.g., lipid and/or polymer) nanoparticles are suitable for use as delivery vehicles in certain embodiments of this disclosure. Non-limiting examples of lipid nanoparticles suitable for use in the methods of the present disclosure include those described in International Patent Application Publications No. WO2022140239, WO2022140252, WO2022140238, WO2022159421, WO2022159472, WO2022159475, WO2022159463, WO2021113365, and WO2021141969, the disclosures of each of which is incorporated herein by reference in its entirety for all purposes.

### Viral Vectors

A base editor described herein can be delivered with a viral vector. In some embodiments, a base editor disclosed herein can be encoded on a nucleic acid that is contained in a viral vector. In some embodiments, one or more components of the base editor system can be encoded on one or more viral vectors.

Viral vectors can include lentivirus (e.g., HIV and FIV-based vectors), Adenovirus (e.g., AD100), Retrovirus (e.g., Maloney murine leukemia virus, MML-V), herpesvirus vectors (e.g., HSV-2), and Adeno-associated viruses (AAVs), or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus.

### Non-Viral Platforms for Gene Transfer

Non-viral platforms for introducing a heterologous polynucleotide into a cell of interest are known in the art.

For example, the disclosure provides a method of inserting a heterologous polynucleotide into the genome of a cell using a Cas9 or Cas12 (e.g., Cas12b) ribonucleoprotein complex (RNP)-DNA template complex where an RNP including a Cas9 or Cas12 nuclease domain and a guide RNA, wherein the guide RNA specifically hybridizes to a target region of the genome of the cell, and wherein the Cas nuclease domain cleaves the target region to create an insertion site in the genome of the cell. A DNA template is then used to introduce a heterologous polynucleotide. In embodiments, the DNA template is a double-stranded or single-stranded DNA template, wherein the size of the DNA template is about 200 nucleotides or is greater than about 200 nucleotides, wherein the 5′ and 3′ ends of the DNA template comprise nucleotide sequences that are homologous to genomic sequences flanking the insertion site. In some embodiments, the DNA template is a single-stranded circular DNA template. In embodiments, the molar ratio of RNP to DNA template in the complex is from about 3:1 to about 100:1.

In some embodiments, the DNA template is a linear DNA template. In some examples, the DNA template is a single-stranded DNA template. In certain embodiments, the single-stranded DNA template is a pure single-stranded DNA template. In some embodiments, the single stranded DNA template is a single-stranded oligodeoxynucleotide (ssODN).

In other embodiments, a single-stranded DNA (ssDNA) can produce efficient HDR with minimal off-target integration. In one embodiment, an ssDNA phage is used to efficiently and inexpensively produce long circular ssDNA (cssDNA) donors. These cssDNA donors serve as efficient HDR templates when used with Cas9 or Cas12 (e.g., Cas12a, Cas12b), with integration frequencies superior to linear ssDNA (QssDNA) donors.

### Pharmaceutical Compositions

In some aspects, the present disclosure provides a pharmaceutical composition comprising any of the cells, polynucleotides, vectors, base editors, base editor systems, guide polynucleotides, fusion proteins, complexes, or the fusion protein-guide polynucleotide complexes described herein. In embodiments, a pharmaceutical composition of the disclosure comprises a modified T_{REG} that has a lower level of, lacks, or has virtually undetectable levels of one or more of the following polypeptides: bone morphogenetic protein/retinoic acid-inducible neural-specific protein 1 (BRINP1), C terminus of HSC70-interacting protein (CHIP), Cluster of Differentiation 70, c-JUN kinase 1 (JNK1), protein kinase C theta (PRKCQ), ring finger protein 20 (RNF20), and sirtuin 1 (SIRT1).

The pharmaceutical compositions of the present disclosure can be prepared in accordance with known techniques. See, e.g., Remington, The Science And Practice of Pharmacy (21st ed. 2005). In general, the cell, or population thereof is admixed with a suitable carrier prior to administration or storage, and in some embodiments, the pharmaceutical composition further comprises a pharmaceutically acceptable carrier. Suitable pharmaceutically acceptable carriers generally comprise inert substances that aid in administering the pharmaceutical composition to a subject, aid in processing the pharmaceutical compositions into deliverable preparations, or aid in storing the pharmaceutical composition prior to administration. Pharmaceutically acceptable carriers can include agents that can stabilize, optimize or otherwise alter the form, consistency, viscosity, pH, pharmacokinetics, solubility of the formulation. Such agents include buffering agents, wetting agents, emulsifying agents, diluents, encapsulating agents, and skin penetration enhancers. For example, carriers can include, but are not limited to, saline, buffered saline, dextrose, arginine, sucrose, water, glycerol, ethanol, sorbitol, dextran, sodium carboxymethyl cellulose, and combinations thereof.

The pharmaceutical compositions of the present disclosure can be prepared in accordance with known techniques. See, e.g., Remington, The Science And Practice of Pharmacy (21st ed. 2005). In general, the cell, or population thereof is admixed with a suitable carrier prior to administration or storage, and in some embodiments, the pharmaceutical composition further comprises a pharmaceutically acceptable carrier. Suitable pharmaceutically acceptable carriers generally comprise inert substances that aid in administering the pharmaceutical composition to a subject, aid in processing the pharmaceutical compositions into deliverable preparations, or aid in storing the pharmaceutical composition prior to administration. Pharmaceutically acceptable carriers can include agents that can stabilize, optimize or otherwise alter the form, consistency, viscosity, pH, pharmacokinetics, solubility of the formulation. Such agents include buffering agents, wetting agents, emulsifying agents, diluents, encapsulating agents, and skin penetration enhancers. For example, carriers can include, but are not limited to, saline, buffered saline, dextrose, arginine, sucrose, water, glycerol, ethanol, sorbitol, dextran, sodium carboxymethyl cellulose, and combinations thereof.

In some embodiments, the pharmaceutical composition is formulated for delivery to a subject. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site (e.g., a site of a graft versus host disease). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In some embodiments, any of the fusion proteins, gRNAs, and/or complexes described herein are provided as part of a pharmaceutical composition. In some embodiments, the pharmaceutical composition comprises any of the fusion proteins or complexes provided herein. In some embodiments pharmaceutical composition comprises a gRNA, a nucleic acid programmable DNA binding protein, a cationic lipid, and a pharmaceutically acceptable excipient. In embodiments, pharmaceutical compositions comprise a lipid nanoparticle and a pharmaceutically acceptable excipient. In embodiments, the lipid nanoparticle contains a gRNA, a base editor, a complex, a base editor system, or a component thereof of the present disclosure, and/or one or more polynucleotides encoding the same. Pharmaceutical compositions can optionally comprise one or more additional therapeutically active substances.

The compositions, as described above, can be administered in effective amounts. The effective amount will depend upon the mode of administration, the particular condition being treated, and the desired outcome. It may also depend upon the stage of the condition, the age and physical condition of the subject, the nature of concurrent therapy, if any, and like factors well-known to the medical practitioner. For therapeutic applications, it is that amount sufficient to achieve a medically desirable result.

In some embodiments, compositions in accordance with the present disclosure can be used for treatment of any of a variety of diseases, disorders, and/or conditions.

### Methods of Treatment

Some aspects of the present disclosure provide methods of treating a subject in need thereof (e.g., a subject having or having a propensity to develop an undesirable immune response, an autoimmune or alloimmune disease, and/or GVHD) the method comprising administering to a subject in need an effective therapeutic amount of a pharmaceutical composition as described herein. More specifically, the methods of treatment include administering to a subject in need thereof one or more pharmaceutical compositions comprising one or more cells having at least one edited gene. In other embodiments, the methods of the disclosure comprise expressing or introducing into a cell a base editor polypeptide and one or more guide RNAs capable of targeting a nucleic acid molecule encoding at least one polypeptide.

One of ordinary skill in the art would recognize that multiple administrations of the pharmaceutical compositions contemplated in particular embodiments may be required to affect the desired therapy. For example, a composition may be administered to the subject 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more.

Administration of the pharmaceutical compositions contemplated herein may be carried out using conventional techniques including, but not limited to, infusion, transfusion, or parenterally. In some embodiments, parenteral administration includes infusing or injecting intravascularly, intravenously, intramuscularly, intraarterially, intrathecally, intratumorally, intradermally, intraperitoneally, transtracheally, subcutaneously, subcuticularly, intraarticularly, subcapsularly, subarachnoidly and intrasternally.

In embodiments, administration of CAR T_{REG} cells of the present disclosure is associated with reduction in one or more symptoms of an autoimmune and/or alloimmune disease or disorder in a subject. For example, in some cases, administration of the CAR T_{REG} cells is associated with a reduction in a symptom of graft versus host disease.

### Kits

The disclosure provides kits for the treatment of a subject having or having a propensity to develop an undesirable immune response, an autoimmune or alloimmune disease, and/or graft versus host disease. In some embodiments, the kit further includes a base editor system or a polynucleotide encoding a base editor system, wherein the base editor polypeptide system a nucleic acid programmable DNA binding protein (napDNAbp), a deaminase, and a guide RNA. In some embodiments, the napDNAbp is Cas9 or Cas12. In some embodiments, the polynucleotide encoding the base editor is a mRNA sequence. In some embodiments, the deaminase is a cytidine deaminase or an adenosine deaminase. In some embodiments, the kit comprises an edited cell and instructions regarding the use of such cell.

The kits may further comprise written instructions for using a base editor, base editor system and/or edited cell as described herein. In other embodiments, the instructions include at least one of the following: precautions; warnings; clinical studies; and/or references. The instructions may be printed directly on the container (when present), or as a label applied to the container, or as a separate sheet, pamphlet, card, or folder supplied in or with the container. In a further embodiment, a kit comprises instructions in the form of a label or separate insert (package insert) for suitable operational parameters. In yet another embodiment, the kit comprises one or more containers with appropriate positive and negative controls or control samples, to be used as standard(s) for detection, calibration, or normalization. The kit can further comprise a second container comprising a pharmaceutically acceptable buffer, such as (sterile) phosphate-buffered saline, Ringer's solution, or dextrose solution. It can further include other materials desirable from a commercial and user standpoint, including other buffers, diluents, filters, needles, syringes, and package inserts with instructions for use.

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the disclosure, and, as such, may be considered in making and practicing embodiments of the disclosure. Particularly useful techniques for particular embodiments will be discussed in the sections that follow.

The following examples are put forth so as to provide those of ordinary skill in the art with a complete disclosure and description of how to make and use the assay, screening, and therapeutic methods of the disclosure, and are not intended to limit the scope of what the inventors regard as their invention.

### EXAMPLES

### Example 1: Base Editing of T_{REG} Cells to Disrupt Expression of Cluster of Differentiation 70 (CD70)

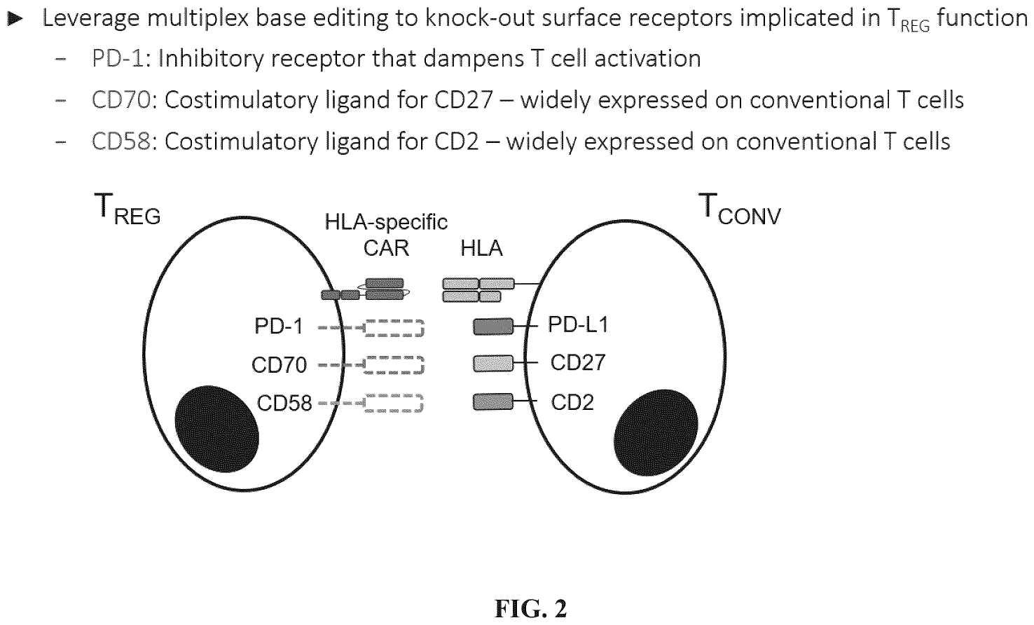

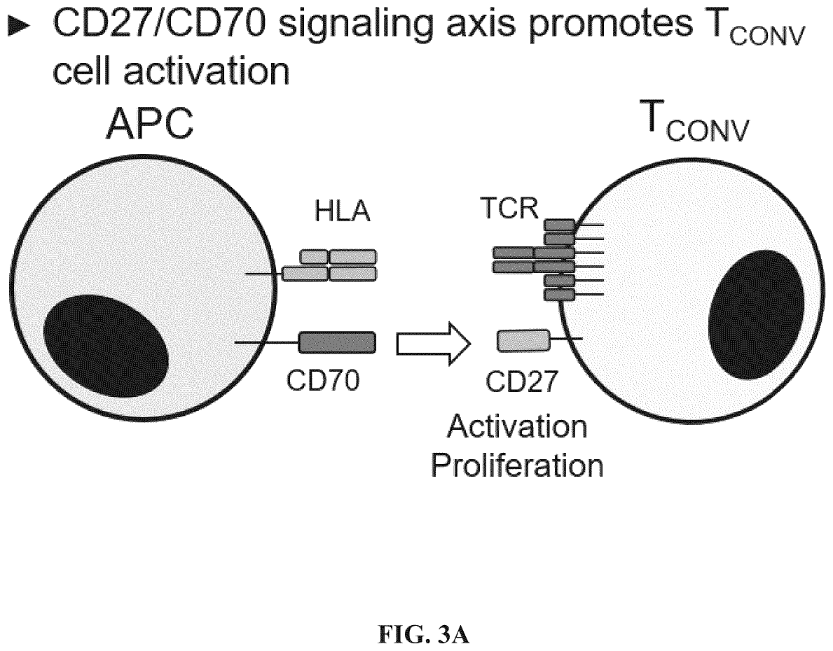

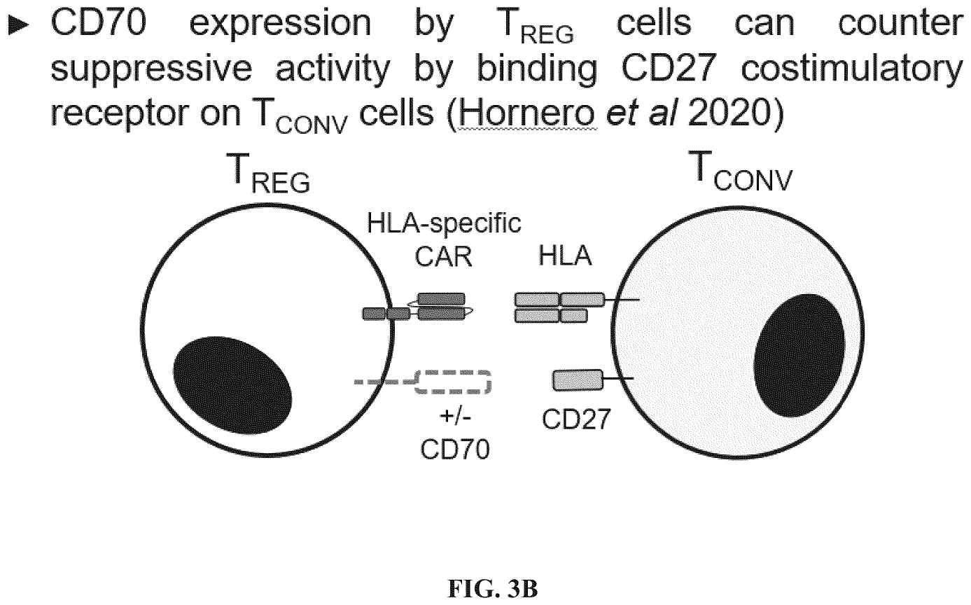

Experiments were undertaken to prepare chimeric antigen receptor (CAR) regulatory T (T_{REG}) cells with improved functionality and lineage stability by using base editing to disrupt surface receptors. As shown in FIG. 2, reducing or eliminating expression of cluster of differentiation 70 (CD70) in T_{REG} cells can improve the function of the T_{REG} cells by preventing stimulation of T_{CONV} cells through the interaction of CD70 with cluster of differentiation 27 (CD27) on the T_{CONV} cells (see FIGS. 3A and 3B).

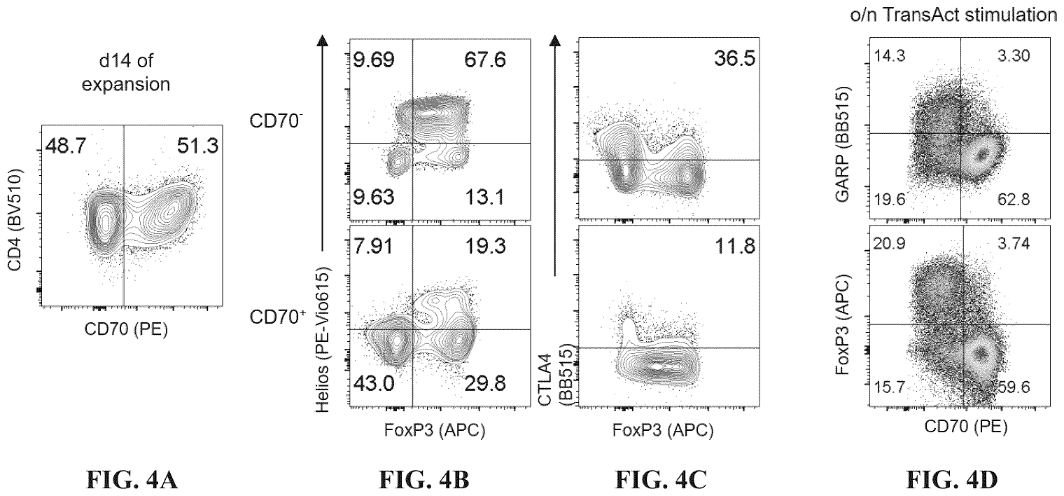

As shown in the flow cytometry scatter plots of FIGS. 4A-4D, CD70− T_{REG} cells showed increased expression of the T_{REG} activation markers FOXP3, GARP, Helios, and CTLA-4. The T_{REG} edited cells showed an increase in Helios and FoxP3 expression relative to unedited T_{REG} cells. The CD70− T_{REG} cells showed an increase in CTLA4 levels.

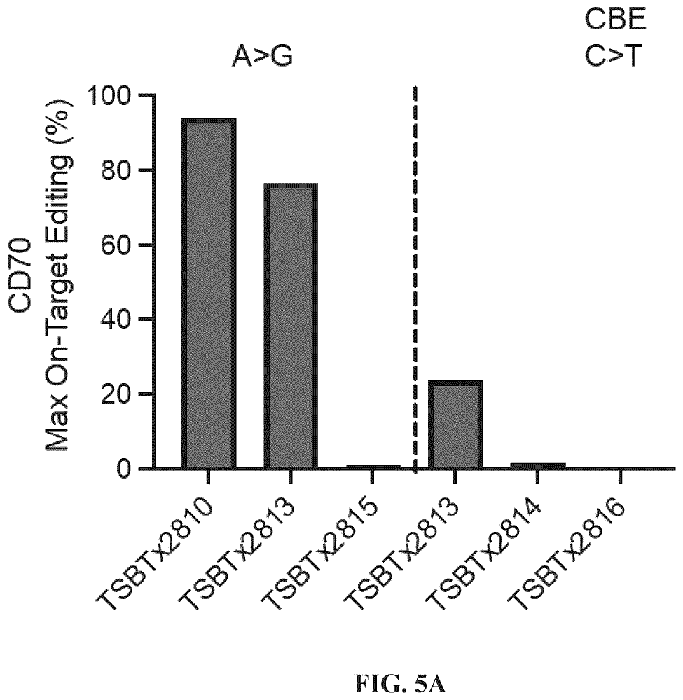

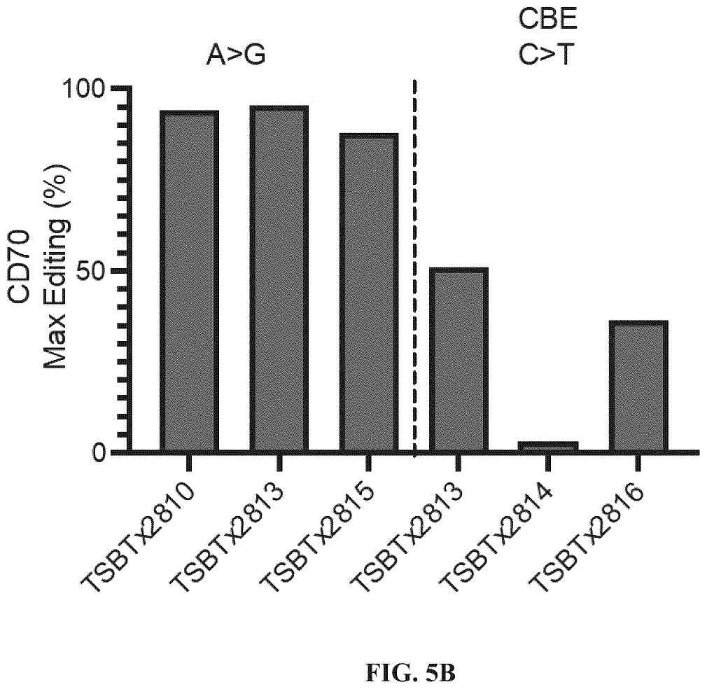

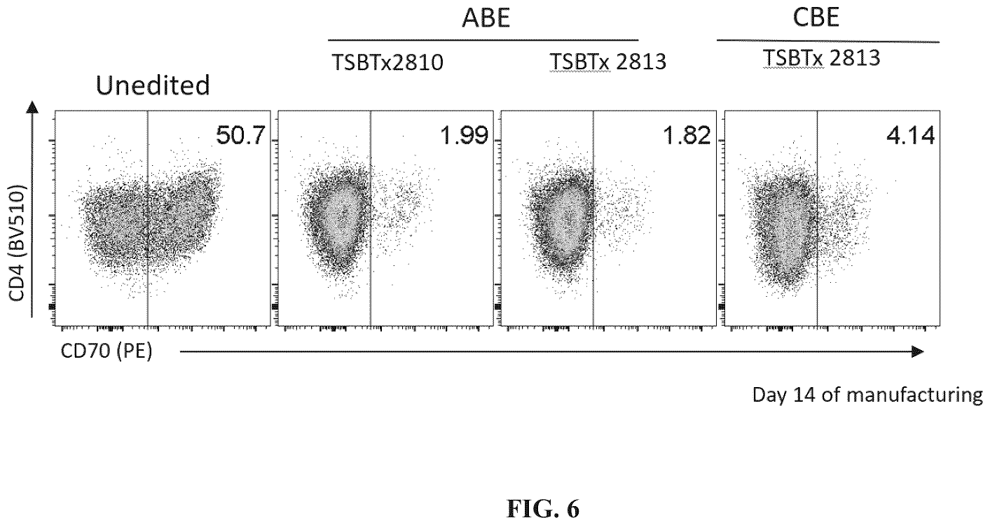

Guide RNA's were designed to facilitate base editing of the CD70 gene in T_{REG} cells (see Tables 1A-2C). Guide RNAs TSBTx2810, TSBTx2813, and TSBTx2815 (see Tables 1A-2C) were transfected into cells along with mRNA encoding an ABE and guide RNAs TSBTx2813, TSBTx2814, and TSBTx2816 (see Tables 1A-2C) were transfected into cells along with mRNA encoding a CBE. The resulting maximum on-target (i.e., “max on-target”) and total (i.e., “max on-target” plus “bystander”) editing activities were measured using next generation sequencing (see FIGS. 5A and 5B). The guide RNAs successfully facilitated editing of the CD70 gene in the T_{REG} cells, which resulted in a reduction in detected surface-expression of CD70 in the cells, as shown in FIG. 6.

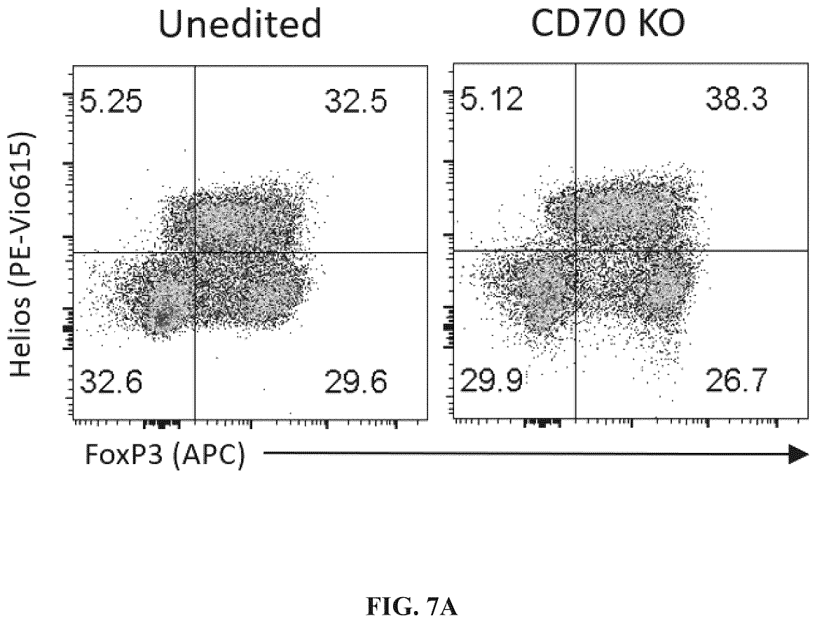

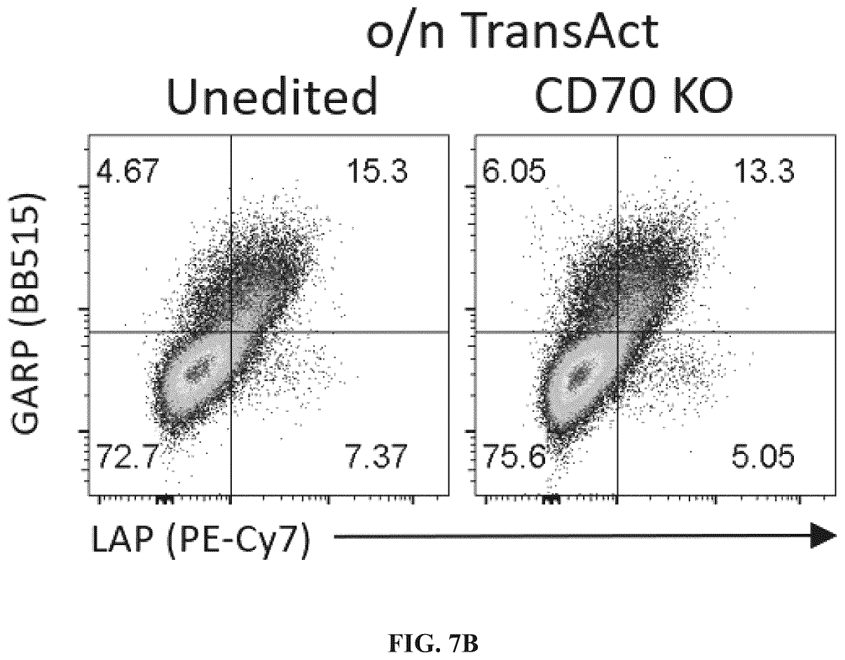

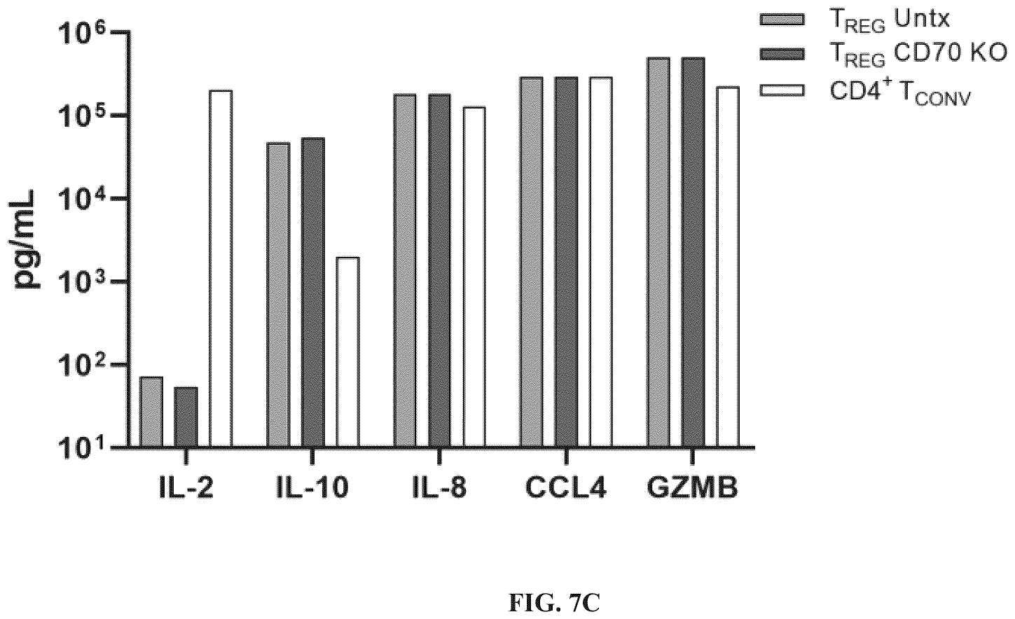

The CD70− base-edited T_{REG} cells were phenotypically indistinguishable from unedited control T_{REG} cells (see FIGS. 7A-7C), as determined through measurement of FoxP3, Helios, GARP, LAP, IL-2, IL-10, IL-8, CCL4, and GZMB polypeptide expression.

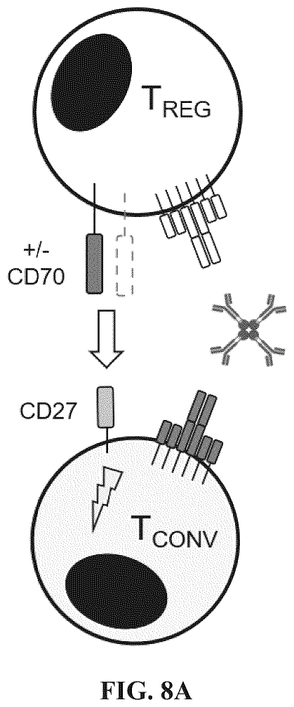

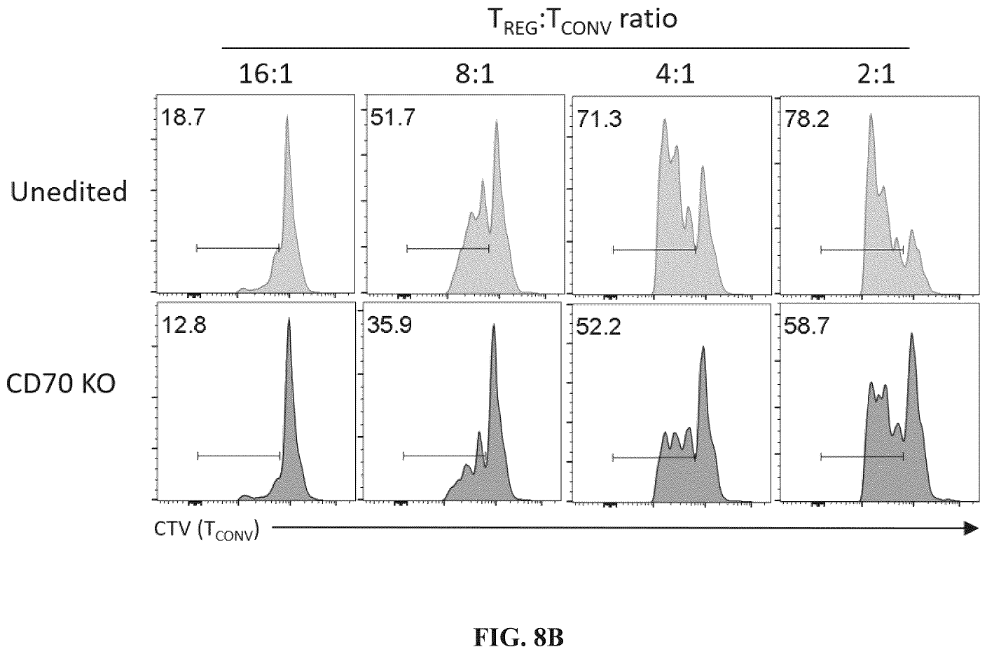

As shown in FIGS. 8A and 8B, T_{CONV} cells showed reduced costimulation when cocultured with the CD70− T_{REG} cells. This was demonstrated by co-culturing CD70+ T_{REG} cells and CD70− T_{REG} cells with T_{CONV} cells and measuring proliferation of the T_{CONV} cells using CellTrace™ Violet dye. Therefore, disruption of CD70 expression in the T_{REG} cells improved T_{REG} cell function by reducing activation of T_{CONV} cells by the T_{REG} cells.

### Example 2: Base Editing of T_{REG} Cells to Disrupt Expression of Cluster of Differentiation 58 (CD58), PD-1, SIRT1, and B2M

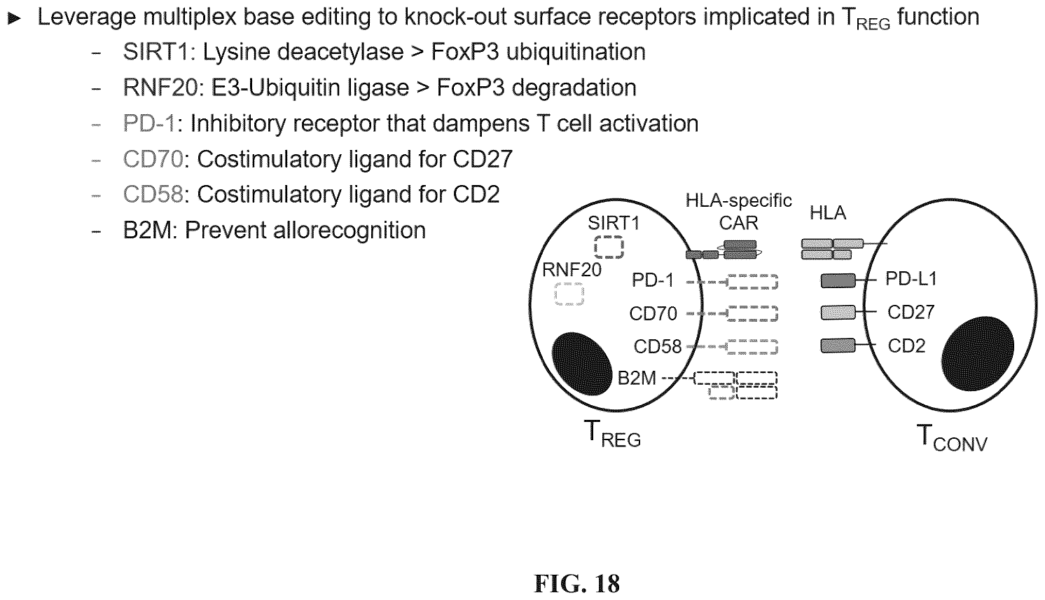

Experiments were undertaken to design guide RNAs suitable for use in base-editing T_{REG} cells to disrupt expression of Cluster of Differentiation 58 (CD58), PD-1, SIRT1, and B2M. As shown in FIG. 2, knockout of PD-1 can prevent dampening of T_{REG} cell activation through the interaction of PD-1 with PD-L1 expressed on T_{CONV} cells, and knockout of CD58 can prevent activation of T_{CONV} cells through the interaction of CD58 with CD2 expressed on the T_{CONV} cells. As shown in FIG. 18, SIRT1 is a lysine deacetylase that is associated with FoxP3 ubiquitination. Therefore, knocking out SIRT1 can be used to increase expression levels of the important T_{REG} transcription factor FoxP3, thereby improving T_{REG} cell function. Knockout of B2M can be used to prevent allorecognition of edited T_{REG} cells (see FIG. 18).

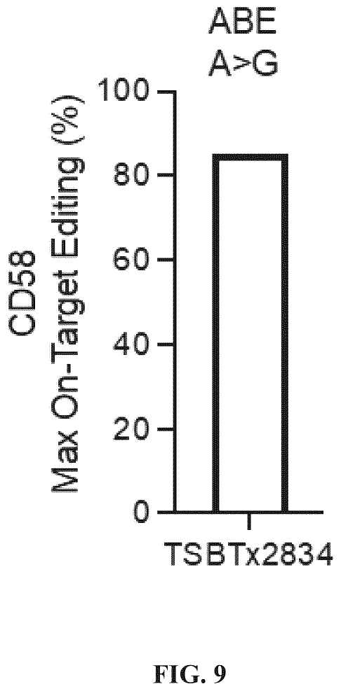

T_{REG} cells were transfected with the guide RNA TSBTx2834 (see Tables 1A-2C) targeting CD58 and mRNA encoding an adenosine base editor (ABE8.20). The maximum on-target base editing of the CD58 gene is shown in FIG. 9. The maximum on-target base editing was greater than 80%.

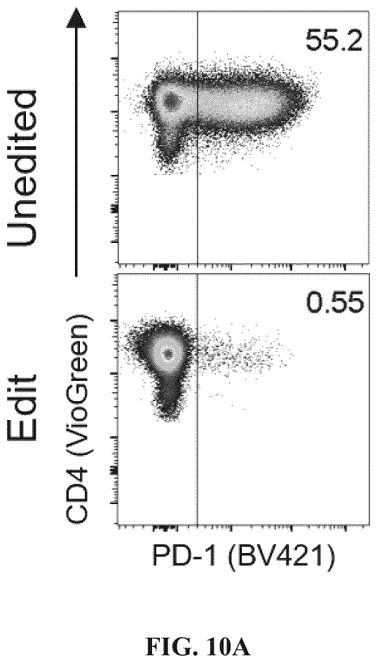

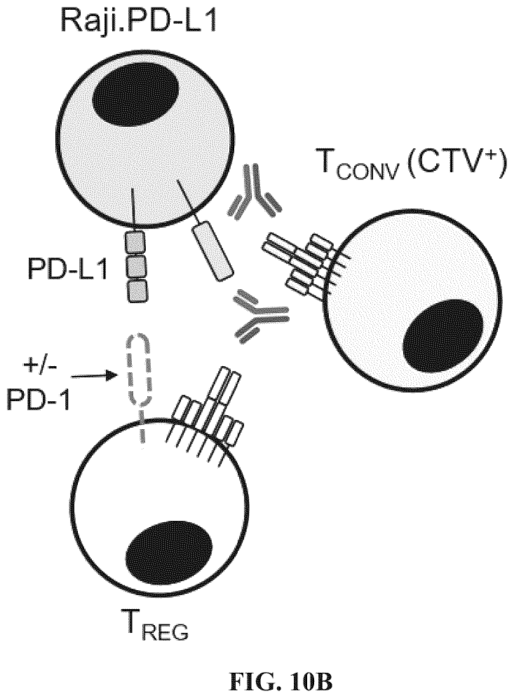

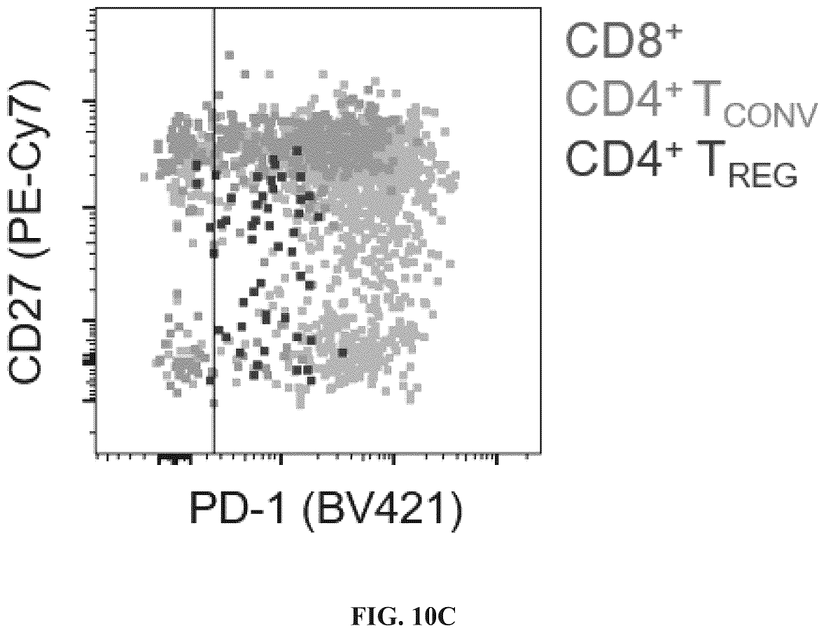

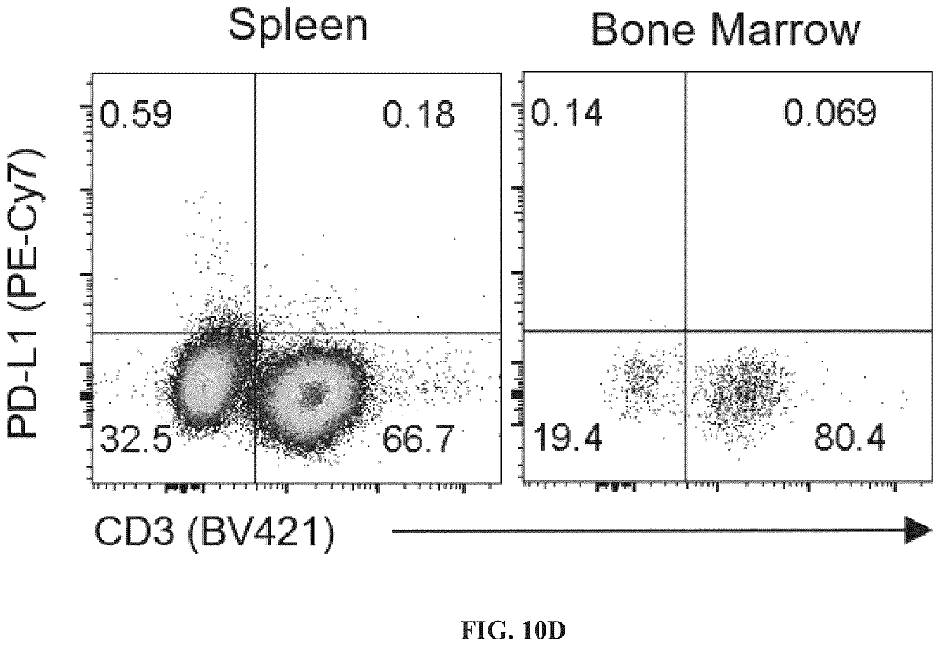

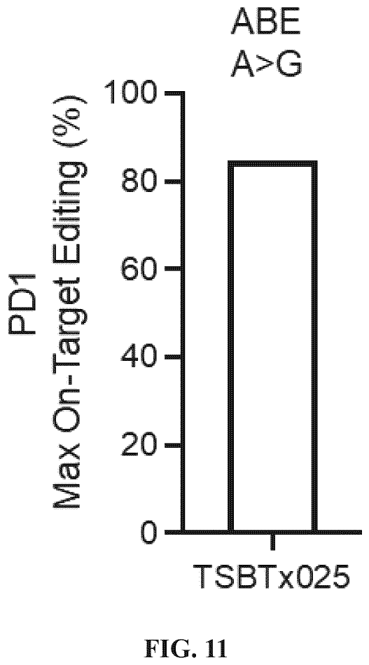

T_{REG} cells were transfected with the guide RNA TSBTx025 (see Tables 1A-2C) targeting PD-1 and mRNA encoding an adenosine base editor (ABE8.20). The maximum on-target base editing of the PD-1 gene is shown in FIG. 11. The maximum on-target base editing was greater than 80%. Reduction of surface expression of PD-1 in the PD-1-edited T_{REG} cells was confirmed using flow cytometry (see FIG. 10A). FIG. 10B illustrates how PD-1 expressed by T_{REG} cells can interact with PD-L1 expressed on the surface of a T cell, which results in inhibition of the T_{REG} cell. FIG. 10C shows that T_{CONV} and T_{REG} cells surface expressed CD27 and PD-1 and FIG. 10D shows that PD-L1 was surface-expressed in T cells. Disruption of PD-1 expression in T_{REG} cells can “take the brakes” off Treg cells and can increase the likelihood of Treg activation in the presence of PD-L1 ligand. PD-1 is an inhibitory receptor that dampens Treg activation.

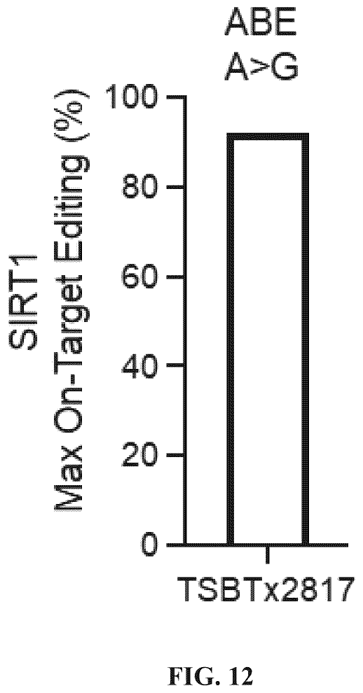

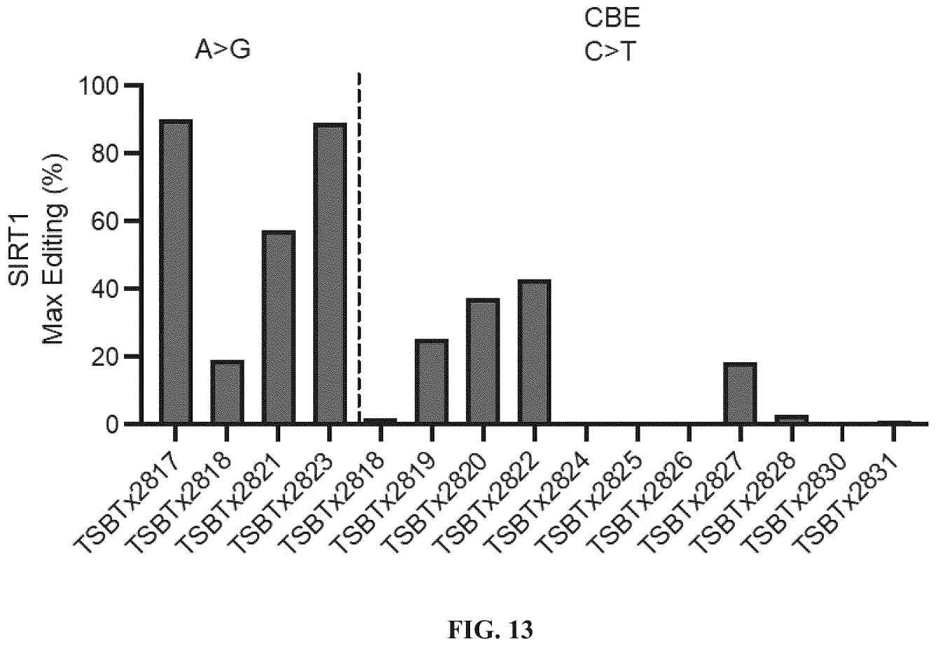

SIRT1 encodes a nicotinamide adenine dinucleotide (NAD+) dependent histone deacetylase. SIRT1 targets histone and non-histone proteins and can function as a transcription factor. Disruption of Sirt1 expression promotes the expression of FoxP3, a key transcription factor in T_{REG} cells. Therefore, T_{REG} cells were transfected with the guide RNA TSBTx2817 (see Tables 1A-2C) targeting SIRT1 and mRNA encoding an adenosine base editor (ABE8.20). The maximum on-target base editing of the SIRT1 gene is shown in FIG. 12. The maximum on-target base editing was greater than 80%. Also, T_{CONV} cells were transfected with guide RNAs TSBTx2817, TSBTx2818, TSBTx2819, TSBTx2820, TSBTx2821, TSBTx2822, TSBTx2823, TSBTx2824, TSBTx2825, TSBTx2826, TSBTx2827, TSBTx2828, TSBTx2830, or TSBTx2831 in combination with mRNA encoding an adenosine deaminase (ABE8.20) or a cytidine deaminase (CBE). The amino acid sequence of the CBE used is provided at SEQ ID NO: 3357 and contained two UGI domains, an SpCas9n domain, and a cytidine deaminase domain. The maximum base editing of the SIRT1 gene in the T_{CONV} cells is shown in FIG. 13. As shown by comparison of FIGS. 12 and 13, base editing efficiencies in T_{CONV} cells were predictive of base editing efficiencies in T_{REG} cells (compare editing for the guide RNA TSBTx2817 in FIGS. 12 and 13).

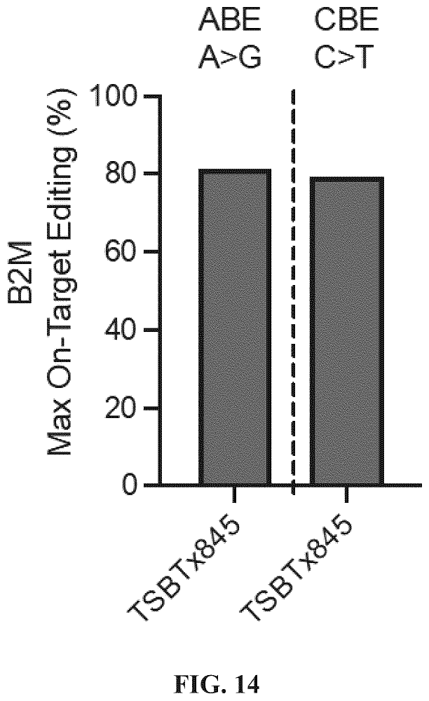

T_{REG} cells were transfected with the guide RNA TSBTx845 (see Tables 1A-2C) targeting CD58 and mRNA encoding an adenosine base editor (ABE8.20) or a cytidine base editor (CBE). The maximum on-target base editing of the CD58 gene is shown in FIG. 14. The maximum on-target base editing was about 80%.

### Example 3: Base Editing of T_{REG} Cells to Disrupt Expression of RNF20 RNF20 is an E3 Ubiquitin Ligase that Functions as a Modulator of FoxP3 Expression

Accordingly, knocking out SIRT1 can be used to increase expression levels of the important T_{REG} transcription factor FoxP3, thereby improving T_{REG} cell function. RNF20 knockout rescues impairment of FoxP3 transcription exhibited by USP-22-null T_{REG} cells, where USP-22 is involved in the deubiquitination module of the SAGA chromatin remodeling complex. RNF20 is a negative regulator of FoxP3. Therefore, experiments were undertaken to design guide RNAs suitable for use in base-editing T_{REG} cells to disrupt expression of RNF20 to modulate Fox-3 expression. Guides RNAs for targeting RNF20 were screened in T cells to identify guides for use in T_{REG} cells.

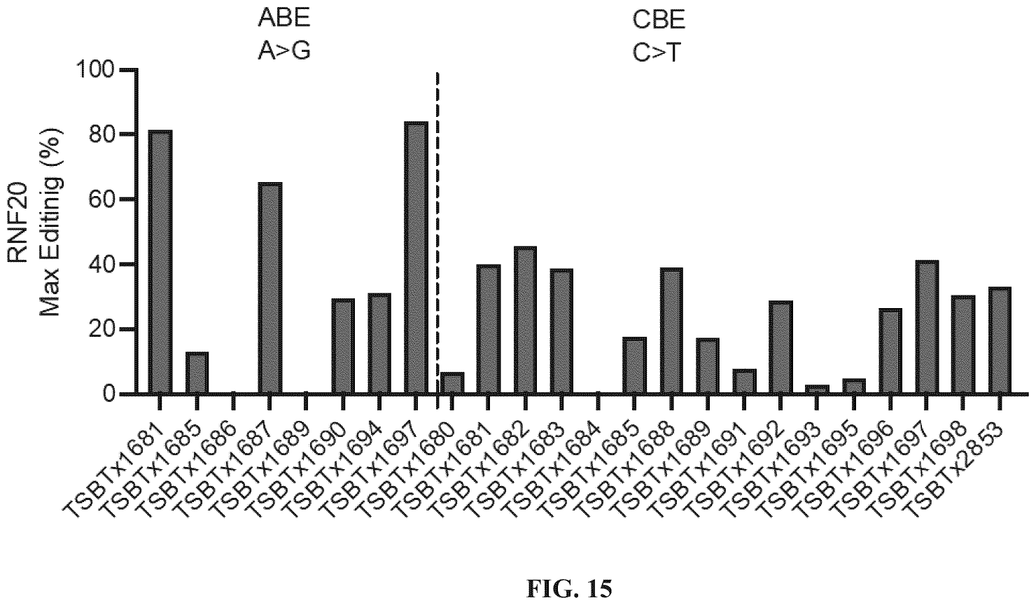

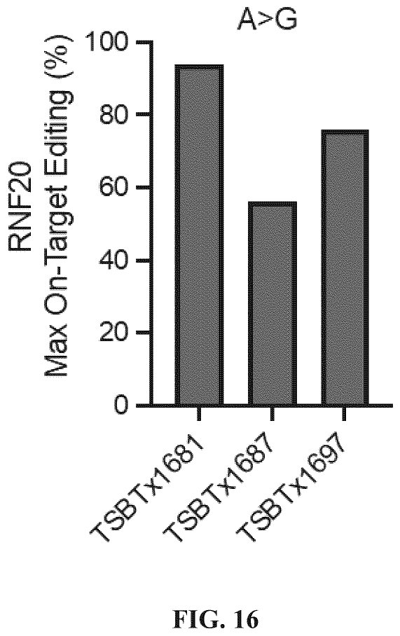

T_{CONV} cells were transfected with the guide RNAs TSBTx1680, TSBTx1681, TSBTx1682, TSBTx1683, TSBTx1684, TSBTx1685, TSBTx1686, TSBTx1687, TSBTx1688, TSBTx1689, TSBTx1690, TSBTx1691, TSBTx1692, TSBTx1693, TSBTx1694, TSBTx1695, TSBTx1696, TSBTx1697, TSBTx1698, or TSBTx2853 (see Tables 1A-2C) targeting RNF20 and mRNA encoding an adenosine base editor (ABE) or a cytidine base editor (CBE). The maximum on-target base editing of the RNF20 gene is shown in FIG. 15. T_{REG} cells were then transfected with the guide RNAs TSBTx1681, TSBTx1687, or TSBTx1697 (see Tables 1A-2C) targeting RNF20 and mRNA encoding an adenosine base editor (ABE). The maximum on-target base editing is shown in FIG. 16. As shown by comparison of FIGS. 15 and 16, base editing efficiencies in T_{CONV} cells were predictive of base editing efficiencies in T_{REG} cells (compare max editing for the guides TSBTx1681, TSBTx1687, and TSBTx1697 in FIGS. 12 and 13).

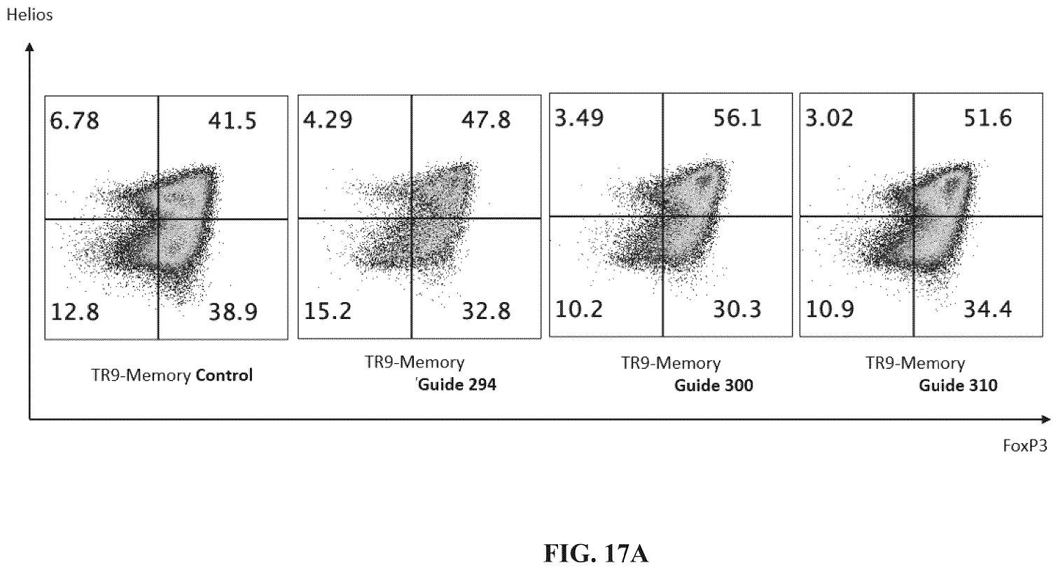

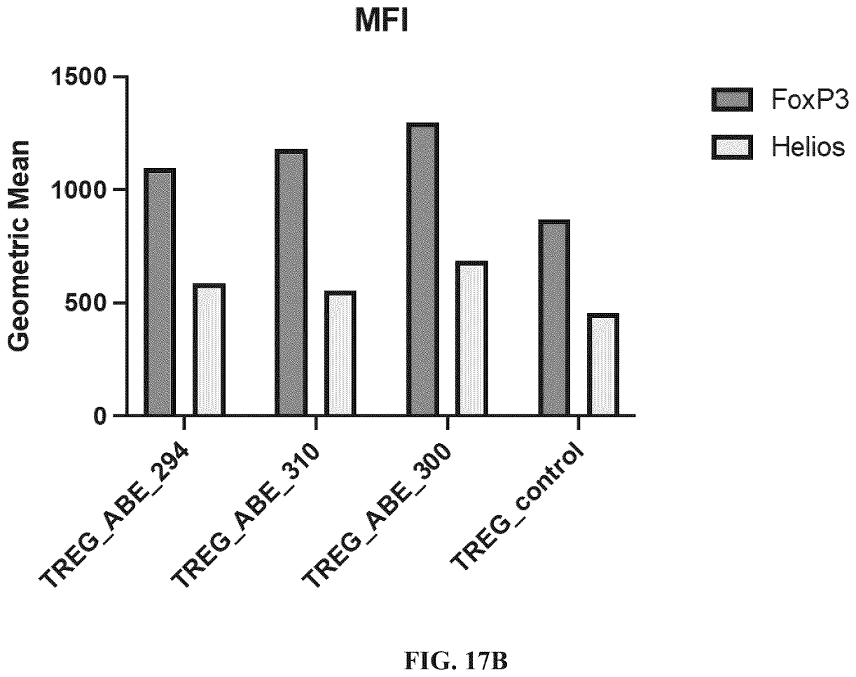

The RNF20-edited T_{REG} cells showed a shift in FoxP3 mean fluorescent intensity (MFI), as measured using flow cytometry, that was consistent with increased expression of FoxP3 relative to unedited T_{REG} cells (see FIGS. 17A and 17B). Increase in FoxP3 expression is indicative of T_{REG} lineage stabilization.

### Example 4: Multiplex Base Editing of Regulatory T (T_{REG}) Cells

Chimeric antigen receptor (CAR) T_{REG} cell function can be improved by disrupting multiple genes encoding surface receptors in the cells (see FIG. 18). For example, disruption of SIRT1 or RNF20 can lead to an increase in the important T_{REG} transcriptional regulator FoxP3, disruption of PD-1, CD70, and/or CD58 can reduce activation of T_{CONV} cells by edited CAR T_{REG} cells, and disruption of B2M in CAR T_{REG} cells can prevent allorecognition of the T_{REG} cells in a subject. Therefore, experiments were undertaken to 5-plex edit CD70, SIRT1, CD58, PD-1, and B2M in T_{REG} cells.

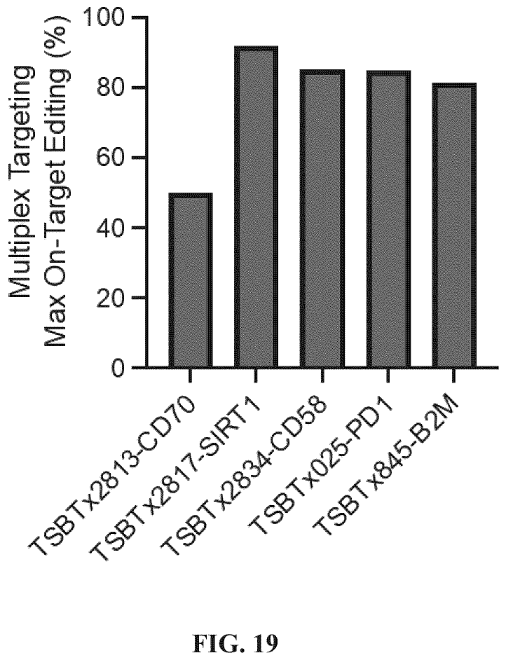

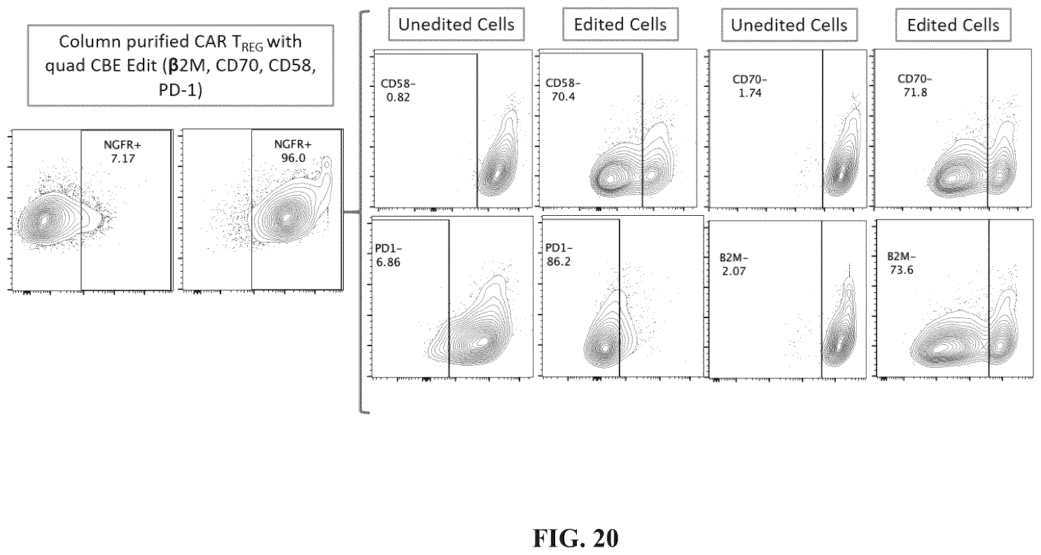

T_{CONV} cells were transfected in parallel with the guide RNAs TSBTx2813, TSBTx2817, TSBTx2834, TSBTx025, and TSBTx845 (see Tables 1A-2C) targeting CD70, SIRT1, CD58, PD-1, and B2M, respectively, and mRNA encoding an adenosine base editor (ABE). The resulting on-target editing efficiencies are shown in FIG. 19. The multiplex-edited cells were transfected with a polynucleotide encoding an anti-NGFR chimeric antigen receptor (CAR) to prepare multi-plex edited CAR T_{REG} cells. Reduced surface expression of the target genes in the multi-plex edited CAR T_{REG} cells was confirmed using flow cytometry (see FIG. 20).

### Example 5: Increasing Lineage Stability of T_{REG} Cells Through Base Editing of BRINP1, JNK1, PRKCQ, and CHIP

Expression of FoxP3 in a regulatory T (T_{REG}) cell can be increased and/or stabilized, and thus the lineage stability of the T_{REG} cell increased, by modifying the cell to reduce or eliminate expression of one or more of BRINP1, JNK1, PRKCQ, and CHIP. Therefore, experiments are undertaken to modify BRINP1, JNK1, PRKCQ, and CHIP genes in T_{REG} cells to reduce or eliminate expression of the BRINP1, JNK1, PRKCQ, and CHIP polypeptides encoded thereby.

Guide RNAs are designed targeting genes encoding BRINP1, JNK1, PRKCQ, and CHIP (see Tables 1A-2C). T_{REG} cells are transfected with guides listed in Tables 1A-2C targeting each of the genes and mRNA encoding an adenosine base editor (ABE) and/or a cytidine base editor (CBE). The edited cells show reduced or eliminated expression of BRINP1, JNK1, PRKCQ, and/or CHIP and increased expression levels of FoxP3 and/or increased lineage stability.

### Example 6: Increasing Lineage Stability of T_{REG} Cells Through Modification of CHIP and RNF20 Genes Using an Endonuclease

Experiments are undertaken to disruption expression and/or activity of genes encoding CHIP and RNF20 in T_{REG} Cells using an endonuclease.

Guide RNAs are designed targeting genes encoding CHIP and RNF20 (see Tables 1A-2C). T_{REG} cells are transfected with guides listed in Tables 1A-2C targeting each of the genes and mRNA encoding a Cas12b polypeptide. The edited cells show reduced or eliminated expression of RNF20 and/or CHIP and increased expression levels of FoxP3 and/or increased lineage stability.

The following methods were employed in the above examples.

### Preparation of Peripheral Blood Mononuclear Cells (PBMCs) and Regulatory T Cells (T_{REG} 's)

As shown in FIG. 1, the preparation of PBMCs and T_{REG}'S involved seven (7) steps: 1) Receive fresh leukopak (HemaCare); 2) Process leukopak by ficoll density gradient centrifugation to isolate PBMCs; 3) Cryopreserve PBMCs for convenient use; 4) Thaw cryopreserved PBMCs; 5) Isolate CD4+ T cells by magnetic selection (i.e., CD4 Microbeads, Miltenyi); 6) Stain CD4+ T cells with fluorochrome conjugated antibodies and FACS sort; and 7) Sort T_{REG} cells: Viable/CD4+/CD25++/CD127. T_{REG} cells were sorted out based upon high CD4 expression, high CD25 expression, and low CD127 expression. The gating strategy used to select for viable T_{REG} cells is presented in the lower portion of FIG. 1 as a series of representative flow cytometry scatter plots.

### Ex Vivo Expansion and Editing of T_{REG}'s

Editing of T_{REG}'S post-sorting included the following five (5) steps: 1) T_{REG}'S were counted and the cell medium was replaced; 2) cells were stimulated with ImmunoCult™ CD3/CD28/CD2 T cell Activator 1:40 (25 μL/mL and 1e6 cells/mL) in XF media with serum replacement; 3) 5 days post-stimulation the cells were edited using a standard T cell editing protocol (500k-1e6 cells per 200 μL reaction); 4) check for editing via Flow and/or next-generation sequencing (NGS) at least 72 hours post-editing; 5) phenotype cells on day 7 and day 14 to verify T_{REG} phenotype over time.

mRNA Production

Editors/nucleases were cloned into a plasmid encoding a dT7 promoter followed by a 5′UTR, Kozak sequence, ORF, and 3′UTR. The dT7 promoter carries an inactivating point mutation within the T7 promoter that prevents transcription from circular plasmid. This plasmid templated a PCR reaction (Q5 Hot Start 2× Master Mix), in which the forward primer corrected the SNP within the T7 promoter and the reverse primer appended a 120A tail to the 3′ UTR. The resulting PCR product was purified on a Zymo Research 25 μg DCC column and used as mRNA template in the subsequent in vitro transcription. The NEB HiScribe High-Yield Kit was used as per the instruction manual but with full substitution of N1-methyl-pseudouridine for uridine and co-transcriptional capping with CleanCap AG (Trilink). Reaction cleanup was performed by lithium chloride precipitation.

In Vitro Transcription of sgRNAs.

Linear DNA fragments containing the CMV promoter followed by the sgRNA target sequence were transcribed in vitro using the TranscriptAid T7 High Yield Transcription Kit (ThermoFisher Scientific) according to the manufacturer's instructions. sgRNA products were purified using the MEGAclear Kit (ThermoFisher Scientific) according to the manufacturer's instructions and quantified by UV absorbance.

### Next-Generation DNA Sequencing

Samples were sequenced on an Illumina MiSeq as previously described (Pattanayak, Nature Biotechnol. 31, 839-843 (2013)).

### Data Analysis

Sequencing reads were automatically demultiplexed using MiSeq Reporter (Illumina), and individual FASTQ files were analyzed with a custom Matlab. Each read was pairwise aligned to the appropriate reference sequence using the Smith-Waterman algorithm. Base calls with a Q-score below 31 were replaced with Ns and were thus excluded in calculating nucleotide frequencies. This treatment yields an expected MiSeq base-calling error rate of approximately 1 in 1,000. Aligned sequences in which the read and reference sequence contained no gaps were stored in an alignment table from which base frequencies could be tabulated for each locus. Indel frequencies were quantified with a custom Matlab script using previously described criteria (Zuris, et al., Nature Biotechnol. 33, 73-80 (2015). Sequencing reads were scanned for exact matches to two 10-bp sequences that flank both sides of a window in which indels might occur. If no exact matches were located, the read was excluded from analysis. If the length of this indel window exactly matched the reference sequence the read was classified as not containing an indel. If the indel window was two or more bases longer or shorter than the reference sequence, then the sequencing read was classified as an insertion or deletion, respectively.

### OTHER EMBODIMENTS

From the foregoing description, it will be apparent that variations and modifications may be made to the aspects and embodiments thereof described herein to adopt them to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference. The application may be related to U.S. Provisional Applications No. 63/233,648, filed 16 Aug. 2021, 63/293,692, filed 24 Dec. 2021, 63/293,722, filed 24 Dec. 2021, or 63/336,109, filed 28 Apr. 2022, the disclosures of which are incorporated herein by reference in their entireties for all purposes.

What is claimed:

1. A method for producing a functionally enhanced and/or lineage stabilized regulatory T (T_{REG}) cell, the method comprising contacting a T_{REG} cell with a base editor, or one or more polynucleotides encoding the base editor, wherein the base editor comprises a polynucleotide programmable DNA binding polypeptide (napDNAbp), and a deaminase, and one or more guide RNAs (gRNAs), or one or more polynucleotides encoding the gRNAs, wherein the one or more gRNAs target the base editor to effect an alteration in a nucleic acid molecule, wherein the nucleic acid molecule encodes a polypeptide and/or comprises a regulatory element associated with expression thereof, and wherein the polypeptide is selected from the group consisting of bone morphogenetic protein/retinoic acid-inducible neural-specific protein 1 (BRINP1), C terminus of HSC70-interacting protein (CHIP), Cluster of Differentiation 70 (CD70), c-JUN kinase 1 (JNK1), protein kinase C theta (PRKCQ), ring finger protein 20 (RNF20), and sirtuin 1 (SIRT1), thereby producing the functionally enhanced and/or lineage stabilized T_{REG} cell.

2. The method of claim 1, further comprising contacting the T_{REG} cell with a gRNA that targets the base editor to effect an alteration in a nucleic acid molecule, wherein the nucleic acid molecule encodes a polypeptide and/or comprises a regulatory element associated with expression thereof, and wherein the polypeptide is selected from the group consisting of beta-2 microglobulin (B2M), Cluster of Differentiation 58, and programmed cell death 1 (PD-1).

3. A method for producing a functionally enhanced and/or lineage stabilized regulatory T (T_{REG}) cell, the method comprising contacting a T_{REG} cell with a base editor, or one or more polynucleotide encoding the base editor, wherein the base editor comprises a polynucleotide programmable DNA binding polypeptide (napDNAbp), and a deaminase, and two or more guide RNAs (gRNAs), or one or more polynucleotides encoding the gRNAs, wherein each gRNA targets the base editor to effect an alteration in a nucleic acid molecule, wherein each nucleic acid molecule encodes a polypeptide and/or comprises a regulatory element associated with expression of the polypeptide, and wherein a first polypeptide is selected from the group consisting of bone morphogenetic protein/retinoic acid-inducible neural-specific protein 1 (BRINP1), C terminus of HSC70-interacting protein (CHIP), c-JUN kinase 1 (JNK1), protein kinase C theta (PRKCQ), ring finger protein 20 (RNF20), and sirtuin 1 (SIRT1), and wherein a second polypeptide is selected from the group consisting of Cluster of Differentiation 58 (CD58), Cluster of Differentiation 70 (CD70), and programmed cell death 1 (PD-1).

4. The method of claim 3 further comprising effecting an alteration in a third nucleic acid molecule, wherein the nucleic acid molecule encodes a beta-2 microglobulin (B2M) polypeptide and/or comprises a regulatory element associated with expression thereof.

5. The method of claim 4, wherein the method comprises contacting the T_{REG} cell with gRNAs comprising each of the following nucleotide sequences: TSBTx2813, TSBTx2817, TSBTx2834, TSBTx025, and TSBTx845 to thereby reduce or eliminate expression of each of CD70, SIRT1, CD58, PD-1, and B2M in the T_{REG} cell.

6. The method of claim 1, wherein the method comprises editing a combination of nucleic acid molecules encoding a combination of two or more polypeptides and/or a regulatory element associated with expression thereof, wherein the combination of polypeptides is selected from the group consisting of: SIRT1, PD-1, CD70, and CD58; SIRT1, PD-1, and CD70; SIRT1, PD-1, and CD58; SIRT1, CD70, and CD58; SIRT1 and PD-1; SIRT1 and CD70; SIRT1 and CD58; SIRT1, PD-1, CD70, CD58, and B2M; SIRT1, PD-1, CD70, and B2M; SIRT1, PD-1, CD58 and B2M; SIRT1, CD70, CD58, and B2M; SIRT1, PD-1, and B2M; SIRT1, CD70, and B2M; SIRT1, CD58, and B2M; RNF20, PD-1, CD70, and CD58; RNF20, PD-1, and CD70; RNF20, PD-1, and CD58; RNF20, CD70, and CD58; RNF20 and PD-1; RNF20 and CD70; RNF20 and CD58; RNF20, PD-1, CD70, CD58, and B2M; RNF20, PD-1, CD70, and B2M; RNF20, PD-1, CD58 and B2M; RNF20, CD70, CD58, and B2M; RNF20, PD-1, and B2M; RNF20, CD70, and B2M; RNF20, CD58, and B2M; SIRT1, RNF20, PD-1, CD70, and CD58; SIRT1, RNF20, PD-1, and CD70; SIRT1, RNF20, PD-1, and CD58; SIRT1, RNF20, CD70, and CD58; SIRT1, RNF20, and PD-1; SIRT1, RNF20, and CD70; SIRT1, RNF20, and CD58; SIRT1, RNF20, PD-1, CD70, CD58, and B2M; SIRT1, RNF20, PD-1, CD70, and B2M; SIRT1, RNF20, PD-1, CD58, and B2M; SIRT1, RNF20, CD70, CD58, and B2M; SIRT1, RNF20, PD-1, and B2M; SIRT1, RNF20, CD70, and B2M; and SIRT1, RNF20, CD58, and B2M.

7. The method of claim 1, wherein each guide RNA comprises a sequence selected from the group consisting of TSBTx2810, TSBTx2813, TSBTx2815, TSBTx2813, TSBTx2814, TSBTx2816, TSBTx2834, TSBTx845, TSBTx025, TSBTx2817, TSBTx2817, TSBTx2818, TSBTx2819, TSBTx2820, TSBTx2821, TSBTx2822, TSBTx2823, TSBTx2824, TSBTx2825, TSBTx2826, TSBTx2827, TSBTx2828, TSBTx2830, or TSBTx2831, TSBTx1680, TSBTx1681, TSBTx1682, TSBTx1683, TSBTx1684, TSBTx1685, TSBTx1686, TSBTx1687, TSBTx1688, TSBTx1689, TSBTx1690, TSBTx1691, TSBTx1692, TSBTx1693, TSBTx1694, TSBTx1695, TSBTx1696, TSBTx1697, TSBTx1698, or TSBTx2853, TSBTx2813, TSBTx2817, TSBTx2834, TSBTx025, and TSBTx845.

8. The method of claim 1, wherein:
the alteration is associated with a reduction of costimulation of a T_{CONV} cell by the functionally enhanced and/or lineage stabilized T_{REG} cell relative to a reference cell;
the deaminase is a cytidine deaminase or an adenosine deaminase;
the napDNAbp is Cas9 or Cas12;
the base editor further comprises one or more uracil glycosylase inhibitors (UGIs); and/or
the base editor further comprises one or more nuclear localization signals (NLS).

9. The method of claim 8, wherein the adenosine deaminase is ABE8.20.

10. The method of claim 1, further comprising expressing a chimeric antigen receptor (CAR) in the T_{REG} cell.

11. A method for producing a functionally enhanced and/or lineage stabilized regulatory T (T_{REG}) cell, the method comprising contacting a T_{REG} cell with a polynucleotide programmable DNA binding polypeptide (napDNAbp), or one or more polynucleotides encoding the napDNAbp, and one or more guide RNAs (gRNAs), or one or more polynucleotides encoding the gRNAs, that target the napDNAbp to cleave a target nucleic acid molecule and effect an alteration in the target nucleic acid molecule, wherein the target nucleic acid molecule encodes a polypeptide and/or comprises a regulatory element associated with expression thereof, and wherein the polypeptide is selected from the group consisting of bone morphogenetic protein/retinoic acid-inducible neural-specific protein 1 (BRINP1), C terminus of HSC70-interacting protein (CHIP), Cluster of Differentiation 70 (CD70), c-JUN kinase 1 (JNK1), protein kinase C theta (PRKCQ), ring finger protein 20 (RNF20), and sirtuin 1 (SIRT1), thereby producing the functionally enhanced and/or lineage stabilized T_{REG} cell.

12. A functionally enhanced and/or lineage stabilized regulatory T (T_{REG}) cell produced according to the method of claim 1.

13. A functionally enhanced and/or lineage stabilized regulatory T (T_{REG}) cell comprising a nucleobase alteration that reduces or eliminates expression of a polypeptide selected from the group consisting of bone morphogenetic protein/retinoic acid-inducible neural-specific protein 1 (BRINP1), C terminus of HSC70-interacting protein (CHIP), Cluster of Differentiation 70 (CD70), c-JUN kinase 1 (JNK1), protein kinase C theta (PRKCQ), ring finger protein 20 (RNF20), and sirtuin 1 (SIRT1).

14. The functionally enhanced and/or lineage stabilized regulatory T (T_{REG}) cell of claim 1, where the T_{REG} cell comprises a nucleobase alteration that reduces or eliminates expression of two or more polypeptides, wherein a first polypeptide is selected from the group consisting of bone morphogenetic protein/retinoic acid-inducible neural-specific protein 1 (BRINP1), C terminus of HSC70-interacting protein (CHIP), c-JUN kinase 1 (JNK1), protein kinase C theta (PRKCQ), ring finger protein 20 (RNF20), and sirtuin 1 (SIRT1), and wherein a second polypeptide is selected from the group consisting of Cluster of Differentiation 58 (CD58), Cluster of Differentiation 70 (CD70), and programmed cell death 1 (PD-1).

15. The functionally enhanced and/or lineage stabilized regulatory T (T_{REG}) cell of any claim 14, wherein the T_{REG} cell comprises an alteration that reduces or eliminates expression of a combination of polypeptides selected from the group consisting of: SIRT1, PD-1, CD70, and CD58; SIRT1, PD-1, and CD70; SIRT1, PD-1, and CD58; SIRT1, CD70, and CD58; SIRT1 and PD-1; SIRT1 and CD70; SIRT1 and CD58; SIRT1, PD-1, CD70, CD58, and B2M; SIRT1, PD-1, CD70, and B2M; SIRT1, PD-1, CD58 and B2M; SIRT1, CD70, CD58, and B2M; SIRT1, PD-1, and B2M; SIRT1, CD70, and B2M; SIRT1, CD58, and B2M; RNF20, PD-1, CD70, and CD58; RNF20, PD-1, and CD70; RNF20, PD-1, and CD58; RNF20, CD70, and CD58; RNF20 and PD-1; RNF20 and CD70; RNF20 and CD58; RNF20, PD-1, CD70, CD58, and B2M; RNF20, PD-1, CD70, and B2M; RNF20, PD-1, CD58 and B2M; RNF20, CD70, CD58, and B2M; RNF20, PD-1, and B2M; RNF20, CD70, and B2M; RNF20, CD58, and B2M; SIRT1, RNF20, PD-1, CD70, and CD58; SIRT1, RNF20, PD-1, and CD70; SIRT1, RNF20, PD-1, and CD58; SIRT1, RNF20, CD70, and CD58; SIRT1, RNF20, and PD-1; SIRT1, RNF20, and CD70; SIRT1, RNF20, and CD58; SIRT1, RNF20, PD-1, CD70, CD58, and B2M; SIRT1, RNF20, PD-1, CD70, and B2M; SIRT1, RNF20, PD-1, CD58, and B2M; SIRT1, RNF20, CD70, CD58, and B2M; SIRT1, RNF20, PD-1, and B2M; SIRT1, RNF20, CD70, and B2M; and SIRT1, RNF20, CD58, and B2M.

16. A pharmaceutical composition comprising a functionally enhanced and/or lineage stabilized regulatory T (T_{REG}) cell of claim 14.

17. The pharmaceutical composition of claim 17, wherein the T_{REG} cell overexpresses an inhibitory receptor selected from the group consisting of Human Leukocyte Antigen-E (HLA-E), Human Leukocyte Antigen-G (HLA-G), Programmed Death Ligand 1 (PD-L1), and Cluster of Differentiation 47 (CD47).

18. A pharmaceutical composition comprising one or more guide RNAs (gRNA) and a polynucleotide encoding a base editor comprising a polynucleotide programmable DNA binding polypeptide (napDNAbp) domain and a deaminase domain, wherein each gRNA comprises a nucleic acid sequence that is complementary to a nucleic acid molecule, wherein each nucleic acid molecule encodes a polypeptide and/or comprises a regulatory element associated with expression of the polypeptide, wherein a first polypeptide is selected from the group consisting of bone morphogenetic protein/retinoic acid-inducible neural-specific protein 1 (BRINP1), C terminus of HSC70-interacting protein (CHIP), c-JUN kinase 1 (JNK1), protein kinase C theta (PRKCQ), ring finger protein 20 (RNF20), and sirtuin 1 (SIRT1), and wherein a second polypeptide is selected from the group consisting of Cluster of Differentiation 58 (CD58), Cluster of Differentiation 70 (CD70), and programmed cell death 1 (PD-1).

19. A kit comprising the functionally enhanced and/or stabilized T_{REG} cell of claim 13.

20. A method of treating an autoimmune or alloimmune disease in a subject, the method comprising administering to the subject an effective amount of a functionally enhanced and/or stabilized T_{REG} cell of claim 13.

21. A guide RNA (gRNA) or a polynucleotide encoding the guide RNA, wherein the guide RNA comprises a nucleotide sequence with at least 70% sequence identity to a sequence selected from one or more of those listed in Tables 1A-1C or 2A-2C, or truncations thereof, wherein the gRNA targets a base editor comprising a nucleic acid programmable DNA binding protein (napDNAbp) to effect an alteration in a nucleic acid molecule encoding a polypeptide and/or comprising a regulatory element associated with expression thereof, and wherein the polypeptide is selected from the group consisting of bone morphogenetic protein/retinoic acid-inducible neural-specific protein 1 (BRINP1), C terminus of HSC70-interacting protein (CHIP), Cluster of Differentiation 70 (CD70), c-JUN kinase 1 (JNK1), protein kinase C theta (PRKCQ), ring finger protein 20 (RNF20), and sirtuin 1 (SIRT1), thereby producing a functionally enhanced and/or lineage stabilized T_{REG} cell.

22. A method for producing a functionally enhanced and/or lineage stabilized regulatory T (T_{REG}) cell, the method comprising contacting a T_{REG} cell with a base editor, or a polynucleotide encoding the base editor, wherein the base editor comprises a polynucleotide programmable DNA binding polypeptide (napDNAbp), and an adenosine deaminase, and one or more of the following guide RNAs, or one or more polynucleotides encoding the one or more guide RNAs:
a) a guide RNA comprising a nucleotide sequence selected from the group consisting of TSBTx2810, TSBTx2813, and TSBTx2815;
b) a guide RNA comprising a nucleotide sequence selected from the group consisting of TSBTx2834 and TSBTx845;
c) a guide RNA comprising the nucleotide sequence TSBTx025;
d) a guide RNA comprising the nucleotide sequence TSBTx845; and
e) a guide RNA comprising a nucleotide sequence selected from the group consisting of TSBTx2817, TSBTx2818, TSBTx2819, TSBTx2820, TSBTx2821, TSBTx2822, TSBTx2823, TSBTx2824, TSBTx2825, TSBTx2826, TSBTx2827, TSBTx2828, TSBTx2830, and TSBTx2831;
thereby reducing or eliminating expression in the T_{REG} of one or more of the following polypeptides: cluster of differentiation 70 (CD70), cluster of differentiation 58 (CD58), programmed cell death 1 (PD-1), beta-2 microglobulin (B2M), and sirtuin 1 (SIRT1).

23. A cell prepared according to the method of claim 23.

24. A pharmaceutical composition comprising the cell of claim 23.
